(19) United States
(12) Patent Application Publication
(10) Pub. No.: US 2005/0272061 A1
(43) Pub. Date: Dec. 8, 2005

(54) Expression profiling in non-small cell lung cancer

(75) Inventors: Joseph M. Petroziello, Greenwich, CT (US); Paul Carter, Mercer Island, WA (US)

Correspondence Address: KLAUBER & JACKSON, 411 HACKENSACK AVENUE, HACKENSACK, NJ, 07601, US

(73) Assignee: Seattle Genetics, Inc., Bothell, WA (US)

(21) Appl. No.: 11/063,343

(22) Filed: Feb. 22, 2005

Related U.S. Application Data
(60) Provisional application No. 60/546,019, filed on Feb. 19, 2004.

(51) Int. Cl. C12Q 1/68; G01N 33/574

(52) U.S. Cl. 435/6; 435/7.23

(57) ABSTRACT

The present invention relates to L genes and gene products that are differentially expressed in cancer tissues and cell lines. In a particular aspect of the invention, L genes and gene products are differentially expressed in lung cancer tissues and cell lines. In accordance with the present invention, L nucleic acid sequences, amino acid sequences and antibodies thereto, and methods of use thereof are presented. The L molecules and methods of the invention may be used to monitor expression levels of L genes, wherein the detection of aberrant levels of L molecules provides a positive diagnostic indicator of lung cancer and/or other L gene associated cancers and a useful prognostic indice of the state of such diseases. Also provided are compounds capable of modulating an L molecule mediated activity, which are identified using the L molecules and methods of the invention. Such L molecule modulating compounds may be used efficaciously to treat patients with lung cancer, or other L antigen positive cancers.

### BACKGROUND OF THE INVENTION

The invention relates generally to the field of cancer diagnosis, prognosis, treatment and prevention. More particularly, the present invention relates to methods of diagnosing, treating and preventing cancer. In particular, aspects of the invention are directed to methods of diagnosing, treating and preventing cancers of the lung, breast, brain, colon, kidney, ovary, pancreas, prostate, rectum, stomach, and uterus. Methods of using a nucleic acid and/or a protein, which are differentially expressed in tumor cells, and antibodies immunospecific for the protein, to treat, diagnose and/or prevent cancer, are provided for by the present invention. The instant invention provides compositions comprising novel L gene products, designated L1-L19, and antibodies thereto, and methods of using novel L gene products and associated splice variants thereof. Such L gene products include L proteins and nucleic acids and variants thereof. Such gene products, as well as their binding partners and antagonists or agonists, can be used for the prevention, diagnosis, prognosis and/or treatment of cancer. The invention also relates to the identification of a genetic expression signature and subsets thereof, which are positive indicators of the presence of a cancer. As such, the detection of a genetic expression signature of the present invention enables rapid diagnosis of a cancer, such as lung cancer, which is associated with the genetic expression signature.

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastases). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins & Angell, 1976, Basic Pathology, 2d Ed., W.B. Saunders Co., Philadelphia, pp. 68-79) The neoplastic lesion may evolve clonally and develop an increasing capacity for growth, metastasis, and heterogeneity, especially under conditions in which the neoplastic cells escape the host's immune surveillance (Roitt, I., Brostoff, J. and Kale, D., 1993, Immunology, 3rd ed., Mosby, St. Louis, pps. 17.1-17.12).

The epidemiology of cancer in the United States is estimated at greater than 1,300,000 new cases and greater than 550,000 deaths (Jemal et al., 2003, CA Cancer J. Clin., 53, 5-26) estimated for 2003. Lung cancer is one of the most common cancers with an estimated 172,000 new cases projected for 2003 and 157,000 deaths (Jemal et al., 2003, CA Cancer J. Clin., 53, 5-26). Lung carcinomas are typically classified as either small-cell lung carcinomas (SCLC) or non-small cell lung carcinomas (NSCLC). SCLC comprises about 20% of all lung cancers with NSCLC comprising the remaining approximately 80%. NSCLC is further divided into adenocarcinoma (AC) (about 30-35% of all cases), squamous cell carcinoma (SCC) (about 30% of all cases) and large cell carcinoma (LCC) (about 10% of all cases). Additional NSCLC subtypes, not as clearly defined in the literature, include adenosquamous cell carcinoma (ASCC), and bronchioalveolar carcinoma (BAC).

Lung cancer is the leading cause of cancer deaths worldwide, and more specifically non-small cell lung cancer accounts for approximately 80% of all disease cases (Cancer Facts and Figures, 2002, American Cancer Society, Atlanta, p. 11.). There are four major types of non-small cell lung cancer, including adenocarcinoma, squamous cell carcinoma, bronchioalveolar carcinoma, and large cell carcinoma. Adenocarcinoma and squamous cell carcinoma are the most common types of NSCLC based on cellular morphology (Travis et al., 1996, Lung Cancer Principles and Practice, Lippincott-Raven, New York, pps. 361-395). Adenocarcinomas are characterized by a more peripheral location in the lung and often have a mutation in the K-ras oncogene (Gazdar et al., 1994, Anticancer Res. 14:261-267). Squamous cell carcinomas are typically more centrally located and frequently carry p53 gene mutations (Niklinska et al., 2001, Folia Histochem. Cytobiol. 39:147-148). A systematic evaluation of gene expression profiling data for each of the corresponding NSCLC subtypes using a combination of suppressive subtractive hybridization (SSH) and DNA arrays may be useful for the identification of additional novel targets of utility in disease detection and as therapeutic targets for lung cancer treatment modalities.

Several genes have been previously described as potential diagnostic markers or prognostic indicators for lung cancer, including: CYFRA 21-1, TPA, and CA125 (Hatzakis et al., 2002, Respiration. 69(1):25-29); CEA (Sawabata et al., 2002, Ann Thorac Surg. 74(1):174-179); p53 and HER2-neu (Han et al., 2002, Hum Pathol. 33(1):105-110); NSE (Kulpa et al., 2002, Clin Chem. 48(11):1931-1937); and IL-8 (Yuan et al., 2000, Am J Respir Crit Care Med. 162:1957-1963).

A marker-based approach to tumor identification and characterization promises improved diagnostic and prognostic reliability. Typically, the diagnosis of lung cancer and other types of cancer requires histopathological proof of the presence of the tumor. In addition to diagnosis, histopathological examinations also provide information about prognosis and selection of treatment regimens. Prognosis may also be established based upon clinical parameters such as tumor size, tumor grade, the age of the patient, and lymph node metastasis.

In clinical practice, accurate diagnosis of various subtypes of cancer is important because treatment options, prognosis, and the likelihood of therapeutic response all vary broadly depending on the diagnosis. Accurate prognosis, or determination of distant metastasis-free survival could allow an oncologist to tailor the administration of adjuvant chemotherapy, with patients having poorer prognoses being given the most aggressive treatment. Furthermore, accurate prediction of poor prognosis would greatly impact clinical trials for new lung cancer therapies, because potential study patients could then be stratified according to prognosis. Trials could then be limited to patients having poor prognosis, in turn making it easier to discern if an experimental therapy is efficacious. To date, no set of satisfactory predictors for prognosis based on the clinical information alone has been identified.

It would, therefore, be beneficial to provide specific methods and reagents for the diagnosis, staging, prognosis, monitoring and treatment of cancer, including lung cancer. It would also be beneficial to provide methods that identify individuals with a predisposition for the onset of lung cancer, and other types of cancer, and hence are appropriate subjects for preventive therapy.

### SUMMARY OF THE INVENTION

Intensive and systematic evaluation of gene expression patterns is essential for understanding the physiological mechanisms associated with cellular transformation and metastasis associated with cancer. Several techniques that permit comparison of gene expression in normal and cancerous cells are known in the art. Examples of these techniques include: Serial Analysis of Gene Expression (SAGE) (Velculescu et al., 1995, Science 270:484-487); Restriction Enzyme Analysis of Differentially Expressed Sequences (READS) (Prasher et al., 1999, Methods in Enzymology 303:258); Amplified Fragment Length Polymorphism (AFLP) (Bachem et al., 1996, Plant Journal 9:745); Representational Difference Analysis (RDA) (Hubank et al., 1994, Nucleic Acid Research 22:(25):5640); differential display (Liang et al., 1992, Cancer Research 52(24):6966); and suppression subtractive hybridization (SSH) (Diatchenko et al., 1996, Proc. Natl. Acad. Sci. USA 93:6025-6030). Such differential expression methods have led the present inventors to the identification and characterization of the L genes and variants thereof, as genes whose expression is associated with lung cancer and other types of cancer. This discovery by the present inventors has made possible the use of L molecules and variants thereof for the treatment, prevention and diagnosis of cancers, including but not limited to lung cancer.

Novel genes designated L1-L19 which display an upregulated expression pattern in cancer tissues and cell lines, e.g., lung cancer tissues and cell lines are shown and described. Also shown and described are L1-19 variants and fragments that retain at least one functional characteristic of a full length, wild type L gene. Methods of using the gene, gene products, and antagonists or agonists of the gene or gene products (L1-L19 and variants thereof, cDNA, RNA, and/or protein) as targets for diagnosis, drug screening and therapies for cancer are also shown and described. Also disclosed is the use of the genes or gene products or derivatives thereof as vaccines against cancer. In one embodiment, methods are provided for using an L protein (i.e., SEQ ID NOs: 20-38), and variants thereof, or nucleic acids that encode said proteins for the treatment, prevention and diagnosis of lung cancer.

In particular, the methods of the present invention include using nucleic acid molecules that encode one of the L proteins and variants thereof, and recombinant DNA molecules, cloned genes or degenerate variants thereof, and in particular naturally occurring variants that encode L related gene products. The methods of the present invention additionally include using cloning vectors, including expression vectors, containing the nucleic acid molecules encoding an L protein and variants thereof, and hosts that contain such nucleic acid molecules. The methods of the present invention also encompass the use of L gene products and variants thereof, including fusion proteins, and antibodies directed against such L gene products or conserved variants or fragments thereof. In one embodiment, a fragment or other derivative of an L protein is at least 10 amino acids long. In another embodiment, a fragment of an L nucleic acid or a variant or derivative thereof is at least 10 nucleotides long.

Nucleotide sequences of human L gene cDNA are provided. Specifically, cDNA sequences of human L genes L1-L19 (SEQ ID NOs: 1-19, respectively; See FIGS. 1A-S) are provided herein. Also provided are amino acid sequences encoded by SEQ ID NOs: 1-19, which are denoted SEQ ID NOs: 20-38 (See FIGS. 2A-S). Also provided are isolated nucleic acids that encode polypeptides comprising one of SEQ ID NOs: 20-38. Full-length L genes were cloned utlizing polymerase chain reaction (PCR) amplification. L gene transcripts are detected at elevated levels in both lung cancer cell lines and lung tumor isolates compared to normal tissues. Elevated transcript levels for L genes L1-L19 were also detected in additional tumor types and cancer cells as described herein below.

The present invention further relates to methods for the diagnostic evaluation and prognosis of cancer in a subject animal. Preferably the subject is a mammal, more preferably the subject is a human. In a preferred embodiment the invention relates to methods for diagnostic evaluation and prognosis of lung cancer. For example, nucleic acid molecules of the invention can be used as diagnostic hybridization probes or as primers for diagnostic PCR analysis for detection of abnormal expression of an L gene.

Antibodies or other binding partners to L genes and variants thereof can be used in a diagnostic test to detect the presence of an L gene product in body fluids, cells or in tissue biopsy. In specific embodiments, measurement of serum or cellular L gene products and variants thereof can be made to detect or stage lung cancer, e.g., adenocarcinoma, squamous cell carcinoma, bronchioalveolar carcinoma, or large cell carcinoma.

The present invention also relates to methods for the identification of subjects having a predisposition to cancer, e.g., lung cancer. The subject can be any animal, but preferably the subject is a mammal, and most preferably the subject is a human. In a non-limiting example nucleic acid molecules of the invention can be used as diagnostic hybridization probes or as primers for quantitative reverse transcriptase-PCR (RT-PCR) analysis to determine expression levels of the L gene products and variants thereof. In another example, nucleic acid molecules of the invention can be used as diagnostic hybridization probes or as primers for diagnostic PCR analysis for the identification of one of the L genes and variants thereof, naturally occurring or non-naturally occurring gene mutations, allelic variations and regulatory defects in one of the L genes (i.e., L1-L19). Imaging methods, for visualizing the localization and/or amounts of L gene products in a patient, are also provided for diagnostic and prognostic use.

Further, methods are presented for the treatment of cancer, including lung cancer. Such methods comprise the administration of compositions that are capable of modulating the level of an L gene and variants thereof, including modulation of L gene expression and/or the level of an L gene product activity in a subject. The subject can be any animal, preferably a mammal, more preferably a human.

Still further, the present invention relates to methods for the use of an L gene and variants thereof for the identification of compounds that modulate L gene expression and/or the activity of an L gene product. Such compounds may be used as agents to prevent and/or treat lung cancer or any L positive cancer wherein an L gene and/or variants thereof are expressed at levels that are elevated with respect to the expression level in corresponding normal tissue. Such compounds can also be used to palliate the symptoms of the disease, and control the metastatic potential of lung cancer or any cancer wherein an L gene and variants thereof are expressed at elevated levels relative to those of normal tissue.

The invention also provides methods for preventing cancer wherein a product of an L gene or variants thereof are administered to a subject in an amount effective to elicit an immune response in the subject. The subject may be any animal, preferably a mammal, more preferably a human. The invention also provides methods for treating or preventing cancer by administering a nucleic acid sequence encoding an L protein or a variant thereof to a subject such that expression of the L protein or variant results in the production of these polypeptides in an amount effective to elicit an immune response. The invention further provides methods for treating or preventing cancer by administering an L protein or a peptide thereof, in an amount effective to elicit an immune response. The immune response may be humoral, cellular, or a combination of both. In a preferred embodiment the invention provides a method of immunizing to confer protection against the onset of lung cancer.

The invention relates to screening assays to identify antagonists or agonists of an L gene or gene product and variants thereof. Thus, the invention relates to methods for identifying agonists or antagonists of an L gene or gene product and variants thereof, and the use of said agonist or antagonist to treat or prevent lung cancer or other types of cancer.

The invention also provides methods for treating cancer by providing therapeutic amounts of an anti-sense nucleic acid molecule. An anti-sense molecule is a nucleic acid molecule that is a complement of all or a part of an L gene sequence and which, therefore, can hybridize to the L gene and variants thereof, or fragments thereof. Accordingly, hybridization of the anti-sense molecule can reduce or inhibit expression of an L gene. In a preferred embodiment the method is used to treat lung cancer.

The invention also includes a kit for assessing whether a patient is afflicted with lung cancer or other types of cancer. This kit comprises reagents for assessing expression levels of an L gene product.

In another aspect, the invention relates to a kit for assessing the suitability of each of a plurality of compounds for inhibiting cancer, including lung cancer, in a patient. The kit comprises a reagent for assessing expression of an L gene product, and may also comprise a plurality of compounds.

In another aspect, the invention relates to a kit for assessing the presence of cancer cells. The kit comprises an antibody, wherein the antibody binds specifically with a protein corresponding to an L gene product and variants thereof. The kit may also comprise a plurality of antibodies, wherein the plurality binds specifically with different epitopes of an L gene product and variants thereof. The kit may also comprise a plurality of antibodies, each one of which is immunologically specific for a different L protein. Accordingly, such kits may comprise at least one antibody that is immunologically specific for each of the nineteen L proteins.

The invention also includes a kit for assessing the presence of cancer cells, wherein the kit comprises a nucleic acid (e.g., oligonucleotide) probe. The probe binds specifically to a transcribed polynucleotide corresponding to an L gene product and variants thereof. The kit may also comprise a plurality of probes, wherein each of the probes binds specifically to a transcribed polynucleotide corresponding to a different region of the mRNA sequence transcribed from an L gene and variants thereof. The kit may also comprise a plurality of probes, each of which binds specifically to a transcribed polynucleotide corresponding to an mRNA sequence transcribed from a different L gene and variants thereof.

Kits for diagnostic use, including primers for use in PCR that can amplify an L gene cDNA and variants thereof, including the corresponding cDNA and/or genes and a standard amount of the L gene cDNA are also provided. Such kits may also comprise PCR primer pairs capable of amplifying different L gene nucleic acid molecules (i.e., L1-L19) and a standard amount of each of the L gene cDNAs in separate containers.

The invention also provides transgenic non-human animals (e.g., mice) that express nucleic acids and proteins encoded by a transgene of one of the L genes. Such transgenic animals can comprise multiple transgenes, each of which corresponds to a different L gene. Transgenic, non-human knockout animals (e.g., mice) of an L gene and variants thereof are also provided. Knockout animals, wherein more than one of the L genes is inactivated or “knocked out” are also provided.

Accordingly, the present invention provides a method of diagnosing cancer in a subject comprising detecting or measuring an L gene product in a sample derived from said subject, wherein said L gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby, in which elevated levels of an L gene product and variants thereof, compared to a non-cancerous sample or a pre-determined standard value for a noncancerous sample, indicates the presence of a cancer in the subject. In one embodiment of the foregoing diagnostic method, the subject is a human. In another embodiment, the cancer is lung cancer. In yet other embodiments, the sample is a tissue sample, a plurality of cells, or a bodily fluid.

The present invention further provides methods of staging cancer in a subject comprising detecting or measuring an L gene product and variants thereof, in a sample derived from said subject, wherein said L gene product and variants thereof, is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby, in which elevated levels of an L gene product and variants thereof, compared to a non-cancerous sample or a pre-determined standard value for a noncancerous sample, indicates an advanced stage of cancer in the subject.

The present invention further provides methods for treating cancer in a subject, comprising administering to the subject an amount of a compound which reduces the level and/or antagonizes the activity of an L gene product and variants thereof, wherein said L gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby. In one embodiment, a gene product whose expression is being decreased is a protein encoded by a nucleic acid comprising a nucleotide sequence with at least 90% sequence identity to one of SEQ ID NOs: 1-19. In another embodiment, the compound decreases expression of an RNA corresponding to one of SEQ ID NOs: 1-19. The antagonist can be (i) a protein; (ii) a peptide; (iii) an organic molecule with a molecular weight of less than 2000 daltons; (iv) an inorganic molecule with a molecular weight of less than 2000 daltons; (v) an antisense oligonucleotide molecule that binds to said RNA and inhibits translation of said RNA; (vi) a ribozyme molecule that targets said RNA and inhibits translation of said RNA; (vii) an antibody that specifically or selectively binds to an L gene product and variants thereof; (viii) a double stranded oligonucleotide that forms a triple helix with a promoter of an L gene and variants thereof, wherein said L gene is a nucleic acid at least 80% homologous to one of SEQ ID NOs: 1-19, or a complement thereof as determined using the NBLAST algorithm; or (ix) a double stranded oligonucleotide that forms a triple helix with a promoter of an L gene, wherein said L gene is a nucleic acid at least 80% homologous to one of SEQ ID NOs: 1-19, or a complement thereof as determined using the NBLAST algorithm. Wherein the compound is an L antagonist antibody, the antibody immunospecifically binds to a protein comprising an amino acid sequence of one of SEQ ID NOs: 20-38, or fragments thereof, and thereby reduces or inhibits an activity of an L protein.

The present invention further provides methods of vaccinating a subject against cancer comprising administering to the subject a molecule that elicits an immune response to an L gene product, wherein said L gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby. In one embodiment, the immune response is a cellular immune response. In another embodiment, the immune response is a humoral immune response. In yet another embodiment, the immune response is both a cellular and a humoral immune response.

The present invention yet further provides methods for determining if a subject is at risk for developing cancer, said method comprising (I) measuring an amount of an L gene product in a sample derived from the subject, wherein said L gene product is: (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm; or a protein encoded thereby; and (II) comparing the amount of said L gene product in the subject with the amount of L gene product present in a non-cancerous sample or predetermined standard for a noncancerous sample, wherein an elevated amount of said L gene product in the subject compared to the amount in the non-cancerous sample or pre-determined standard for a noncancerous sample indicates a risk of developing cancer in the subject.

The present invention yet further provides methods for determining if a subject suffering from cancer is at risk for metastasis of said cancer, said method comprising measuring an amount of an L gene product in a sample derived from the subject, wherein said gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby, wherein an elevated amount of L gene products in the subject compared to the amount in the non-cancerous sample, or in a sample from a subject with a non-metastasizing cancer, or the amount in a predetermined standard for a noncancerous or non-metastasizing sample, indicates a risk of developing metastasis of said cancer in the subject.

The present invention yet further provides methods of screening for a compound that binds with an L gene molecule, said method comprising (I) contacting the L gene molecule with a candidate agent, wherein said L gene molecule is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby and (II) determining whether or not the candidate agent binds the L gene molecule. The screening assay can be performed in vitro. In one embodiment, the L gene molecule, or a variant thereof, is anchored to a solid phase. In another embodiment, the candidate agent is anchored to a solid phase. In other embodiments, the screening assay is performed in liquid phase. In yet other embodiments, the L gene molecule and variants thereof, are expressed on the surface of a cell or in the cytosol of a cell in step (I). In the latter embodiments, the L gene molecule or variants thereof, are expressed naturally in the cell; alternatively, a cell can be engineered to express the L gene molecule or variants thereof. In the foregoing screening methods, the candidate agent is preferably labeled, for example radioactively or enzymatically.

The present invention provides methods of screening for a cellular protein that interacts with an L gene product, said method comprising (I) immunoprecipitating the L gene product from a cell lysate, wherein said L gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby; and (II) determining whether or not any cellular proteins bind to or form a complex with the L gene product in the immunoprecipitate.

The present invention yet further provides methods of screening for a candidate agent that modulates the expression level of an L gene, and/or variants thereof, said method comprising (I) contacting said L gene with a candidate agent, wherein said L gene is a nucleic acid at least 80% homologous to one of SEQ ID NOs: 1-19, as determined using the NBLAST algorithm; and (II) measuring the level of expression of an L gene product, said gene product selected from the group consisting of an mRNA corresponding to one of SEQ ID NOs: 1-19, or a protein comprising one of SEQ ID NOs: 20-38, wherein an increase or decrease in said level of expression relative to said level of expression in the absence of said candidate agent indicates that the candidate agent modulates expression of an L gene.

The present invention yet further provides a vaccine formulation for preventing or delaying the onset of cancer comprising (I) an immunogenic amount of an L gene product, wherein said L gene product is: (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ ID NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby; and (II) a pharmaceutically acceptable excipient.

The present invention yet further provides an immunogenic composition comprising (I) a purified L gene product in an amount effective for eliciting an immune response, wherein said gene product is (a) an RNA corresponding to one of SEQ ID NOs: 1-19, or a nucleic acid derived therefrom; (b) a protein comprising one of SEQ ID NOs: 20-38; (c) a nucleic acid comprising a sequence hybridizable to one of SEQ I) NOs: 1-19, or a complement thereof under conditions of high stringency, or a protein comprising a sequence encoded by said hybridizable sequence; (d) a nucleic acid at least 90% homologous to one of SEQ ID NOs: 1-19, or a complement thereof as determined using the NBLAST algorithm, or a protein encoded thereby; and (II) an excipient.

The present invention yet further provides a pharmaceutical composition comprising an antibody that specifically or selectively binds to a protein consisting essentially of one of SEQ ID NOs: 20-38; and a pharmaceutically acceptable carrier.

The present invention yet further provides pharmaceutical compositions comprising (I) an L gene product, wherein said gene product of the present invention yet further provides a pharmaceutical composition comprising an antibody which specifically or selectively binds to a protein comprising one of SEQ ID NOs: 20-38; and a pharmaceutically acceptable carrier; and (II) a pharmaceutically acceptable carrier.

The present invention yet further provides a pharmaceutical composition comprising (I) a purified nucleic acid comprising one of SEQ ID NOs: 1-19, and (II) a pharmaceutically acceptable carrier.

The pharmaceutical compositions of the present invention can be formulated, inter alia, for delivery as an aerosol, for parenteral delivery, or for oral delivery.

The present invention yet further provides methods of diagnosing cancer in a subject comprising (I) administering to said subject a compound that specifically binds a protein comprising one the amino acid sequences of SEQ ID NOs: 20-38, wherein said compound is bound to an imaging agent; and (II) obtaining an internal image of said subject by visualizing said imaging agent; wherein the localization or amount of said imaging agent indicates whether or not cancer is present in said subject. In a preferred embodiment, the compound is an antibody. In a preferred mode of the embodiment, the antibody is conjugated to a radioactive metal and said visualizing step comprises recording a scintographic image obtained from the decay of the radioactive metal.

The present invention yet further provides kits that are useful for practicing the present methods. In one embodiment, such a kit comprises, a pair of oligonucleotide primers, each primer comprising a nucleotide sequence with at least 5 complementary nucleotides to a different strand of a double-stranded nucleic acid comprising one of SEQ ID NOs: 1-19, and, a purified double-stranded nucleic acid comprising the corresponding SEQ ID NO: (e.g., one of SEQ ID NOs: 1-19). In specific modes of the embodiment, each primer comprises a nucleotide sequence with at least 8, more preferably at least 10, yet more preferably at least 12, and most preferably at least 15 complementary nucleotides to a different strand of a double-stranded nucleic acid comprising one of SEQ ID NOs: 1-19.

The present invention yet further provides transgenic non-human animals which express from a transgene an L gene product, for example, an RNA corresponding to one of SEQ ID NOs: 1-19, or a protein comprising one of SEQ ID NOs: 20-38.

The present invention yet further provides a method for testing the effects of a candidate therapeutic compound comprising administering said compound to a transgenic non-human animal which expresses from a transgene an L gene product and determining any effects of said compound upon said transgenic non-human animal.

The present invention further provides host cells comprising nucleic acids encoding the polypeptides of the invention operably linked to a promoter, and methods of expressing such polypeptides and variants thereto by culturing the host cells under conditions in which the nucleic acid molecule is expressed.

Also encompassed is a genetic expression signature for detecting a cancer, said genetic expression signature comprising each of SEQ ID NOs: 1-19 or fragments thereof and designated herein genetic expression signature 1d (GES1d), which is shown in Table 11. The present invention also includes an array comprising a genetic expression signature comprising each of SEQ ID NOs: 1-19 or fragments thereof, wherein said genetic expression signature is anchored to a solid phase. GES1d may also comprise any subset of SEQ ID NOs: 1-19 or fragments thereof and arrays comprising such subsets.

In an embodiment of the invention, a method for diagnosing a cancer in a subject is presented, said method comprising detecting at least one molecule of a genetic expression signature comprising SEQ ID NOs: 1-19, said method comprising:
(a) contacting a biological sample of said subject with a plurality of probes, wherein each of said probes is capable of binding specifically to a nucleic acid sequence corresponding to one of SEQ ID NOs: 1-19; and
(b) detecting binding of said probes to nucleic acid sequences of said biological sample,
   wherein detecting binding of at least one of said probes to a nucleic acid sequence of said biological sample is a positive indicator of a cancer in said subject.

In another embodiment of the invention, a method for diagnosing a cancer in a subject is presented, said method comprising detecting at least one molecule of a genetic expression signature comprising SEQ ID NOs: 20-38, said method comprising:
(a) contacting a biological sample of said subject with a plurality of probes, wherein each of said probes is capable of binding specifically to a polypeptide corresponding to one of SEQ ID NOs: 20-38; and
(b) detecting binding of said probes to polypeptides of said biological sample,
   wherein detecting binding of at least one of said probes to a polypeptide of said biological sample is a positive indicator of a cancer in said subject.

Also presented is a method for diagnosing a cancer in a subject, said method comprising detecting at least one molecule of a genetic expression signature comprising SEQ ID NOs: 20-38, said method comprising:
(a) contacting a biological sample of said subject with a plurality of probes, wherein each of said probes is capable of binding specifically to a nucleic acid sequence encoding a polypeptide corresponding to one of SEQ ID NOs: 20-38; and
(c) detecting binding of said probes to nucleic acid sequences of said biological sample,
   wherein detecting binding of at least one of said probes to a nucleic acid sequence of said biological sample is a positive indicator of a cancer in said subject.

The present invention also encompasses genetic expression signatures for detecting a cancer, said genetic expression signatures comprising one of GES1 (comprising genes/polypeptides listed in Table 7 or fragments thereof, wherein said genetic expression signature GES1 excludes a molecule comprising a nucleic or amino acid sequence corresponding to GenBank Accession No. BC052957); GES1a (comprising genes/polypeptides listed in Table 8 or fragments thereof, wherein said genetic expression signature GES1a excludes a molecule comprising a nucleic or amino acid sequence corresponding to GenBank Accession No. BC052957); GES1b (comprising genes/polypeptides listed in Table 9 or fragments thereof); GES1c (comprising genes/polypeptides listed in Table 10 or fragments thereof, wherein said genetic expression signature GES1 excludes a molecule comprising a nucleic or amino acid sequence corresponding to GenBank Accession No. BC052957); GES1e (comprising genes/polypeptides listed in Table 12 or fragments thereof, wherein said genetic expression signature GES1e excludes a molecule comprising a nucleic or amino acid sequence corresponding to GenBank Accession No. BC052957); GES1f (comprising genes/polypeptides listed in Table 13 or fragments thereof, wherein said genetic expression signature GES1f excludes a molecule comprising a nucleic or amino acid sequence corresponding to GenBank Accession No. BC052957); GES1g (comprising genes/polypeptides listed in Table 14 or fragments thereof, wherein said genetic expression signature GES1g excludes a molecule comprising a nucleic or amino acid sequence corresponding to GenBank Accession No. BC052957); and GES1h (comprising genes/polypeptides listed in Table 15 or fragments thereof, wherein said genetic expression signature GES1h excludes a molecule comprising a nucleic or amino acid sequence corresponding to GenBank Accession No. BC052957).

Also encompassed are arrays comprising one of the genetic expression signatures of the invention (e.g., one of GES1 or GES1a-h), wherein said genetic expression signature is anchored to a solid phase.

In an aspect of the invention, a method for diagnosing a cancer in a subject is provided, said method comprising detecting at least 80% of molecules comprising a genetic expression signature of the invention (e.g., one of GES1 or GES1a-h), said method comprising:
(a) contacting a biological sample of said subject with a plurality of probes, wherein each of said probes is capable of binding specifically to a nucleic acid sequence corresponding to a gene of the selected GES; and
(b) detecting binding of said probes to nucleic acid sequences of said biological sample,
   wherein detecting binding of at least 80% of said probes to a nucleic acid sequence of said biological sample is a positive indicator of a cancer in said subject.

In another aspect of the invention, a method for diagnosing a cancer in a subject is provided, said method comprising detecting at least 80% of molecules comprising a genetic expression signature of the invention (e.g., one of GES1 or GES1a-h), said method comprising:
(a) contacting a biological sample of said subject with a plurality of probes, wherein each of said probes is capable of binding specifically to a polypeptide of the selected GES; and
(b) detecting binding of said probes to polypeptides of said biological sample,
   wherein detecting binding of at least 80% of said probes to a polypeptide of said biological sample is a positive indicator of a cancer in said subject.

In another aspect of the invention, a method for diagnosing a cancer in a subject is provided, said method comprising detecting at least 80% of molecules comprising a genetic expression signature of the invention (e.g., one of GES1 or GES1a-h), said method comprising:
(a) contacting a biological sample of said subject with a plurality of probes, wherein each of said probes is capable of binding specifically to a nucleic acid sequence encoding a polypeptide of the selected GES; and
(b) detecting binding of said probes to nucleic acid sequences of said biological sample,
   wherein detecting binding of at least 80% of said probes to a nucleic acid sequence of said biological sample is a positive indicator of a cancer in said subject.

### BRIEF DESCRIPTION OF THE DRAWINGS

FIGS. 1A-S show nucleic acid sequences of L genes L1-L19 (SEQ ID NOs: 1-19)

FIGS. 2A-S show amino acid sequences of L proteins (SEQ ID NOs: 20-38) encoded by corresponding L genes L1-L19 (SEQ ID NOs: 1-19).

### DETAILED DESCRIPTION OF THE INVENTION

The following definitions are set forth to clarify aspects of the invention: SPECIFIC OR SELECTIVE: a nucleic acid used in a reaction, such as a probe used in a hybridization reaction, a primer used in a PCR, or a nucleic acid present in a pharmaceutical preparation, is referred to as “selective” if it hybridizes or reacts with the intended target more frequently, more rapidly, or with greater duration than it does with alternative substances. Similarly, a polypeptide is referred to as “selective” if it binds an intended target, such as a ligand, hapten, substrate, antibody, or other polypeptide more frequently, more rapidly, or with greater duration than it does to alternative substances. An antibody is referred to as “selective” if it binds via at least one antigen recognition site to the intended target more frequently, more rapidly, or with greater duration than it does to alternative substances. A marker is selective to a particular cell or tissue type if it is expressed predominantly in or on that cell or tissue type, particularly with respect to a biological sample of interest.

VARIANT (S): A variant (v) of polynucleotides or polypeptides, as the term is used herein, are polynucleotides or polypeptides that are different from a reference polynucleotide or polypeptide, respectively.

Variant polynucleotides are generally limited so that the nucleotide sequence of the reference and the variant are closely related overall and, in many regions, identical. Changes in the nucleotide sequence of the variant may be silent. That is, they may not alter the amino acid sequence encoded by the polynucleotide. Where alterations are limited to silent changes of this type a variant will encode a polypeptide with the same amino acid sequence as the reference. Alternatively, changes in the nucleotide sequence of the variant may alter the amino acid sequence of a polypeptide encoded by the reference polynucleotide. Such nucleotide changes may result in amino acid substitutions, additions, deletions, fusions, and truncations in the polypeptide encoded by the reference sequence.

Variant polypeptides are generally limited so that the sequences of the reference and the variant are closely similar overall and, in many regions, identical. For example, a variant and reference polypeptide may differ in amino acid sequence by one or more substitutions, additions, deletions, fusions, and truncations, which may be present or absent in any combination.

CORRESPOND OR CORRESPONDING: Between nucleic acids, “corresponding” means homologous to or complementary to a particular sequence or portion of the sequence of a nucleic acid. As between nucleic acids and polypeptides, “corresponding” refers to amino acids of a peptide in an order derived from the sequence or portion of the sequence of a nucleic acid or its complement. As between polypeptides (or peptides and polypeptides), “corresponding” refers to amino acids of a first polypeptide (or peptide) in an order derived from the sequence or portion of the sequence of a second polypeptide.

L GENE: As used herein, unless otherwise indicated, refers to one of the nineteen novel genes of the present invention which are designated L1-L19. In some aspects of the invention, the term “L gene” may also include all 147 genes discovered herein to be up-regulated in certain cancers (e.g., lung cancer) and subsets thereof.

L GENE PRODUCT: As used herein, unless otherwise indicated, an L gene product is: an RNA corresponding to one of SEQ ID NOs: 1-19, or a nucleic acid derived therefrom; a protein comprising one of SEQ ID NOs: 20-38; a nucleic acid comprising a sequence hybridizable to one of SEQ ID NOs: 1-19 or a complement thereof under conditions of high stringency, or a protein comprising a sequence encoded by said hybridizable sequence; a nucleic acid at least 90% homologous to one of SEQ ID NOs: 1-19 or a complement thereof as determined using the NBLAST algorithm; a nucleic acid at least 90% homologous to one of SEQ ID NOs: 1-19 or a fragment or derivative of any of the foregoing proteins or nucleic acids.

CONTROL ELEMENTS: As used herein refers collectively to promoter regions, polyadenylation signals, transcription termination sequences, upstream regulatory domains, origins of replication, internal ribosome entry sites (“IRES” ), enhancers, and the like, which collectively provide for the replication, transcription and translation of a coding sequence in a recipient cell. Not all of these control elements is required so long as the selected coding sequence is capable of being replicated, transcribed and translated in an appropriate host cell.

PROMOTER REGION: Is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

OPERABLY LINKED: As used herein refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof.

EXOGENOUS: As used herein the term exogenous refers to that which is derived or originated externally. When used in the context of exogenous expression of a gene or protein, the term refers to a gene or protein that is being expressed in a cell or tissue that does not normally express the gene or protein. When used in the context of nucleic acid sequences, for example, the term may also be used to refer to an association of two or more nucleic acid sequences that have been operably linked, but are not normally operably linked in a native state.

TO TREAT A CANCER OR A TUMOR: As used herein, the phrase “to treat a cancer or a tumor” or “treating a cancer or a tumor” in a mammal means one or more of alleviating a symptom of, correcting an underlying molecular or physiological disorder of, or reducing the frequency or severity of a pathological or deleterious physiological consequence of a cancer or a tumor in a mammal. By way of example, and not by limitation, the deleterious physiological consequences of a cancer or a tumor can include uncontrolled proliferation, metastasis and invasion of other tissues, and suppression of an immune response.

TO STAGE A TUMOR: As used herein, to “stage a tumor” or to “determine the stage of progression of a tumor” means to ascertain the stage of progression of a tumor along the continuum from non-invasive to invasive, or from non-metastatic to metastatic. Typically tumors are staged from grades I-IV with IV being the most malignant or metastatic.

IMMUNOLOGICALLY SPECIFIC: With respect to antibodies of the invention, the term “immunologically specific” refers to antibodies that bind to one or more epitopes of a protein of interest (e.g., L1 protein), but which do not substantially recognize and bind other molecules in a sample containing a mixed population of antigenic biological molecules.

CONSISTING ESSENTIALLY OF: The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence. Such an amino acid sequence exhibits 80% homology or greater to that of the given SEQ ID NO:.

MODULATE: As used herein, a compound which is capable of increasing or decreasing the level and/or activity of an L molecule may be referred to herein as an L molecule modulator.

ANTAGONIST: As used herein, a compound capable of reducing the level and/or activity of an L molecule or a variant thereof may be referred to herein as an L molecule antagonist.

AGONIST: As used herein, a compound capable of increasing the level and/or activity of an L molecule or a variant thereof may be referred to herein as an L molecule agonist.

L GENE ACTIVITY: As used herein, the terms “L gene activity”, “L gene product activity”, or “L mediated activity” refer to an acitivity associated with the expression of an L gene and/or L gne product. Such activities include, but are not limited to changes in cellular proliferation, cellular motility, cellular differentiation, and/or cellular adhesion associated with changes in L gene or gene product expression levels

ELEVATED L MOLECULE LEVELS: As used herein the terms “elevated”, “over-expressed”, “up-regulated”, or “increased” L molecule levels refer to an approximately two-fold or greater increase in the expression of L transcript and/or protein as compared to that of a control tissue, which expresses a baseline level of an L molecule.

L POSITIVE CANCER: As used herein, the phrase “L positive cancer” refers to a cancer wherein the expression of at least one L gene is elevated as compared to that of a non-cancerous or control tissue.

GENETIC SIGNATURE: As used herein, the terms “genetic signature” or “genetic expression signature” refer to an expression pattern indicative of a particular condition. As such, detection of a genetic signature is a positive indicator of the presence of a particular condition in a subject wherein the genetic signature is identified. The present invention describes genetic expression signatures for NSCLC, the detection of which serves as a diagnostic indicator of NSCLC in a subject. It is to be understood that detection of greater than 80% of the genes or gene products that comprise a comprehensive genetic expression signature can serve as a diagnostic indicator of a condition associated with the genetic signature.

MOLECULES: As used herein, the term “molecule” refers a polynucleotide or polypeptide or a variant or derivative thereof. The term may also be used to refer to a macromolecule comprising a polynucleotide or polypeptide or a variant or derivative thereof

### ASPECTS OF THE INVENTION

Intensive and systematic evaluation of gene expression patterns is crucial for understanding the physiological mechanisms associated with cellular transformation and metastasis. Currently, several technical platforms are being used to address the correlation between gene expression pattern and carcinogenic transformation and progression of disease. Such techniques include: SAGE (Velculescu et al., 1995, Science 270: 484-487); Restriction Enzyme Analysis of Differentially Expressed Sequences (READS) (Prasher et al., 1999, Methods Enzymol. 303: 258); Amplified Fragment Length Polymorphism (AFLP) (Bachem et al., 1996, Plant J. 9: 745); Representational Difference Analysis (RDA) (Hubank et al., 1994, Nucleic Acid Res. 22(25): 5640); Differential Display (Liang et al., 1992, Cancer Res. 52(24): 6966); and SSH (Diatchenko et al., 1996, Proc. Natl. Acad. Sci. 93: 6025-6030) as detailed in this text. SSH is very similar to RDA but includes an additional normalization step that serves to increase the relative abundance of rare transcripts.

A combination of SSH and cDNA microarrays offers several advantages over the aforementioned technologies for the discovery of novel tumor-associated proteins and antigens (TAA's). The use of SSH for identifying novel cancer targets is an attractive approach because it does not rely on previously characterized cDNA sets. SSH efficiently normalizes both frequent and rare transcripts at equivalent levels and preferentially amplifies only those which are differentially expressed. The use of expression arrays further increases the chances of identifying lead targets by examining thousands of genes in a single experiment.

The results presented herein validate the effectiveness of this combinatorial approach involving both SSH and expression profiling techniques for identifying NSCLC-associated molecules. Utilization of a mixture of normal tissues in the subtraction procedure further promoted the successful enrichment of unique tumor-selective genes while eliminating common redundant sequences. As described by way of example herein, 147 NSCLC-associated molecules have been identified that comprise a novel genetic expression signature characteristic of NSCLC. Of the 147 NSCLC-associated molecules whose expression is up-regulated in NSCLC, 48 genes have been previously linked to lung cancer, 53 genes have been linked to cancers other than lung cancer, and 46 genes have not previously been linked to any cancer. The subset of 46 genes not previously associated with a cancer includes 19 novel genes. The NSCLC-associated molecules described herein have, therefore, been organized into several distinct functional categories. Accordingly, in addition to the comprehensive genetic expression signature which comprises all 147 NSCLC-associated molecules (designated herein as an NSCLC genetic expression signature 1; GES 1), a genetic expression signature comprising 99 molecules never previously associated with lung cancer is described and designated herein GES1a. Also provided is a genetic expression signature comprising 46 up-regulated molecules never previously associated with any cancer and designated herein GES1b. A genetic expression signature comprising 53 known cancer associated, but not lung cancer associated, molecules is designated GES1c. A genetic expression signature comprising 19 novel L genes (designated herein as L1-L19) is also described and designated GES1d. A genetic expression signature comprising 13 molecules displaying≧10-fold tumor: normal ratios is described and designated GES1e. A genetic expression signature comprising 45 molecules displaying≧5-fold tumor: normal ratios is described and designated GES1f. A genetic expression signature comprising 66 molecules displaying≧4-fold tumor: normal ratios is described and designated GES1g. A genetic expression signature comprising 103 molecules displaying≧3-fold tumor: normal ratios and designated GES1h.

Details concerning the isolation and characterization of each of the L gene cDNAs, its expression level in various cancer cell lines and tissues, and the significance of its expression in carcinogenic processes are described in detail in the examples provided.

As described in the Example section, tumor-selectivity for a subset of these genes was further validated by additional independent assays using tumor cell lines and patient tissue samples. The overall relevance of these genes as potential therapeutic targets for NSCLC, and/or other cancers, is a subject of the present invention.

The present invention encompasses methods for the diagnosis, prognosis and staging of lung cancer and other cancers, as well as methods for treating a patient with cancer and/or monitoring of the effect of a therapeutic treatment. Further provided are methods for the use of the L gene products in the identification of compounds that modulate the expression of L gene or the activity of an L gene product. As described herein, expression of an L gene and variants thereof, is upregulated in various types of cancer cells including lung cancer cell lines and tissues. As such, L gene products can be involved in the mechanisms underlying the onset and development of lung cancer and other types of cancer as well as the regional infiltration and metastatic spread of cancer. Thus, the present invention also provides methods for the prevention and/or treatment of lung cancer and other types of cancer, and for the control of metastatic spread of lung cancer and other types of cancer, wherein such regimens are based on modulating the expression and/or activity of an L molecule. In a specific embodiment, the invention is directed to methods wherein antagonists or agonists of an L molecule mediated activity are used to efficaciously treat a cancer patient.

The invention further provides for screening assays and methods of identifying agonists and antagonists of an L gene or gene product. The invention also provides methods for vaccinating an individual against cancer (e.g., lung cancer), by administering an amount of an L gene, gene product, or fragment thereof, in an amount that effectively elicits an immune response in a subject who has cancer or is at risk of developing cancer.

The L Genes

In accordance with the present invention, there are provided nucleic and amino acid sequences of nineteen novel L genes, L1-L19, the expression of which is upregulated in NSCLC. A nucleotide sequence comprising an open reading frame which includes the termination stop triplet sequence (either TAG, TGA, TAA) corresponding to each of the 19 L genes is described herein. Each of the L gene cDNAs was cloned by PCR using gene-specific primers. An L1 sequence comprises an open reading frame (SEQ ID NO: 1) of 975 nucleotides that encodes a protein of 324 amino acids SEQ ID NO: 20. An L2 sequence comprises an open reading frame (SEQ ID NO: 2) of 1935 nucleotides that encodes a protein of 644 amino acids SEQ ID NO: 21. An L3 sequence comprises an open reading frame (SEQ ID NO: 3) of 861 nucleotides that encodes a protein of 286 amino acids SEQ ID NO: 22. An L4 sequence comprises an open reading frame (SEQ ID NO: 4) of 666 nucleotides that encodes a protein of 221 amino acids SEQ ID NO: 23. An L5 sequence comprises an open reading frame (SEQ ID NO: 5) of 336 nucleotides that encodes a protein of 111 amino acids SEQ ID NO: 24. An L6 sequence comprises an open reading frame (SEQ ID NO: 6) of 408 nucleotides that encodes a protein of 135 amino acids SEQ ID NO: 25. An L7 sequence comprises an open reading frame (SEQ ID NO: 7) of 1902 nucleotides that encodes a protein of 633 amino acids SEQ ID NO: 26. An L8 sequence comprises an open reading frame (SEQ ID NO: 8) of 828 nucleotides that encodes a protein of 275 amino acids SEQ ID NO: 27. An L9 sequence comprises an open reading frame (SEQ ID NO: 9) of 1791 nucleotides that encodes a protein of 596 amino acids SEQ ID NO: 28. An L10 sequence comprises an open reading frame (SEQ ID NO: 10) of 978 nucleotides that encodes a protein of 325 amino acids SEQ ID NO: 29. An L11 sequence comprises an open reading frame (SEQ ID NO: 11) of 573 nucleotides that encodes a protein of 190 amino acids SEQ ID NO: 30. An L12 sequence comprises an open reading frame (SEQ ID NO: 12) of 1473 nucleotides that encodes a protein of 490 amino acids SEQ ID NO: 31. An L13 sequence comprises an open reading frame (SEQ ID NO: 13) of 1299 nucleotides that encodes a protein of 432 amino acids SEQ ID NO: 32. An L14 sequence comprises an open reading frame (SEQ ID NO: 14) of 2160 nucleotides that encodes a protein of 719 amino acids SEQ ID NO: 33. An L15 sequence comprises an open reading frame (SEQ ID NO: 15) of 690 nucleotides that encodes a protein of 229 amino acids SEQ ID NO: 34. An L16 sequence comprises an open reading frame (SEQ ID NO: 16) of 4173 nucleotides that encodes a protein of 1390 amino acids SEQ ID NO: 35. An L17 sequence comprises an open reading frame (SEQ ID NO: 17) of 723 nucleotides that encodes a protein of 240 amino acids SEQ ID NO: 36. An L18 sequence comprises an open reading frame (SEQ ID NO: 18) of 2790 nucleotides that encodes a protein of 929 amino acids SEQ ID NO: 37. An L19 sequence comprises an open reading frame (SEQ ID NO: 19) of 1518 nucleotides that encodes a protein of 505 amino acids SEQ ID NO: 38.

The L nucleic acids and derivatives used in the present invention include but are not limited to RNA corresponding to one of SEQ ID NOs: 1-19, or a nucleic acid derived therefrom, including but not limited to RNAs comprising one of SEQ ID NOs: 1-19; a nucleic acid comprising a sequence hybridizable to one of SEQ ID NOs: 1-19, or a complement thereof of any one of the foregoing nucleic acids; a nucleic acid at least 90% homologous to one of SEQ ID NOs: 1-19, or at least 90% homologous to the complement of any of the foregoing nucleic acids (e.g., as determined using the NBLAST algorithm under default parameters). As used herein an “RNA corresponding to one of SEQ ID NOs: 1-19, means an RNA comprising a sequence that is the same or the (inverse) complement of one of SEQ ID NOs: 1-19, except that thymidines (T's) are be replaced with uridines (U's). Such RNAs corresponding to one of SEQ ID NOs: 1-19, include for example RNA encoded by one of SEQ ID NOs: 1-19 in either the sense or anti-sense orientation. A nucleic acid derived from such RNA includes but is not limited to cDNA of said RNA, and cRNA (e.g., RNA that is derived from said cDNA; see, e.g., U.S. Pat. Nos. 5,545,522; 5,891,636; 5,716,785). In the present invention, the ability to hybridize may be determined under low, moderate, or high stringency conditions and preferably is under conditions of high stringency.

An L protein and derivatives used in the present invention include, but are not limited to proteins (and other molecules) comprising one of SEQ ID NOs: 20-38, proteins comprising a sequence encoded by the hybridizable (complementary) portion of a nucleic acid hybridizable to one of SEQ ID NOs: 1-1 9, or a complement thereof, and proteins encoded by a nucleic acid at least 90% homologous to one of SEQ ID NOs: 1-19, or a complement thereof, e.g., as determined using the NBLAST algorithm.

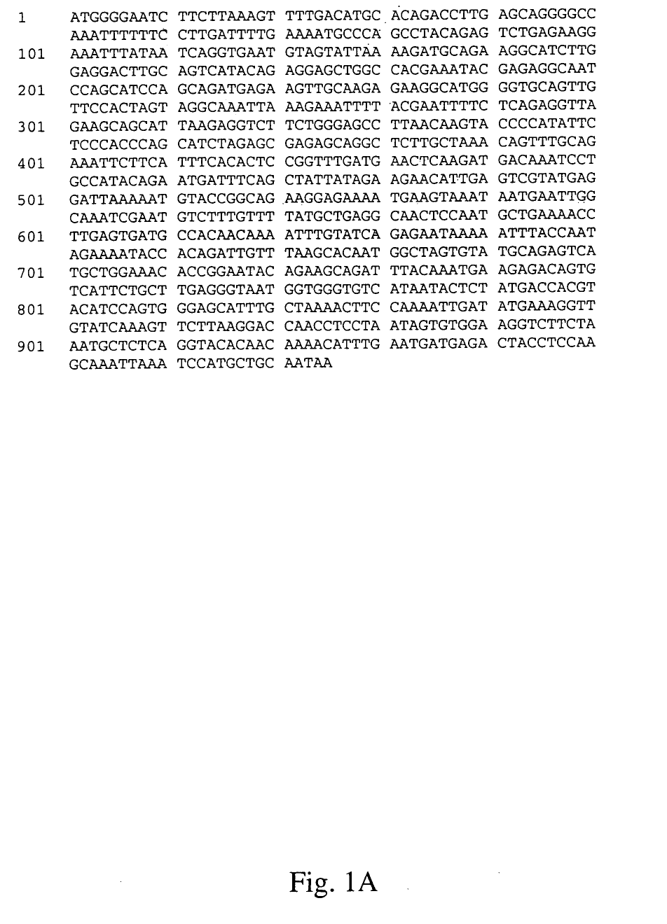

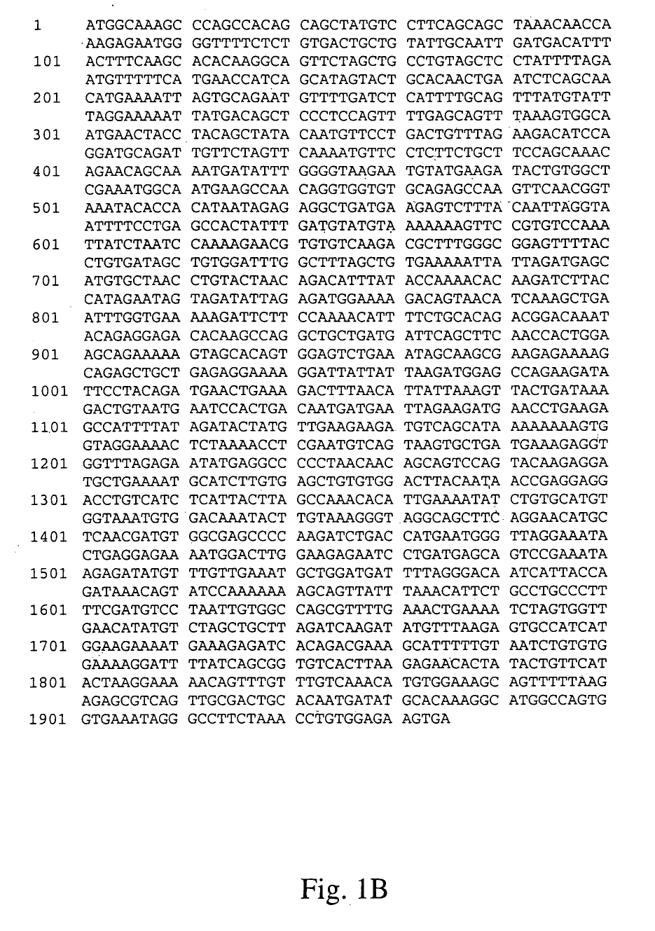

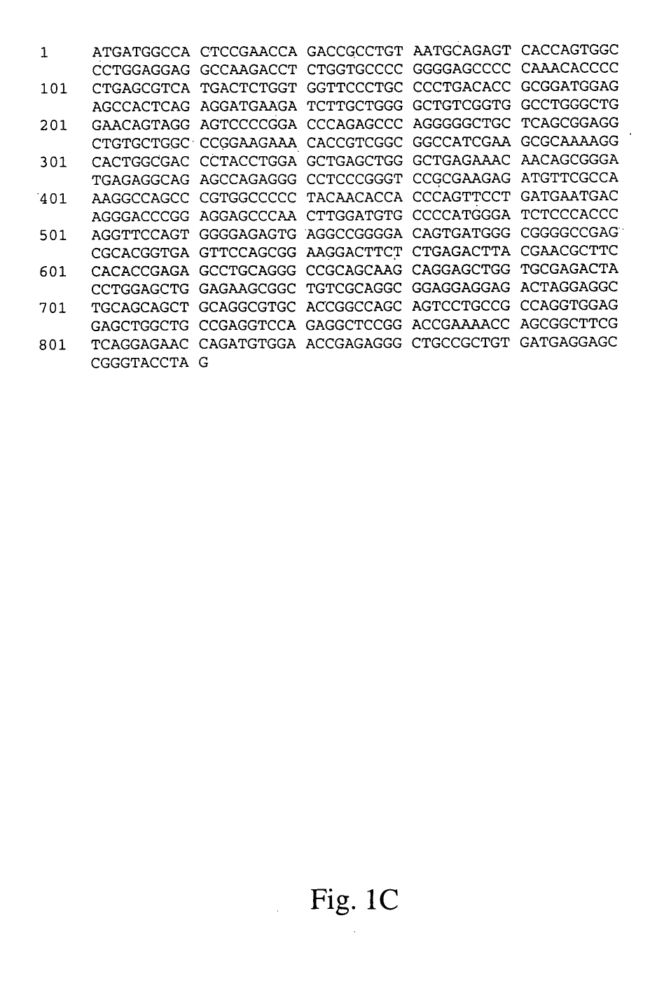

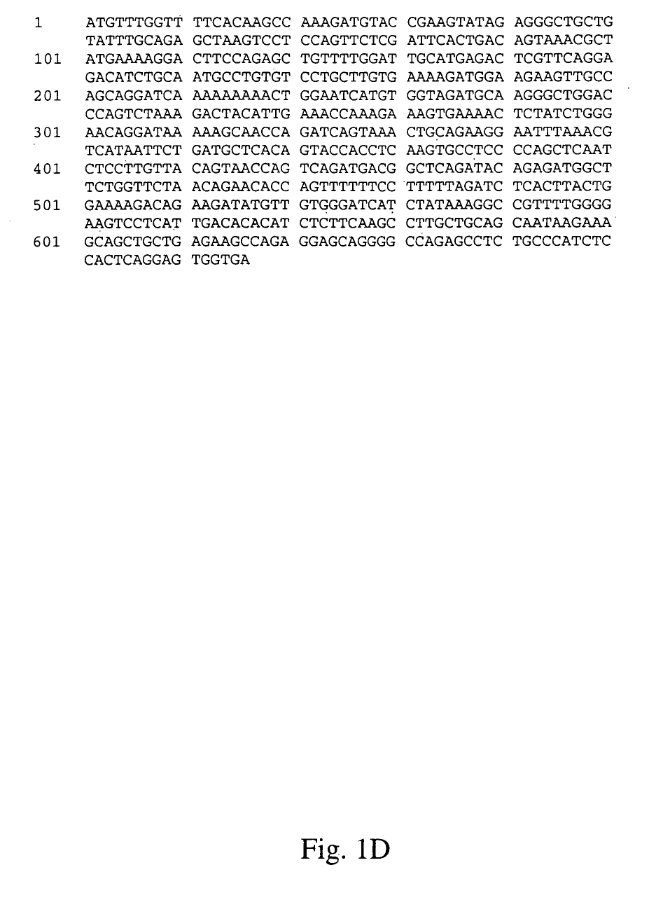

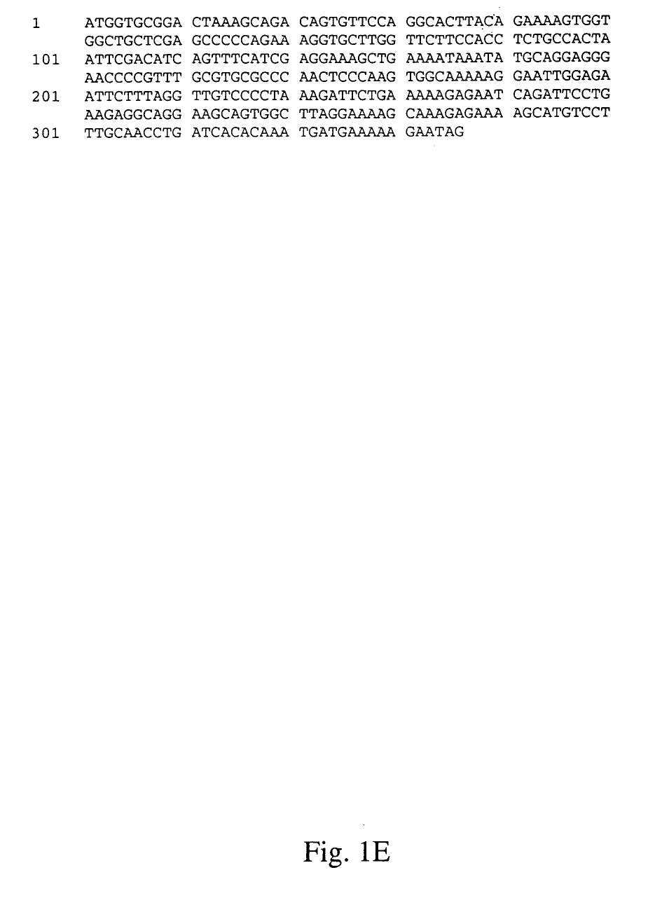

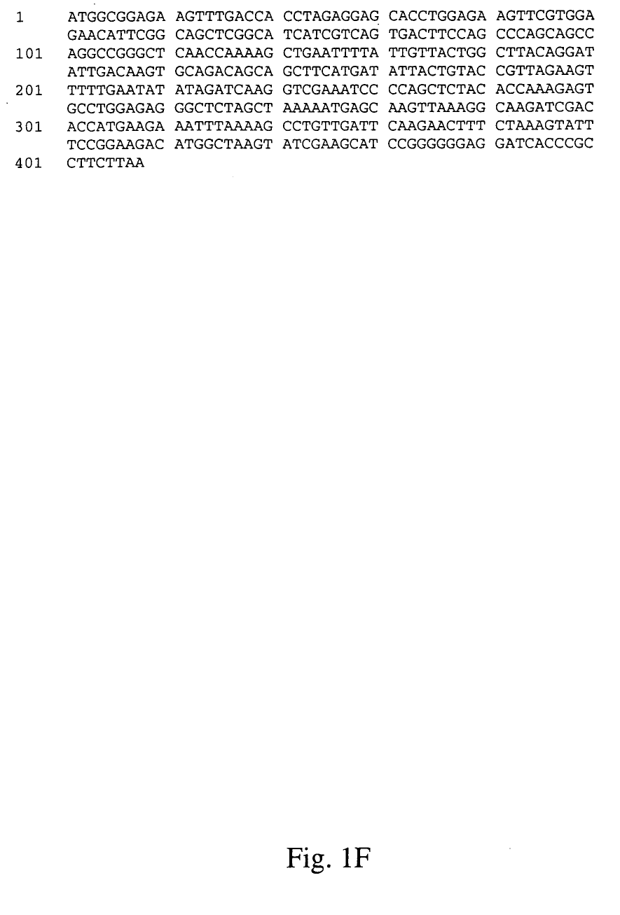

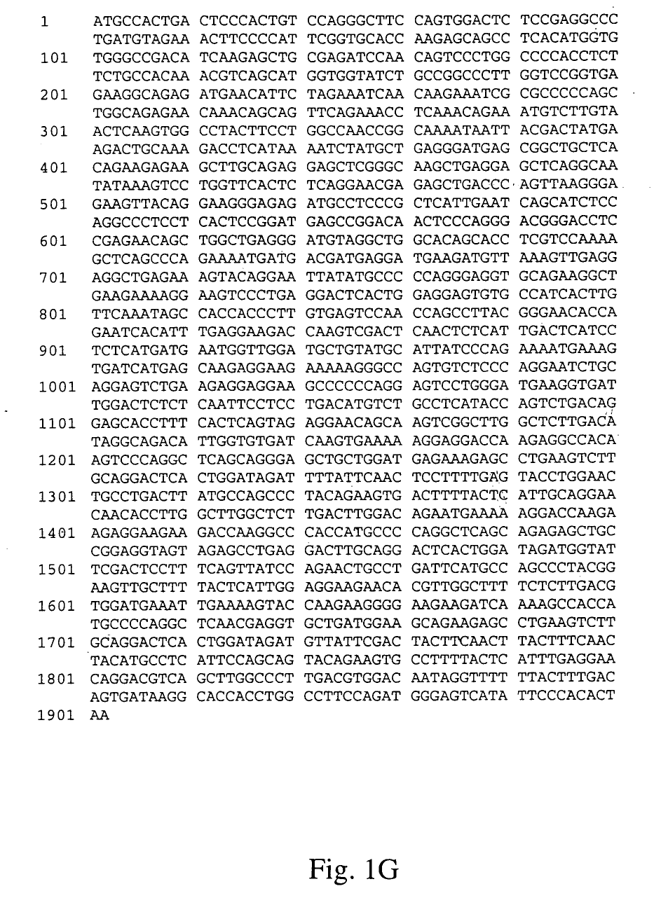

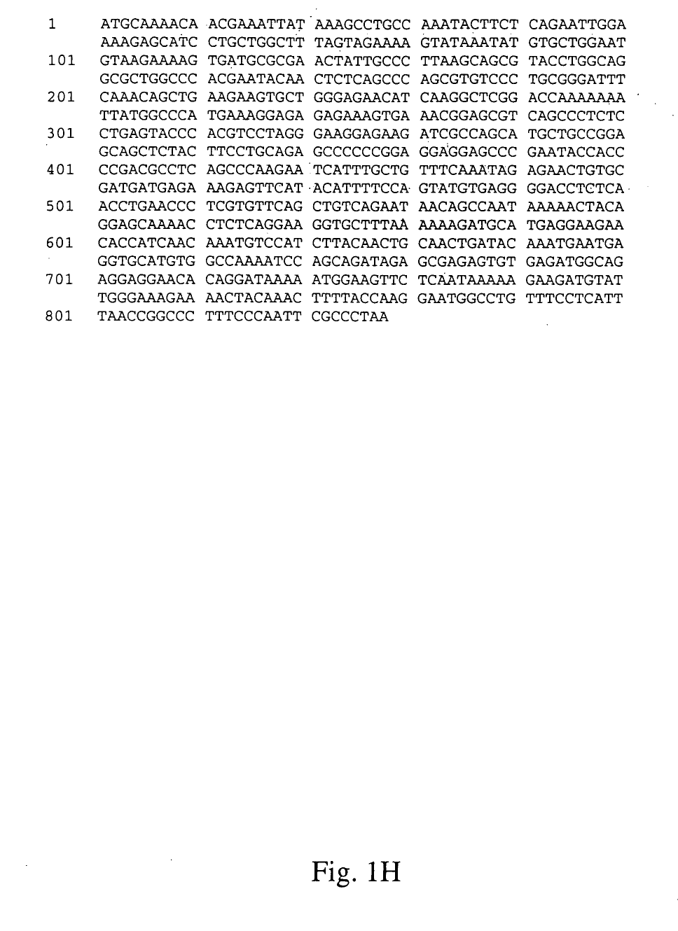

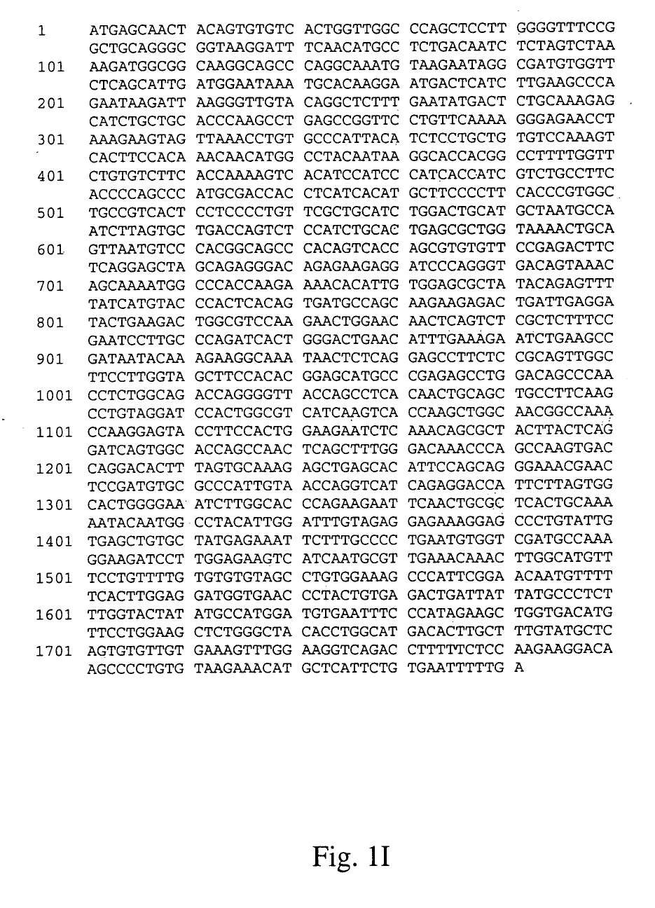

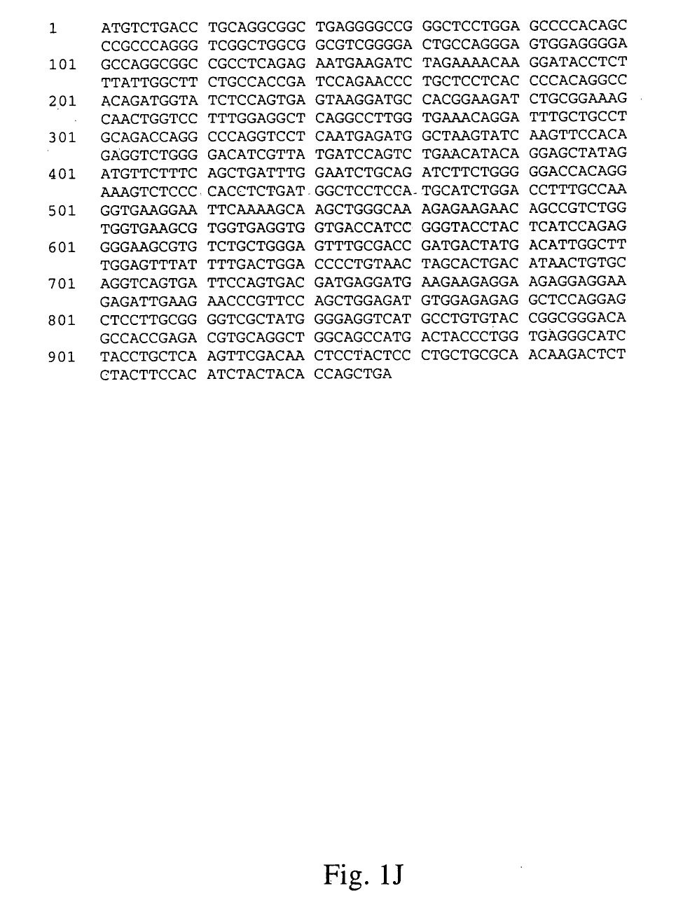

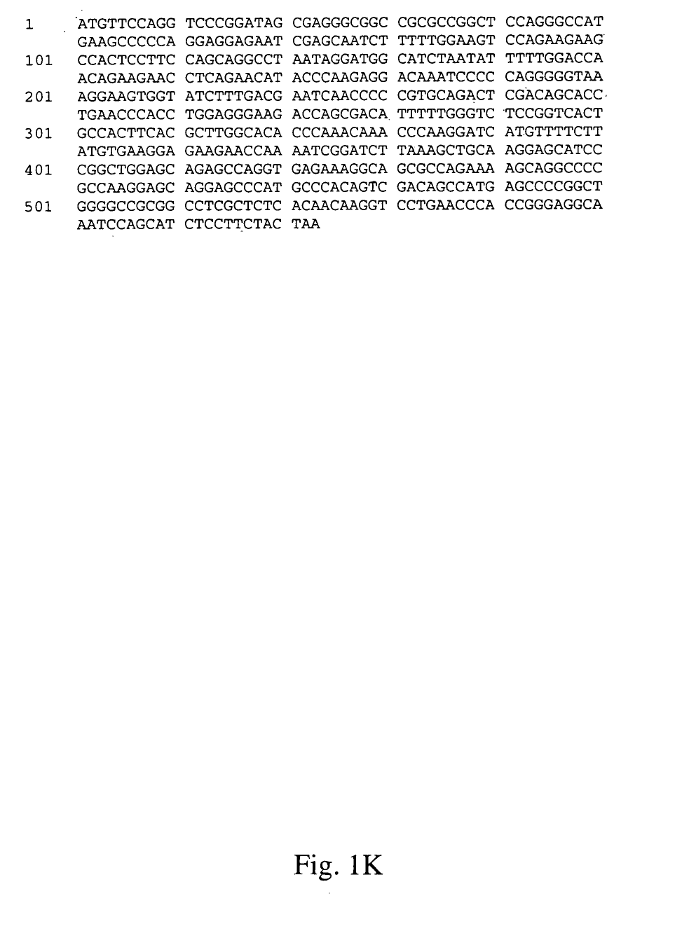

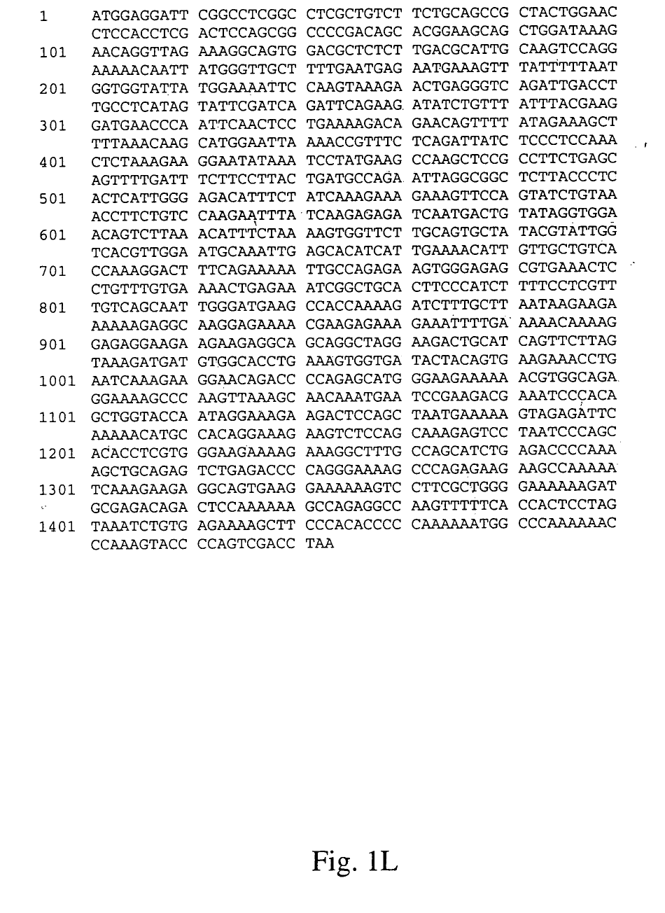

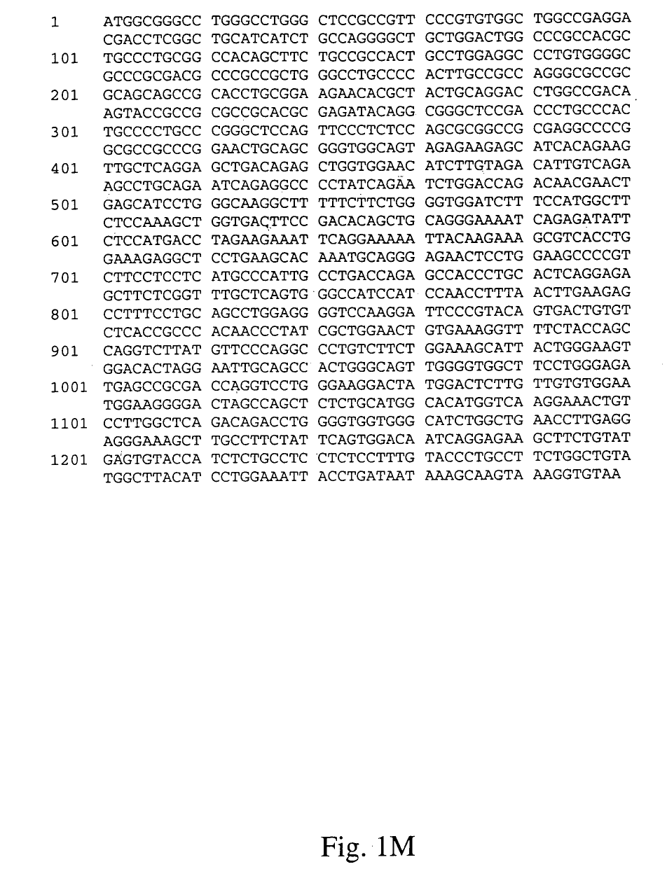

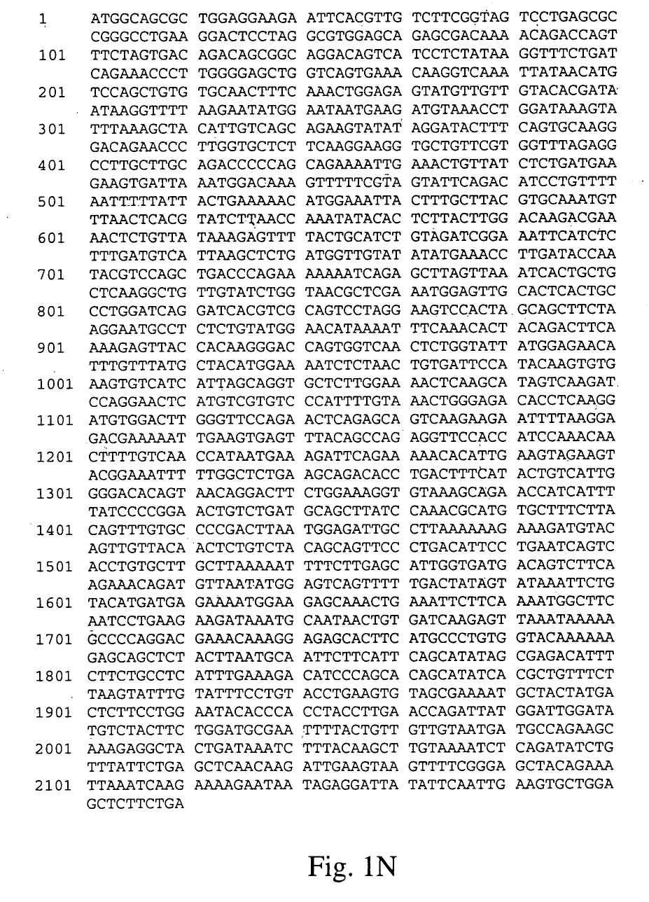

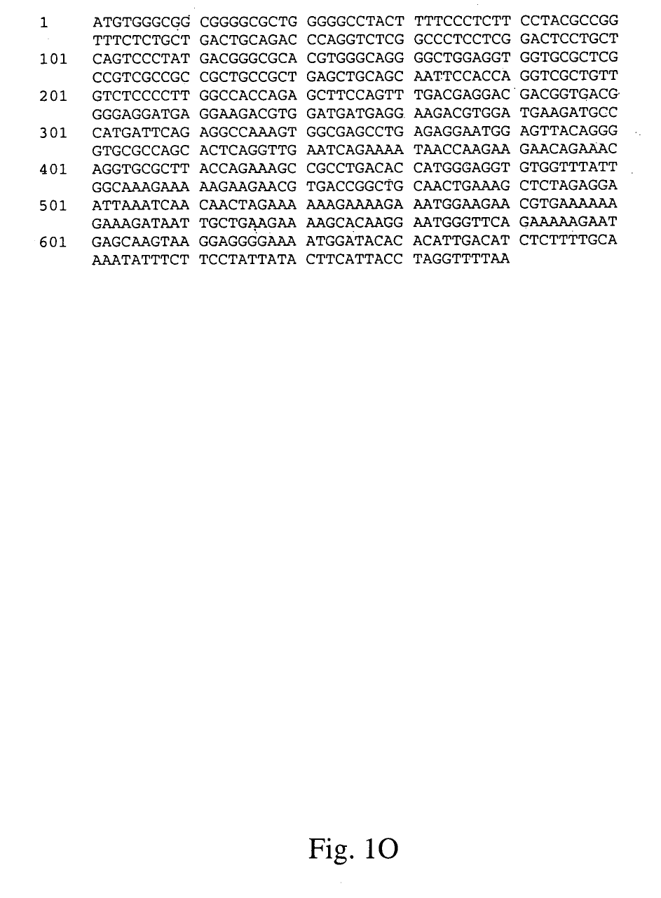

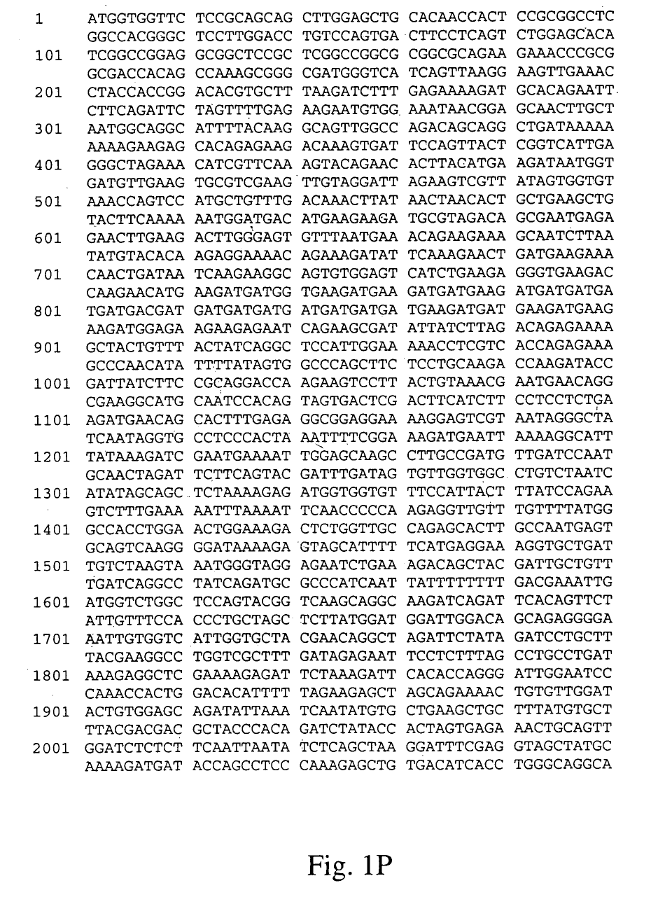

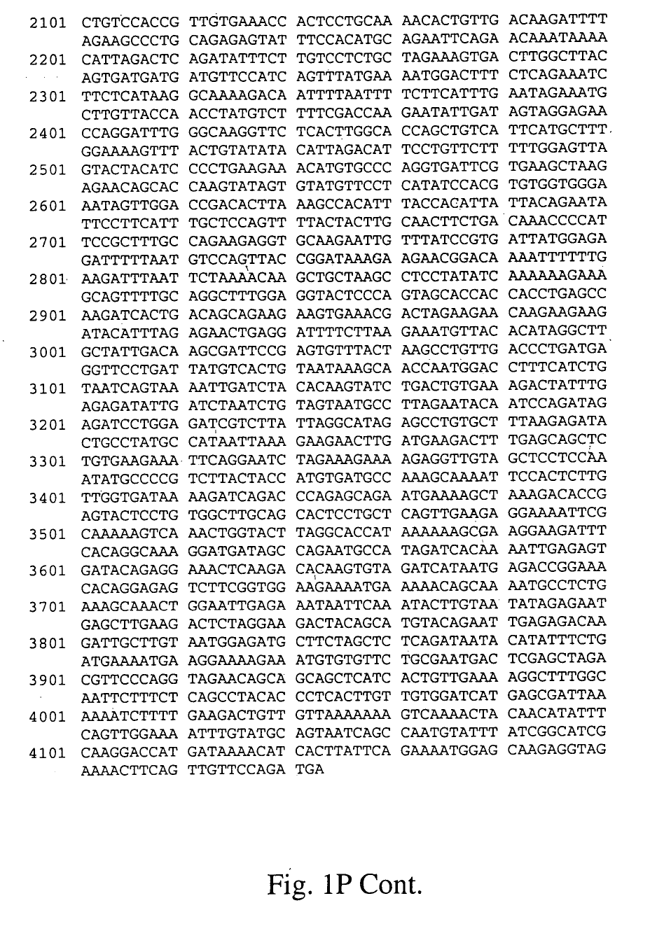

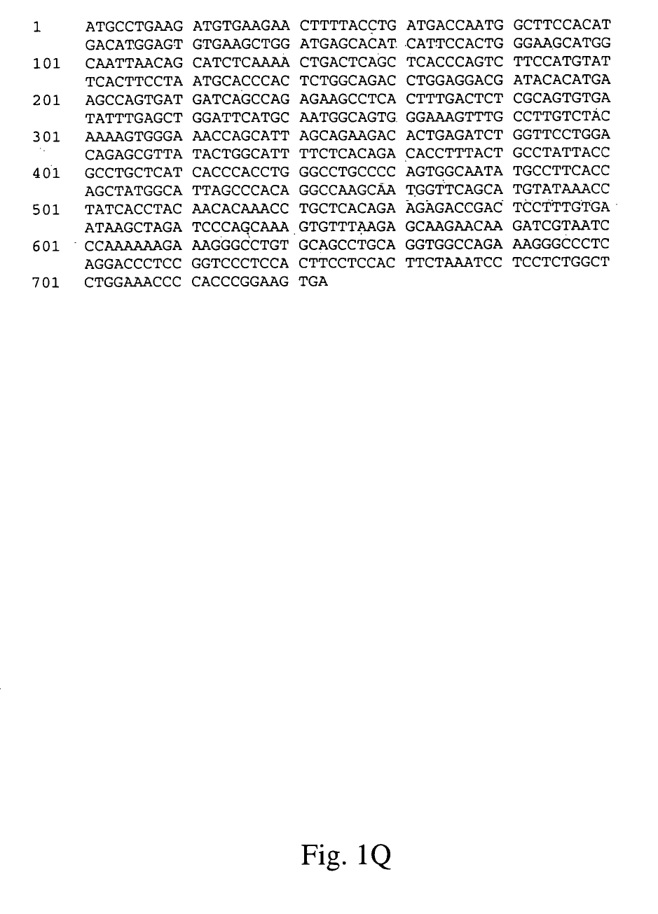

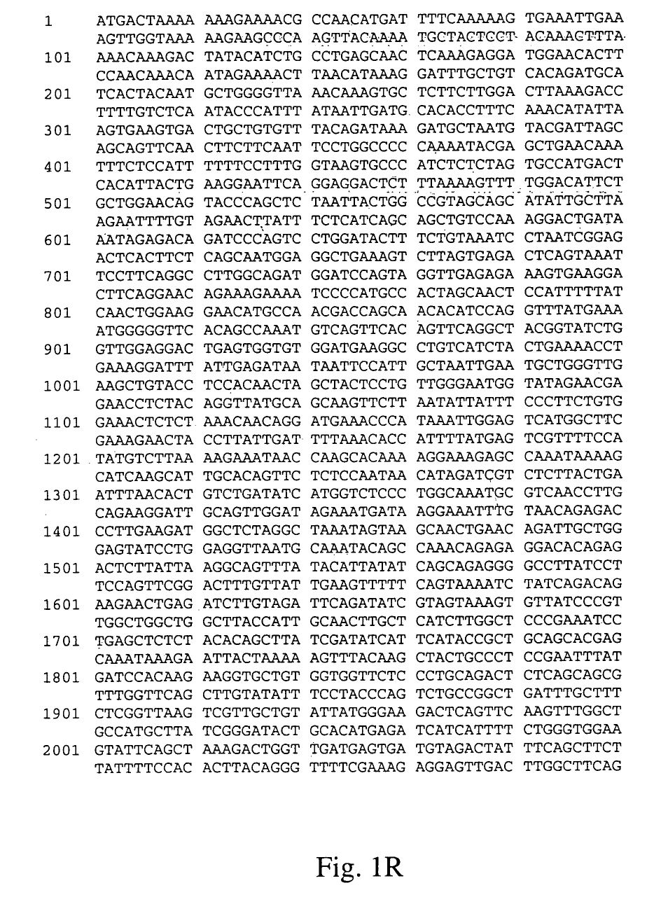

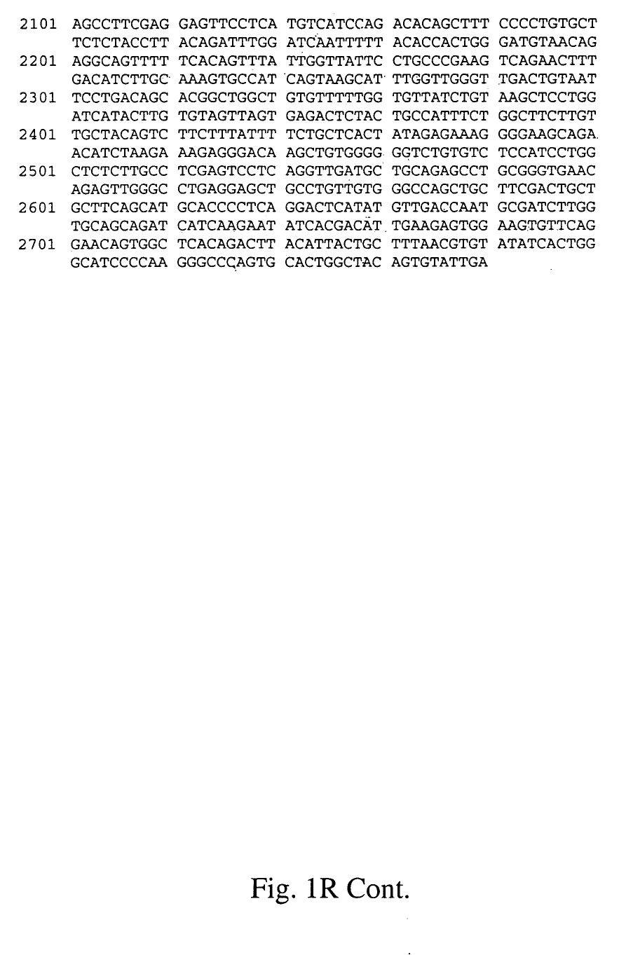

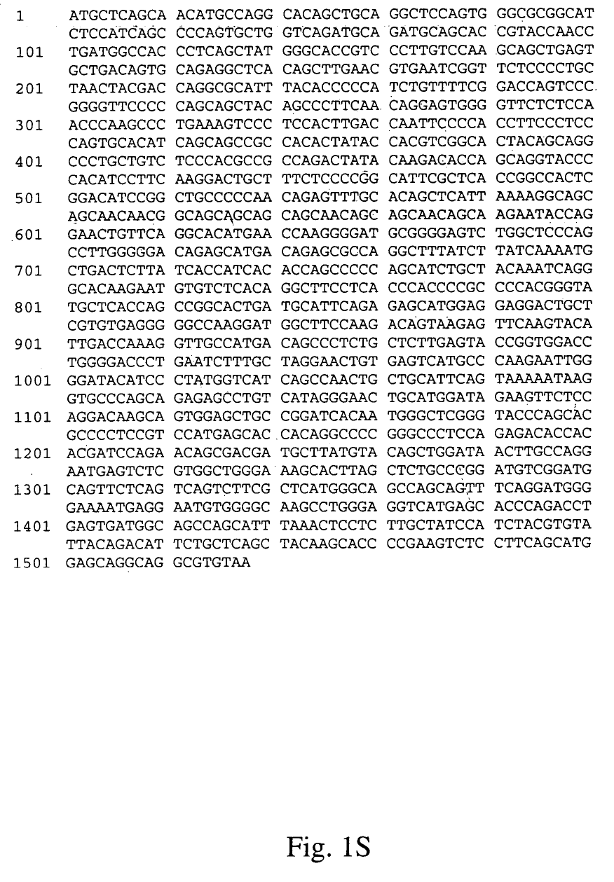

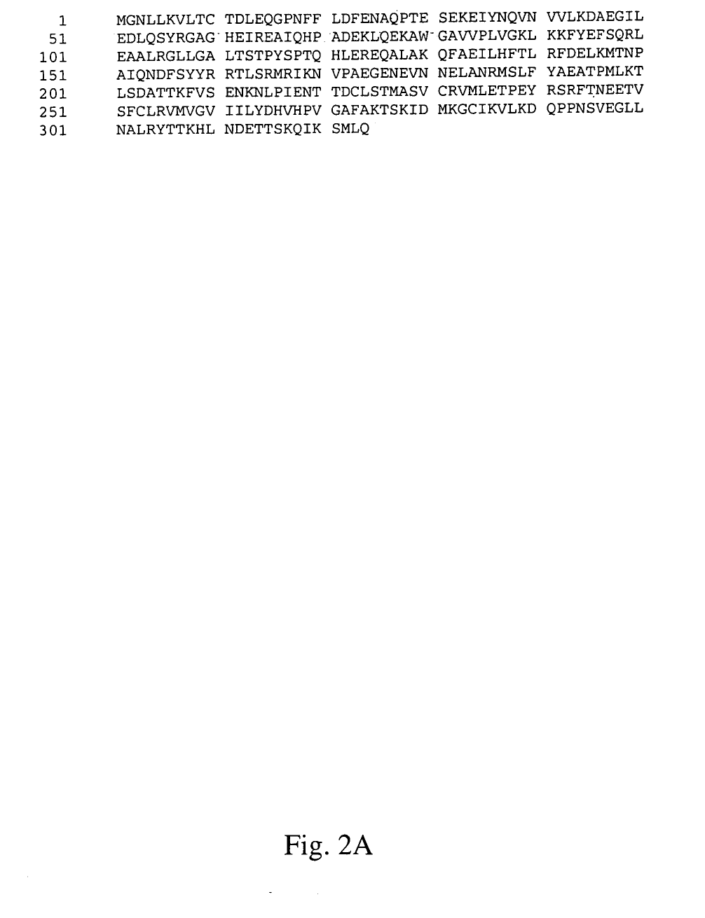

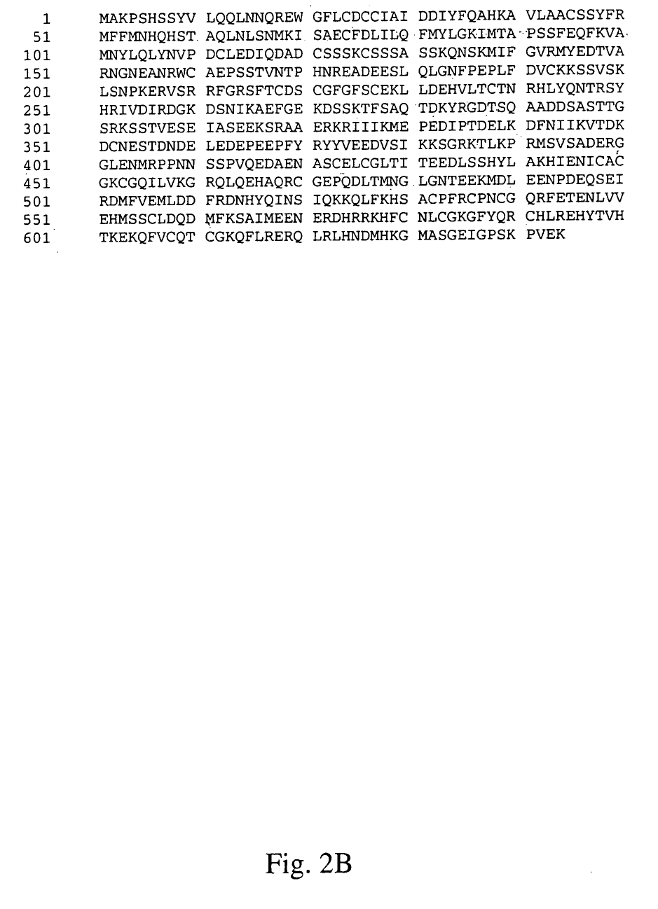

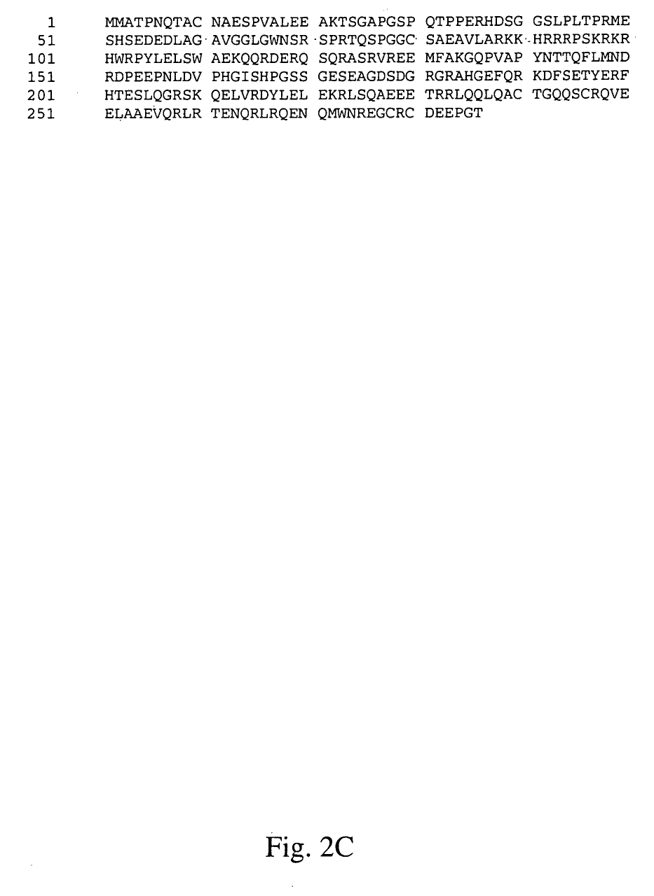

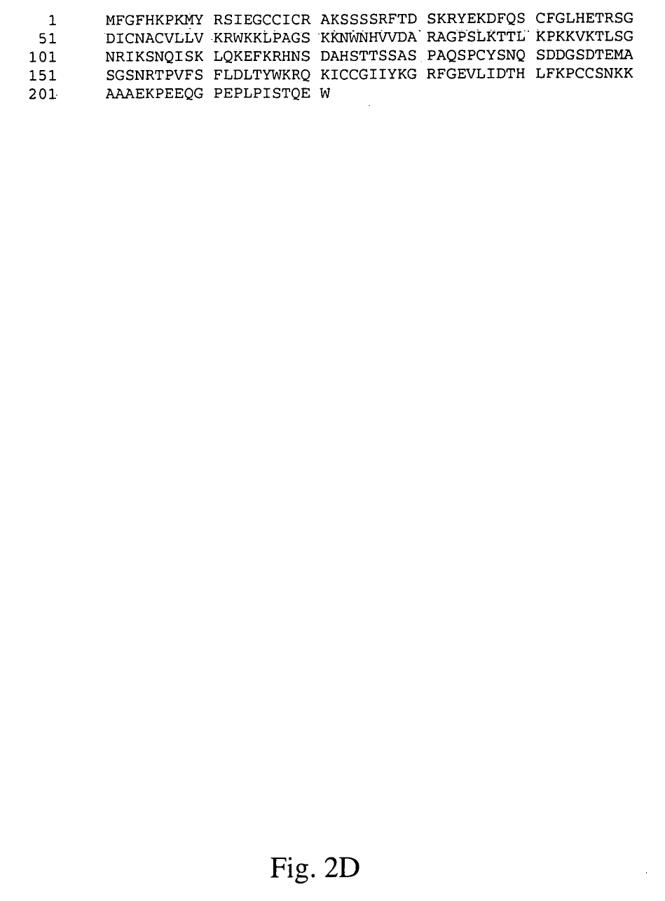

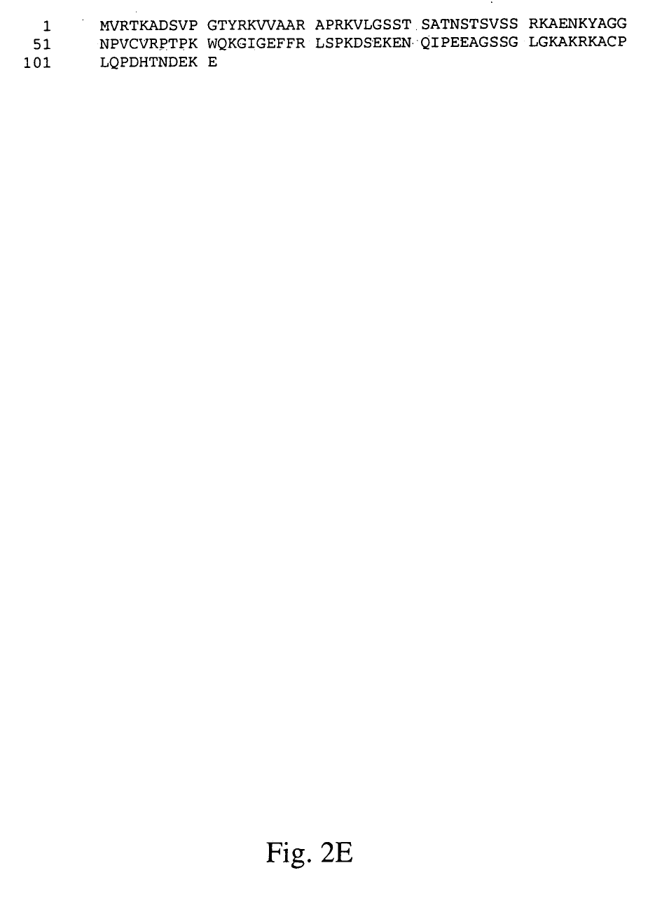

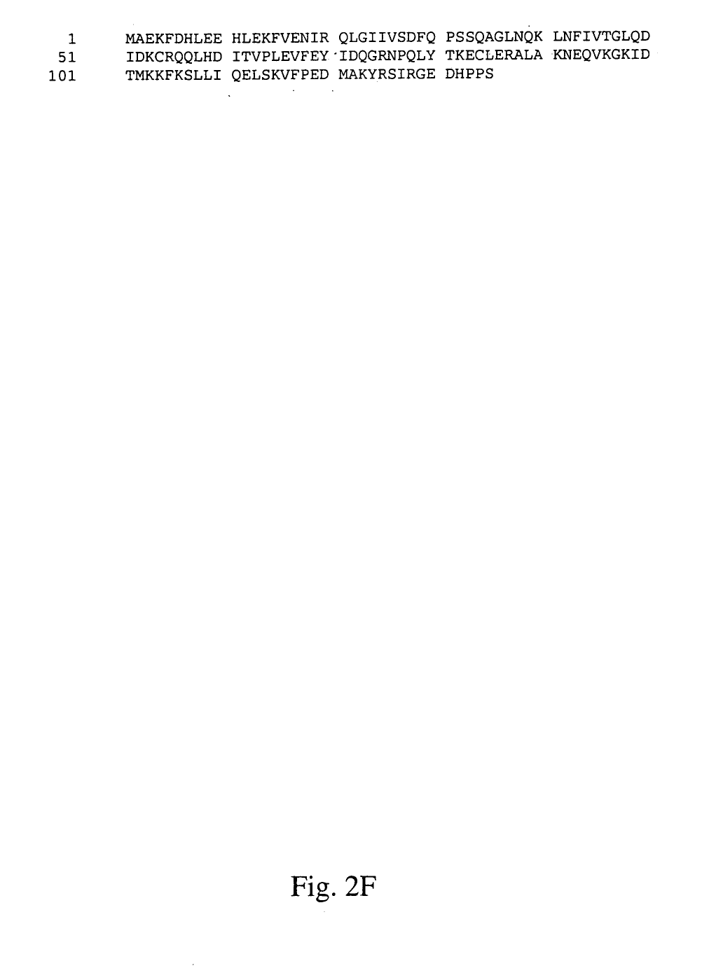

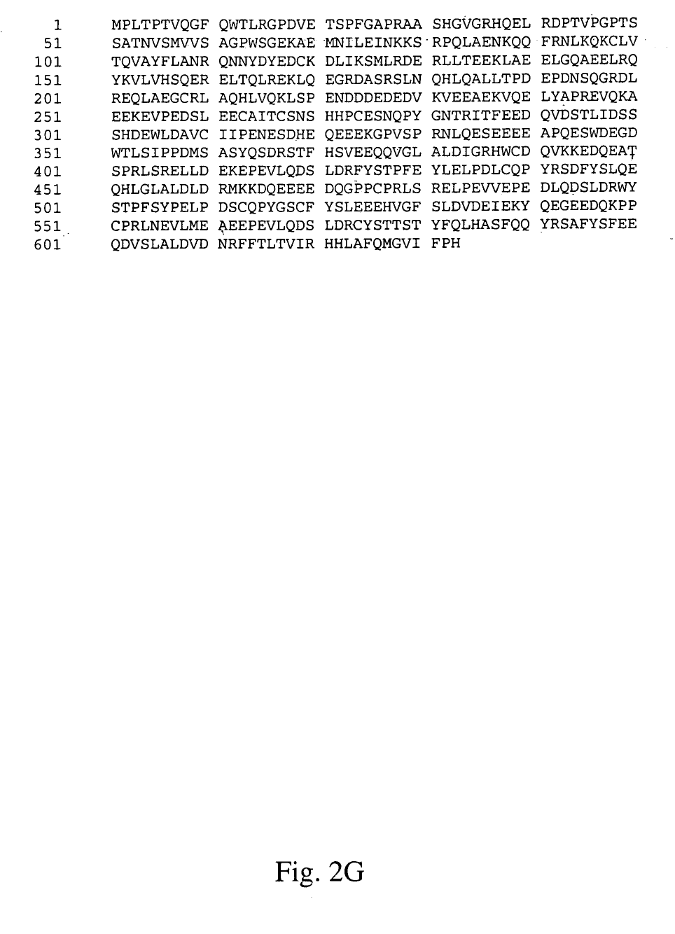

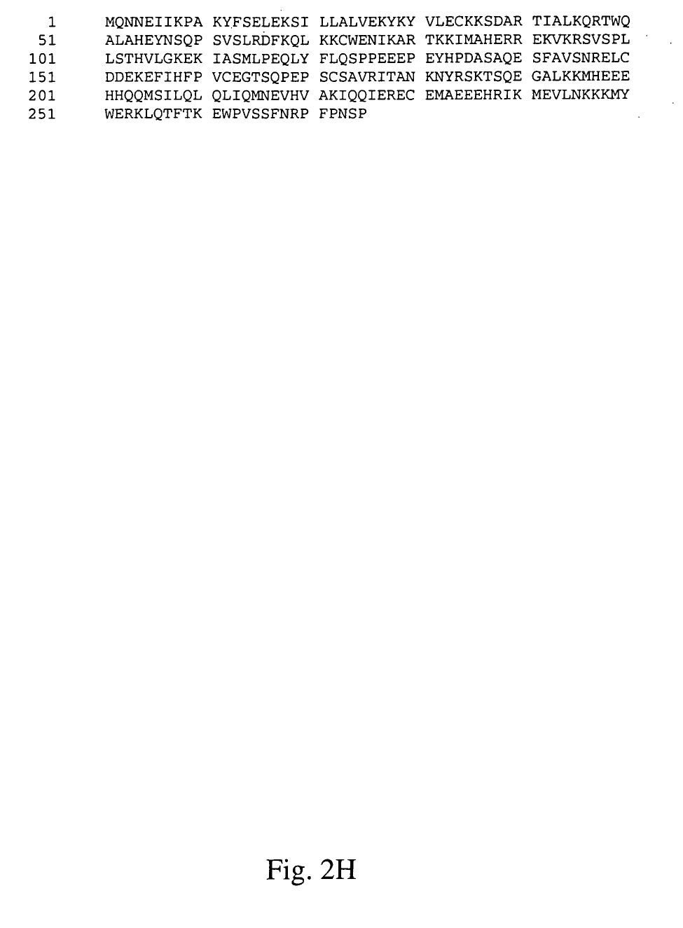

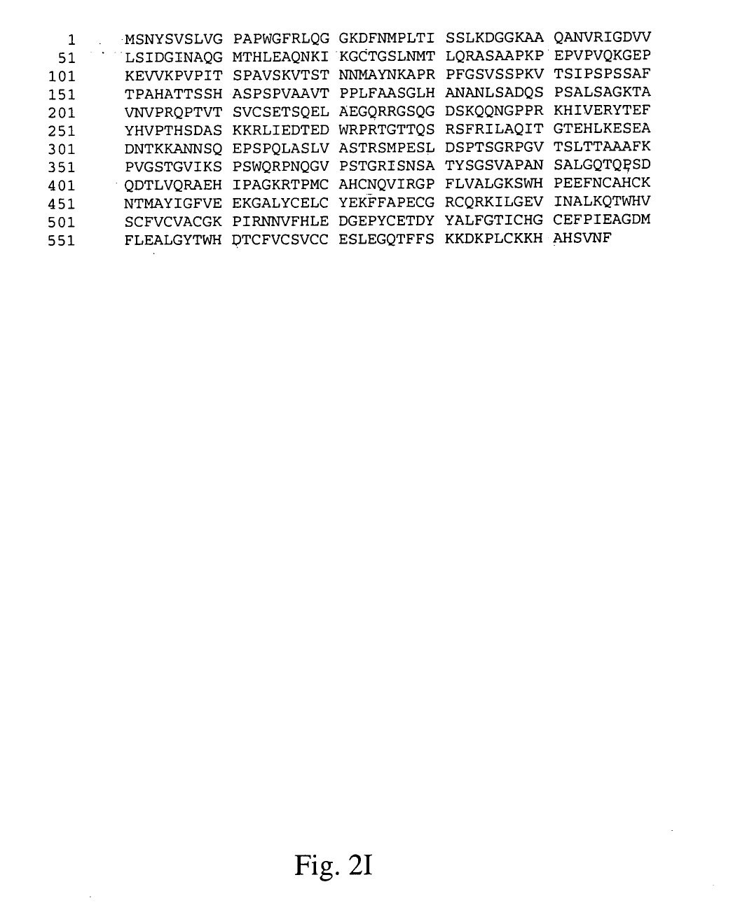

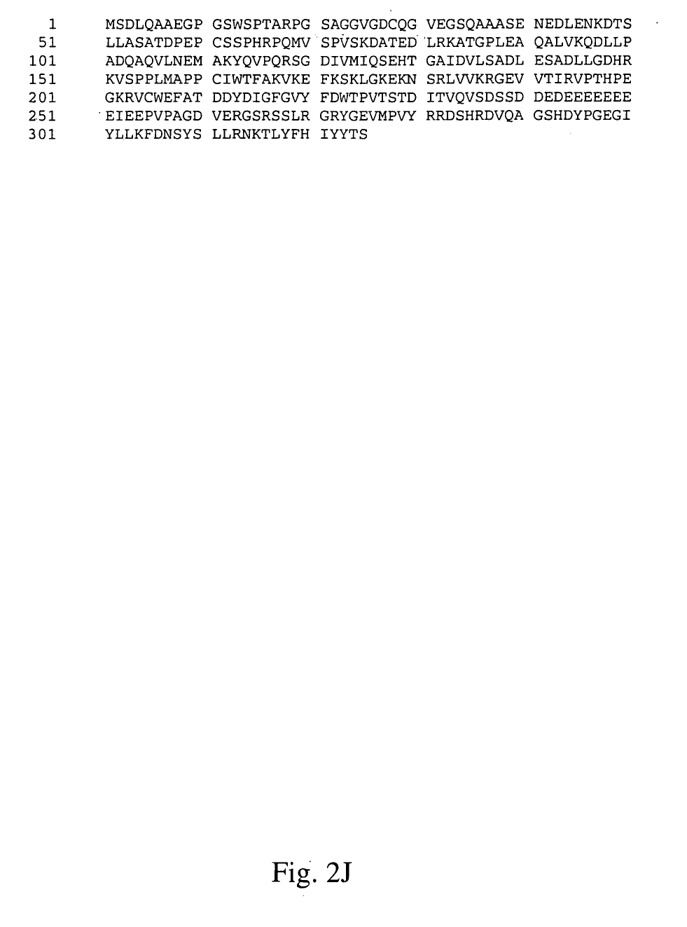

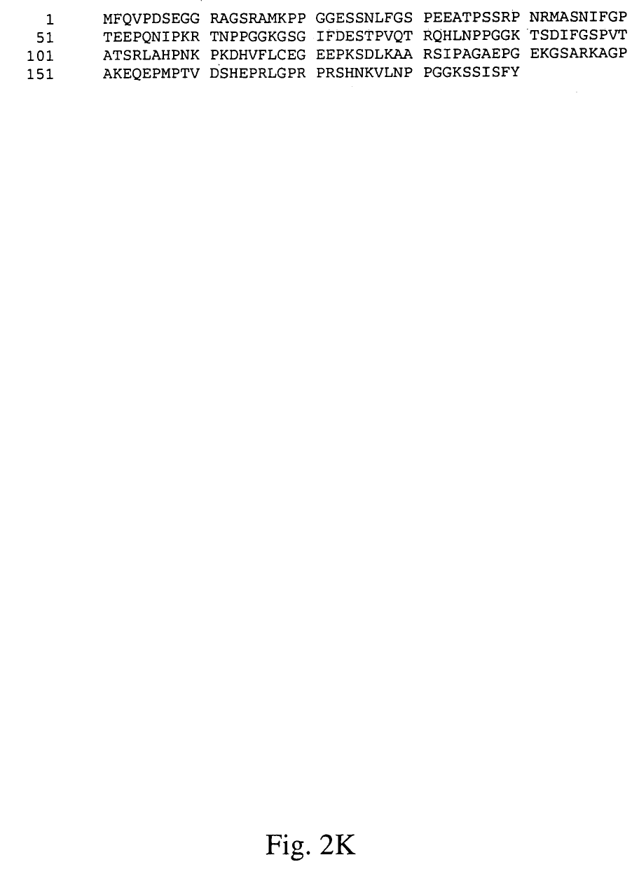

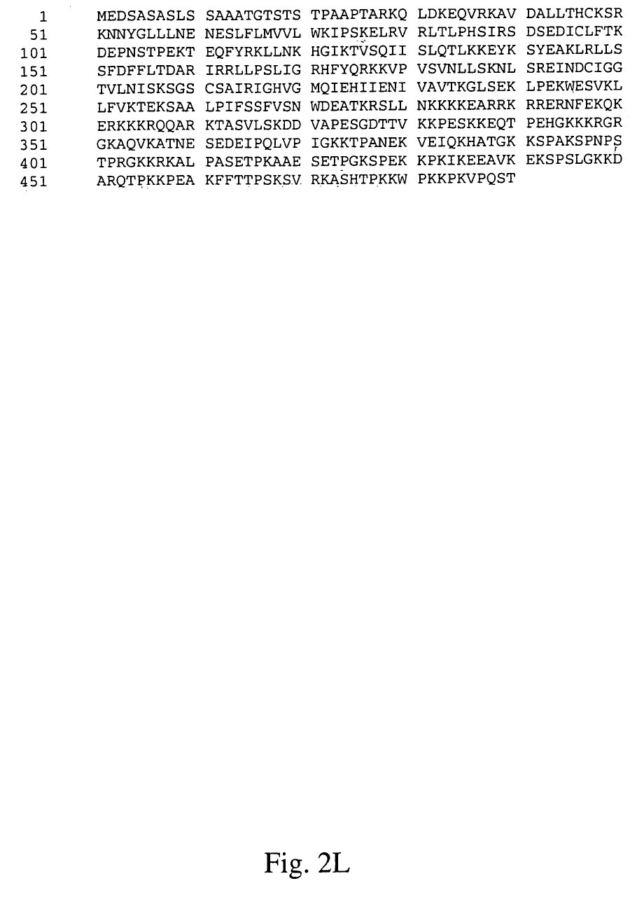

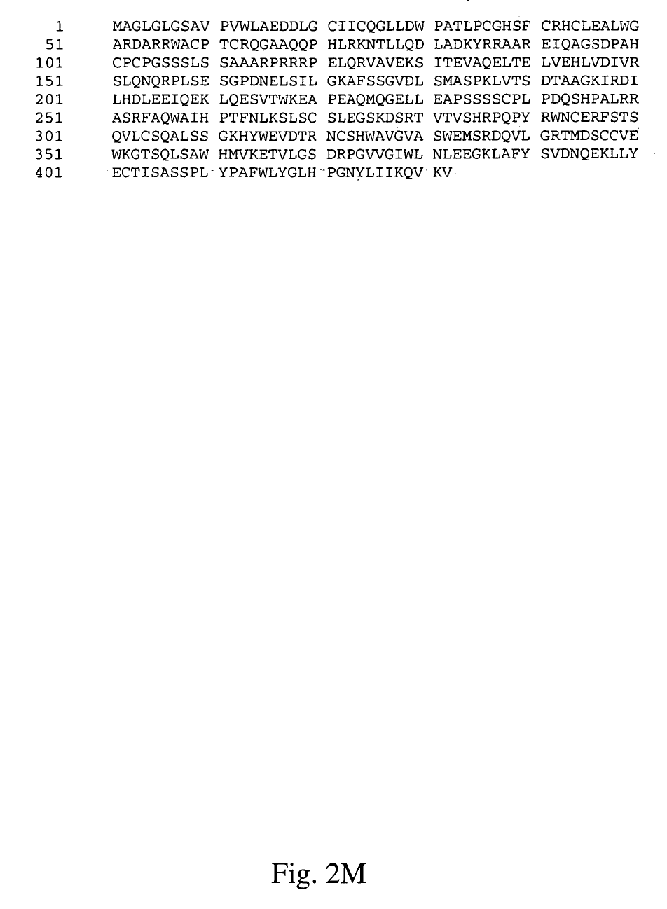

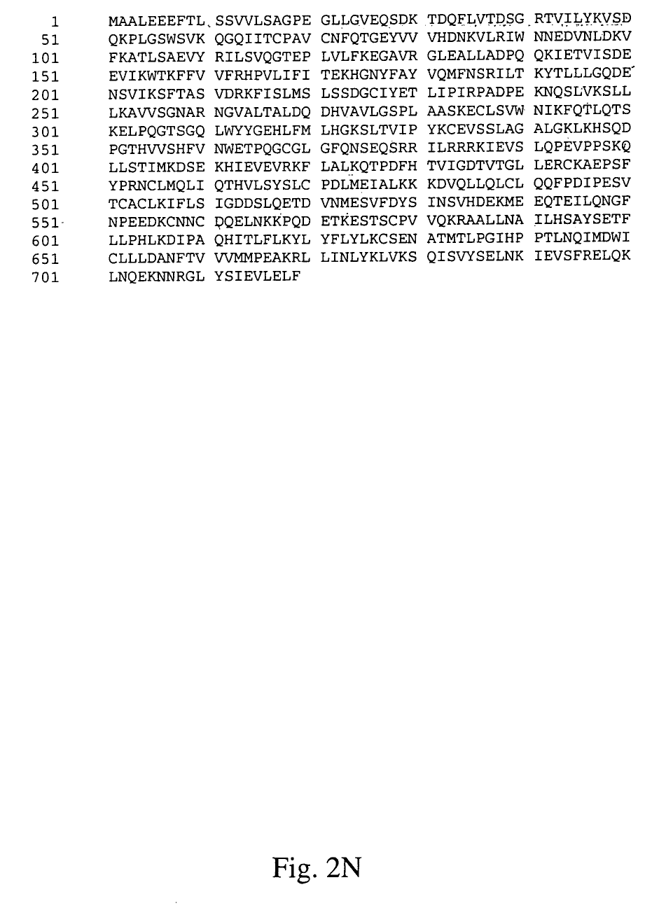

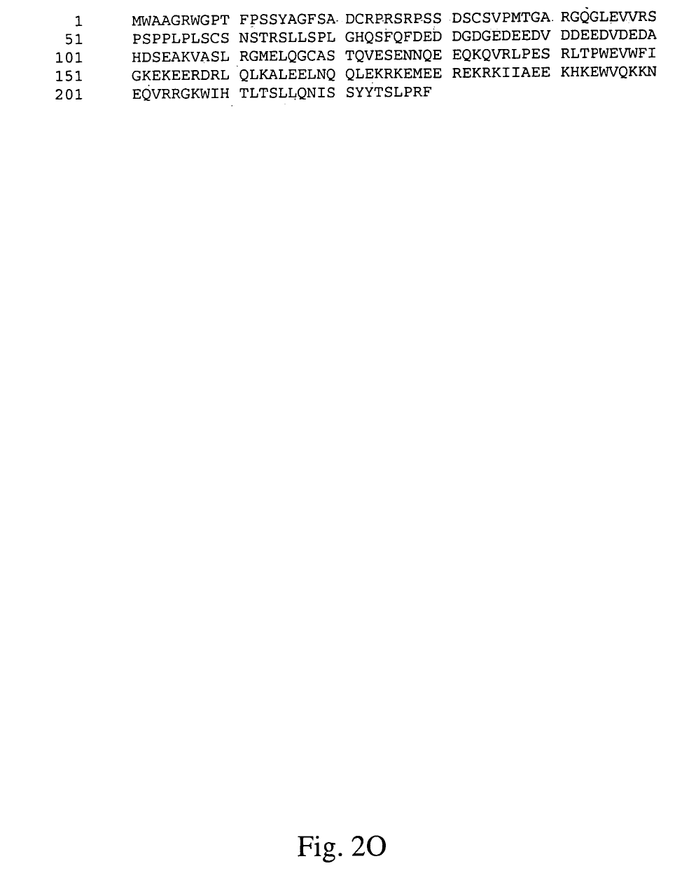

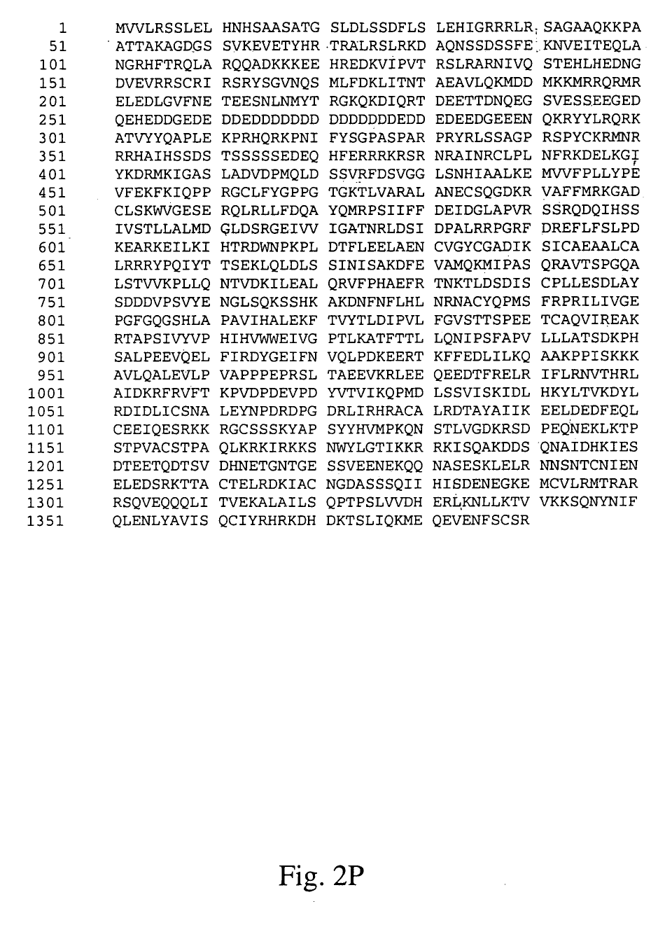

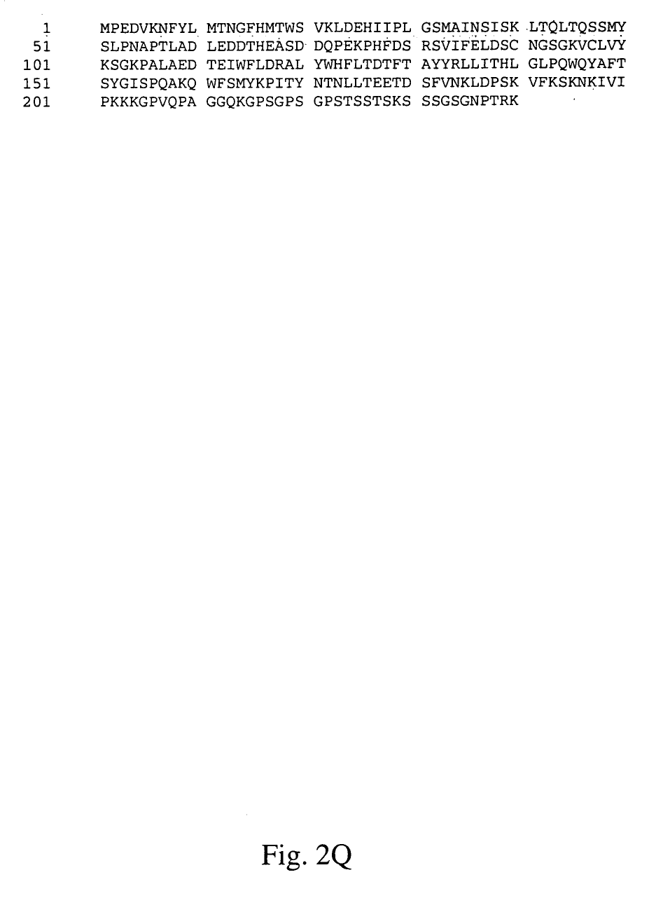

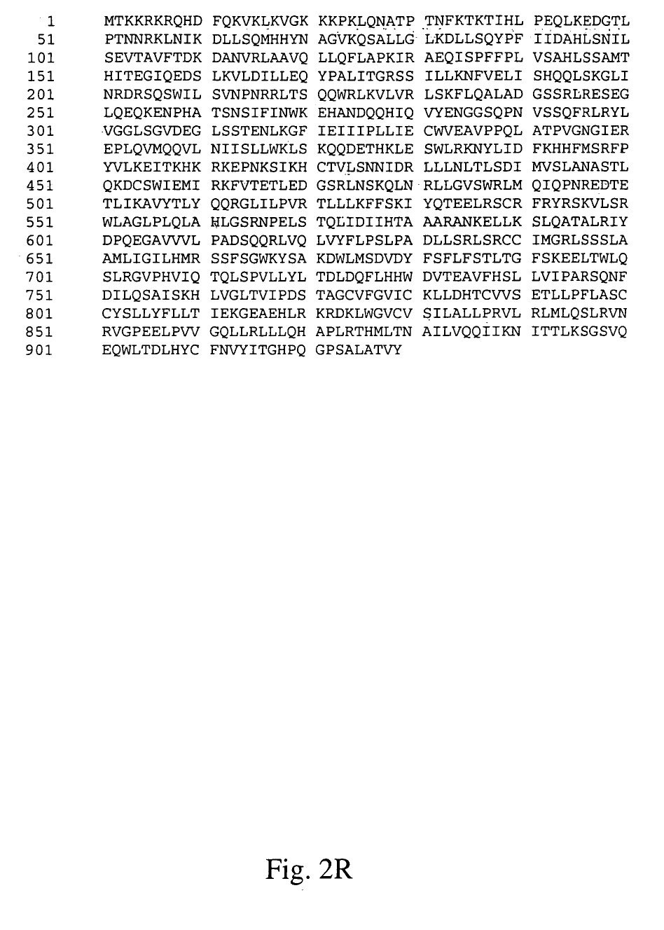

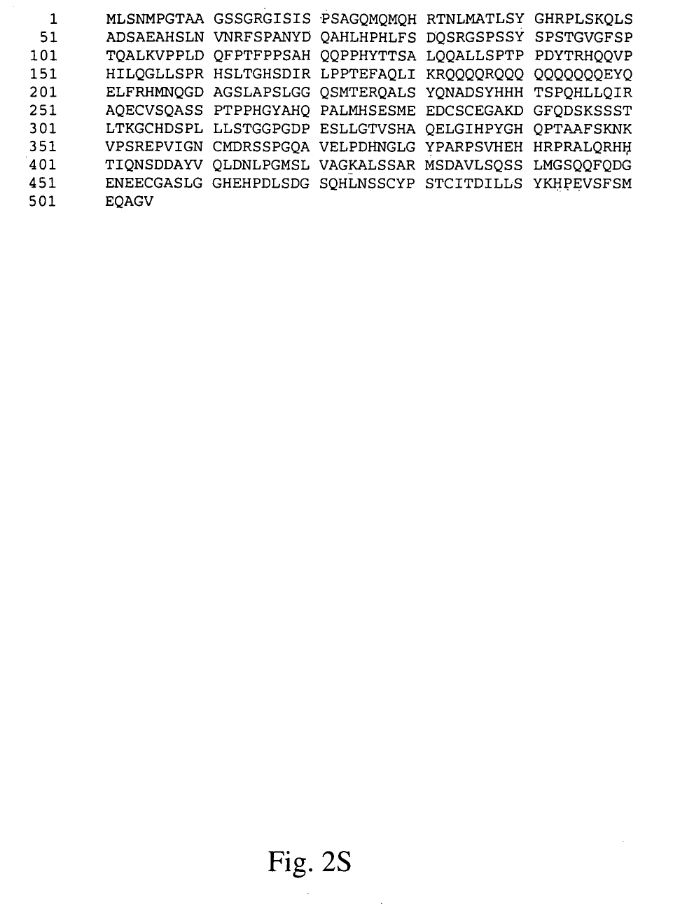

L gene nucleic acids used in the present invention include but are not limited to (a) a DNA comprising the DNA sequence shown in one of FIGS. 1A-S (SEQ ID NOs: 1-19), or a complement thereof; (b) any DNA sequence that hybridizes to one of these DNA sequences, or a complement thereof, that encodes one of the amino acid sequences shown in FIGS. 2A-S, under low, moderate or highly stringent conditions, as disclosed infra herein below. In a specific embodiment, nucleic acids used in the invention encode a gene product that comprises at least one conservative or silent substitution. The encoded proteins are also provided for use. Additional molecules that can be used in the invention include, but are not limited to, protein derivatives that comprise at least one substitution, addition or deletion, and nucleic acid sequences encoding such protein derivatives. Due to the degenerate nature of the nucleotide coding sequences, other DNA sequences that encode substantially the same amino acid sequence as an L gene or cDNA can be used in the practice of the present invention. These include but are not limited to nucleotide sequences comprising all or portions of the an L nucleic acid sequence that are altered by the substitution of at least one different codon that encodes the same amino acid residue observed in the wild type or native sequence; such substitutions or mutations are, therefore, silent with regard to the the amino acid sequence encoded therefrom. Likewise, the derivatives of the invention include, but are not limited to, those containing, as a primary amino acid sequence, all or part of the amino acid sequence of a component protein, including altered sequences in which functionally equivalent amino acid residues are substituted for residues within the sequence resulting in a silent change with respect to function. For example, one or more amino acid residues within the sequence can be substituted by another amino acid of a similar polarity (a “conservative amino acid substitution”) that acts as a functional equivalent, resulting in a conservative alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

The invention includes the use of any one of the L gene coding sequences which preferably hybridize under highly stringent or moderately stringent conditions as described infra herein below to at least about 6, preferably about 12, more preferably about 18 consecutive nucleotides of an L gene sequence as being useful for the detection of an L gene product for the diagnosis and prognosis of cancer, e.g., an RNA corresponding to one of SEQ ID NOs: 1-19, or a nucleic acid derived therefrom; a nucleic acid comprising a sequence hybridizable to one of SEQ ID NOs: 1-19, or a complement thereof under conditions of high stringency; a nucleic acid comprising a sequence hybridizable to one of SEQ ID NOs: 1-19, or a complement thereof under conditions of high stringency; a nucleic acid at least 90% homologous one of SEQ ID NOs: 1-19, or a complement thereof as determined using the NBLAST algorithm.

The invention also includes the use of nucleic acid molecules, preferably DNA molecules, that preferably hybridize under highly stringent or moderately stringent conditions as described herein below to one of the L nucleic acid sequences described herein, which are inverse complements of these L molecules. These nucleic acid molecules may encode or act as L gene coding sequence antisense molecules useful, for example, in L gene regulation. With respect to L gene regulation, such techniques can be used to modulate, for example, the phenotype and metastatic potential of lung cancer or other cancer cells. Further, such sequences may be used as part of ribozyme and/or triple helix sequences, also useful for L gene regulation and thus may be used for the treatment and/or prevention of cancer.

In one embodiment, the invention encompasses methods of using an L gene coding sequence or fragments and degenerate variants of DNA sequences which encode an L gene product, including naturally occurring and non-naturally occurring variants thereof. A non-naturally occurring variant is one that is engineered by man. A naturally occurring L gene, gene product, or variant thereof is one that is not engineered by man. In the methods of the invention wherein an L gene product in a sample derived from a subject is detected or measured, naturally occurring L gene products are detected, including, but not limited to wild-type L gene products as well as mutants, allelic variants, splice variants, polymorphic variants, etc. In general, such mutants and variants are believed to be highly homologous to one of SEQ ID NOs: 1-19, or at least 90% homologous and/or hybridizable under high stringency conditions. In specific embodiments, the mutants and variants being detected or measured may comprise not more than 1, 2, 3, 4, or 5 point mutations (substitutions) relative to one of SEQ ID NOs: 1-19 for nucleic acid sequences or relative to one of SEQ ID NOs: 20-38 for amino acid sequences. Such nucleic or amino acid sequences may encode or comprise silent and/or conservative amino acid substitutions with respect to a wild type L molecule.

In other methods of the invention, wild-type, or naturally occurring variant, or non-naturally occurring variant L sequences may be used in the methods of the invention (e.g., in vaccination, immunization, antisense, or ribozyme procedures).

An L gene fragment may be a complementary DNA (cDNA) molecule or a genomic DNA molecule that may comprise one or more intervening sequences or introns, as well as regulatory regions located beyond the 5′ and 3′ ends of the coding region or within an intron.

The present invention provides methods encode for using isolated nucleic acid molecules encoding an L protein, polypeptide, or fragments, derivatives, and variants thereof that include, both naturally occurring and non-naturally occurring variants or mutants. The invention also contemplates, for use in the methods of the invention, the use of 1) any nucleic acid that encodes an L polypeptide of the invention; 2) any nucleic acid that hybridizes to a complement of one of the sequences disclosed herein, preferably under highly stringent conditions as disclosed herein below, and encodes a functionally equivalent gene product; and/or 3) any nucleic acid sequence that hybridizes to the complement of the sequences disclosed herein, preferably under moderately stringent conditions, as disclose herein below and which still encodes a gene product that displays a functional activity of L.

As discussed above, the invention also contemplates the use of isolated nucleic acid molecules that encode a variant protein or polypeptide. The variant protein or polypeptide can occur naturally or non-naturally. It can be engineered by introducing nucleotide substitutions, e.g., point mutations, or additions or deletions into a nucleotide sequence of one of SEQ ID NOs: 1-19. In a specific embodiment, one or more, but not more than 5, 10, or 25 amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. Following mutagenesis, the encoded protein can be expressed recombinantly and the activity of the protein can be determined.

In a specific embodiment, the invention provides for the use of L molecule derivatives and analogs of the invention which are functionally active, i.e., they are capable of displaying one or more known functional activities associated with a (wild-type) L encoded protein. Such functional activities include but are not limited to antigenicity/immunogenicity (ability to bind or compete with an L molecule for binding to an anti-L molecule antibody or ability to generate antibody which binds to an L molecule), ability to bind or compete with an L molecule for binding to other proteins or fragments thereof, such as proteins capable of forming complexes with an L molecule (i.e., L molecule binding partners).

Using all or a portion of one of the nucleic acid sequences of SEQ ID NOs: 1-19, or a portion thereof, as a hybridization probe, nucleic acid molecules encoding an L gene product can be isolated using standard hybridization and cloning techniques. See, e.g., Sambrook et al., Molecular Cloning: A Laboratory Manual, 2nd ed., Cold Spring Harbor Laboratory, Cold Spring Harbor Laboratory Press, Cold Spring Harbor, N.Y., 1989) for use in the methods of the invention.

In addition, gene products encoded by an L gene, including L protein peptide fragments, as well as specific or selective antibodies thereto, can be used for construction of fusion proteins to facilitate recovery, detection, or localization of another protein of interest. In addition, L genes (e.g., L1-19) and gene products encoded by an L gene (e.g., L20-38) can be used as research reagents, e.g., for genetic mapping.

Additionally, the present invention contemplates use of the nucleic acid molecules, polypeptides, and/or antagonists or agonists of gene products encoded by one of the L genes to screen, diagnose, prevent and/or treat disorders characterized by aberrant expression or activity of an L polypeptide, which include, cancers, such as but not limited to cancer of the lung, breast, pancreas, prostate, ovary, brain, and gastric system.

The present invention encompasses the use of L nucleic acid molecules comprising cDNA, genomic DNA, introns, exons, promoter regions, 5′ and 3′ regulatory regions of the gene, RNA, hnRNA, mRNA, regulatory regions within RNAs, and degenerate variants thereof in the methods of the invention. Promoter sequences for an L gene can be determined by promoter-reporter gene assays and in vitro binding assays.

In one embodiment, the invention comprises the use of a variant L gene nucleic acid sequence that hybridizes to a naturally-occurring or non-naturally occurring variant an L nucleic acid molecule under stringent conditions as described herein below. In another embodiment, the invention contemplates the use of an L gene variant nucleic acid sequence that hybridizes to a naturally-occurring or non-naturally occurring variant L nucleic acid molecule under moderately stringent conditions as described herein below.

A nucleic acid molecule is intended to include DNA molecules (e.g., cDNA, genomic DNA), RNA molecules (e.g., hnRNA, pre-mRNA, mRNA), and DNA or RNA analogs generated using nucleotide analogs. The nucleic acid molecule can be single-stranded or double-stranded.

L sequences used in the methods of the invention are of human origin, however, homologs of an L gene isolated from other mammals may also be used in the methods of the invention. Thus, the invention also includes the use of L gene homologs isolated from non-human animals such as: non-human primates; rats; mice; farm animals including, but not limited to: cattle; horses; goats; sheep; pigs; etc.; household pets including, but not limited to: cats; dogs; etc. in the methods of the invention.

Still further, such molecules may be used as components of diagnostic and/or prognostic methods whereby, for example, the presence of a particular L gene allele or alternatively spliced L gene transcript responsible for causing or predisposing a person to lung cancer or other cancer may be detected.

The invention also includes the use of transcriptional regulators that control the level of expression of an L gene product. A transcriptional regulator can include, e.g., a protein which binds a DNA sequence and which up-regulates or down-regulates the transcription of an L gene. A transcriptional regulator can also include a nucleic acid sequence that can be either upstream or downstream from an L gene and which binds an effector molecule that enhances or suppresses L gene transcription.

Still further, the invention encompasses the use of L gene coding sequences or fragments thereof as a screen in an engineered yeast system, including, but not limited to, the yeast two hybrid system as a method to identify proteins, peptides or nucleic acids related to the onset and/or metastatic spread of cancer, including lung cancer.

The invention also encompasses the use of (a) DNA vectors comprising any of the foregoing L gene coding sequences and/or their complements (e.g., antisense); (b) DNA expression vectors comprising any of the foregoing L gene coding sequences operatively associated with or operably linked to a regulatory element that directs the expression of the coding sequences; and (c) genetically engineered host cells comprising any of the foregoing L gene coding sequences operatively associated with a regulatory element that directs the expression of the coding sequences in the host cell. Cell lines and/or vectors which comprise and/or express an L gene or fragment thereof can be used to produce an L gene product for use in the methods of the invention, e.g., vaccination against lung cancer or other cancers in which expression of an L molecule is up-regulated or elevated and screening assays for antagonists and agonists that bind, or interact with an L molecule or suppress or enhance expression of an L molecule.

As used herein, regulatory elements include, but are not limited to inducible and non-inducible promoters, enhancers, operators and other elements known to those skilled in the art that drive and regulate expression. Such regulatory elements include but are not limited to the cytomegalovirus (hCMV) immediate early promoter, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention includes the use of fragments or derivatives of any of the nucleic acids disclosed herein in any of the methods of the invention. In various embodiments, a fragment or derivative comprises 10, 20, 50, 100, or 200 nucleotides of one of SEQ ID NOs: 1-19 or encodes all or a fragment of one of SEQ ID NOs: 20-38. In the same or alternative embodiments, a nucleic acid is not more than 500 to 10,000 nucleotides in size.

In addition to the use of L gene sequences as described above, homologs of such sequences, exhibiting extensive homology to an L gene product present in other species can be identified and readily isolated, and used in the methods of the invention without undue experimentation, by molecular biological techniques well known in the art. Further, there can exist homologous genes at other genetic loci within the genome that encode proteins that have extensive homology to an L protein. Alternatively, such homologous genes can encode a single protein with homology to an L protein. These genes can also be identified via similar techniques and used in the methods of the invention. Still further, there can exist alternatively spliced variants of an L gene. The invention thus includes the use of any of these homologs in the methods of the invention.

As an example, in order to clone a mammalian L gene ortholog or homolog or variants using isolated human L gene sequences as disclosed herein, such human L gene sequences are labeled and used to screen a cDNA library constructed from mRNA obtained from appropriate cells or tissues (e.g., bronchial epithelial cells) derived from the organism of interest. With respect to the cloning of such a mammalian L ortholog or homolog, a mammalian lung cancer cell cDNA library may, for example, be used for screening. In one embodiment, such a screen employs a probe corresponding to all or a portion of an L gene open reading frame. In yet another embodiment, such a screen would employ one or more probes corresponding to all or a portion of the coding sequence for an L gene (e.g., SEQ ID NO: 1). The hybridization and wash conditions used should be of a low stringency, as described herein below when the cDNA library is derived from a different type of organism than the one from which the labeled sequence was derived. Alternatively, the labeled fragment may be used to screen a genomic library derived from an organism of interest, again, using appropriately stringent conditions well known to those of skill in the art.

Further, an L gene otholog or homolog may be isolated from nucleic acid of an organism of interest by performing PCR using two degenerate oligonucleotide primer pools designed on the basis of amino acid sequences of an L protein. The template for the reaction may be cDNA obtained by reverse transcription of either total RNA or mRNA prepared from, for example, mammalian cell lines or tissue known or suspected to express an L gene allele, homolog, or ortholog.

The PCR product may be subcloned and sequenced to ensure that the amplified sequences represent the sequences of an L-related nucleic acid sequence. The PCR fragment may then be used to isolate a cDNA clone of an L-related nucleic acid sequence by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may be utilized to isolate cDNA sequences. For example, RNA may be isolated, following standard procedures, from an appropriate cellular or tissue source (e.g., one known, or suspected, to express an L gene, such as, for example, lung cancer cell lines). A reverse transcription reaction may be performed on the RNA using an oligonucleotide primer specific or selective for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of PCR technology and cloning strategies which may be used, see, e.g., PCR Primer, 1995, Dieffenbach et al., ed., Cold Spring Harbor Laboratory Press; Sambrook et al., 1989, supra.

L gene coding sequences may additionally be used to isolate L gene alleles and mutant L gene alleles. Such mutant alleles may be isolated from individuals either known or susceptible to or predisposed to have a genotype that contributes to the development of cancer, e.g., lung cancer, including metastasis. Such mutant alleles may also be isolated from individuals either known or susceptible to or predisposed to have a genotype that contributes to resistance to the development of cancer, e.g., lung cancer, including metastasis. Mutant alleles and mutant allele products may then be utilized in the screening, therapeutic and diagnostic methods and systems described herein. Additionally, such L gene sequences can be used to detect L gene regulatory (e.g., promoter) defects that can affect the development and outcome of cancer. Mutants can be isolated by any technique known in the art, e.g., PCR, screening genomic libraries, screening expression libraries.

As described below, the invention also relates to the use of an L gene coding sequence or gene product in the methods of the invention. An L gene coding sequence or gene product includes, but is not limited to an RNA corresponding to one of SEQ ID NOs: 1-19, a nucleic acid derived therefrom, a protein comprising one of SEQ ID NOs: 20-38, or a nucleic acid comprising a sequence hybridizable to one of SEQ ID NOs: 1-19, under conditions of high stringency, or a protein comprising a sequence encoded by said hybridizable sequence or a nucleic acid at least 90% homologous to one of SEQ ID NOs: 1-19, as determined using the NBLAST algorithm or a protein encoded thereby.

Hybridization Conditions

A nucleic acid which is hybridizable to an L gene nucleic acid (e.g., having a sequence as set forth in one of SEQ ID NOs: 1-19, or a reverse complement thereof, or to a nucleic acid encoding an L derivative, or a reverse complement thereof) under conditions of low stringency can be used in the methods of the invention to detect the presence of an L gene and/or presence or expression level of an L gene product. By way of example and not limitation, procedures using such conditions of low stringency are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×1⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

A nucleic acid which is hybridizable to an L nucleic acid (e.g., having a sequence as set forth in one of SEQ ID NOs: 1-19, or a reverse complement thereof, or to a nucleic acid encoding an L derivative, or a reverse complement thereof) under conditions of high stringency is also provided for use in the methods of the invention. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.01×SSC at 50° C. for 45 min before autoradiography. Other conditions of high stringency that may be used are well known in the art.

A nucleic acid which is hybridizable to an L nucleic acid (e.g., having a sequence as set forth in one of SEQ ID NOs: 1-19, or a reverse complement thereof, or to a nucleic acid encoding an L derivative, or a reverse complement thereof) under conditions of moderate stringency is also provided for use in the methods of the invention. For example, but not limited to, procedures using such conditions of moderate stringency are as follows: filters comprising immobilized DNA are pretreated for 6 hours at 5520 C. in a solution containing 6×SSC, 5× Denhardt'solution, 0.5% SDS and 100 μg/ml denatured salmon sperm DNA. Hybridizations are carried out in the same solution with 5-20×10⁶ cpm ³²P-labeled probe. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Washing of filters is done at 37° C. for 1 hour in a solution containing 2×SSC, 0.1 % SDS. Other conditions of moderate stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

Protein Products of L Genes

In another embodiment, the present invention provides for the use of L gene products, including, for example, L1, or peptide fragments thereof which can be used for the generation of antibodies, in diagnostic assays, or for the identification of other cellular gene products involved in the development of cancer, such as, for example, lung cancer.

The amino acid sequences depicted in FIGS. 2A-S represent examples of L gene products (SEQ ID NOs: 20-38), e.g., L1 (SEQ ID NO: 20). L1 gene products, for example, sometimes referred to herein as an “L1 protein” or “L1polypeptide,” may additionally include those gene products encoded by an L1 gene sequence (SEQ ID NO: 1) described hereinabove.

In addition, L protein derivatives may include proteins that have conservative amino acid substitution(s) and/or display a functional activity of an L gene product. Such a derivative may comprise deletions, additions or substitutions of amino acid residues within the amino acid sequence encoded by an L gene sequence described herein above, but which result in a silent change, thus producing a functionally equivalent L gene product.

In a specific embodiment, the invention provides a functionally equivalent protein that exhibits a substantially similar in vivo activity as an endogenous L gene product encoded by an L gene sequence described herein above. An in vivo activity of an L gene product can be exhibited by, for example, preneoplastic and/or neoplastic transformation of a cell upon overexpression of the gene product, such as for example, may occur in the onset and progression and metastasis of lung cancer. An L gene product sequence preferably comprises an amino acid sequence that exhibits at least about 65% sequence similarity to an L protein, more preferably exhibits at least 70% sequence similarity to an L protein, yet more preferably exhibits at least about 75% sequence similarity to an L protein. In other embodiments, an L gene product sequence preferably comprises an amino acid sequence that exhibits at least 85% sequence similarity to an L protein, yet more preferably exhibits at least 90% sequence similarity to an L protein, and most preferably exhibits at least about 95% sequence similarity to an L protein.

The determination of percent identity between two sequences can be accomplished using a mathematical algorithm. A preferred, non-limiting example of a mathematical algorithm utilized for the comparison of two sequences is the algorithm of Karlin and Altschul (1990) Proc Natl Acad Sci. 87:2264-2268, modified as in Karlin and Altschul (1993) Proc Natl Acad Sci. 90:5873-5877. Such an algorithm is incorporated into the NBLAST and XBLAST programs of Altschul et al. (1990) J. Mol. Biol. 215:403-410. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search that detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2. The percent identity between two sequences can be determined using techniques similar to those described above, with or without allowing gaps. In calculating percent identity, only exact matches are counted. However, conservative substitutions should be considered in evaluating sequences that have a low percent identity with an L sequence of the present invention.

In a specific embodiment, molecules or protein comprising at least 10, 20, 30, 40 or 50 amino acids of one of SEQ ID NOs: 20-38, or at least 10, 20, 30, 40, 50, 75, 100, or 200 amino acids of one of SEQ ID NOs: 20-38 are used in the present invention.

Fusion Proteins

L gene products also include fusion proteins comprising an L gene product sequence as described above operatively associated with or operably linked to a heterologous, component, e.g., peptide for use in the methods of the invention. Heterologous components can include, but are not limited to sequences that facilitate isolation and purification of fusion protein, or label components. Heterologous components can also include sequences that confer stability to an L gene product. Such isolation and label components are well known to those of skill in the art.

The present invention encompasses the use of fusion proteins comprising a protein or fragment thereof encoded by an L gene open reading frame such as one of SEQ ID NOs: 1-19 operably linked to a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The fusion can be direct, but may occur through linker sequences. The heterologous polypeptide may be fused to the N-terminus or C-terminus of an L gene product.

A fusion protein can comprise an L gene product fused to a signal sequence at its N-terminus. Various signal sequences are commercially available. Eukaryotic heterologous signal sequences include, but are not limited to, the secretory sequences of honeybee melittin (Invitrogen Corporation; Carlsbad, Calif.) and human placental alkaline phosphatase (Stratagene; La Jolla, Calif.). Prokaryotic heterologous signal sequences useful in the methods of the invention include, but are not limited to, the phoA secretory signal (Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989) and the protein A secretory signal (Pharmacia Biotech; Piscataway, N.J.).

An L protein or fragment thereof encoded by an L open reading frame such as one of SEQ ID NOs: 1-19 or a fragment thereof can be fused to nucleic acid sequences encoding a tag sequence, e.g., a hexa-histidine peptide, such as the tag provided in a pQE vector (QIAGEN, Inc., Chatsworth, Calif., 91311). Additional tag moieties are commercially available and may be used to advantage to in the methods of the invention. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification and detection of recombinantly produced polypeptides of the invention.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

An affinity label can also be fused at its amino terminal to the carboxyl terminal of the protein or fragment thereof encoded by an L gene open reading frame such as one of SEQ ID NOs: 1-19 for use in the methods of the invention. The precise site at which the fusion is made in the carboxyl terminal is not critical. The optimal site can be determined by routine experimentation. An affinity label can also be fused at its carboxyl terminal to the amino terminal of an L gene product for use in the methods of the invention.

A variety of affinity labels known in the art may be used, such as, but not limited to, the immunoglobulin constant regions, (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an L gene product, e.g., green fluorescent protein and the like. Other affinity labels are recognized by specific binding partners and thus facilitate isolation by affinity binding to the binding partner that can be immobilized onto a solid support. Some affinity labels may afford the L gene product novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when an L gene product is intended for in vivo use in humans. Many DNA encoding immunoglobulin light or heavy chain constant regions are known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, an L gene product-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate an L gene product containing the affinity label. In many instances, there is no need to develop specific or selective antibodies to an L gene product.

A fusion protein can comprise an L gene product fused to the Fc domain of an immunoglobulin molecule or a fragment thereof for use in the methods of the invention. A fusion protein can also comprise an L gene product fused to the CH2 and/or CH3 region of the Fc domain of an immunoglobulin molecule. Furthermore, a fusion protein can comprise an L gene product fused to the CH2, CH3, and hinge regions of the Fc domain of an immunoglobulin molecule (see Bowen et al., 1996, J. Immunol. 156:442-49). This hinge region contains three cysteine residues that are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of an L gene product from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting L gene product expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (Maclntyre et al., 1990, Mol. Gen. Genet. 221:466-74; Kaiser et al., 1987, Science, 235:312-317).

A fusion protein can comprise an L gene product and a cell permeable peptide, which facilitates the transport of a protein or polypeptide across the plasma membrane for use in the methods of the invention. Examples of cell permeable peptides include, but are not limited to, peptides derived from hepatitis B virus surface antigens (e.g., the PreS2-domain of hepatitis B virus surface antigens), herpes simplex virus VP22, antennapaedia, 6H, 6K, and 6R. See, e.g., Oess et al., 2000, Gene Ther. 7:750-758, DeRossi et al., 1998, Trends Cell Biol 8(2):84-7, and Hawiger, 1997, J. Curr Opin Immunol 9(2): 189-94.

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector that contains the necessary elements for the transcription and translation of the inserted protein-coding sequence. The expression of a fusion protein may be regulated by a constitutive, inducible, tissue-specific, or selective promoter. It will be understood by the skilled artisan that fusion proteins, which can facilitate solubility and/or expression, and can increase the in vivo half-life of an L protein or fragment thereof (such as one of SEQ ID NOs: 20-38) and thus are useful in the methods of the invention. L gene products or peptide fragments thereof, or fusion proteins can be used in any assay that detects or measures L gene products or in the calibration and standardization of such assays.

The methods of the invention encompass the use of L gene products or peptide fragments thereof, which may be produced by recombinant DNA technology using techniques well known in the art. Thus, methods for preparing L gene polypeptides and peptides of the invention by expressing nucleic acid containing L gene sequences are described herein. Methods that are well known to those skilled in the art can be used to construct expression vectors containing L gene product coding sequences including but not limited to one of SEQ ID NOs: 1-19 and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding L gene product sequences may be chemically synthesized using, for example, synthesizers (see e.g., the techniques described in Oligonucleotide Synthesis, 1984, Gait, M. J. ed., IRL Press, Oxford).

Expression Systems

A variety of host-expression vector systems may be utilized to express L gene coding sequences for use in the methods of the invention. Such host-expression systems represent vehicles by which the coding sequences of interest may be produced and subsequently purified, but also represent cells which may, when transformed or transfected with the appropriate nucleotide coding sequences, express an L gene product of the invention in situ. These include but are not limited to microorganisms such as bacteria (e.g., E. coli, B. subtilis) transformed with recombinant bacteriophage DNA, plasmid DNA or cosmid DNA expression vectors comprising L gene product coding sequences; yeast (e.g., Saccharomyces, Pichia) transformed with recombinant yeast expression vectors comprising L gene product coding sequences; insect cell systems infected with recombinant virus expression vectors (e.g., baculovirus) comprising L gene product coding sequences; plant cell systems infected with recombinant virus expression vectors (e.g., cauliflower mosaic virus, CaMV; tobacco mosaic virus, TMV) or transformed with recombinant plasmid expression vectors (e.g., T1 plasmid) comprising L gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for an L gene product being expressed. For example, when a large quantity of such a protein is to be produced for the generation of pharmaceutical compositions of an L protein or for raising antibodies to an L protein, vectors that direct the expression of high levels of fusion protein products that are readily purified may be desirable. Such vectors include, but are not limited, to the E. coli expression vector pUR278 (Ruther et al., 1983, EMBO J. 2:1791), in which an L gene product coding sequence may be ligated into the vector in frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose bead followed by elution in the presence of free glutathione. The pGEX vectors are designed to include, e.g.,thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. An L gene coding sequence may be cloned into a non-essential region (for example the polyhedrin gene) of the virus and placed under control of an AcNPV promoter (for example the polyhedrin promoter). Successful insertion of an L gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, an L gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing an L gene product in infected hosts. (See, e.g., Logan & Shenk, 1984, Proc. Natl. Acad. Sci. USA 81:3655). Specific initiation signals may also be required for efficient translation of inserted L gene product coding sequences. These signals include the ATG initiation codon and adjacent sequences. In cases where an entire L gene, including its own initiation codon and adjacent sequences, is inserted into the appropriate expression vector, no additional translational control signals may be needed. However, in cases where only a portion of an L gene coding sequence is inserted, exogenous translational control signals, including, perhaps, the ATG initiation codon, may be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, and in particular, lung cancer cell lines such as, for example, A549, NCI-H920, NCI-H969, NCI-H23, NCI-H226, NCI-H647, NCI-H1869, NCI-HH1385, NCI-H460, NCI-H1155, NCI-H358, and NCI-H650.

For long-term, high-yield production of recombinant proteins, stable expression is preferred. For example, cell lines that stably express an L gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may be used advantageously to engineer cell lines that express an L gene product. Such engineered cell lines may be particularly useful in the screening and evaluation of compounds that affect the endogenous activity of an L gene product.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., 1980, Proc Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G418 (Colberre-Garapin et al., 1981, J. Mol. Biol. 150: 1); and hygro, which confers resistance to hygromycin (Santerre et al., 1984, Gene 30:147).

L Gene Transgenic Animals

L gene products can also be expressed in transgenic animals. Animals of any species, including, but not limited to, mice, rats, rabbits, guinea pigs, sheep, pigs, micro-pigs, goats, and non-human primates, e.g., baboons, monkeys, and chimpanzees may be used to generate L gene transgenic animals.

Transgenic animals that over- or mis-express an L gene product may be used in any of the methods of the invention. For example transgenic animals may be used to study the in vivo effects of enhanced expression levels of an L gene and the onset, diagnosis or prognosis of cancer. Transgenic animals would be useful for screening compounds to identify antagonists or agonists of an L gene activity. Transgenic animals could be used to screen the in vivo effects of anti-sense or ribozyme therapeutic molecules in the treatment of cancer. Transgenic animals could be used to screen for methods of vaccinating against cancer using an L gene product or a portion thereof.

Further, L gene knock out animals are also useful in the methods of the invention. For example, animals with disruptions in a single L gene can be useful in assessing the relative contribution of its gene products to the cancer state, as well as assessing the positive effect of a cancer therapeutic candidate.

For over- or mis-expression of an L gene product, any technique known in the art may be used to introduce an L gene product into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten et al., 1985, Proc. Natl. Acad. Sci. USA 82:6148); gene targeting in embryonic stem cells (Thompson et al., 1989, Cell 56:313); electroporation of embryos (Lo, 1983, Mol Cell. Biol. 3:1803); and sperm-mediated gene transfer (Lavitrano et al., 1989, Cell 57:717); etc. For a review of such techniques, see Gordon, 1989, Transgenic Animals, Intl. Rev. Cytol. 115: 171.

The methods of the invention provide for the use of transgenic animals that carry an L gene transgene in all their cells, as well as animals which carry the transgene in some, but not all their cells, i.e., mosaic animals.

The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko et al., 1992, Proc. Natl. Acad. Sci. USA 89:6232). The regulatory sequences required for such a cell-type specific activation will depend upon the particular cell type of interest, and will be apparent to those of skill in the art.

When it is desired that an L gene transgene be integrated into the chromosomal site of the endogenous L gene to disrupt the expression of the endogenous L gene, for example, targeting is preferred. Briefly, when such a technique is to be utilized, vectors containing some nucleotide sequences homologous to an endogenous L gene are designed for the purpose of promoting integration into the endogenous gene via homologous recombination. Such chromosomal integration may partially or wholly disrupt the function of the nucleotide sequence of the endogenous L gene. The transgene may also be selectively introduced into a particular cell type, thus inactivating the endogenous L gene in only that cell type, by following, for example, the teaching of Gu et al. (Gu et al., 1994, Science 265:103). The regulatory sequences required for such a cell-type specific inactivation will depend upon the particular cell type of interest, and will be apparent to those of skill in the art.

Methods for the production of single-copy transgenic animals with chosen sites of integration are also well known to those of skill in the art. See, for example, Bronson et al. (Bronson, S. K. et al., 1996, Proc. Natl. Acad. Sci. USA 93:9067).

Once transgenic animals have been generated, the expression of the recombinant L gene may be assayed utilizing standard techniques. Initial screening may be accomplished by Southern blot or PCR analysis of tissue derived from experimental animals to determine which animals possess an integrated transgene. The level of mRNA expression of the transgene in the tissues of the transgenic animals may also be assessed using techniques which include but are not limited to Northern blot analysis, in situ hybridization analysis, and RT-PCR. Samples of L transgene-expressing samples may also be evaluated immunocytochemically using antibodies specific or selective for an L gene product.

Antibodies to L Gene Products

The methods of the present invention encompass the use of antibodies or fragments thereof capable of specifically or selectively recognizing one or more specific L gene product epitopes or epitopes of conserved variants or peptide fragments of the L gene products. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above.

Such antibodies may be used, for example, in the detection of an L gene product in a biological sample and may, therefore, be utilized as part of a diagnostic or prognostic technique whereby patients may be tested for abnormal levels of L gene products, and/or for the presence of abnormal forms of the such gene products. Such antibodies may also be included as a reagent in a kit for use in a diagnostic or prognostic technique. Such antibodies may also be utilized in conjunction with, for example, compound screening methods, as described herein below, for the evaluation of the effect of test compounds on L gene product levels and/or activity. Additionally, such antibodies can be used in conjunction with gene therapy techniques described herein below, for example, to evaluate the normal and/or engineered L gene expressing cells prior to their introduction into the patient.

Antibodies to an L gene gene product may additionally be used in a method for the inhibition of L gene product activity. Thus, such antibodies may, therefore, be utilized as part of cancer treatment methods.

Described herein are methods for the production of antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against an L gene product, various host animals may be immunized by injection with a specific L gene product (e.g., SEQ ID NO: 20), or a portion thereof. Such host animals may include but are not limited to rabbits, mice, and rats, for example. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as an L gene product, or an antigenic functional derivative thereof. For the production of polyclonal antibodies, host animals such as those described above, may be immunized by injection with an L gene product supplemented with adjuvants as described above.

Monoclonal antibodies, which are homogeneous populations of antibodies to a particular antigen or epitope thereof, may be obtained by any technique that provides for the production of antibody molecules by continuous cell lines in culture. These include, but are not limited to, the hybridoma technique of Kohler and Milstein, (1975, Nature 256:495; and U.S. Pat. No. 4,376,110), the human B-cell hybridoma technique (Kosbor et al., 1983, Immunology Today 4:72; Cole et al., 1983, Proc. Natl. Acad. Sci. USA 80:2026), and the EBV-hybridoma technique (Cole et al., 1985, Monoclonal Antibodies And Cancer Therapy, Alan R. Liss, Inc., pp. 77). Such antibodies may be of any immunoglobulin class including IgG, IgM, IgE, IgA, IgD and any subclass thereof. The hybridoma producing the mAb of this invention may be cultivated in vitro or in vivo. Production of high titers of mabs in vivo renders this method a particularly preferred method of production of L polypeptide antibodies.

Techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature 314, 452-454), whereby the genes from a mouse antibody molecule of appropriate antigen specificity are spliced to genes from a human antibody molecule of appropriate biological activity, are also encompassed by the present invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for an L gene product.

Examples of techniques that have been developed for the production of humanized antibodies are known in the art. (See, e.g., Queen, U.S. Pat. No. 5,585,089 and Winter, U.S. Pat. No. 5,225,539) An immunoglobulin light or heavy chain variable region consists of a “framework” region interrupted by three hypervariable regions, referred to as complementarity-determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and framework regions from a human immunoglobulin molecule. The invention includes the use of humanized antibodies that are specific or selective for an L gene product in the methods of the invention.

The methods of the invention encompass the use of an antibody or derivative thereof comprising a heavy or light chain variable domain, said variable domain comprising (a) a set of three complementarity-determining regions (CDRs), in which said set of CDRs are from a monoclonal antibody to a gene product encoded by an L gene nucleic acid sequence (e.g., SEQ ID NO: 20), and (b) a set of four framework regions, in which said set of framework regions differs from the set of framework regions in the L protein monoclonal antibody, and in which said antibody or derivative thereof immunospecifically binds to a gene product encoded by an L gene sequence. Preferably, the set of framework regions is from a human monoclonal antibody, e.g., a human monoclonal antibody that does not bind the gene product encoded by the L gene sequence.

Phage display technology can be used to increase the affinity of an antibody to an L gene product. This technique is useful in obtaining high affinity antibodies to an L gene product that could be used for the diagnosis and/or prognosis of a subject with cancer. The technology, referred to as affinity maturation, employs mutagenesis or CDR walking and re-selection using an L gene product antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contact with the immobilized mutants containing labeled antigen. Any screening method known in the art can be used to identify mutant antibodies with increased avidity to the antigen (e.g., ELISA) (See Wu et al., 1998, Proc Natl. Acad Sci. USA 95:6037; Yelton et al., 1995, J. Immunology 155:1994). CDR walking which randomizes the light chain is also possible (See Schier et al., 1996, J. Mol. Bio. 263:551).

Alternatively, techniques described for the production of single chain antibodies (U.S. Pat. 4,946,778; Bird, 1988, Science 242:423; Huston et al., 1988, Proc. Natl. Acad. Sci. USA 85:5879; and Ward et al., 1989, Nature 334:544) can be adapted to produce single chain antibodies against an L gene product. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:1038).

The methods of the invention include using an antibody to an L polypeptide, peptide or other derivative, or analog thereof that is a bispecific antibody (see generally, e.g., Fanger and Drakeman, 1995, Drug News and Perspectives 8:133-137). Bispecific antibodies can be used for example to treat or prevent cancer in a subject that expresses elevated levels of an L gene product. Such a bispecific antibody is genetically engineered to recognize both (1) an epitope and (2) one of a variety of “trigger” molecules, e.g., Fc receptors on myeloid cells, and CD3 and CD2 on T-cells, that have been identified as being able to cause a cytotoxic T-cell to destroy a particular target. Such bispecific antibodies can be prepared either by chemical conjugation, hybridoma, or recombinant molecular biology techniques known to the skilled artisan.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

Uses of the L Genes, Gene Products, and Antibodies

In various embodiments, the present invention provides various uses of the L genes, L polypeptides and peptide fragments thereof, and of antibodies directed against the L polypeptides and peptide fragments. Such uses include, for example, prognostic and diagnostic evaluation of cancer, and the identification of subjects with a predisposition to a cancer, as described, below. The invention also includes methods of treating and preventing cancer. The invention includes methods for vaccinating against cancer. The methods of the invention can be used for the treatment, prevention, vaccination, diagnosis, staging and/or prognosis of any cancer or tumor, including those listed below in Table 1, which is provided by way of non-limiting example.

Malignancies and related disorders that may be treated according to the methods of the present invention, include but are not limited to those listed in Table 1 (for a review of such disorders, see Fishman et al., 1985, Medicine, 2d Ed., J. B. Lippincott Co., Philadelphia):

TABLE 1
MALIGNANCIES AND RELATED DISORDERS

Leukemia

acute leukemia

acute lymphocytic leukemia
acute myelocytic leukemia

myeloblastic
promyelocytic
myelomonocytic
monocytic
erythroleukemia

chronic leukemia

chronic myelocytic (granulocytic) leukemia
chronic lymphocytic leukemia

Polycythemia vera
Lymphoma

Hodgkin's disease
non-Hodgkin's disease

Multiple myeloma
Waldenström's macroglobulinemia
Heavy chain disease
Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
testicular tumor
lung carcinoma
small cell lung carcinoma
bladder carcinoma
epithelial carcinoma
glioma
astrocytoma
medulloblastoma
craniopharyngioma
ependymoma
pinealoma
hemangioblastoma
acoustic neuroma
oligodendroglioma
menangioma
melanoma
neuroblastoma
retinoblastoma

In a preferred embodiment, the methods of the invention are directed to diagnosis, prognosis, treatment and prevention of lung cancer. In other embodiments, the cancer is breast cancer, brain cancer, ovarian cancer, prostate cancer, gastric cancer, skin cancer, or cancer of the lymphoid system.

The invention further provides for screening assays to identify antagonists or agonists of an L gene or gene product. Thus, the invention relates to methods to identify molecules that modulate (e.g., increase or decrease) the expression and/or activity of L molecules.

The nucleic acid molecules, proteins, protein homologs, and antibodies described herein may be used in one or more of the following methods, including but not limited to: a) screening assays; b) detection assays (e.g., chromosomal mapping, tissue typing); c) predictive medicine (e.g., diagnostic assays, prognostic assays, monitoring efficacy of clinical trials, and pharmacogenomics); and d) methods of treatment (e.g., therapeutic and prophylactic). For example, an L gene product can be used to modulate (i) cellular proliferation; (ii) cellular differentiation; and/or (iii) cellular adhesion. Isolated nucleic acid molecules that encode an L gene or a fragment thereof can be used to express proteins (e.g., via a recombinant expression vector in a host cell in gene therapy applications), to detect mRNA (e.g., in a biological sample) or a genetic lesion, and/or to modulate expression/activity of an L polypeptide. In addition, an L gene product may be used to screen drugs or compounds to identify drugs or compounds capable of modulating the expression or activity of an L gene product. Such drugs or compounds may be used to treat disorders characterized by insufficient or excessive production of the L gene product or production of a form an L gene product which has decreased or aberrant activity as compared to that of the wild type protein. In addition, the antibodies that specifically or selectively bind to an L gene product may be used to detect, isolate, and modulate activity of the L gene product.

In one embodiment, the present invention provides a variety of methods for the diagnostic and prognostic evaluation of cancer, including lung cancer. Such methods may, for example, utilize reagents such as the L gene nucleotide sequences described herein above, and antibodies directed against L gene products, including peptide fragments thereof, as described herein. Specifically, such reagents may be used, for example, for: (1) the detection of the presence of L gene mutations, or the detection of aberrant expression of an L gene mRNA, relative to that of normal cells, or the qualitative or quantitative detection of other allelic forms of L gene transcripts which may correlate with lung cancer or susceptibility toward neoplastic changes, and (2) the detection of an over-abundance of an L gene product relative to the non-disease state or relative to a predetermined non-cancerous standard or the presence of a modified (e.g., less than full-length) L gene product which correlates with a neoplastic state or a progression toward neoplasia or metastasis.

The methods described herein may be performed, for example, by utilizing pre-packaged diagnostic test kits comprising at least one specific or selective L gene nucleic acid or anti-L antibody reagent described herein, which may be conveniently used, e.g., in clinical settings or in home settings, to diagnose patients exhibiting preneoplastic or neoplastic abnormalities, and to screen and identify those individuals exhibiting a predisposition to such neoplastic changes.

Nucleic acid-based and peptide detection techniques are described herein below.

Detection of L Gene Nucleic Acid Molecules

In a preferred embodiment, the invention involves methods to assess quantitative and qualitative aspects of L gene expression. In one example, the increased expression of an L gene or gene product indicates a predisposition for the development of cancer. Alternatively, enhanced expression levels of an L gene or gene product can indicate the presence of cancer in a subject or the risk of metastasis of said cancer in said subject. Techniques well known in the art, e.g., quantitative or semi-quantitative RT PCR or Northern blot, can be used to measure expression levels of an L gene. Methods that describe both qualitative and quantitative aspects of L gene or gene product expression are described in detail in the examples infra. The measurement of L gene expression levels may include measuring naturally occurring L transcripts and variants thereof as well as non-naturally occurring variants thereof. The diagnosis and/or prognosis of cancer in a subject, however, is preferably directed to detecting a naturally occurring L gene product or variant thereof. Thus, the invention relates to methods of diagnosing and/or predicting cancer in a subject by measuring the expression of an L gene in a subject. For example the increased level of mRNA encoded by an L gene (e.g., SEQ ID NO: 1 or SEQ ID NO: 2), or other gene product, as compared to a non-cancerous sample or a non-cancerous predetermined standard would indicate the presence of cancer in said subject or the increased risk of developing cancer in said subject.

In another aspect of the invention, the increased level of mRNA encoded for by an L gene (e.g., SEQ ID NO: 1 or SEQ ID NO: 2), or other related gene product, as compared to that of a non-cancerous sample or a non-cancerous predetermined standard would indicate the stage of disease or the risk of metastasis of the cancer in said subject or the likelihood of a poor prognosis in said subject.

In another example, RNA from a cell type or tissue known, or suspected, to express an L gene, such as lung cancer cells, or other types of cancer cells, may be isolated and tested utilizing hybridization or PCR techniques as described above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the L gene. Such analyses may reveal both quantitative and qualitative aspects of the expression pattern of the L gene, including activation or suppression of L gene expression and the presence of alternatively spliced L gene transcripts.

In one embodiment of such a detection scheme, a cDNA molecule is synthesized from an RNA molecule of interest by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the L gene nucleic acid reagents described herein. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

RT-PCR techniques can be utilized to detect differences in L gene transcript size that may be due to normal or abnormal alternative splicing. Additionally, such techniques can be performed using standard techniques to detect quantitative differences between levels of L gene transcripts detected in normal individuals relative to those individuals having cancer or exhibiting a predisposition toward neoplastic changes.

In the case where detection of particular alternatively spliced species is desired, appropriate primers and/or hybridization probes can be used, such that, in the absence of such a sequence, for example, no amplification would occur. Alternatively, primer pairs may be chosen utilizing the sequence data depicted, for example, in FIGS. 1A-S which will yield fragments of differing size depending on whether a particular exon is present in or absent from an L gene transcript.

As an alternative to amplification techniques, standard Northern analyses can be performed if a sufficient quantity of the appropriate cells can be obtained. The preferred length of a probe used in a Northern analysis is 15-50 nucleotides. Utilizing such techniques, quantitative as well as size related differences between L transcripts can also be detected.

Additionally, it is possible to perform such L gene expression assays in situ, i.e., directly upon tissue sections (fixed and/or frozen) of patient tissue obtained from biopsies or resections, such that no nucleic acid purification is necessary. Nucleic acid reagents such as those described herein may be used as probes and/or primers for such in situ procedures (see, e.g., Nuovo, G. J., 1992, PCR In Situ Hybridization: Protocols And Applications, Raven Press, NY).

Mutations or polymorphisms within an L gene can be detected by utilizing a number of techniques. Nucleic acid from any nucleated cell (e.g., genomic DNA) can be used as the starting point for such assay techniques, and may be isolated according to standard nucleic acid preparation procedures that are well known to those of skill in the art. For the detection of L transcripts or L gene products, any cell type or tissue in which the L gene is expressed, such as, for example, lung cancer cells may be utilized.

Genomic DNA may be used in hybridization or amplification assays of biological samples to detect abnormalities involving L gene structure, including point mutations, insertions, deletions and chromosomal rearrangements. Such assays may include, but are not limited to, direct sequencing (Wong, C. et al., 1987, Nature 330:384), single stranded conformational polymorphism analyses (SSCP; Orita, M. et al., 1989, Proc. Natl. Acad. Sci. USA 86:2766), heteroduplex analysis (Keen, T. J. et al., 1991, Genomics 11:199; Perry, D. J. & Carrell, R. W., 1992), denaturing gradient gel electrophoresis (DGGE; Myers, R. M. et al., 1985, Nucl. Acids Res. 13:3131), chemical mismatch cleavage (Cotton, R. G. et al., 1988, Proc. Natl. Acad Sci. USA 85:4397) and oligonucleotide hybridization (Wallace, R. B. et al., 1981, Nucl. Acids Res. 9:879; Lipshutz, R. J. et al., 1995, Biotechniques 12:442).

Diagnostic methods for the detection of L gene nucleic acid molecules, in patient samples or other appropriate cell sources, may involve the amplification of specific gene sequences, e.g., by PCR (See Mullis, K. B., 1987, U.S. Pat. No. 4,683,202), followed by the analysis of the amplified molecules using techniques well known to those of skill in the art, such as, for example, those listed above. Utilizing analysis techniques such as these, the amplified sequences can be compared to those that would be expected if the nucleic acid being amplified contained only normal copies of an L gene in order to determine whether an L gene mutation exists.

Further, well-known genotyping techniques can be performed to type polymorphisms that are in close proximity to mutations in the L gene itself. These polymorphisms can be used to identify individuals in families likely to carry mutations. If a polymorphism exhibits linkage disequilibrium with mutations in an L gene, it can also be used to identify individuals in the general population likely to carry mutations. Polymorphisms that can be used in this way include restriction fragment length polymorphisms (RFLPs), which involve sequence variations in restriction enzyme target sequences, single-nucleotide polymorphisms (SNPs) and simple sequence repeat polymorphisms (SSLPs).

For example, Weber (U.S. Pat. No. 5,075,217) describes a DNA marker based on length polymorphisms in blocks of (dC-dA)n(dG-dT)n short tandem repeats. The average separation of (dC-dA)n-(dG-dT)n blocks is estimated to be 30,000-60,000 bp. Markers that are so closely spaced exhibit a high frequency co-inheritance, and are extremely useful in the identification of genetic mutations, such as, for example, mutations within an L gene, and the diagnosis of diseases and disorders related to L gene mutations.

Also, Caskey et al. (U.S. Pat. No. 5,364,759), describe a DNA profiling assay for detecting short tri- and tetra-nucleotide repeat sequences. The process includes extracting the DNA of interest, such as an L gene, amplifying the extracted DNA, and labeling the repeat sequences to form a genotypic map of the individual's DNA.

An L gene probe could be used to directly identify RFLPs. Additionally, an L gene probe or primers derived from an L gene sequence could be used to isolate genomic clones such as YACs, BACs, PACs, cosmids, phage or plasmids. The DNA contained in these clones can be screened for single-base polymorphisms or simple sequence length polymorphisms (SSLPs) using standard hybridization or sequencing procedures.

Alternative diagnostic methods for the detection of L gene expression, L gene mutations or polymorphisms can include hybridization techniques which involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described herein, under conditions favorable for the specific or selective annealing of these reagents to their complementary sequences within an L gene. Preferably, the lengths of these nucleic acid reagents are at least 15 to 50 nucleotides. After incubation, all non-annealed nucleic acids are removed from the L hybrid molecule. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads or to a glass surface such as a microscope slide. In this case, after incubation, non-annealed, labeled nucleic acid reagents are easily removed. Detection of the remaining, annealed, labeled L nucleic acid reagents is achieved using standard techniques well known to those in the art. The L gene sequences to which the nucleic acid reagents have annealed can be compared to the annealing pattern expected from a normal L gene sequence in order to determine whether an L gene mutation is present.

Detection of L Encoded Proteins

Detection of an L gene product includes the detection of the proteins encoded by one of SEQ ID NOs: 1-19. L proteins of the invention include SEQ ID NOs: 20-38 and functional fragments thereof. Detection of elevated levels of an L protein or polypeptides thereof, as compared to a non-cancerous sample or a non-cancerous predetermined standard, can indicate the presence of, or predisposition to developing cancer in a subject. Detection of elevated levels of an L protein or polypeptides thereof, in a subject as compared to a non-cancerous sample or a non-cancerous predetermined standard can also indicate the likelihood of metastasis of a cancer in the subject, and/or poor prognosis for the subject. The diagnosis and/or prognosis of cancer relates to the detection of naturally occurring L polypeptides in a subject. Detection of an L polypeptide may be achieved by any method known in the art.

Antibodies directed against naturally occurring L, or naturally occurring variants thereof or peptide fragments thereof, may be used as diagnostics and prognostics, as described herein. Such diagnostic methods may be used to detect abnormalities in the level of L gene expression, or abnormalities in the structure and/or temporal, tissue, cellular, or subcellular location of an L encoded polypeptide. Antibodies, or fragments of antibodies, such as those described herein, may be used to screen therapeutic compounds in vitro to identify compounds capable of modulating L gene expression, L encoded polypeptide production and activity thereto. Compounds capable of modulating L activity and identified using the methods of the invention may be tested to determine their utility as therapeutic compounds for the treatment of cancer patients (e.g., lung cancer patients). Accordingly, a skilled practitioner could determine a therapeutically effective dose range for a cancer patient based on a number of parameters, including but not limited to the age, weight, and condition of the patient, the type and severity of the disease, and the treatment history of the patient.

The tissue or cell type to be analyzed will generally include those which are known, or suspected, to express an L gene, such as, for example, cancer cells including lung cancer cells, breast cancer cells, brain cancer cells, ovarian cancer cells, prostate cancer cells, gastric cancer cells, skin cancer cells, lymphoid cancer cells, and metastatic forms thereof. The protein isolation methods employed herein may, for example, be such as those described in Harlow and Lane (Harlow, E. and Lane, D., 1988, Antibodies: A Laboratory Manual, Cold Spring Harbor Laboratory Press, Cold Spring Harbor, N.Y.). The isolated cells can be derived from cell culture or from a patient. The analysis of cells taken from culture may be a necessary step to test the effect of compounds on the expression of an L gene.

Preferred diagnostic methods for the detection of L gene products or conserved variants or peptide fragments thereof, may involve, for example, immunoassays wherein L gene products or conserved variants, including gene products which are the result of alternatively spliced transcripts, or peptide fragments are detected by their interaction with an anti-L gene product-specific or -selective antibody.

For example, antibodies, or fragments of antibodies, such as those described herein above, useful in the present invention may be used to quantitatively or qualitatively detect the presence of an L gene encoded polypeptides or naturally occurring variants or peptide fragments thereof. The antibodies (or fragments thereof) useful in the present invention may, additionally, be employed histologically, as in immunofluorescence or immunoelectron microscopy, for in situ detection of L gene products or conserved variants or peptide fragments thereof. In situ detection may be accomplished by removing a histological specimen from a subject, such as paraffin embedded sections of tissue, e.g., lung tissues and applying thereto a labeled antibody of the present invention. The antibody (or fragment) is preferably applied by overlaying the labeled antibody (or fragment) onto a biological sample. If an L gene product appears to be expressed predominantly as an intracellular protein, it may be desirable to introduce the antibody inside the cell, for example, by permeabilizing the cell membrane. If an L polypeptide is expressed on the cell surface, cells can be directly labeled by applying antibodies that are specific or selective for the L polypeptide or fragment thereof to the cell surface.

Through the use of such procedures, it is possible to determine not only the presence of an L gene product, or naturally occurring variants thereof or peptide fragments, but also the distribution of these molecules in the examined tissue. Using the methods of the present invention, those of ordinary skill will readily perceive that any of a wide variety of histological methods (such as staining procedures) can be modified in order to achieve such in situ detection.

Immunoassays for L encoded polypeptides or conserved variants or peptide fragments thereof will typically comprise contacting a sample, such as a biological fluid, tissue or a tissue extract, freshly harvested cells, or lysates of cells which have been incubated in cell culture, in the presence of an antibody that specifically or selectively binds to an L gene product, e.g., a detectably labeled antibody capable of identifying an L polypeptide or a conserved variant or peptide fragment thereof, and detecting the bound antibody by any of a number of techniques well-known in the art (e.g., Western blot, ELISA, FACS).

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled antibody that selectively or specifically binds to an L polypeptide. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified cellulose, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

An anti-L antibody can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.); Voller, A. et al., 1978, J. Clin. Pathol. 31: 507-520; Butler, J. E., 1981, Meth. Enzymol. 73:482; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme that is bound to the antibody will react with an appropriate substrate, preferably a chromogenic substrate, in such a manner as to produce a chemical moiety that can be detected, for example, by spectrophotometric, fluorimetric or other visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect L encoded polypeptides through the use of radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence emission. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody can also be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In various embodiments, the present invention provides methods for the measurement of L polypeptides, and the uses of such measurements in clinical applications using L-specific or -selective antibodies.

The measurement of L polypeptides of the invention can be valuable in detecting and/or staging lung cancer and other cancers in a subject, in screening of lung cancer and other cancers in a population, in differential diagnosis of the physiological condition of a subject, and in monitoring the effect of a therapeutic treatment on a subject.

The present invention also provides for detecting, diagnosing, or staging lung cancer and other cancers, or for monitoring the treatment of lung cancer and other cancers by measuring the level of expression of an L polypeptide. In addition to the L polypeptide, at least one other marker, such as receptors or differentiation antigens can also be measured. For example, serum markers selected from, for example but not limited to, carcinoembryonic antigen (CEA), CA15-3, CA549, CAM26, M29, CA27.29 and MCA can be measured in combination with the L polypeptide to detect, diagnose, stage, or monitor treatment of lung cancer and other cancers. In another embodiment, the prognostic indicator is the observed change in different marker levels relative to one another, rather than the absolute levels of the markers present at any one time. These measurements can also aid in predicting therapeutic outcome and in evaluating and monitoring the overall disease status of a subject.

In a specific embodiment of the invention, soluble L polypeptide alone or in combination with other markers can be measured in any body fluid of the subject including but not limited to blood, serum, plasma, milk, urine, saliva, pleural effusions, synovial fluid, spinal fluid, tissue infiltrations and tumor infiltrates. In another embodiment, an L polypeptide is measured in tissue samples or cells directly. The present invention also contemplates a kit for measuring the level of L polypeptide expression in a biological sample and the use of said kit to diagnose a subject with cancer. Alternatively said kit could be used to determine the prognosis of a cancer patient or the risk of metastasis of said cancer.

Any of numerous immunoassays can be used in the practice of the methods of the instant invention, such as those described herein below. Antibodies, or antibody fragments containing the binding domain, which can be employed include, but are not limited to, suitable antibodies among those in described above and other antibodies known in the art or those which can be obtained by procedures standard in the art such as those described herein above.

In Vivo Imaging Using Antibodies to an L Polypeptide

Current diagnostic and therapeutic methods make use of antibodies to target imaging agents or therapeutic substances, e.g., to tumors. Thus, labeled antibodies specific or selective for an L polyppeptide can be used in the methods of the invention for the in vivo imaging, detection, and treatment of cancer in a subject.

Antibodies may be linked to chelators such as those described in U.S. Pat. No. 4,741,900 or U.S. Pat. No. 5,326,856. The antibody-chelator complex may then be radiolabeled to provide an imaging agent for diagnosis or treatment of disease. The antibodies may also be used in the methods that are disclosed in U.S. Pat. No. 5,449,761 for creating a radiolabeled antibody for use in imaging or radiotherapy.

In in vivo diagnostic applications, specific tissues or even specific cellular disorders, e.g., cancer, may be imaged by administration of a sufficient amount of a labeled antibody using the methods of the instant invention.

A wide variety of metal ions suitable for in vivo tissue imaging have been tested and utilized clinically. For imaging with radioisotopes, the following characteristics are generally desirable: (a) low radiation dose to the patient; (b) high photon yield which permits a nuclear medicine procedure to be performed in a short time period; (c) ability to be produced in sufficient quantities; (d) acceptable cost; (e) simple preparation for administration; and (f) no requirement that the patient be sequestered subsequently. These characteristics generally translate into the following: (a) the radiation exposure to the most critical organ is less than 5 rad; (b) a single image can be obtained within several hours after infusion; (c) the radioisotope does not decay by emission of a particle; (d) the isotope can be readily detected; and (e) the half-life is less than four days (Lamb and Kramer, “Commercial Production of Radioisotopes for Nuclear Medicine”, In Radiotracers For Medical Applications, Vol. 1, Rayudu (Ed.), CRC Press, Inc., Boca Raton, pp. 17-62). Preferably, the metal is technetium-99m.

By way of illustration, the targets that one may image include any solid neoplasm, certain organs such a lymph nodes, parathyroids, spleen and kidney, sites of inflammation or infection (e.g., macrophages at such sites), myocardial infarction or thromboses (neoantigenic determinants on fibrin or platelets), and the like evident to one of ordinary skill in the art. Furthermore, the neoplastic tissue may be present in bone, internal organs, connective tissue, or skin.

As is also apparent to one of ordinary skill in the art, one may use the methods of the present invention for in vivo therapeutics (e.g., using radiotherapeutic metal complexes), especially after having diagnosed a diseased condition via the in vivo diagnostic method described above, or in in vitro diagnostic application (e.g., using a radiometal or a fluorescent metal complex).

Accordingly, a method for diagnosing cancer by obtaining an image of an internal region of a subject is contemplated by the instant invention which comprises administering to a subject an effective amount of an antibody composition specific or selective for an L polypeptide conjugated with a metal which is radioactively labeled, and recording the scintigraphic image obtained from the decay of the radioactive metal. Likewise, a method is contemplated for enhancing a magnetic resonance image (MRI) of an internal region of a subject which comprises administering to a subject an effective amount of an antibody composition containing a paramagnetic metal, and recording the MRI of an internal region of the subject.

Other methods include a method of enhancing a sonographic image of an internal region of a subject comprising administering to a subject an effective amount of an antibody composition containing a metal and recording the sonographic image of an internal region of the subject. In this latter application, the metal is preferably any non-toxic heavy metal ion. A method of enhancing an X-ray image of an internal region of a subject is also provided which comprises administering to a subject an antibody composition containing a metal, and recording the X-ray image of an internal region of the subject. A radioactive, non-toxic heavy metal ion is preferred.

Detecting and Staging Cancer in a Subject

The methods of the present invention include measurement of naturally occurring L polypeptides, or naturally occurring variants thereof, or fragments thereof, soluble L polypeptides or intracellular L polypeptides to detect lung cancer or other cancers in a subject or to stage lung cancer or other cancers in a subject.

Staging refers to the grouping of patients according to the extent of their disease. Staging is useful in choosing treatment for individual patients, estimating prognosis, and comparing the results of different treatment programs. Staging of lung cancer for example is performed initially on a clinical basis, according to a physical examination and laboratory radiologic evaluation. The most widely used clinical staging system is the one adopted by the International Union against Cancer (UICC) and the American Joint Committee on Cancer (AJCC) Staging and End Results Reporting. It is based on the tumor-nodes-metastases (TNM) system as detailed in the 1988 Manual for Staging of Cancer. The revised International System for Staging Lung Cancer was completed in 1997 by the American Joint Committee on Cancer and the Union Internationale Contre le Cancer (Mountain et al., 1997, Chest. 111(6):1710-1717). Lung cancer diseases or conditions that may be detected and/or staged in a subject according to the present invention include but are not limited to those listed in Table 2.

TABLE 2
TNM Classification for Lung Cancer

| | Classi- | |
|---|---|---|
| Stage | fication | Definition |
| T | TX | Primary tumor not visual by imaging or bronchoscopy |
| T | T0 | No evidence of primary tumor |
| T | Tis | Carcinoma in situ |
| T | T1 | Tumor is < or = 3 cm |
| T | T2 | Tumor is >3 cm |
| T | T3 | Tumor of any size that invades the chest wall or the |
| | | structures of the chest's center |
| T | T4 | Tumor of any size that invades vital structures, such as |
| | | soft tissues of the mediastinum and the vertebral body |
| N | NX | Regional lymph nodes can't be assessed |
| N | N0 | No regional lymph node metastasis |
| N | N1 | Metastasis to ipsilateral peribronchial and/or ipsilateral |
| | | nodes, and intrapulmonary nodes including involvement |
| | | by extension of the primary tumor |
| N | N2 | Metastasis to ipsilateral mediastinal and/or subcarinal |
| | | lymph nodes |
| N | N3 | Metastasis to contralateral mediastinal, contralateral |
| | | Hilar, ipsilateral or contralateral scalene, or |
| | | supraclavicular lymph nodes |
| M | MX | Distant metastasis can't be assessed |
| M | M0 | No distant metastasis |
| M | M1 | Presence of distant metastasis |

Any immunoassay, such as those described herein above can be used to measure the amount of L polypeptide or soluble L polypeptide and compare the measured level to that of a baseline level. This baseline level can be the amount that is established to be present in the non-cancerous tissue or body fluid (e.g., unaffected tissue) of subjects with various degrees of the disease or disorder. An amount present in the tissue or body fluid of the subject that is similar to a standard amount, established to be normally present in the tissue or body fluid of a subject during a specific stage of cancer or lung cancer, is indicative of the stage of the disease in the subject. The baseline level could also be the level present in the subject prior to the onset of disease or the amount present during remission of the disease.

In specific embodiments of this aspect of the invention, measurements of levels of an L polypeptide or soluble L polypeptide can be used in the detection of infiltrative ductal carcinoma (IDC) or the presence of metastases or both. Increased levels of L polypeptides or soluble L polypeptides may be associated with metastases.

In another embodiment of the invention, the measurement of soluble L polypeptide, intracellular L polypeptide, fragments thereof or immunologically related molecules can be used to differentially diagnose in a subject a particular disease phenotype or physiological condition from other phenotypes or physiological conditions. For example, measurements of L polypeptide or soluble L polypeptide levels may be used in the differential diagnosis of infiltrative ductal carcinoma, as distinguished from ductal carcinoma in situ or benign fibroadenomas. To this end, for example, the measured amount of L polypeptide is compared with the amount of the molecule normally present in the tissue, cells or body fluid of a subject with one of the suspected physiological conditions. A measured amount of the L polypeptide similar to the amount normally present in a subject with one of the physiological conditions, and not normally present in a subject without this condition, serves as a positive indicator or diagnostic of the presence of the physiological condition in the tested subject.

As an alternative to measuring levels of L polypeptides in the foregoing staging methods, levels of L gene transcript can be measured, for example by the methods described herein above.

Monitoring the Effect of a Therapeutic Treatment

The present invention provides a method for monitoring the effect of a therapeutic treatment on the disease state of a subject.

The need for a clinical procedure(s) that can be used to monitor the efficacy of a cancer treatment is well recognized. As described herein, the detection of L gene transcripts and encoded polypeptides in lung cancer and other cancers associated with aberrant L gene regulation provide a sensitive assay system with which to monitor therapeutic regimens. Therapeutic treatments that may be evaluated according to the present invention include, but are not limited to, radiotherapy, surgery, chemotherapy, vaccine administration, endocrine therapy, immunotherapy, and gene therapy, etc. The chemotherapeutic regimens include, but are not limited to administration of drugs such as, for example, methotrexate, fluorouracil, cyclophosphamide, doxorubicin, and taxol. The endocrine therapeutic regimens include, but are not limited to administration of tamoxifen and progestins.

The method of the invention comprises measuring at suitable time intervals before, during, or after therapy, the amount of an L gene transcript or polypeptide (including soluble polypeptide), or any combination of the foregoing. Any change or absence of change in the absolute or relative amounts of the L gene products can be identified and correlated with the effect of the treatment on the subject.

In particular, the serum- or cell-associated levels of an L polypeptide may bear a direct relationship with the severity of a lung cancer, or other cancer, the risk of metastasis of said cancer and poor prognosis. Since serum- or cell-associated L polypeptide levels are generally undetectable or negligible in normal individuals and up-regulated in cancer patients (e.g., lung cancer patients), generally, a decrease in the level of detectable L polypeptide after a therapeutic treatment is associated with efficacious treatment.

In a preferred aspect, the levels of soluble or cell-associated L polypeptide levels may be measured at different time points and compared to baseline levels. The baseline level(s) may be established as the level present prior to treatment, during remission of disease, or during periods of stability. For some applications, the baseline level may correlate with the level of the L polypeptide present in normal, disease free individuals. Comparisons to baseline levels may be used to establish ratios of change (or relative comparisons), which may be correlated with the disease course or treatment outcome.

Prognostic Assays

The methods described herein can furthermore be utilized as prognostic assays to identify subjects having or at risk of developing cancer or another disease or disorder associated with aberrant expression or activity of an L polypeptide. For example, the assays described herein, such as the preceding diagnostic assays or the following assays, can be utilized to identify a subject having or at risk of developing cancer, e.g., lung cancer, or another disorder associated with aberrant expression or activity of an L polypeptide. Thus, the present invention provides a method in which a test sample is obtained from a subject and an L polypeptide or nucleic acid (e.g., mRNA) of the invention is detected, wherein the presence of the polypeptide or nucleic acid is diagnostic for a subject having or at risk of developing a disease or disorder associated with aberrant expression or activity of the L polypeptide, e.g., cancer. As used herein, a “test sample” refers to a biological sample obtained from a subject of interest. For example, a test sample can be a biological fluid (e.g., serum), cell sample, or tissue.

The prognostic assays described herein, for example, can be used to identify a subject having or at risk of developing disorders such as cancers, for example, hormone-sensitive cancer such as lung cancer.

In another example, prognostic assays described herein can be used to identify a subject having or at risk of developing related disorders associated with expression of polypeptides or nucleic acids of the invention.

Furthermore, the prognostic assays described herein can be used to determine whether a subject can be administered an agent (e.g., an agonist, antagonist, peptidomimetic, protein, peptide, nucleic acid, small molecule, or other drug candidate) to treat cancer or another disease or disorder associated with aberrant expression or activity of an L polypeptide. For example, such methods can be used to determine whether a subject can be treated effectively with a specific agent or class of agents (e.g., agents of a type which decrease activity or expression level of an L transcript or polypeptide). Thus, the present invention provides methods for determining whether a subject can be effectively treated with an agent for a disorder associated with aberrant expression or activity of the L transcript or polypeptide. Such methods may involve steps whereby a test sample is obtained and the L polypeptide or nucleic acid encoding the L polypeptide is detected. The presence of the polypeptide or nucleic acid in the sample indicates that the subject is a candidate for treatment with agents of the present invention.

The methods of the invention can also be used to detect genetic lesions or mutations in an L gene, thereby determining if a subject with the lesioned gene is at increased or reduced risk for a disorder characterized by aberrant expression or activity of a polypeptide of the invention, e.g., cancer. In one embodiment, the methods include detecting, in a sample of cells from the subject, the presence or absence of a genetic lesion or mutation characterized by at least one of an alteration affecting the integrity of a gene encoding an L polypeptide, or the mis-expression of the gene encoding an L polypeptide. For example, such genetic lesions or mutations can be detected by ascertaining the existence of at least one of: 1) a deletion of one or more nucleotides from an L gene; 2) an addition of one or more nucleotides to an L gene; 3) a substitution of one or more nucleotides of an L gene, i.e., a point mutation; 4) a chromosomal rearrangement of an L gene; 5) an alteration in the level of a messenger RNA transcript of an L gene; 6) an aberrant modification of an L gene, such as of the methylation pattern of the genomic DNA; 7) the presence of a non-wild type splicing pattern of a messenger RNA transcript of an L gene; 8) a non-wild type level of the protein encoded by an L gene; 9) an allelic loss of an L gene; and 10) an inappropriate post-translational modification of a protein encoded by an L gene. As described herein, there are a large number of assay techniques known in the art that can be used for detecting lesions in a gene.

In certain embodiments, methods for the detection of the lesion involve the use of a probe/primer in a polymerase chain reaction (PCR) (See, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077; and Nakazawa et al. (1994) Proc Natl Acad Sci. USA 91:360), the latter of which can be particularly useful for detecting point mutations in a gene (see, e.g., Abravaya et al. (1995) Nucleic Acids Res. 23:675). These methods are useful in the diagnosis and prognosis of cancer in a subject. This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene or gene product (if present) occurs, and detecting the presence or absence of an amplification product, or detecting the size of the amplification product and comparing the length to a control sample. It is anticipated that PCR and/or LCR may be used as a preliminary amplification step in conjunction with any of the techniques used for detecting mutations described herein.

Mutations in a selected gene from a sample cell or tissue can also be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, methods are provided whereby genetic mutations can be identified by hybridizing a sample and control nucleic acids, e.g., DNA or RNA, to high density arrays comprising hundreds or thousands of oligonucleotides probes (Cronin et al.1996, Human Mutation 7:244; Kozal et al. 1996, Nature Medicine 2:753). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Sequencing reactions known in the art can be used to sequence the selected gene and detect mutations in an L gene by comparing the sequence of the sample nucleic acids with the corresponding wild-type (control) sequence. Examples of sequencing reactions include those based on techniques developed by Maxim and Gilbert (Maxim and Gilbert, 1977, Proc Natl Acad Sci. USA 74:560) or Sanger (Sanger et al. 1977, Proc Natl Acad Sci. USA 74:5463). Such methods are useful in the diagnosis and prognosis of a subject with cancer. It is also contemplated that any of a variety of automated sequencing procedures can be utilized when performing the diagnostic assays (Naeve et al., 1995, BioTechniques 19:448), including sequencing by mass spectrometry (see, e.g., PCT Publication No. WO 94/16101; Cohen et al. 1996, Adv. Chromatogr. 36: 127; and Griffin et al., 1993, Appl. Biochem. Biotechnol. 38:147).

Furthermore, the presence of an L nucleic acid molecule or polypeptide of the invention can be correlated with the presence or expression level of other cancer-related proteins, such as for example, the androgen receptor, the estrogen receptor, adhesion molecules (e.g., E-cadherin), proliferation markers (e.g., MIB-1), tumor-suppressor genes (e.g., TP53, retinoblastoma gene product), vascular endothelial growth factor (Lissoni et al., 2000, Int J Biol Markers. 15(4):308), Rad5l (Maacke et al., 2000, Int J Cancer. 88(6):907), cyclin D1, BRCA1, BRCA2, or carcinoembryonic antigen.

The methods described herein may be performed, for example, by utilizing pre-packaged diagnostic kits comprising at least one nucleic acid probe or antibody reagent described herein, which may be conveniently used, e.g., in clinical settings to diagnose patients exhibiting symptoms or assess a family history of a disease or illness involving a gene encoding a polypeptide of the invention. Furthermore, any cell type or tissue, e.g., preferably cancerous lung cells or tissue, in which an L gene is expressed may be utilized in the prognostic assays described herein.

Screening for L Gene Activity

The present invention further provides methods for the identification of compounds that may, through their interaction with an L gene or L gene product, affect the onset, progression and metastatic spread of lung cancer and/or other cancers.

The following assays are designed to identify: (i) compounds that bind to L gene products; (ii) compounds that bind to other proteins that interact with an L gene product; (iii) compounds that interfere with the interaction of the L gene product with other proteins; and (iv) compounds that modulate the activity of an L gene (i.e., modulate the level of L gene expression, including transcription of the L gene and/or translation of its encoded transcript, and/or modulate the level of L-encoded polypeptide activity). Proteins that interact with L molecules may, for example, be involved in the onset, development and metastatic spread of lung cancer or other cancers.

Assays may additionally be utilized which identify compounds that bind to L gene regulatory sequences (e.g., promoter sequences), which may modulate the level of L gene expression (see e.g., Platt, K. A., 1994, J. Biol. Chem. 269:28558).

The present invention also provides methods of using isolated L nucleic acid molecules, or derivatives thereof, as probes that can be used to screen for DNA-binding proteins, including but not limited to proteins that affect DNA conformation or modulate transcriptional activity (e.g., enhancers, transcription factors). In another embodiment, such probes can be used to screen for RNA-binding factors, including but not limited to proteins, steroid hormones, or other small molecules. In yet another embodiment, such probes can be used to detect and identify molecules that bind or affect the pharmacokinetics or activity (e.g., enzymatic activity) of an L gene or gene product. The proteins or nucleic acid binding factors or transcriptional modulators identified by a screening assay would provide an appropriate target for anti-cancer therapeutics.

In one embodiment, a screening assay of the invention can identify a test compound that is useful for increasing or decreasing the translation of an L gene ORF, for example, by binding to one or more regulatory elements in the 5′ untranslated region, the 3′ untranslated region, or the coding regions of the mRNA. Compounds that bind to mRNA can, inter alia, increase or decrease the rate of mRNA processing, alter its transport through the cell, prevent or enhance binding of the mRNA to ribosomes, suppressor proteins or enhancer proteins, or alter mRNA stability. Accordingly, compounds that increase or decrease mRNA translation can be used to treat or prevent disease. For example, diseases such as cancer, associated with overproduction of L proteins, can be treated or prevented by decreasing translation of the mRNA that codes for the overproduced protein, thus inhibiting production of the protein.

Accordingly, in one embodiment, a compound identified by a screening assay of the invention inhibits the production of an L protein. In a further embodiment, the compound inhibits the translation of an L mRNA. In yet another embodiment, the compound inhibits transcription of the L gene.

The invention provides a method for identifying modulators, i.e., candidate or test compounds or agents (e.g., peptides, peptidomimetics, small molecules or other drugs) which bind to an L product or fragments thereof or have a stimulatory or inhibitory effect on, for example, expression or activity of an L gene product or fragment thereof. Compounds identified via assays such as those described herein may be useful, for example, in elaborating the biological function of an L gene product, and for ameliorating symptoms of lung cancer or other types of cancer. Techiniques for identifying L molecule modulatory compounds and assays for testing their effectiveness are described herein below. It is to be noted that the compositions of the invention include pharmaceutical compositions comprising one or more of the compounds identified via such methods. Such pharmaceutical compositions can be formulated, for example, as discussed herein below.

In Vitro Screening for Compounds that Bind to an L Gene

In vitro systems may be designed to identify compounds capable of interacting with, e.g., binding to, an L gene product of the invention. Compounds identified may be useful, for example, in modulating the activity of wild type and/or mutant L gene products, may be useful in elaborating the biological function of an L gene product, may be utilized in screens for identifying compounds that disrupt normal L gene product interactions, or may in themselves disrupt such interactions. Thus, said compounds would be useful for treating, preventing and/or diagnosing cancer. In a particular embodiment said compounds are useful in the treatment, prevention and diagnosis of lung cancer.

The principle of assays used to identify compounds that interact with an L gene product involves preparing a reaction mixture of an L gene product and a test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay would involve anchoring an L gene product or the test substance onto a solid phase and detecting L gene product/test compound complexes anchored on the solid phase at the end of the reaction. In one embodiment of such a method, the L gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific or selective for the protein to be immobilized may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific or selective for an L gene product or the test compound to anchor any complexes formed in solution, and a labeled antibody specific or selective for the other component of the possible complex to detect anchored complexes.

Assays for Proteins that Interact with and L Gene

Any method suitable for detecting protein-protein interactions may be employed for identifying L protein-protein interactions. Proteins that interact with an L protein are potential therapeutics for the treatment of cancer. Thus, the assays described below are useful for identifying proteins that can be used in methods to treat cancer. Proteins that interact with an L protein can also be used for the diagnosis of cancer. Thus, the assays described below are also useful in methods to diagnose cancer.

Traditional methods for the detection of protein-protein interactions include, without limitation, co-immunoprecipitation, crosslinking, and co-purification through gradients or chromatographic columns (e.g., size exclusion chromatography). Utilizing procedures such as these allows for the isolation of cellular proteins that interact with L gene products (L gene product specific binding partners). Once isolated, such cellular proteins can be identified and can, in turn, be used, in conjunction with standard techniques, to identify additional proteins with which the specific binding partner of an L gene product interacts. For example, at least a portion of the amino acid sequence of an L gene product specific binding partner (L gene product spb) can be ascertained using techniques well known to those of skill in the art, such as via the Edman degradation technique (see, e.g., Creighton, 1983, Proteins: Structures and Molecular Principles, W.H. Freeman & Co., N.Y., pp. 34-49). The amino acid sequence obtained may be used as a guide for the generation of oligonucleotide mixtures that can be used to screen for gene sequences encoding such cellular proteins. Screening may be accomplished, for example, by standard hybridization or PCR techniques. Techniques for the generation of oligonucleotide mixtures and screening are well known in the art. (See, e.g., Ausubel, supra, and PCR Protocols: A Guide to Methods and Applications, 1990, Innis, M. et al., eds. Academic Press, Inc., New York).

Additionally, methods may be employed which result in the simultaneous identification of genes which encode a protein interacting with an L protein. These methods include, for example, probing expression libraries with labeled L protein, using L protein in a manner similar to the technique of antibody probing of λgt11 libraries.

One method that detects protein interactions in vivo, the two-hybrid system, may also be used to advantage. Many versions of this system have been described (See e.g., Chien et al., 1991, supra). The system described by Chien et al. (1991, supra) is commercially available from Clontech (Palo Alto, Calif.).

Assays for Compounds that Alter L Gene Product Interactions

An L gene product may interact with one or more macromolecules in vivo, such as proteins or nucleic acids. With regard to the present invention, such macromolecules are referred to herein as “interacting partners” or “specific binding partners”. Compounds that disrupt L interactions are useful agents for regulating the activity of L gene products, including mutant L gene products. Such compounds may include, but are not limited to molecules such as peptides, and the like, as described, for example, herein above. Thus, the assays described below are useful for identifying proteins and/or nucleic acids that can be used in methods to treat cancer. Proteins and nucleic acids that interact with L gene products can also be used in the diagnosis of cancer, e.g., lung cancer. Thus, the assays described below are also useful for methods to diagnose cancer, e.g., lung cancer.

The basic principle of the assay systems used to identify compounds that interfere with the interaction between an L gene product and its interacting partner or partners involves preparing a reaction mixture containing an L gene product, and the interacting partner under conditions and for a time sufficient to allow the two to interact and bind, thus forming a complex. In order to test a compound for inhibitory activity, the reaction mixture is prepared in the presence or absence of the test compound. The test compound may be initially included in the reaction mixture, or may be added at a time subsequent to the addition of L gene product and its interacting partner(s). Control reaction mixtures are incubated without the test compound or with a placebo. The formation of any complexes between an L protein and an interacting partner is then detected. The formation of a complex in the control reaction, but not in the reaction mixture containing the test compound, indicates that the compound interferes with the interaction of the L protein and the interacting partner. Additionally, complex formation within reaction mixtures containing the test compound and normal L protein may also be compared to complex formation within reaction mixtures containing the test compound and a mutant L protein. This comparison may be important in those cases wherein it is desirable to identify compounds that disrupt interactions of mutant but not normal L gene proteins.

An assay for compounds that interfere with the interaction of an L gene product or protein and interacting partners can be conducted in a heterogeneous or homogeneous format. Heterogeneous assays involve anchoring either the L gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between L gene products and interacting partners, e.g., by competition, can be identified by conducting the reaction in the presence of the test substance; i.e., by adding the test substance to the reaction mixture prior to or simultaneously with the L gene protein and interacting partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the L gene product or the interacting partner is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of an L gene product or interacting partner and drying. Alternatively, an immobilized antibody specific or selective for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the interacting components to anchor any complexes formed in solution, and a labeled antibody specific or selective for the other partner to detect anchored complexes. Again, depending upon the order of addition of reactants to the liquid phase, test compounds which inhibit complex formation or which disrupt preformed complexes can be identified.

In an alternate embodiment of the invention, a homogeneous assay can be used. In this approach, a preformed complex of an L gene protein and the interacting partner is prepared in which either the L gene product or its interacting partner is labeled, but the signal generated by the label is quenched due to complex formation (see, e.g., U.S. Pat. No. 4,109,496 by Rubenstein). The addition of a test substance that competes with and displaces one of the species from the preformed complex will result in the generation of a signal above background. In this way, test substances that disrupt L gene product/interacting partner interaction can be identified.

In a particular embodiment, an L gene product can be prepared for immobilization using recombinant DNA techniques such as those described herein above. For example, an L coding region can be fused to a glutathione-S-transferase (GST) gene using a fusion vector, such as pGEX-5X-1, in such a manner that its interacting activity is maintained in the resulting fusion protein. An interacting partner can be purified and used to raise a monoclonal antibody, using methods routinely practiced in the art and described herein above. This antibody can be labeled with the radioactive isotope ¹²⁵I, for example, by methods routinely practiced in the art. In a heterogeneous assay, e.g., the GST-L or GST-L fusion protein can be anchored to glutathione-agarose beads. The interacting partner can then be added in the presence or absence of the test compound in a manner that allows interaction, e.g., binding, to occur. At the end of the reaction period, unbound material can be washed away, and the labeled monoclonal antibody can be added to the system and allowed to bind to the complexed components. The interaction between the L protein and the interacting partner can be detected by measuring the amount of radioactivity that remains associated with the glutathione-agarose beads. A successful inhibition of the interaction by a test compound will result in a decrease in measured radioactivity.

Alternatively, s GST-L or GST-L fusion protein and the interacting partner can be mixed together in liquid in the absence of the solid glutathione-agarose beads. The test compound can be added either during or after complex formation. This mixture can then be added to the glutathione-agarose beads and unbound material is washed away. The extent of inhibition of L gene product/interacting partner interaction can be detected by the addition of a labeled antibody and measuring the radioactivity associated with the beads.

Cell-Based Assays for L Activity

Cell-based methods are presented herein which identify compounds capable of treating lung cancer and other cancers by modulating L molecule activity and/or expression levels. Specifically, such assays identify compounds that affect L molecule dependent processes, such as but not limited to changes in cell morphology, cell division, differentiation, adhesion, motility, phosphorylation, or dephosphorylation of cellular proteins. Such assays can also identify compounds that affect L molecule expression levels or gene activity directly. Compounds identified via such methods can, for example, be utilized in methods for treating lung cancer and other cancers and metastasis thereof.

In one embodiment, an assay is a cell-based assay in which a cell expressing a membrane-bound form of an L gene product, or a biologically active portion thereof, on the cell surface is contacted with a test compound and the ability of the test compound to bind to the polypeptide determined. In another embodiment an L gene product is cytosolic. The cell, for example, may be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to bind preferentially to the polypeptide or a biologically active portion thereof as compared to the known compound.

In another embodiment, the cell-based assays are based on expression of an L gene product in a mammalian cell and measuring L gene-dependent processes. Any mammalian cells that can express an L gene and wherein the L gene product(s) is functional can be used, in particular, cancer cells derived from the lung, such as A549, NCI-H920, NCI-H969, NCI-H23, NCI-H226, NCI-H647, NCI-H1869, NCI-HH1385, NCI-H460, NCI-HI155, NCI-H358, and NCI-H650. Normal bronchial cell lines such as, for example, HBECs and SAECs, may also be used provided that an L gene product is produced. Other mammalian cell lines that can be used include, but are not limited to CHO, HeLa, NIH3T3, and Vero cells. Recombinant expression of an L gene in these cells can be achieved by methods described herein above. In these assays, cells producing functional L gene products are exposed to a test compound for an interval sufficient for the compound to modulate the activity of an L gene product. The activity of an L gene product can be measured directly or indirectly through the detection or measurement of L gene-dependent cellular processes. As a control, a cell not producing the L gene product may be used for comparisons. Depending on the cellular process, any techniques known in the art may be applied to detect or measure it.

In another embodiment, a cell or cell line that is capable of expressing an L gene is contacted with a test compound that is believed to modulate expression of the L gene. Expression levels of the L gene can be monitored in the presence or absence of the test compound. Alternatively, expression levels can be monitored in the presence of a test compound as compared to expression levels of the L gene in the presence of a control compound or a placebo. Any method known in the art can be used to monitor L gene expression. As an example, but not as a limitation, such methods can include Western blot, Northern Blot, and real-time quantitative RT-PCR.

In yet another embodiment, cells which express an L gene product are permeabilized, e.g., by treatment with a mild detergent and exposed to a test compound. Binding of the test compound can be detected directly (e.g., radioactively labeling the test compound) or indirectly (antibody detection) or by any means known in the art.

Any compound can be used in a cell-based assay to test if it affects an L mediated activity or expression levels. The compound can be a protein, a peptide, a nucleic acid, an antibody or fragment thereof, a small molecule, an organic molecule or an inorganic molecule. (e.g., steroid, pharmaceutical drug). A small molecule is considered a non-peptide compound with a molecular weight of less than 500 daltons.

Methods for Treatment of Cancer

Described below are methods and compositions for treating cancer, e.g., lung cancer, using an L gene or gene product as a therapeutic target. The outcome of a treatment is to at least produce in a treated subject a healthful benefit, which in the case of cancer, including lung cancer, includes but is not limited to remission of the cancer, palliation of the symptoms of the cancer, and/or control of metastatic spread of the cancer.

All such methods comprise methods that modulate L gene activity and/or expression, that in turn, modulate the phenotype of the treated cell.

As discussed herein above, successful treatment of lung cancer or other cancers can be brought about by techniques that serve to decrease L gene activity. Activity can be decreased by, for example, directly decreasing L gene product activity and/or by decreasing the level of L gene expression. Thus, the invention provides methods for treating a subject with cancer by administering to said subject an effective amount of a compound that antagonizes an L gene product.

For example, compounds that decrease L activity (identified using assays described herein above) can be used in accordance with the invention to treat lung cancer or other cancers. As indicated, such molecules can include, but are not limited to proteins, nucleic acids, peptides, including soluble peptides, and small organic or inorganic molecules, and can be referred to as L antagonists or agonists. Techniques for the determination of effective doses and administration of such compounds are described herein below.

Further, antisense and ribozyme molecules which inhibit L gene expression can also be used in accordance with the invention to reduce the level of L gene expression, thus effectively reducing the level of an L gene product present, thereby decreasing the level of L mediated activity. The invention therefore relates to a pharmaceutical composition comprising an L gene product. Still further, triple helix molecules can be utilized for reducing the level of L gene activity. Such molecules can be designed to reduce or inhibit either wild type, or if appropriate, mutant target gene activity. Small organic or inorganic molecules can also be used to inhibit L gene expression and/or inhibit production or activity of an L gene product. Techniques for the production and use of such molecules are well known to those of skill in the art.

Antisense Molecules

Anti-sense nucleic acid molecules which are complementary to nucleic acid sequences contained within an L gene as shown in FIGS. 1A-S (SEQ ID NOs: 1-19), including but not limited to anti-sense nucleic acid molecules complementary to one of SEQ ID NOs: 1-19, can be used to treat any cancer, in which the expression level of an L gene is elevated in cancerous cells or tissue as compared to that of normal cells or tissue or a predetermined non-cancerous standard. Thus, in one embodiment of the invention a method for treating lung cancer is provided whereby a patient suffering from lung cancer is treated with an effective amount of an L gene anti-sense nucleic acid molecule.

Antisense approaches involve the design of oligonucleotides (either DNA or RNA) that are complementary to L gene mRNA. The antisense oligonucleotides bind to L gene mRNA transcripts and thereby prevent translation. Absolute complementarity, although preferred, is not required. A sequence “complementary” to a portion of an RNA, as referred to herein, means a sequence having sufficient complementarity to be able to hybridize with the non-poly A portion of the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches it may comprise and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, are considered preferred for antisense applications because, in general, they efficiently inhibit translation. However, sequences complementary to the 3′ untranslated sequences of mRNAs have also been shown to be effective at inhibiting translation of mRNAs as well. (See generally, Wagner, R., 1994, Nature 372:333). Thus, oligonucleotides complementary to the 5′-non-translated region, the 3′-non-translated region, or any other suitable region of the transcript (e.g., part of a coding region) could be used in an antisense approach to inhibit translation of endogenous L gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′-, or coding region of an gene mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared to those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. USA 86:6553; Lemaitre et al., 1987, Proc. Natl. Acad. Sci. USA 84:648; PCT Publication No. WO88/09810, published Dec. 15, 1988) or the blood-brain barrier (see, e.g., PCT Publication No. WO89/10134, published Apr. 25, 1988), hybridization-triggered cleavage agents. (see, e.g., Krol et al., 1988, BioTechniques 6:958) or intercalating agents. (see, e.g., Zon, 1988, Pharm. Res. 5:539). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625). The oligonucleotide is a 2′-0-methylribonucleotide (Inoue et al., 1987, Nucl. Acids Res. 15:6131), or a chimeric RNA-DNA analogue (Inoue et al., 1987, FEBS Lett. 215:327).

An L gene antisense nucleic acid sequence can comprise the complement of any contiguous segment within the sequence of one of the L genes of the invention (SEQ ID NOs: 1-19).

In one embodiment of the present invention, an L antisense nucleic acid sequence is about 50 bp in length. In certain specific embodiments, an L antisense nucleic acid sequence comprises a sequence complementary to any contiguous 50 bp stretch of nucleotides of any one of SEQ ID NOs: 1-19.

In another embodiment an L antisense nucleic acid sequence is about 100 bp in length. In certain specific embodiments, an L antisense nucleic acid sequence comprises a sequence complementary to any contiguous 100 bp stretch of nucleotides of any one of SEQ ID NOs: 1-19.

In another embodiment an L antisense nucleic acid sequence is about 200 bp in length. In a particular embodiment, an L antisense nucleic acid sequence comprises a sequence complementary to any contiguous 200 bp stretch of nucleotides of any one of SEQ ID NOs: 1-19.

In another embodiment an L antisense nucleic acid sequence is about 400 bp in length. In a particular embodiment, an L antisense nucleic acid sequence comprises a sequence complementary to any contiguous 400 bp stretch of nucleotides of any one of SEQ ID NOs: 1-19.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad Sci. U.S.A. 85:7448), etc.

While antisense nucleotides complementary to an L coding region could be used, those complementary to the transcribed untranslated region are most preferred.

Antisense molecules are delivered to cells that express the L gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

It is often difficult, however, to achieve intracellular concentrations of the antisense sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the patient results in the transcription of sufficient amounts of single stranded RNAs that form complementary base pairs with the endogenous L gene transcripts and thereby prevent translation of the L gene mRNA. For example, a vector can be introduced in vivo such that it can be taken up by a cell and direct the transcription of an antisense RNA. Such a vector may remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be effected by any promoter known in the art to act in mammalian, preferably human cells. Such promoters can be inducible or constitutive. Such promoters include but are not limited to: the SV40 early promoter region (Bernoist and Chambon, 1981, Nature 290:304), the promoter contained in the 3′ long terminal repeat of Rous sarcoma virus (Yamamoto et al., 1980, Cell 22:787), the herpes thymidine kinase promoter (Wagner et al., 1981, Proc. Natl. Acad. Sci. USA 78:1441), the regulatory sequences of the metallothionein gene (Brinster et al., 1982, Nature 296:39), etc. Any type of plasmid, cosmid, YAC or viral vector can be used to prepare the recombinant DNA construct that can be introduced directly into the tissue site. Alternatively, viral vectors can be used which selectively infect the desired tissue.

An effective dose of an L antisense oligonucleotide to be administered during a treatment cycle ranges from about 0.01 to 0.1, 0.1 to 1, or 1 to 10 mg/kg/day. The dose of L antisense oligonucleotide to be administered can be dependent on the mode of administration. For example, intravenous administration of an L antisense oligonucleotide would likely result in a significantly higher systemic dose than a systemic dose resulting from a local implant containing a pharmaceutical composition comprising an L antisense oligonucleotide. In one embodiment, an L antisense oligonucleotide is administered subcutaneously at a dose of 0.01 to 10 mg/kg/day. In another embodiment, an L antisense oligonucleotide is administered intravenously at a dose of 0.01 to 10 mg/kg/day. In yet another embodiment, an L antisense oligonucleotide is administered locally at a dose of 0.01 to 10 mg/kg/day. It will be evident to one skilled in the art that local administrations may result in lower systemic or total body doses. For example, local administration methods such as intratumor administration, intraocular injection, or implantation, can produce locally high concentrations of L antisense oligonucleotide, but represent a relatively low dose with respect to total body weight. Thus, in such cases, local administration of an L antisense oligonucleotide is contemplated to result in a total body dose of about 0.01 to 5 mg/kg/day.

In another embodiment, a particularly high dose of an L antisense oligonucleotide, which ranges from about 10 to 50 mg/kg/day, is administered during a treatment cycle.

Moreover, the effective dose of a particular L antisense oligonucleotide may depend on additional factors, including the type of disease, the disease state or stage of disease, the oligonucleotide's toxicity, the oligonucleotide's rate of uptake by cancer cells, as well as the weight, age, and health of the individual to whom the antisense oligonucleotide is to be administered. Because of the many factors present in vivo that may interfere with the action or biological activity of an L antisense oligonucleotide, one of ordinary skill in the art can appreciate that an effective amount of an L antisense oligonucleotide may vary for each individual.

In another embodiment, an L antisense oligonucleotide is administered at a dose which results in circulating plasma concentrations of an L antisense oligonucleotide that are at least 50 nM (nanomolar). As will be apparent to the skilled artisan, lower or higher plasma concentrations of an L antisense oligonucleotide may be preferred depending on the mode of administration. For example, plasma concentrations of an L antisense oligonucleotide of at least 50 nM can be appropriate in connection with, e.g., intravenous, subcutaneous, intramuscular, controlled release, and oral administration methods. In another example, relatively low circulating plasma levels of an L antisense oligonucleotide can be desirable, however, when using local administration methods such as, for example, intratumor administration, intraocular administration, or implantation, which nevertheless can produce locally high, clinically effective concentrations of L antisense oligonucleotide.

A high dose may also be achieved by several administrations per cycle. Alternatively, the high dose may be administered in a single bolus administration. A single administration of a high dose may result in circulating plasma levels of L antisense oligonucleotide that are transiently much higher than 50 nM.

Additionally, the dose of an L antisense oligonucleotide may vary according to the particular L antisense oligonucleotide used. The dose employed is likely to reflect a balancing of considerations, among which are stability, localization, cellular uptake, and toxicity of the particular L antisense oligonucleotide. For example, a particular chemically modified L antisense oligonucleotide may exhibit greater resistance to degradation, or may exhibit higher affinity for the target nucleic acid, or may exhibit increased uptake by the cell or cell nucleus; all of which may permit the use of low doses. In yet another example, a particular chemically modified L antisense oligonucleotide may exhibit lower toxicity than other antisense oligonucleotides, and therefore can be used at high doses. Thus, for a given L antisense oligonucleotide, an appropriate dose to administer can be relatively high or low. The invention contemplates the continued assessment of optimal treatment schedules for particular species of L antisense oligonucleotides. The daily dose can be administered in one or more treatments.

A “low dose” or “reduced dose” refers to a dose that is below the normally administered range, i.e., below the standard dose as suggested by the Physicians' Desk Reference, 54^{th} Edition (2000) or a similar reference. Such a dose can be sufficient to inhibit cell proliferation, or demonstrates ameliorative effects in a human, or demonstrates efficacy with fewer side effects as compared to standard cancer treatments. Normal dose ranges used for particular therapeutic agents and standard cancer treatments employed for specific diseases can be found in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

Reduced doses of an L nucleic acid molecule, an L polypeptide, an L antagonist, and/or a combination therapeutic may demonstrate reduced toxicity, such that fewer side effects and toxicities are observed in connection with administering an L antagonist and one or more cancer therapeutics for shorter duration and/or at lower doses when compared to other treatment protocols and dosage formulations, including the standard treatment protocols and dosage formulations as described in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

A “treatment cycle” or “cycle” refers to a period during which a single therapeutic or sequence of therapeutics is administered. In some instances, one treatment cycle may be desired, such as, for example, in the case where a significant therapeutic effect is obtained after one treatment cycle. The present invention contemplates at least one treatment cycle, generally preferably more than one treatment cycle.

Other factors to be considered in determining an effective dose of an L antisense oligonucleotide include whether the oligonucleotide will be administered in combination with other therapeutics. In such cases, the relative toxicity of the other therapeutics may indicate the use of an L antisense oligonucleotide at low doses. Alternatively, treatment with a high dose of L antisense oligonucleotide can result in combination therapies with reduced doses of therapeutics. In a specific embodiment, treatment with a particularly high dose of L antisense oligonucleotide can result in combination therapies with greatly reduced doses of cancer therapeutics. For example, treatment of a patient with 10, 20, 30, 40, or 50 mg/kg/day of an L antisense oligonucleotide can further increase the sensitivity of a subject to cancer therapeutics. In such cases, the particularly high dose of L antisense oligonucleotide is combined with, for example, a greatly shortened radiation therapy schedule. In another example, the particularly high dose of an L antisense oligonucleotide produces significant enhancement of the potency of cancer therapeutic agents.

Additionally, the particularly high doses of L antisense oligonucleotide may further shorten the period of administration of a therapeutically effective amount of L antisense oligonucleotide and/or additional therapeutic, such that the length of a treatment cycle is much shorter than that of the standard treatment.

The invention contemplates other treatment regimens depending on the particular L antisense oligonucleotide to be used, or depending on the particular mode of administration, or depending on whether an L antisense oligonucleotide is administered as part of a combination therapy, e.g., in combination with a cancer therapeutic agent. The daily dose can be administered in one or more treatments.

Ribozyme Molecules

Ribozyme molecules that are complementary to RNA sequences transcribed from an L gene (shown in FIGS. 1A-S) may be used to treat any cancer, including lung cancer. Ribozymes are enzymatic RNA molecules capable of catalyzing the specific cleavage of RNA (For a review see, for example Rossi, J., 1994, Current Biology 4:469). The mechanism of ribozyme action involves sequence specific or selective hybridization of the ribozyme molecule to a complementary target RNA, followed by endonucleolytic cleavage. The composition of ribozyme molecules generally includes one or more sequences complementary to the target gene mRNA and the well known catalytic sequence responsible for mRNA cleavage (See U.S. Pat. No. 5,093,246). As such, within the scope of the invention are engineered hammerhead motif ribozyme molecules that specifically and efficiently catalyze endonucleolytic cleavage of RNA sequences encoding target gene proteins. Ribozyme molecules designed to cleave L mRNA transcripts catalytically also prevent translation of L mRNA to protein. (See, e.g., PCT International Publication WO90/11364, published Oct. 4, 1990; Sarver et al., 1990, Science 247:1222). While ribozymes that cleave mRNA at site-specific recognition sequences can be used to destroy L mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature 334:585. Preferably the ribozyme is engineered such that the cleavage recognition site is located near the 5′ end of an L mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Cech and collaborators (Zaug et al., 1984, Science 224:574; Zaug and Cech, 1986, Science 231:470; Zaug et al., 1986, Nature 324:429; published International patent application No. WO 88/04300 by University Patents Inc.; Been and Cech, 1986, Cell 47:207). The Cech-type ribozymes have an eight base pair active site that hybridizes to a target RNA sequence whereafter cleavage of the target RNA takes place. The invention encompasses Cech-type ribozymes that target eight base-pair active site sequences that are incorporated into an L gene transcript.

As in the antisense approach, the ribozymes can be composed of modified oligonucleotides (e.g., for improved stability, targeting, etc.) and should be delivered to cells that express an L gene in vivo. A preferred method involves delivery (e.g., by transfection) of a DNA construct “encoding” the ribozyme under the control of a strong constitutive pol III or pol II promoter, successful delivery of which results in expression of sufficient quantities of the ribozyme in recipient cells to destroy endogenous L gene messages thereby inhibiting L protein translation. Ribozymes, unlike antisense molecules, are catalytic and therefore function effectively at lower intracellular concentrations.

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules can be generated by in vitro or in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications can be introduced into the DNA molecules to increase intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

Therapeutic Antibodies

Antibodies exhibiting the ability to downregulate L gene product activity can be utilized to treat lung cancer and other cancers wherein L gene expression levels are elevated. Such antibodies can be generated against wild type or mutant L proteins, or against peptides corresponding to portions of the proteins using standard techniques as described herein above. The antibodies include but are not limited to polyclonal, monoclonal, Fab fragments, single chain antibodies, chimeric antibodies, and the like.

Antibodies that recognize any epitope of an L protein can be used as therapeutic reagents for the treatment of a patient with a cancer associated with aberrant L activity.

For L genes that are generally expressed as intracellular proteins, it is preferred that internalizing antibodies are used. However, lipofectin or liposomes can be used to deliver an L antibody or an L binding fragment of the Fab region into cells. When fragments of an L antibody are used, the smallest inhibitory fragment that binds to an L molecule is preferred. For example, peptides having an amino acid sequence corresponding to the domain of the variable region of an antibody that binds to an L molecule can be used. Such peptides can be synthesized chemically or produced via recombinant DNA technology using methods well known in the art (e.g., see Creighton, 1983, supra; and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889).

Also contemplated by the methods of the invention are antibodies that are conjugated to a cytostatic and/or a cytotoxic agent. Such conjugated antibodies are useful for treating a patient with cancer because they target cancer cells expressing the antigen for which the antibody is specific, thereby inhibiting the proliferation of these cells and/or killing these cells. A useful class of such cytotoxic or cytostatic agents includes, but is not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids.

Individual cytotoxic or cytostatic agents encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a preferred embodiment, the cytotoxic or cytostatic agent is an antimetabolite. The antimetabolite can be a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, and trifluridine.

Techniques for conjugating such therapeutic moieties to proteins, and in particular to antibodies, are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson et al. (eds.), pp. 623-53 (Marcel Dekker, Inc., 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Targeted Disruption of L Gene Expression

As briefly described herein above, endogenous L gene expression can be reduced by inactivating or “knocking out” the L gene or its promoter using targeted homologous recombination. (e.g., see Smithies et al., 1985, Nature 317:230; Thomas & Capecchi, 1987, Cell 51:503; Thompson et al., 1989 Cell 5:313). For example, a mutant, non-functional L gene (or a completely unrelated DNA sequence) flanked by DNA homologous to an endogenous L gene (either the coding regions or regulatory regions of an L gene) can be used, with or without a selectable marker and/or a negative selectable marker, to transfect cells that express the L gene in vivo. Insertion of the DNA construct, via targeted homologous recombination, results in inactivation of the L gene. Such approaches are particularly useful for modifications to ES (embryonic stem) cells that can be used to generate animal offspring with an inactive L gene homolog (e.g., see Thomas & Capecchi 1987 supra and Thompson 1989, supra). Such techniques can also be utilized to generate animal models of lung cancer and other types of cancer. It should be noted that this approach can be adapted for use in humans, provided the recombinant DNA constructs are directly administered or targeted to the required site in vivo using appropriate vectors, e.g., herpes virus vectors, retrovirus vectors, adenovirus vectors, or adeno associated virus vectors.

Alternatively, endogenous L gene expression can be reduced by targeting deoxyribonucleotide sequences complementary to the regulatory region of an L gene (i.e., an L gene promoter and/or enhancers) to form triple helical structures that prevent transcription of an L gene in target cells in the body. (See generally, Helene, 1991, Anticancer Drug Des. 6(6):569; Helene et al., 1992, Ann, N.Y. Acad. Sci. 660:27; and Maher, 1992, Bioassays 14(12):807).

Combination Therapies

Administration of an L molecule antagonist can potentiate the effect of anti-cancer agents. In a preferred embodiment, the invention further encompasses the use of combination therapy to prevent or treat cancer. In one embodiment, an L gene antagonist selectively or specifically antagonizes L gene expression and/or activity.

In one embodiment, lung cancer and other cancers (e.g., of the breast, brain, prostate, ovary, gastric system, pancreas, colon,) can be treated with a pharmaceutical composition comprising an L molecule antagonist in combination with 5-fluorouracil, cisplatin, docetaxel, doxorubicin, Herceptin®, gemcitabine (Seidman, 2001, Oncology 15:11-14), IL-2, paclitaxel, and/or VP-16 (etoposide).

These combination therapies can also be used to prevent cancer, prevent the recurrence of cancer, or prevent the spread or metastasis or cancer.

Combination therapy also includes, in addition to administration of an L molecule antagonist, the use of one or more molecules, compounds or treatments that aid in the prevention or treatment of cancer (i.e., cancer therapeutics), which molecules, compounds or treatments include, but are not limited to, chemoagents, immunotherapeutics, cancer vaccines, anti-angiogenic agents, cytokines, hormone therapies, gene therapies, and radiotherapies.

In one embodiment, one or more chemoagents, in addition to an L molecule antagonist, is administered to treat a cancer patient. A chemoagent (or “anti-cancer agent” or “anti-tumor agent” or “cancer therapeutic”) refers to any molecule or compound that assists in the treatment of tumors or cancer. Examples of chemoagents contemplated by the present invention include, but are not limited to, cytosine arabinoside, taxoids (e.g., paclitaxel, docetaxel), anti-tubulin agents (e.g., paclitaxel, docetaxel, epothilone B, or its analogues), macrolides (e.g., rhizoxin ) cisplatin, carboplatin, adriamycin, tenoposide, mitozantron, discodermolide, eleutherobine, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), antimetabolites (e.g., methotrexate, 6-mercaptopurine, 6-thioguanine, cytarabine, flavopiridol, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, coichine, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gamcitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath-1, straplozocin, thioguanine, tretinoin, vinorelbine, or any fragments, family members, or derivatives thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone.

In one embodiment, said chemoagent is gemcitabine at a dose ranging from 100 to 1000 mg/m²/cycle. In one embodiment, said chemoagent is dacarbazine at a dose ranging from 200 to 4000 mg/m²/cycle. In a preferred embodiment, said dose ranges from 700 to 1000 mg/m²/cycle. In another embodiment, said chemoagent is fludarabine at a dose ranging from 25 to 50 mg/m²/cycle. In another embodiment, said chemoagent is cytosine arabinoside (Ara-C) at a dose ranging from 200 to 2000 mg/m²/cycle. In another embodiment, said chemoagent is docetaxel at a dose ranging from 1.5 to 7.5 mg/kg/cycle. In another embodiment, said chemoagent is paclitaxel at a dose ranging from 5 to 15 mg/kg/cycle. In yet another embodiment, said chemoagent is cisplatin at a dose ranging from 5 to 20 mg/kg/cycle. In yet another embodiment, said chemoagent is 5-fluorouracil at a dose ranging from 5 to 20 mg/kg/cycle. In yet another embodiment, said chemoagent is doxorubicin at a dose ranging from 2 to 8 mg/kg/cycle. In yet another embodiment, said chemoagent is epipodophyllotoxin at a dose ranging from 40 to 160 mg/kg/cycle. In yet another embodiment, said chemoagent is cyclophosphamide at a dose ranging from 50 to 200 mg/kg/cycle. In yet another embodiment, said chemoagent is irinotecan at a dose ranging from 50 to 75, 75 to 100, 100 to 125, or 125 to 150 mg/m²/cycle. In yet another embodiment, said chemoagent is vinblastine at a dose ranging from 3.7 to 5.4, 5.5 to 7.4, 7.5 to 11, or 11 to 18.5 mg/m²/cycle. In yet another embodiment, said chemoagent is vincristine at a dose ranging from 0.7 to 1.4, or 1.5 to 2 mg/m²/cycle. In yet another embodiment, said chemoagent is methotrexate at a dose ranging from 3.3 to 5, 5 to 10, 10 to 100, or 100 to 1000 mg/m²/cycle.

In a preferred embodiment, the invention further encompasses the use of low doses of chemoagents when administered as part of an L molecule antagonist treatment regimen. For example, initial treatment with an L molecule antagonist increases the sensitivity of a tumor to subsequent challenge with a dose of chemoagent, which dose is near or below the lower range of dosages when the chemoagent is administered without an L molecule antagonist. In one embodiment, an L molecule antagonist and a low dose (e.g., 6 to 60 mg/m²/day or less) of docetaxel are administered to a cancer patient. In another embodiment, an L molecule antagonist and a low dose (e.g., 10 to 135 mg/m²/day or less) of paclitaxel are administered to a cancer patient. In yet another embodiment, an L molecule antagonist and a low dose (e.g., 2.5 to 25 mg/m²/day or less) of fludarabine are administered to a cancer patient. In yet another embodiment, an L molecule antagonist and a low dose (e.g., 0.5 to 1.5 g/m²/day or less) of cytosine arabinoside (Ara-C) are administered to a cancer patient.

The invention, therefore, contemplates the use of one or more L molecule antagonists or agonists, which is administered prior to, subsequently, or concurrently with low doses of chemoagents, for the prevention or treatment of cancer.

In one embodiment, said chemoagent is gemcitabine at a dose ranging from 10 to 100 mg/m²/cycle.

In another embodiment, said chemoagent is cisplatin, e.g., PLATINOL™ or PLATINOL-AQ™(Bristol Myers), at a dose ranging from 5 to 10, 10 to 20, 20 to 40, or 40 to 75 mg/m²/cycle. In another embodiment, a dose of cisplatin ranging from 7.5 to 75 mg/m²/cycle is administered to a patient with ovarian cancer or other cancer. In another embodiment, a dose of cisplatin ranging from 5 to 50 mg/m²/cycle is administered to a patient with bladder cancer or other cancer.

In another embodiment, said chemoagent is carboplafin, e.g., PARAPLATIN™(Bristol Myers), at a dose ranging from 2 to 4, 4 to 8, 8 to 16, 16 to 35, or 35 to 75 mg/m²/cycle. In another embodiment, a dose of carboplatin ranging from 7.5 to 75 mg/m²/cycle is administered to a patient with ovarian cancer or other cancer. In another embodiment, a dose of carboplatin ranging from 5 to 50 mg/m²/cycle is administered to a patient with bladder cancer or other cancer. In another embodiment, a dose of carboplatin ranging from 2 to 20 mg/m²/cycle is administered to a patient with testicular cancer or other cancer.

In another embodiment, said chemoagent is docetaxel, e.g., TAXOTERE™ (Rhone Poulenc Rorer) at a dose ranging from 6 to 10, 10 to 30, or 30 to 60 mg/m²/cycle.

In another embodiment, said chemoagent is paclitaxel, e.g., TAXOL™ (Bristol Myers Squibb), at a dose ranging from 10 to 20, 20 to 40, 40 to 70, or 70 to 135 mg/kg/cycle.

In another embodiment, said chemoagent is 5-fluorouracil at a dose ranging from 0.5 to 5 mg/kg/cycle.

In another embodiment, said chemoagent is doxorubicin, e.g., ADRLAMYCIN™ (Pharmacia & Upjohn), DOXIL (Alza), RUBEX™ (Bristol Myers Squibb), at a dose ranging from 2 to 4, 4 to 8, 8 to 15, 15 to 30, or 30 to 60 mg/kg/cycle.

In another embodiment, an L molecule antagonist is administered in combination with one or more immunotherapeutic agents, such as antibodies and immunomodulators, which include, but are not limited to, Herceptin®, Retuxan®, OvaRex, Panorex, BEC2, IMC-C225, Vitaxin, Campath I/H, Smart MI95, LymphoCide, Smart I D10, and Oncolym, rituxan, rituximab, gemtuzumab, or trastuzumab.

In another embodiment, an L molecule antagonist is administered in combination with one or more anti-angiogenic agents, which include, but are not limited to, angiostatin, thalidomide, kringle 5, endostatin, Serpin (Serine Protease Inhibitor) anti-thrombin, 29 kDa N-terminal and a 40 IcDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (Maione et al., 1990, Cancer Res. 51:2077), a 14-amino acid peptide corresponding to a fragment of collagen I (Tolma et al., 1993, J. Cell Biol. 122:497), a 19 amino acid peptide corresponding to a fragment of Thrombospondin I (Tolsma et al., 1993, J. Cell Biol. 122:497), a 20-amino acid peptide corresponding to a fragment of SPARC (Sage et al., 1995, J. Cell. Biochem. 57: 1329-), or any fragments, family members, or derivatives thereof, including pharmaceutically acceptable salts thereof.

Other peptides that inhibit angiogenesis and correspond to fragments of laminin, fibronectin, procollagen, and EGF have also been described (See the review by Cao, 1998, Prog. Mol. Subcell. Biol. 20:161). Monoclonal antibodies and cyclic pentapeptides, which block certain integrins that bind RGD proteins (i.e., possess the peptide motif Arg-Gly-Asp), have been demonstrated to have anti-vascularization activities (Brooks et al., 1994, Science 264:569; Hammes et al., 1996, Nature Medicine 2:529). Moreover, inhibition of the urokinase plasminogen activator receptor by antagonists or agonists inhibits angiogenesis, tumor growth and metastasis (Min et al., 1996, Cancer Res. 56:2428-33; Crowley et al., 1993, Proc Natl Acad Sci. USA 90:5021). Use of such anti-angiogenic agents in combination with L molecule modulators is also contemplated by the present invention.

In another embodiment, an L molecule antagonist is administered in combination with a regimen of radiation.

In another embodiment, an L molecule antagonist is administered in combination with one or more cytokines, which include, but are not limited to, lymphokines, tumor necrosis factors, tumor necrosis factor-like cytokines, lymphotoxin-α, lymphotoxin-β, interferon-α, interferon-β, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Fas ligand, 4-1BBL, endothelial monocyte activating protein or any fragments, family members, or derivatives thereof, including pharmaceutically acceptable salts thereof.

In yet another embodiment, an L molecule antagonist is administered in combination with a cancer vaccine. Examples of cancer vaccines include, but are not limited to, autologous cells or tissues, non-autologous cells or tissues, carcinoembryonic antigen, alpha-fetoprotein, human chorionic gonadotropin, BCG live vaccine, melanocyte lineage proteins (e.g., gp100, MART-1/MelanA, TRP-1 (gp75), tyrosinase, widely shared tumor-associated, including tumor-specific, antigens (e.g., BAGE, GAGE-1, GAGE-2, MAGE-1, MAGE-3, N-acetylglucosaminyltransferase-V, p15), mutated antigens that are tumor-associated (β-catenin, MUM-1, CDK4), nonmelanoma antigens (e.g., HER-2/neu (breast and ovarian carcinoma), human papillomavirus-E6, E7 (cervical carcinoma), MUC-1 (breast, ovarian and pancreatic carcinoma). For human tumor antigens recognized by T-cells, see generally Robbins and Kawakami, 1996, Curr. Opin. Immunol. 8:628. Cancer vaccines may or may not be purified preparations.

In yet another embodiment, an L molecule antagonist is used in association with a hormonal treatment. Hormonal therapeutic treatments comprise hormonal agonists, hormonal antagonists (e.g., flutamide, tamoxifen, leuprolide acetate (LUPRON), LH—RH antagonists), inhibitors of hormone biosynthesis and processing, and steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

In yet another embodiment, an L molecule antagonist is used in association with a gene therapy program in the treatment of cancer. In one embodiment, gene therapy with recombinant cells secreting interleukin-2 is administered in combination with an L molecule antagonist to prevent or treat cancer, particularly lung cancer (See, e.g., Deshmukh et al., 2001, J. Neurosurg. 94:287).

In one embodiment, an L molecule antagonist is administered, in combination with at least one cancer therapeutic agent, for a short treatment cycle to a cancer patient. The duration of treatment with the cancer therapeutic agent may vary according to the particular cancer therapeutic agent used. The invention also contemplates discontinuous administration or daily doses divided into several partial administrations. Appropriate treatment time-lines for cancer therapeutic agents will be appreciated by those skilled in the art, and the invention contemplates the continued assessment of optimal treatment schedules for each cancer therapeutic agent.

The present invention contemplates at least one cycle, preferably more than one cycle during which a single therapeutic or sequence of therapeutics is administered. An appropriate period of time for one cycle will be appreciated by the skilled artisan, as will the total number of cycles, and the interval between cycles. The invention contemplates the continued assessment of optimal treatment schedules for each L molecule antagonist and cancer therapeutic agent.

Pharmaceutical Preparations and Methods of Administration

The compounds, proteins, peptides, nucleic acid sequences and fragments thereof, described herein can be administered to a patient at therapeutically effective doses to treat cancer, e.g., lung cancer wherein the expression level of an L gene is elevated compared to a non-cancerous sample or a predetermined non-cancerous standard. A therapeutically effective amount or dose refers to that amount of a compound sufficient to result in a healthful benefit in the treated subject.

Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD50/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

Formulations and Use

The invention relates to pharmaceutical compositions, including, but not limited to pharmaceutical compositions comprising an L gene product, or antagonists or agonists thereof, for the treatment or prevention of cancer.

Pharmaceutical compositions for use in accordance with the present invention, e.g., methods to treat or prevent cancer, can be formulated in a conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can also be suitably formulated to provide controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Vaccine Therapy

L gene nucleic acids and L polypeptides and peptides encoded therefrom and fragments thereof, may be used as vaccines by administering to an individual at risk for developing cancer an amount of said protein, peptide, or nucleic acid that effectively stimulates an immune response against an L-encoded polypeptide and protects that individual from cancer. The invention thus contemplates a method of vaccinating a subject against cancer wherein said subject is at risk for developing cancer.

Many methods may be used to introduce the vaccine formulations described herein above, these include but are not limited to intranasal, intratracheal, oral, intradermal, intramuscular, intraperitoneal, intravenous, and subcutaneous route. Various adjuvants may be used to increase the immunological response, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Nucleic acid sequences of the invention, including variants and derivatives, can be used as vaccines, e.g., by genetic immunization. Genetic immunization is particularly advantageous as it stimulates a cytotoxic T-cell (CTL) response. Genetic immunization is not, however, coupled with the potential risks associated with the use of live attenuated vaccines, which are also capable of triggering a CTL response, but can revert to a virulent form and infect the host. As used herein, genetic immunization comprises inserting the nucleotides of the invention into a host cell, wherein the encoded proteins are expressed. These translated proteins are then either secreted or processed by the host cell for presentation to immune cells and an immune reaction is stimulated. Preferably, the immune reaction is a CTL response, however, a humoral response or macrophage stimulation is also useful in preventing initial or additional tumor growth and metastasis or spread of the cancer. A skilled artisan will appreciate that there are various methods for introducing foreign nucleotides into a host animal and subsequently into cells for genetic immunization, for example, by intramuscular injection of about 50 mg of plasmid DNA encoding the proteins of the invention solubilized in 50 ml of sterile saline solution, with a suitable adjuvant (See, e.g., Weiner and Kennedy, 1999, Scientific American 7:50-57; Lowrie et al., 1999, Nature 400:269-271).

The invention thus provides a vaccine formulation for the prevention of cancer comprising an immunogenic amount of an L gene product. The invention further provides for an immunogenic composition comprising a purified L gene product.

Kits

The invention includes a kit for assessing the presence of cancer cells including lung cancer cells (e.g., in a sample such as a patient sample). The kit comprises a plurality of reagents, each of which is capable of binding specifically with a nucleic acid or polypeptide corresponding to a marker of the invention, e.g., an L gene or gene product or fragment thereof. Suitable reagents for binding to a polypeptide corresponding to a marker of the invention include antibodies, antibody derivatives, labeled antibodies, antibody fragments, and the like. Suitable reagents for binding to a nucleic acid (e.g., a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound to a substrate, PCR primer pairs, molecular beacon probes, and the like.

The kit of the invention may optionally comprise additional components useful for performing the methods of the invention. By way of example, the kit may comprise fluids (e.g., SSC buffer) suitable for annealing complementary nucleic acids or for binding an antibody to a protein for which it is immunologically specific, one or more sample compartments, an instructional material which describes performance of a method of the invention, a sample of normal cells, a sample of cancer cells, and the like.

### EXAMPLES

In the development of lung neoplasia and other cancers, subsets of genes are specifically and differentially expressed at various stages of disease progression. Some of these genes/gene subsets are critical for progression of the cancer, and are associated with a particular stage of the disease, for example, metastasis. While several NSCLC-specific gene expression studies have been previously reported, (Beer et al., 2002, Nat. Med., 8, 816-824; Bhattacharjee et al., 2001, Proc. Natl. Acad. Sci. USA, 98, 13790-13795; Garber et al., 2001, Proc. Natl. Acad. Sci. USA, 98, 13784-13789; Heighway et al., 2002, Oncogene, 21, 7749-7763; Nacht et al., 2001, Proc. Natl. Acad. Sci. USA., 98, 15203-15208) there remains a dearth of NSCLC targets useful for diagnostic and/or therapeutic applications. Several technologies are currently being utilized for gene expression profiling in cancer, including: Serial Analysis of Gene Expression (SAGE) (Velculescu et al., 1995, Science,270, 484-487), Suppression Subtractive Hybridization (SSH) (Diatchenko et al., 1996, Proc. Natl. Acad. Sci. USA, 93, 6025-6030), cDNA arrays (DeRisi et al., 1996, Nat. Genet., 14, 457-460), and oligonucleotide chips (Lockhart et al., 1996, Nat. Biotechnol., 14, 1675-1680). Independently, each of these techniques can be effective for identifying differentially expressed genes. In the present study, a combination of SSH and cDNA arrays was used to identify NSCLC-associated genes.

Materials and Methods

Cell Culture: NSCLC cell lines including: A549, NCI-H23, NCI-H920, NCI-H969, NCI-H647, NCI-H226, NCI-H1869, NCI-H1385, NCI-H460, NCI-H1 155, NCI-H358, and NCI-H650 (ATCC, Manassas, Va.) were grown in SAGM medium® (Clonetics, San Diego, Calif.) supplemented with 0.5% fetal bovine serum (Sigma, St. Louis, Mo.). All tumor cell lines were passaged once per week by trypsinization and replated at 2500-3000 cells/cm² (Clonetics, San Diego, Calif.). Normal human bronchial epithelial cells (NHBEs) (Clonetics, San Diego, Calif.) were grown in SAGM medium® supplemented with 0.5% fetal bovine serum.

RNA isolation: Total RNA was isolated from cultured cells using RNA-Bee™ (Tel-Test, Inc., Friendswood, Tex.). Poly A+RNA was extracted using the Oligotex mRNA Midi kit® (Qiagen, Inc., Valencia, Calif.).

Generation of SSH Libraries: Two NSCLC-specific SSH cDNA libraries were constructed as described by Diatchenko et al., 1996, Proc. Natl. Acad. Sci. 93:6025-6030. Library one (NSCLC-1) was constructed using a pool of NSCLC cell lines including: A549, NCI-H23, NCI-H226, and NCI-H460 (tester RNA) vs. a pool of normal patient tissue RNAs (driver RNA) including colon, kidney, lung, liver (Origene, Inc., Rockville, Md.), and pancreas (Clontech, Palo Alto, Calif.), and cultured NHBEs. Library two (NSCLC-2) was constructed using a pool of NSCLC cell lines including: A549, NCI-H23, NCI-H920, NCI-H969, NCI-H358, and NCI-H650 (tester RNA) vs. a pool of normal patient tissue RNAs (driver RNA) including: colon, kidney, lung, liver (Origene, Inc., Rockville, Md.), and pancreas and spleen (Clontech, Palo Alto, Calif.).

Driver cDNA was synthesized from poly A+RNA using 1 ul of 10 uM cDNA synthesis primer 5′-TTTTGTACAAGCTT₃₀N₁N-3′ (SEQ ID NO: 39) and 1 ul of 200 u/ul Superscript II Reverse Transcriptase® (Invitrogen, Carlsbad, Calif.). The resulting cDNA pellets were pooled and digested with 1.5 ul of 10u/ul of Rsa I restriction enzyme. Driver cDNAs were precipitated with 100 ul of 10M Ammonium Acetate (Sigma, St. Louis, Mo.), 3 ul of 20 mg/ml glycogen (Roche Molecular Biochemicals, Indianapolis, Ind.) and 1 ml of ethanol (Sigma, St. Louis, Mo.). The cDNA preparations were then resuspended in 5 ul of diethyl pyrocarbonate (DEPC) treated water.

Tester cDNA was synthesized from poly A+RNA as described above for the driver. The resulting cDNA pellets were pooled and digested with 1.5 ul of 10 u/ul of Rsa I restriction enzyme. Rsa I digested tester cDNA was diluted in 5 ul of DEPC treated water prior to adaptor ligation. Diluted tester cDNA (2 ul) was ligated to 2 ul of 10 uM adaptor 1 (5′-CTAATACGACTCACTATAGGGCTCGAGCGGCCGCCCGGGCAGGT-3′) (SEQ ID NO: 40) and 2 ul of 10 uM adaptor 2R (5′-CTAATACGACTCACTATAGGGCAGCGTGGTCGCGGCCGAGGT-3′) (SEQ ID NO: 41) in separate reactions using 0.5 units of T4 DNA ligase (Invitrogen, Carlsbad, Calif.).

Driver cDNA (600 ng) was added separately to each of the two tubes containing adaptor-1 ligated tester (20 ng) and adaptor 2R ligated tester (20 ng). The samples were mixed, ethanol precipitated as described above, and resuspended in 1.5 ul of hybridization buffer (50 mM Hepes pH 8.3, 0.5 M NaCl/0.0.2 mM EDTA pH 8.0). The reaction mixture was placed in hot start PCR tubes, (Molecular BioProducts, San Diego, Calif.), denatured at 95° C. for 1.5 min. and then incubated at 68° C. for 8 hrs. After this initial hybridization, the samples were combined and excess heat denatured driver cDNA (150 ng) was added. This secondary reaction mixture was incubated overnight at 68° C. The final hybridization mixture was diluted in 200 ul of dilution buffer (20 mM Hepes pH 8.3, 50 mM NaCl, 0.2 mM EDTA) and stored at −20° C.

Two rounds of PCR amplification were performed for each SSH library. The primary PCR was performed in 25 ul. The reaction mixture contained 1 ul of diluted subtracted cDNA, 1 ul of 10 uM PCR primer 1 (5′-CTAATACGACTCACTATAGGGC-3′) (SEQ ID NO: 42), 10×PCR buffer consisting of (166 mM NH₄C₂H₃O₂, 670 mM Tris pH 8.8, 67 mM MgCl₂, and 100 mM 2-mercaptoethanol), 1.5 ul of 10 mM dNTP's, 1.5 ul dimethyl sulfoxide (DMSO) (Sigma, St. Louis, Mo.), and 0.25 ul of 5 u/ul of Taq polymerase (Brinkmann, Westbury, N.Y.). PCR was performed with the following cycling conditions: 75° C. for 7 min.; 94° C. for 2 min.; 94° C. for 30 sec., and 72° C. for 1.5 min.; and a final extension at 72° C. for 5 min. A secondary PCR was performed using 1 ul of the primary PCR as template with the same reaction components as above. Nested PCR primers NP1 (5′-TCGAGCGGCCGCCCGGGCAGGT-3′) (SEQ ID NO: 43) and NP2R (5′-AGCGTGGTCGCGGCCGAGGT-3′) (SEQ ID NO: 44) were used in place of PCR primer 1. The secondary PCR was performed with the following cycling conditions: 94° C. for 2 min.; 94° C. for 30 sec., 68° C. for 30 sec., and 72° C. for 1.5 min.; and a final extension at 72° C. for 5 min. products were analyzed on 1.5% ultrapure agarose gels (Invitrogen, Carlsbad, Calif.) and visualized by ethidium bromide (Fisher Chemical, Fair Lawn, N.J.). Subtraction efficiency was confirmed by PCR depletion of EF-1 and Tubulin. EF-1 primers were EF-1 (5′-CTGTTCCTGTTGGCCGAGTC-3′) (SEQ ID NO: 45) and EF-2 (5′-CGATGCATTGTTATCATTAAC-3′) (SEQ ID NO: 46). Tubulin primers were Tu-1 (5′-CACCCTGAGCAGCTCATCAC-3′) (SEQ ID NO: 47) and Tu2 (5′-GGCCAGGGTCACATTTCACC-3′) (SEQ ID NO: 48).

Cloning of SSH Pools Into pCR4-TOPO: The SSH-cDNA pools were cloned into the pCR4-TOPO® vector (Invitrogen, Carlsbad, Calif.) and transformed into chemically competent TOP 10 cells® (Invitrogen, Carlsbad, Calif.). The library was plated on LB agar plates (Becton Dickinson, Sparks, Md.) containing 50 μg/ul kanamycin (Sigma, St. Louis, Mo.). Cloning efficiency and size distribution for each library was determined by amplification using M13 (-20) (5′-GTAAAACGACGGCCAGT-3′) (SEQ ID NO: 49) and M1 3R (5′-CAGGAAACAGCTATGACC-3′) (SEQ ID NO: 50) universal primers.

Custom Array Generation: SSH clones containing cDNA sequences of interest were amplified using M13 (-20) and M13R universal primers. PCR products were purified using 96-well MultiScreen PCR Purification Plates (Millipore, Bedford, Mass.). Microarrays were prepared by spotting targets in duplicate on positively charged nylon membranes (Hybond-XL®, Amersham Pharmacia Biotech, Piscataway, N.J.) at concentrations of 2 ng DNA/spot using a Biomek 2000 Robot® (Beckman Coulter Inc., Fullerton, Calif.). For probe construction, mRNA was isolated from cell lines as described above. Poly A+RNA (1 ug) was converted to cDNA and labeled with (α-P32) dCTP (Amersham Pharmacia Biotech, Piscataway, N.J.) by reverse transcription using Superscript II RT® (Invitrogen, Carlsbad, Calif.). Hybridizations were performed overnight at 42° C. in 6× Saline Sodium Citrate (SSC), 0.1% sodium dodecyl sulfate (SDS), 50% deionized formamide, and 5× Denhardt's solution (1% Ficoll Type 400, 1% polyvinylpyrrolidone, and 1% bovine serum albumin) (Research Genetics, Huntsville, Ala.). Wash conditions were 4 times in 2×SSC/0.1% SDS for 10 min. each at room temperature, followed by 4 high stringency washes in 0.1×SSC/0.1% SDS at 65° C. for 30 min. each.

Array Data Analysis: Hybridization Intensities were quantitated on the Phosphorlmager SI® (Molecular Dynamics, Sunnyvale, Calif.) using ArrayVision 6.0 Software® (Imaging Research, St. Catharines, ON, CA). Average signal intensities were determined for each set of duplicate spots. For each membrane analyzed, relative quantitative values were determined based on normalization to multiple housekeeping genes spotted at various locations on each membrane.

Expression Profiling: A total of 3072 combined cDNA inserts from NSCLC-1 and NSCLC-2 libraries were PCR amplified using M13F and M13R universal primers. Amplified clones were visualized using 1.2% agarose gels and stained with ethidium bromide. PCR products were purified using 96-well purification plates (Millipore, Bedford, Mass.) and deposited in equal concentrations (2 ng/spot) onto nylon membranes (Hybond-XL, Amersham Pharmacia Biotech, Piscataway, N.J.) using the Biomek 2000 Laboratory Automation Workstation (Beckman Coulter, Fullerton, Calif.). Membranes were denatured in 1 N NaOH, 2 M NaCl, and 25 mM EDTA, neutralized in 2×SSC, and UV cross-linked.

Array hybridizations were performed using poly (A)+RNA converted to cDNA using Superscript II RT (Invitrogen) and labeled with [α-³²P] dCTP (Amersham). Hybridizations were performed overnight at 42° C. in 6×SSC, 0.1% SDS, 50% formamide, and 5× Denhardt's solution (Research Genetics, Huntsville, Ala.). Membrane wash conditions included 4 times with 2×SSC/0.1% SDS for 10 min at room temperature followed by 4 times with 0.1×SSC/0.1%SDS for 30 min at 65° C.

Array differentials were calculated using PhosPhorlmager SI (Molecular Dynamics, Sunnyvale, Calif.) and ArrayVision 6.0 software (Imaging Research, St. Catharines, ON, CA). Housekeeping control genes β-actin, EF-1, α-tubulin, and cyclophilin were used to assess hybridization signal equivalence. Quantitative differentials were calculated based on normalization with EF-1α. The reproducibility of expression array screening was ensured by the inclusion of duplicate controls at various locations on the membrane. In addition, several previously described NSCLC genes were identified multiple times in the screening process.

Quantitative Real-Time PCR: The ABI PRISM® 7000 Real-Time PCR Sequence Detection System (Applied Biosystems, Foster City, Calif.) was used to determine the cancer-selectivity for novel L genes. EF-1 was used as the normalization gene for all ABI PRISM® 7000 experiments.

The Comparative Ct Method (Applied Biosystems, Foster City, Calif.) was used in calculating tumor vs. normal ratios. The amount of target, normalized to an endogenous reference gene (EF-1) and relative to a calibrator, is given by the arithmetic formula: 2^{-ΔΔct} where ΔΔCt is the change in threshold cycle between target and reference.

Cancer Profiling Arrays: Cancer profiling arrays (CPA) (Clontech) were used in calculating differential expression levels using multiple tumor and adjacent normal tissues. Hybridization signal intensities were analyzed using ArrayVision 6.0 (Imaging Research). The housekeeping control gene EF-1 was used in evaluating sample loading equivalence.

Serial Analysis Of Gene Expression: SAGE (Velculescu et al., 1995, Science,270, 484-487) was used as an additional validation tool in confirming tumor-selective expression for L genes beyond the initial observed NSCLC-specificity. SAGE tag sequences were identified and evaluated using SAGEmap (Lash et al., 2000) and SAGE anatomic viewer (Boon et al., 2002) public resources for evaluating relative abundance in both tumor cell line and tumor tissue-specific libraries.

Bioinformatics Analysis: After completion of the array data analysis sorting process, interesting novel targets were retained and analyzed further using several computational programs. Novel genes (L genes) were analyzed using Vector NTI Suite 6.0® (InforMax, Inc., Bethesda, Md.). Transmembrane (TM) domain and protein localization analysis was performed using the ExPASγ Proteomics Tools Programs® (Swiss Institute of Bioinformatics, Geneve, Switzerland). The PSORT algorithm (Nakai et al., 1999, Trends Biochem. Sci. 24(1):34) was used for subcellular localization prediction (Table 3). Genes were found to encode proteins for several subcellular compartments: cytoplasmic (n=63), extracellular matrix (n=2), mitochondrial (n=10), nuclear (n=54), plasma membrane (n=10), and endoplasmic reticulum (n=8).

### RESULTS

Cloning of L-Genes: Genes L1-L19 (FIG. 1) were amplified from lung carcinoma cell line RNA using gene-specific primers and cloned into the pCR 4.0® TOPO TA vector (Invitrogen, Carlsbad, Calif.). L1-L19 sequences (FIG. 1) were sequence verified using custom primers (Sigma-Genosys, Woodlands, Tex.) and automated fluorescent sequencing (PE Applied Biosystems, Foster City, Calif.). Amino acid sequences are reported for each of the 19 L-genes (FIG. 2).

Expression Profiling and Identification of NSCLC-Specific Genes: A total of 3,072 clones from NSCLC-1 and NSCLC-2 were pre-screened by colony PCR. Clones lacking inserts, and clones with small inserts (<500 base pairs) or multiple inserts were eliminated from the pool of amplifying clones to be used for array comparisons. Evaluation of genes that are differentially expressed in NSCLC was undertaken using ArrayVision 6.0 as previously described in other studies (Ji et al., 2003, Nucl. Acids Res., 31, 2534-2543). Following background subtraction and subsequent normalization to control gene EF-1, tumor: normal ratios were calculated based on the average of duplicate spots.

Expression profiling identified genes that were over-expressed in NSCLC including 13 genes≧10-fold, 45 genes ≧5-fold; 66 genes ≧4-fold, 103 genes≧3-fold, and 147 genes≧2-fold (Table 3). These 147 NSCLC-associated genes are predicted to encode many different functional classes of proteins including enzymes (n=33), cell cycle regulators (n=21), and ribosomal proteins (n=15), as well as genes of unknown function (n=26). Literature searches for each of the 147 NSCLC-associated genes were performed using the PubMed database and a combination of search terms including the “gene name” together with “non-small cell lung cancer” or “cancer” (Table 3). Of the 147 genes over-expressed by ≧2-fold in NSCLC cell lines, 46 have not previously been reported as associated with any neoplasm including 19 “novel” genes of unassigned identities designated herein L1 to L19. A set of 53 genes previously associated with cancer, but not previously described as associated with NSCLC was also identified.

The validity of the strategy used here to identify genes upregulated in NSCLC is strongly supported by the observation that 48 of the 147 genes identified have been previously associated with NSCLC using a variety of alternative methodologies (Table 3). Some of the most notable of these genes are PGP9.5 (Hibi et al., 1999, Ann. J. Pathol., 155, 711-715), cytokeratin 18 (Young et al., 2002, Lung Cancer, 36, 133-141), aldehyde dehydrogenase 1 (Schnier et al., 1999, FEBS Lett., 454,100-104), LDHA (Beer et al., 2002, Nat. Med., 8, 816-824), and aldo-keto reductase 1 (Palackal et al., 2002, J. Biol. Chem., 277, 24799-24808). LDHA, displaying the highest recovery frequency at 51, is associated with c-Myc-induced transformation (Lewis et al., 2000, Cancer Res., 60,6178-6183). LDHA-specific probes were used to prescreen expression arrays to eliminate sequence redundancy.

TABLE 3
Summary of genes over-expressed in NSCLC

| | Re- | | | | | | |
|---|---|---|---|---|---|---|---|
| | covery | | | Chro- | Pre- | | |
| | fre- | | Unigene ID | mosomal | dicted | References | |
| T:N | quency | Identity | (Hs.) | location | location | NSCLC | Other cancers |
| 40.2 | 5 | BCMP101 | 124951 | 8q24 | N | | (Adam et al., 2003, J. Biol. |
| | | | | | | | Chem., 278, 6482-6489) |
| 35.4 | 7 | keratin hair basic 1 | 170925 | 12q13 | N | | (Cribier et al., 2001, Br. J. |
| | | | | | | | Dermatol., 144, 977-982) |
| 29.7 | 13 | aldehyde dehydrogenase 1 | 76392 | 9q21 | C | (Schnier et al., 1999, FEBS | |
| | | | | | | Lett., 454, 100-104) | |
| 25.1 | 10 | signal peptidase complex | 9534 | 15q25 | C | | (Dubuisson et al., 1994, J. |
| | | | | | | | Virol., 68, 6147-6160) |
| 22.9 | 8 | NAD(P)H dehydrogenase | 406515 | 16q22 | C | | (Cullen et al., 2003, Cancer |
| | | | | | | | Res., 63, 5513-5520) |
| 22.6 | 7 | aldo-keto reductase 1 | 116724 | 7q33 | C | (Palackal et al., 2002, | |
| | | | | | | Supra) | |
| 19.6 | 5 | ATIC | 90280 | 2q35 | C | | (Colleoni et al., 2000, Am. J. |
| | | | | | | | Pathol., 156, 781-789) |
| 18.3 | 11 | L1 | 369973 | 8q24 | C | (Hibi et al., 1999, Am. J. | |
| | | | | | | Pathol., 155, 711-715) | |
| 14.8 | 2 | PGP 9.5 | 76118 | 4p14 | C | (Traverso et al., 1998, J. | |
| | | | | | | Cell. Sci., 111, (Pt. 10), | |
| | | | | | | 1405-1418) | |
| 14.8 | 17 | annexin 1 | 287558 | 9q11 | C | | |
| 13.4 | 2 | L2 | 511938 | 14q23 | N | | |
| 13.4 | 7 | CXC member 5 | 89714 | 4q12 | EM | | (Behrens et al., 2003, Apoptosis, |
| | | | | | | | 8, 39-44) |
| 11.1 | 2 | CSE1 | 90073 | 20q13 | C | | (Maeda et al., 2003, J. Hepatol., |
| | | | | | | | 38, 615-622) |
| 9.9 | 7 | aspartate beta-hydroxylase | 413557 | 8q12 | C | (Beer et al., 2002, Nat. | |
| | | | | | | Med., 8, 816-824) | |
| 9.8 | 51 | LDHA | 2795 | 11q15 | ER | | (Dalerba et al., 2001, Int. J. |
| | | | | | | | Cancer, 93, 85-90) |
| 8.2 | 1 | GAGE-4 | 278606 | Xp11 | N | (Young et al., 2002, Lung | |
| | | | | | | Cancer, 36, 131-141) | |
| 7.4 | 5 | cytokeratin 18 | 406013 | 12q13 | M | | |
| 7.3 | 2 | METTL3 | 168799 | 14q11 | N | | (Gombart et al., 2002, Blood 99, |
| | | | | | | | 1332-1340) |
| 7.2 | 3 | bZIP | 155291 | 2q33 | M | | |
| 7.0 | 1 | calpastatin | 359682 | 5q15 | N | (Gil-Parrado et al., 2002, J. | |
| | | | | | | Biol. Chem., 277, 27217-27226) | |
| 6.9 | 1 | ANAPC5 | 7101 | 12q24 | C | | (Yu et al., 1998, Science, 279, |
| | | | | | | | 1219-1222) |
| 6.8 | 1 | cysteine-rich inducer 61 | 8867 | 1p31 | M | (Tong et al., 2001, J. | |
| | | | | | | Biol. Chem., 276, 47709-47714) | |
| 6.7 | 9 | TFP inhibitor 2 | 438231 | 7q31 | N | (Ganapathi et al., 1988, Br. | |
| | | | | | | J. Cancer, 58, 335-340) | |
| 6.6 | 8 | thioredoxin reductase 1 | 13046 | 12q23 | C | (Soini et al., 2001, Clin. | |
| | | | | | | Cancer Res., 7, 1750-1757) | |
| 6.5 | 1 | GAGE8 | 251406 | Xp11 | N | | (Dalerba et al., 2001, Int. J. |
| | | | | | | | Cancer, 93, 85-90) |
| 6.5 | 2 | met proto-oncogene | 419124 | 7q31 | PM | (Lorenzato et al., 2002, | |
| | | | | | | Cancer Res., 62, 7025-7030) | |
| 6.2 | 1 | L3 | 56382 | 17q21 | N | | |
| 6.0 | 1 | L4 | 356223 | 12p11 | N | (Rao et al., 2001, | |
| | | | | | | Biochem., 40, 2096-2103) | |
| 5.9 | 7 | K-aplha-1 tubulin | 334842 | 12q13 | C | | |
| 5.9 | 1 | splicing factor3 | 405144 | 6p21 | N | (Kikuchi et al., 2003, | |
| | | | | | | Oncogene, 22, 2192-2205) | |
| 5.8 | 1 | PTTG-1 | 350966 | 5q35.1 | N | | (Shibata et al., 2002, Jpn. J. |
| | | | | | | | Clin. Oncol., 32, 233-237) |
| 5.7 | 7 | tranaketolase | 89643 | 3p14.3 | C | | (Heinrich et al., 1976, Cancer |
| | | | | | | | Res., 36, 3189-3197) |
| 5.6 | 4 | thioredoxin | 395309 | 9q31 | C | (Soini et al., 2001, Clin. | |
| | | | | | | Cancer Res., 7, 1750-1757) | |
| 5.6 | 3 | NM23A | 118638 | 7q21 | C | | (Kikuchi et al., 2003, Oncogene, |
| | | | | | | | 22, 2192-2205) |
| 5.5 | 1 | MAD2L1 | 122346 | 4q27 | C | | (Percy et al., 2000, Genes |
| | | | | | | | Chrom. Cancer, 29, 356-362) |
| 5.4 | 1 | epiregulin | 115263 | 4q21 | ER | | (Zhu et al., 2000, Biochem. |
| | | | | | | | Biophys. Res. Commun., 273, |
| | | | | | | | 1019-1024) |
| 5.3 | 1 | TCF6L1 | 75133 | 10q21 | N | | (Yoshida et al., 2003, Cancer |
| | | | | | | | Res., 63, 3729-3734) |
| 5.3 | 1 | L5 | 81892 | 15q22 | M | | (Luo et al., 2002, Br. J. Cancer, |
| | | | | | | | 8, 339-343) |
| 5.3 | 1 | RPL8 | 178551 | 8q24 | C | | (Denis et al., 1993, Int. J. |
| | | | | | | | Cancer, 55, 275-280) |
| 5.3 | 1 | RPS13 | 446588 | 11p15 | C | | |
| 5.2 | 3 | H2AFZ | 119192 | 4q24 | C | | (Larramendy et al., 2002, |
| | | | | | | | Haematologica, 87, 569-577) |
| 5.2 | 6 | NPM1 | 411098 | 5q35 | N | | (Bertram et al., 1998, Eur. J. |
| | | | | | | | Cancer, 34, 731-736) |
| 5.1 | 1 | RPL5 | 469653 | 1p22 | C | | |
| 5.0 | 2 | FER1L3 | 362731 | 10q24 | C | | |
| 5.0 | 5 | CYP24A1 | 89663 | 20q13 | C | | (Ponnazhagan & Kwon, 1992, |
| | | | | | | | Pigment Cell Res., 5, 155-161) |
| 4.9 | 1 | TEBP | 355693 | 12q13 | N | (Ferrigno et al., 2003, | |
| | | | | | | Lung Cancer, 41, 311-320) | |
| 4.9 | 1 | enolase 1 | 433455 | 1p36 | C | (Rintoul et al., 2002, Mol. | |
| | | | | | | Biol. Cell., 13, 2841-2852) | |
| 4.8 | 5 | CD98 | 79748 | 11q13 | C | | |
| 4.8 | 2 | RPS10 | 406620 | 6p21 | N | | (Salicioni et al., 2000, |
| | | | | | | | Genomics, 69, 54-62) |
| 4.7 | 1 | RBM8A | 10283 | 1q12 | C | | (Dalerba et al., 2001, Int. J. |
| | | | | | | | Cancer, 93, 85-90) |
| 4.7 | 1 | MAGEA3 | 417816 | Xq28 | ER | | (Jiang et al., 1997, Oncogene, |
| | | | | | | | 14, 473-480) |
| 4.7 | 1 | RPL23A | 419463 | 17q11 | C | (Oshita et al., 2002, | |
| | | | | | | Anticancer Res., 22, 1065-1070) | |
| 4.7 | 1 | CD29 | 287797 | 10p11 | PM | | |
| 4.5 | 1 | cytokeratin 8 | 356123 | 12q13 | M | (Fukunaga et al., 2002, | |
| 4.4 | 2 | XRN2 | 268555 | 20p11 | N | Lung Cancer, 38, 31-38) | |
| 4.3 | 2 | ARPC-1B | 433506 | 7q22 | C | | (Kress et al., 1998, J. Cancer |
| | | | | | | | Res. Clin. Oncol., 124, 315-320) |
| 4.2 | 4 | PKM2 | 198281 | 15q22 | C | (Malusecka et al., 2001, | |
| | | | | | | Anticancer Res., 21, 1015-1021) | |
| 4.2 | 1 | HSP70 | 8997 | 10q25 | N | | (Kanner et al., 1990, Proc. Natl. |
| | | | | | | | Acad. Sci. USA, 87, 3328-3332) |
| 4.2 | 5 | EIF3S10 | 389559 | 10q26 | N | | |
| 4.2 | 2 | TRIM16 | 241305 | 17p12 | C | | |
| 4.2 | 2 | L6 | 13885 | 5p15 | C | | (Kanner et al., 1990, Proc. Natl. |
| | | | | | | | Acad. Sci. USA, 87, 3328-3332) |
| 4.1 | 1 | EIF3S3 | 127149 | 8q24 | N | | |
| 4.1 | 1 | L7 | 325422 | 1p36 | N | | |
| 4.1 | 1 | cytokeratin 7 | 23881 | 12q12 | M | (Kummar et al., 2002, Br. | |
| | | | | | | J. Cancer, 86, 1884-1887) | |
| 4.0 | 1 | tubulin beta 5 | 356729 | 6p21 | C | | (Kikuchi et al., 2003, Oncogene, |
| | | | | | | | 22, 2192-2205) |
| 4.0 | 2 | PPIA | 401787 | 7p13 | C | | |
| 3.9 | 4 | CCT7 | 6456 | 12q14 | C | (Kikuchi et al., 2003, | |
| | | | | | | Oncogene, 22, 2192-2205) | |
| 3.9 | 3 | nucleolin | 79110 | 2q12 | N | | (Grinstein et al., 2002, J. Exp. |
| | | | | | | | Med., 196, 1067-1078) |
| 3.9 | 1 | EEF1A1 | 439552 | 6q14 | C | | (Su et al., 1998, Proc. Natl. |
| | | | | | | | Acad. Sci. USA, 95, 1764-1769) |
| 3.9 | 1 | L8 | 162669 | 9q31 | N | | |
| 3.9 | 1 | cyclin B1 | 23960 | 5q12 | M | (Soria et al., 2000, Cancer | |
| | | | | | | Res., 60, 4000-4004) | |
| 3.8 | 2 | YWHAG | 25001 | 7q11 | N | (Kikuchi et al., 2003, | |
| | | | | | | Oncogene, 22, 2192-2205) | |
| 3.8 | 2 | BNIP3 | 79428 | 14q11 | PM | | (Lai et al., 2003, Br. J. Cancer, |
| | | | | | | | 88, 270-276) |
| 3.8 | 6 | UNRIP | 3727 | 12p13 | C | | (Matsuda et al., 2000, Cancer |
| | | | | | | | Res., 60, 13-17) |
| 3.8 | 1 | HSP90 | 446579 | 14q32 | C | (Zhong et al., 2003, Cancer | |
| | | | | | | Detect. Prev., 27, 285-290) | |
| 3.8 | 1 | TRIP12 | 388373 | 2q37 | M | | |
| 3.6 | 2 | L9 | 154103 | 4q22 | N | | (Le Roux & Moroianu, 2003, |
| | | | | | | | Supra) |
| 3.5 | 1 | KPNA2 | 159557 | 17q23 | ER | | |
| 3.5 | 1 | TUBG1 | 21635 | 17q21 | C | | |
| 3.4 | 1 | L10 | 212992 | 14q24 | C | | (Henry et al., 1993, Cancer Res., |
| | | | | | | | 53, 1403-1408) |
| 3.4 | 2 | RPL19 | 381061 | 17q11 | C | | (Hansel et al., 2003, Am. J. |
| | | | | | | | Pathol., 163, 217-229) |
| 3.4 | 1 | CTP synthase | 251871 | 1p34 | C | | |
| 3.4 | 1 | NSEP1 | 74497 | 1p34 | N | (Zajac-Kaye, 2001, Lung | |
| | | | | | | Cancer, 34 Suppl. 2, S43-S46) | |
| 3.4 | 3 | v-Myc | 202453 | 8q24 | N | | |
| 3.4 | 1 | NOL5A | 376064 | 20p13 | N | | (Farquhar et al., 2003, Neurosci, |
| | | | | | | | Lett., 346, 53-36) |
| 3.4 | 1 | PSEN1 | 3260 | 14q24 | PM | | (Deloulme et al., 2000, J. Biol. |
| | | | | | | | Chem., 275, 35302-35310) |
| 3.3 | 1 | S100 A11 protein | 417004 | 1q21 | N | | |
| 3.3 | 2 | L11 | 437433 | 16p13 | N | | |
| 3.3 | 2 | DNAJC8 | 433540 | 1p35 | N | | |
| 3.3 | 1 | MORF4L2 | 411358 | Xq22 | N | | |
| 3.3 | 1 | RPL28 | 356371 | 19q13 | N | | |
| 3.2 | 1 | AASDHPPT | 64595 | 11q22 | C | (Garcia-Lora et al., 2003, | |
| | | | | | | Int. J. Cancer, 106, 521-27) | |
| 3.2 | 2 | calnexin | 155560 | 5q35 | PM | | |
| 3.2 | 7 | TOP2A | 156346 | 17q21 | N | (Wikman et al., 2002, | |
| | | | | | | Oncogene, 21, 5804-5813) | |
| 3.2 | 4 | SET translocation | 436687 | 9q34 | N | | |
| 3.1 | 1 | NIFIE 14 | 9234 | 19q13 | PM | | |
| 3.1 | 1 | ubiquitin carrier protein | 396393 | 19q13 | C | | (Xue et al., 2003, Cancer Res., |
| | | | | | | | 63, 980-986) |
| 3.1 | 2 | RRM-2 | 226390 | 2p25 | C | (Yazawa et al., 2003, | |
| | | | | | | Pathol. Int. 53, 58-65) | |
| 3.1 | 2 | phosphoglycerate kinase 1 | 78771 | Xq13 | C | (Redondo et al., 1998, Eur. | |
| | | | | | | J. Immunogenet., 25, 385-391) | |
| 3.0 | 2 | actin, gamma | 14376 | 17q25 | C | | (Le Roux & Moroianu, 2003, |
| | | | | | | | Supra) |
| 3.0 | 2 | karyopherin beta 1 | 180446 | 17q21 | C | | (Li et al., 2003, Prostate, 56, |
| | | | | | | | 98-105) |
| 3.0 | 1 | GSTP1 | 411509 | 11q13 | C | | (Loging & Reisman, 1999, |
| | | | | | | | Cancer Epidemiol. Biomarkers |
| | | | | | | | Prev. 8, 1011-1016) |
| 3.0 | 1 | RPL37 | 80545 | 5p13 | C | | |
| 2.9 | 2 | PSAT1 | 286049 | 9q21 | C | | |
| 2.9 | 1 | CLCP1 | 173374 | 3q12.2 | PM | (Koshikawa et al., 2002, | |
| | | | | | | Oncogene, 21, 2822-2828) | |
| 2.9 | 1 | CYP1B1 | 89663 | 2p21 | ER | (Hukkanen et al., 2000, | |
| | | | | | | Am. J. Respir. Cell Mol. | |
| | | | | | | Biol., 22, 360-366) | |
| 2.9 | 1 | FTH1 | 448738 | 11q13 | N | | (Pietsch et al., 2003, J. Biol. |
| | | | | | | | Chem., 278, 2361-2369) |
| 2.9 | 4 | PAI-1 | 165998 | 1p31 | N | (Robert et al., 1999, Clin. | |
| | | | | | | Cancer Res., 5, 2094-2102) | |
| 2.9 | 1 | L12 | 85963 | 16p13 | ER | | (Karan et al., 2002, |
| | | | | | | | Carcinogenesis, 23, 967-975) |
| 2.9 | 1 | RPS16 | 397609 | 19q13 | C | | (Cong et al., 2003, BMC Mol. |
| | | | | | | | Biol., 4, 10) |
| 2.9 | 1 | F-box protein | 321687 | 1p36 | C | | |
| 2.9 | 1 | HNRPA1 | 376844 | 12p13 | C | | (Sykes et al., 2003, Leuk. |
| | | | | | | | Lymphoma, 44, 1187-1199) |
| 2.9 | 1 | RRM1 | 383396 | 11p15 | C | (Pitterle et al., 1999, | |
| | | | | | | Mamm. Genome, 10, 916,-922) | |
| 2.9 | 7 | LDHB | 234489 | 12p12 | ER | | (Maekawa et al., 2003, Clin. |
| | | | | | | | Chem., 49, 1518-1520) |
| 2.9 | 2 | osteopontin | 313 | 4q21 | M | (Zhang et al., 2001, Cancer | |
| | | | | | | Lett., 171, 215-222) | |
| 2.9 | 2 | Ras G3BP | 220689 | 5q33 | N | (Silini et al., 1994, | |
| | | | | | | Virchows. Arch. 424, 367-373) | |
| 2.8 | 1 | ACTN-1 | 11900 | 7q21 | M | (Echchakir et al., 2001, | |
| | | | | | | Cancer Res., 61, 4078-4083) | |
| 2.8 | 1 | L13 | 29874 | 17q11 | N | | |
| 2.8 | 2 | CENPF | 77204 | 1q32 | N | (Rattner et al., 1997, Clin. | |
| | | | | | | Invest. Med., 20, 308-319) | |
| 2.8 | 1 | E2F6 | 42287 | 22q11 | N | | (Cullen et al., 2003, Cancer |
| | | | | | | | Res., 63, 5513-5520) |
| 2.8 | 1 | PGM1 | 1869 | 1p31 | C | (Tsybikova et al., 1996, | |
| | | | | | | Vestn. Ross. Akad. Med. | |
| | | | | | | Nauk., 12, 3-7) | |
| 2.8 | 1 | integrin beta 5 | 149846 | 3q21 | PM | (Smythe et al., 1995, | |
| | | | | | | Cancer Metastasis Rev., | |
| | | | | | | 14, 229-239) | |
| 2.8 | 1 | MSCP | 283716 | 8p21 | N | | |
| 2.8 | 1 | RPS3 | 414990 | 11q13 | N | (McDoniels-Silvers et al., | |
| | | | | | | 2002, Clin. Cancer Res., 8, | |
| | | | | | | 1127-1138) | |
| 2.8 | 1 | LDB1 | 26002 | 10q24 | N | | (Mizunuma et al., 2003, Supra) |
| 2.8 | 2 | RAC2 | 301175 | 22q13 | C | | (Gabig et al., 1995, Blood, 85, |
| | | | | | | | 804-811) |
| 2.6 | 1 | PC4 | 229641 | 5p13 | N | (Latif et al., 1997, Hum. | |
| | | | | | | Genet., 99, 334-341) | |
| 2.6 | 1 | PTP4A2 | 82911 | 1p35 | C | | (Cates et al., 1996, Cancer Lett., |
| | | | | | | | 110, 49-55) |
| 2.5 | 1 | CCT5 | 1600 | 5p15 | C | (Kikuchi et al., 2003, | |
| | | | | | | Oncogene, 22, 2192-2205) | |
| 2.5 | 1 | L14 | 26761 | 17q24 | C | (Niehans et al., 1996, | |
| | | | | | | Supra) | |
| 2.4 | 2 | CD59 | 278573 | 11p13 | EM | | (Yan-Sanders et al., 2002, |
| | | | | | | | Cancer Lett., 183, 215-220) |
| 2.4 | 1 | HNRPA2 | 232400 | 7p15 | C | (Taxman et al., 2003, | |
| | | | | | | Cancer Res., 63, 5095-5104) | |
| 2.4 | 1 | GRO1 | 789 | 4q21 | N | | |
| 2.3 | 1 | L15 | 335951 | 11p14 | N | | |
| 2.3 | 1 | CNAP1 | 5719 | 12p13 | N | | |
| 2.3 | 1 | L16 | 298646 | 8q24 | N | | |
| 2.3 | 1 | MCM6 | 444118 | 2q21 | C | | |
| 2.2 | 1 | ATP5B | 406510 | 12p13 | C | | |
| 2.2 | 1 | L17 | 22981 | 18q12 | N | (Schraml et al., 1997, | |
| | | | | | | Cancer Res., 57, 3669-3671) | |
| 2.2 | 1 | HMGB1 | 434102 | 13q12 | N | | |
| 2.2 | 1 | RPS26 | 355957 | 12q13 | C | (Hurbin et al., 2002, J. | |
| | | | | | | Biol. Chem., 277, 49127-49133) | |
| 2.2 | 1 | amphiregulin | 270833 | 4q13 | PM | | |
| 2.1 | 1 | L18 | 180236 | 9q31 | PM | | |
| 2.1 | 1 | L19 | 444909 | 11q23 | N | | (Dalerba et al., 2001, Int. J. |
| | | | | | | | Cancer, 93, 85-90) |
| 2.1 | 2 | MAGEA6 | 441113 | Xq28 | ER | | |
| 2.0 | 1 | tropomyosin 3 | 178468 | 1q21 | N | | |
| 2.0 | 1 | MKRN-1 | 7838 | 7q34 | N | | |

T: N represents differential expression levels for individual array comparisons. Recovery frequency illustrates the number of times each clone was identified in the study. Identity, Unigene ID, and Chromosomal location are assigned using the NCBI database. Subcellular localization is predicted using PSORT II (Horton & Nakai, 1997, Proc. Int. Conf. Intell. Syst. Mol. Biol., 5, 147-152): C, cytoplasmic; EM, extracellular matrix; ER, endoplasmic reticulum; M, mitochondrial; N, nuclear; PM, plasma membrane. References were identified using PubMed. Novel genes are designated L1-L19.

Quantitative Real-Time PCR (QPCR): Since the NSCLC subtracted cDNA libraries (NSCLC-1 and NSCLC-2) were generated using tumor cell lines, it was essential to confirm differential expression using independent assays to evaluate expression in both cell lines and patient tissue samples. QPCR was utilized as an independent method with which to investigate the association of the identified genes with NSCLC tumor cell lines and extend the observations to patient samples (Table 4). Five of the novel genes, namely, L4, L5, L7, L11, and L16 were chosen for analysis by QPCR. These genes were selected on the basis of their lack of sequence homology with “known” genes and relatively low expression levels in normal tissue. CXC-5 as included as a positive control gene based on its previous association with lung cancer.

The comparative threshold cycle (Ct) method (where Ct is the cycle threshold of detectable logarithmic amplification) (Aarskog & Vedeler, 2000. Hum Genet.,107, 494-498) was used in calculating differential expression ratios (amount of NSCLC target mRNA/amount of EF-1 mRNA) expressed as ΔCₜ=(C_{t NSCLC target}-C_{t EF-1}). Relative tumor: normal ratios as well as specified normal tissue: normal lung comparisons were calculated using the formula 2^{-ΔΔCT}.

NSCLC cell line expression levels were compared to those of normal small airway epithelial cells (SAEC; Table 4). L7 is over-expressed in each of the NSCLC cell line subtypes. LA is over-expressed in SCC, LCC, and BAC. L5 is over-expressed in AC, SCC, and LCC. L11 is over-expressed in SCC, LCC, and BAC. L16 is over-expressed in SCC, LCC, and BAC. NSCLC tumor tissue expression levels were also compared to paired adjacent normal tissues (Table 4). L5 and L11 are over-expressed in AC and SCC patient samples. L4, L7, and L16 are primarily SCC-specific, while displaying over-expression in a single AC sample.

TABLE 4
QPCR data using NSCLC cell lines and patient tissues

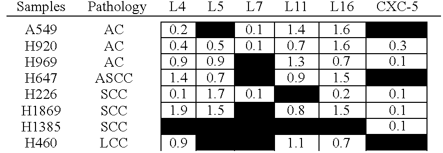

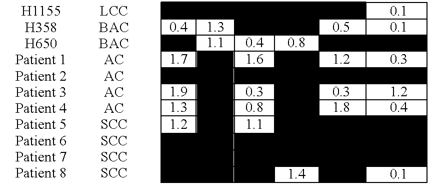

NSCLC tumor cell lines: A549, H920, H969, H647, H226, H1869, H1385, H460, H1155, H358, and H650; and patient tissues, designated 1-8, were used in calculating tumor: normal ratios. Tumor cell lines were compared versus normal SAEC. Patient tumor tissues were compared versus adjacent normal samples. Pathology abbreviations: AC, adenocarcinoma; ASCC, adenosquamous cell carcinoma; SCC, squamous cell carcinoma; LCC, large cell carcinoma; and BAC, bronchioalvoelar carcinoma. The comparative threshold cycle (Ct) method (where Ct is the cycle threshold of detectable logarithmic amplification) (Aarskog and Vedeler, 2000, Hum. Genet., 107, 494-498) was used in calculating differential expression ratios (amount of NSCLC target/amount of EF-1) expressed as ΔC_{T}=(C_{t NSCLC target}-C_{t EF-}1) for individual comparisons. Tumor: normal ratios were calculated using the formula 2^{-ΔΔCT}. Data shaded in black show >2-fold ratios for tumor: normal comparisons.

Cancer Profiling Arrays: Cancer profiling arrays (CPA) were used as an additional independent validation tool for probing the tumor-associated up-regulation of a subset of L genes (Table 5). A subset of the L genes exhibit elevated expression levels for multiple samples of several tumor types. Observed tumor-selectivity includes: L4 in breast, uterine, colon, stomach, ovarian, lung, and rectal cancers; L5 in breast, uterine, stomach, ovarian, lung, and kidney cancers; L7 in breast, and uterine cancers; L11 in breast, uterine, and lung cancers; L16 in breast, uterine, colon, stomach, ovarian, and lung cancers; and L17 in breast, uterine, ovarian cancers.

TABLE 5
Cancer profiling array data for L genes

| | Breast | Uterus | Colon | Stomach | Ovary | Lung | Kidney | Rectum |
|---|---|---|---|---|---|---|---|---|
| Gene | (n = 50) | (n = 42) | (n = 35) | (n = 27) | (n = 14) | (n = 21) | (n = 20) | (n = 18) |
| L4 | 6 | 5 | 15 | 8 | 4 | 11 | 1 | 7 |
| L5 | 36 | 31 | 3 | 11 | 12 | 15 | 9 | 1 |
| L7 | 8 | 4 | 3 | 2 | 0 | 2 | 2 | 0 |
| L11 | 13 | 10 | 1 | 1 | 2 | 5 | 3 | 2 |
| L16 | 17 | 10 | 11 | 8 | 5 | 11 | 2 | 3 |
| L17 | 6 | 9 | 1 | 1 | 3 | 1 | 0 | 0 |
| L18 | 1 | 1 | 2 | 2 | 0 | 1 | 0 | 0 |

Data is reported based on the number of samples having ≧2-fold tumor: normal expression ratios. Total samples for each tumor and corresponding adjacent normal tissue pair are included.

Serial Analysis of Gene Expression: SAGE tag sequences were identified and evaluated using SAGEmap (Lash et al., 2000) and SAGE anatomic viewer (Boon et al., 2002) (Table 6). This enabled rapid validation of targets based on relative transcript abundance within multiple cell line-specific and tissue-specific SAGE libraries. SAGE data is reported with particular emphasis on libraries with a minimum threshold >100 tags/million (Table 6).

Elevated SAGE values which support cancer-specific expression include: L1 in breast; L5 in breast and prostate; L6 in breast and pancreatic; L11 in colon, ovarian, pancreatic, and prostate; L17 in breast; and L18 in breast. Elevated tumor tissue-specific SAGE values include: L1 in brain and breast; L2 in brain; L4 in brain, breast, gastric, and ovarian; L5 in brain; L8 in brain; L9 in brain; and L11 in lung, ovarian, and prostate.

TABLE 6
SAGE analysis using public X profiler and anatomic viewer resources

| A | | B | | | | C | | | |
|---|---|---|---|---|---|---|---|---|---|
| Gene | SAGE tag | Gene | Cell Line | Pathology | Tags/million | Gene | Tissue | Pathology | Tags/million |
| L1 | TCTAAAGAAT | L1 | MCF7 | Breast cancer | 178 | L1 | H154 | Brain cancer | 115 |
| L2 | TGAATTCTAC | L5 | LacZ | Breast cancer | 269 | | IDC4 | Breast cancer | 292 |
| L3 | GGATCTCCCA | | MDA-MB453 | Breast cancer | 210 | L2 | MHH1 | Brain cancer | 102 |
| L4 | CACTTTGTAT | | ZR75 | Breast cancer | 193 | L4 | IDC4 | Breast cancer | 261 |
| L5 | TCCTTTGCAA | | LNCaP | Prostate cancer | 107 | | G189 | Gastric cancer | 189 |
| L6 | CCTATAATAA | L6 | LacZ | Breast cancer | 107 | | OC14 | Ovarian cancer | 171 |
| L7 | ATATCAGGAC | | CAPAN1 | Pancreatic cancer | 131 | | P494 | Brain cancer | 160 |
| L8 | GACTTGGCGG | L11 | LNCaP | Prostate cancer | 176 | | H972 | Brain cancer | 150 |
| L9 | ATAAATATAA | | CaCo2 | Colon cancer | 162 | L5 | H341 | Brain cancer | 223 |
| L10 | AGAAATGCTA | | Panc1 | Pancreatic cancer | 160 | | P608 | Brain cancer | 205 |
| L11 | CAAGCCCTGC | | A2780 | Ovarian cancer | 134 | L8 | H392 | Brain cancer | 100 |
| L12 | GTGGGAGGCA | | HCT116 | Colon cancer | 132 | L9 | H392 | Brain cancer | 100 |
| L13 | GTCAAGGAAA | L17 | MDA-MB453 | Breast cancer | 105 | L11 | Lung9 | Lung cancer | 190 |
| L14 | AACCTGTTCT | L18 | MDA-MB453 | Breast cancer | 105 | | Lung10 | Lung cancer | 180 |
| L15 | TTGTTTTGCA | | | | | | PR317 | Prostate cancer | 153 |
| L16 | ATTTGCGGGA | | | | | | OVT6 | Ovarian cancer | 117 |
| L17 | AAACCTCTCA | | | | | | | | |
| L18 | CACCCCTCAG | | | | | | | | |
| L19 | TTTGCTTGTA | | | | | | | | |

SAGE tags were selected and analyzed using SAGEmap (Lash et al., 2000) and SAGE anatomic viewer (Boon et al., 2002). Data is reported based on tumor cell lines and tumor tissue-specific expression levels displaying a minimum threshold value of 100 tags/million.

Genetic Expression Signature (GES) Sets: Detection of a genetic expression signature is a positive indicator for the presence of a particular tumor-selective condition in a subject wherein the genetic signature or a subset thereof is identified. The NSCLC-associated molecules described herein have, therefore, been organized into several distinct functional categories. Accordingly, in addition to the comprehensive genetic expression signature which comprises all 147 NSCLC-associated molecules excluding molecules corresponding to GenBank Accession No. BC052957 (designated herein as an NSCLC genetic expression signature 1; GES1) (Table 7), a genetic expression signature comprising 99 genes never previously associated with lung cancer is described and designated herein GES1a (Table 8, excluding molecules corresponding to GenBank Accession No. BC052957). Also provided is a genetic expression signature comprising 46 up-regulated genes never previously associated with any cancer is designated herein GES1b (Table 9). A genetic expression signature comprising 53 known cancer associated, but not lung cancer associated, genes is described and designated GES1c (Table 10, excluding molecules corresponding to GenBank Accession No. BC052957). A genetic expression signature comprising 19 novel L genes (designated herein as L1-L19) is also described and designated GES1d (Table 11). A genetic expression signature comprising 13 genes displaying≧10-fold tumor: normal ratios is described and designated GES1e (Table 12, excluding molecules corresponding to GenBank Accession No. BC052957). A genetic expression signature comprising 45 genes displaying≧5-fold tumor: normal ratios is described and designated GES1f (Table 13, excluding molecules corresponding to GenBank Accession No. BC052957). A genetic expression signature comprising 66 genes displaying≧4-fold tumor: normal ratios is described and designated GES1g (Table 14, excluding molecules corresponding to GenBank Accession No. BC052957). A genetic expression signature comprising 103 genes displaying≧3-fold tumor: normal ratios is described and designated GES1h (Table 15, excluding molecules corresponding to GenBank Accession No. BC052957).

For all tables presented herein, the heading Reference Sequence (Ref. Seq.) refers to the GenBank Accession No. corresponding to the indicated gene.

TABLE 7
Sequence listings for 147 NSCLC-associated genes included in GES1
References

| T:N | Identity | Ref. Seq. | NSCLC | Other Cancers |
|---|---|---|---|---|
| 40.2 | BCMP101 | BC052957 | | (Adam et al. 2003, J. Biol. |
| | | | | Chem., 278, 6482-6489) |
| 35.4 | keratin hair basic 1 | NM_002281 | | (Cribier et al., 2001, Br. J. |
| | | | | Dermatol., 144, 977-982) |
| 29.7 | aldehyde dehydrogenase 1 | NM_000689 | (Schnier et al., 1999, FEBS | |
| | | | Lett., 454, 100-104) | |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res., 63, 5513-5520) |
| 22.6 | aldo-keto reductase 1 | NM_020299 | (Palackal et al., 2002, J. Biol. | |
| | | | Chem., 277, 24799-24808) | |
| 19.6 | ATIC | NM_004044 | | (Colleoni et al., 2000, Am. J. |
| | | | | Pathol., 156, 781-789) |
| 18.3 | L1 | SEQ ID NO: 1 | | |
| 14.8 | PGP 9.5 | NM_004181 | (Hibi et al., 1999, Am. J. | |
| | | | Pathol., 155, 711-715) | |
| 14.8 | annexin 1 | NM_000700 | (Traverso et al., 1998, Supra) | |
| 13.4 | L2 | SEQ ID NO: 2 | | |
| 13.4 | CXC member 5 | NM_002994 | | (Behrens et al., 2003, |
| | | | | Apoptosis, 8, 39-44) |
| 11.1 | CSE1 | NM_001316 | | |
| 9.9 | aspartate beta-hydroxylase | U03109 | | (Maeda et al., 2003, Supra) |
| 9.8 | LDHA | NM_005566 | (Beer et al., 2002, Nat. Med., | |
| | | | 8, 816-824) | |
| 8.2 | GAGE-4 | NM_001474 | | (Dalerba et al., 2001, Int. J. |
| | | | | Cancer, 93, 85-90) |
| 7.4 | cytokeratin 18 | NM_000224 | (Young et al., 2002, Supra) | |
| 7.3 | METTL3 | NM_019852 | | (Gombart et al., 2002, Blood |
| | | | | 99, 1332-1340) |
| 7.2 | bZIP | NM_014670 | (Gil-Parrado et al., 2002, J. | |
| | | | Biol. Chem., 277, 27217-27226) | |
| 7 | calpastatin | AF327443 | | (Yu et al., 1998, Science, 279, |
| | | | | 1219-1222) |
| 6.9 | ANAPC5 | NM_016237 | (Tong et al., 2001, Supra) | |
| 6.8 | cysteine-rich inducer 61 | NM_001554 | (Ganapathi et al., 1988, Br. J. | |
| | | | Cancer, 58, 335-340) | |
| 6.7 | TFP inhibitor 2 | NM_006528 | (Soini et al., 2001, Clin. | |
| | | | Cancer Res., 7, 1750-1757) | |
| 6.6 | thioredoxin reductase 1 | AJ001050 | | (Dalerba et al., 2001, Int. J. |
| | | | | Cancer, 93, 85-90) |
| 6.5 | GAGE8 | NM_001468 | (Lorenzato et al., 2002, | |
| | | | Cancer Res., 62, 7025-7030) | |
| 6.5 | met proto-oncogene | NM_000245 | | |
| 6.2 | L3 | SEQ ID NO: 3 | | |
| 6 | L4 | SEQ ID NO: 4 | | |
| 5.9 | K-aplha-1 tubulin | NM_032704 | (Rao et al., 2001, Supra) | |
| 5.9 | splicing factor3 | NM_003017 | (Kikuchi et al., 2003, | |
| | | | Oncogene, 22, 2192-2205) | |
| 5.8 | PTTG-1 | NM_004219 | | (Shibata et al., 2002, Supra) |
| 5.7 | transketolase | NM_001064 | | (Heinrich et al., 1976, Cancer |
| | | | | Res., 36, 3189-3197) |
| 5.6 | thioredoxin | NM_003329 | (Soini et al., 2001, Clin. | |
| | | | Cancer Res., 7, 1750-1757) | |
| 5.6 | NM23A | NM_000269 | | (Kikuchi et al., 2003, |
| | | | | Oncogene, 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., 273, |
| | | | | 1019-1024) |
| 5.3 | TCF6L1 | NM_003201 | | (Yoshida et al., 2003, Cancer |
| | | | | Res., 63, 3729-3734) |
| 5.3 | L5 | SEQ ID NO: 5 | | |
| 5.3 | RPL8 | Z28407 | | (Luo et al., 2002, Br. J. Cancer, |
| | | | | 87, 339-343) |
| 5.3 | RPS13 | NM_001017 | | (Denis et al., 1993, Int. J. |
| | | | | Cancer, 55, 275-280) |
| 5.2 | H2AFZ | NM_002106 | | (Larramendy et al., 2002, |
| | | | | Supra) |
| 5.2 | NPM1 | NM_002520 | | |
| 5.1 | RPL5 | NM_000969 | | (Bertram et al., 1998, Eur. J. |
| | | | | Cancer, 34, 731-736) |
| 5 | FER1L3 | NM_013451 | | |
| 5 | CYP24A1 | NM_000782 | | (Ponnazhagan & Kwon, 1992, |
| | | | | Supra) |
| 4.9 | TEBP | NM_006601 | (Ferrigno et al., 2003, Lung | |
| | | | Cancer, 41, 311-320) | |
| 4.9 | enolase 1 | NM_001428 | | |
| 4.8 | CD98 | NM_002394 | (Rintoul et al., 2002, Supra) | |
| 4.8 | RPS10 | NM_001014 | | |
| 4.7 | RBM8A | NM_005105 | | (Salicioni et al., 2000, Supra) |
| 4.7 | MAGEA3 | NM_005362 | | (Dalerba et al., 2001, Int. J. |
| | | | | Cancer, 93, 85-90) |
| 4.7 | RPL23A | NM_000984 | | (Jiang et al., 1997, Oncogene, |
| | | | | 14, 473-480) |
| 4.7 | CD29 | NM_002211 | (Oshita et al., 2002, Supra) | |
| 4.5 | cytokeratin 8 | NM_002273 | (Fukunaga et al., 2002, Lung | |
| | | | Cancer, 38, 31-38) | |
| 4.4 | XRN2 | NM_012255 | | |
| 4.3 | ARPC-1B | NM_005720 | | |
| 4.2 | PKM2 | M26252 | | |
| | | | | (Kress et al., 1998, J. Cancer |
| | | | | Res. Clin. Oncol., 124, 315-320) |
| 4.2 | HSP70 | M11717 | (Malusecka et al., 2001, | |
| | | | Supra) | |
| 4.2 | EIF3S10 | NM_003750 | | (Kanner et al., 1990, Proc. Natl |
| | | | | Acad. Sci, USA, 87, 3328-3332) |
| 4.2 | TRIM16 | NM_006470 | | |
| 4.2 | L6 | SEQ ID NO: 6 | | |
| 4.1 | EIF3S3 | NM_003756 | | (Kanner et al., 1990, Proc. Natl. |
| | | | | Acad. Sci. USA, 87, 3328-3332) |
| 4.1 | L7 | SEQ ID NO: 7 | | |
| 4.1 | cytokeratin 7 | NM_005556 | (Kummar et al., 2002, Br.J. | |
| | | | Cancer, 86, 1884-1887) | |
| 4 | tubulin beta 5 | AF141349 | | (Kikuchi et al., 2003, |
| | | | | Oncogene, 22, 2192-2205) |
| 4 | PPIA | NM_021130 | | |
| 3.9 | CCT7 | NM_006431 | (Kikuchi et al., 2003, | |
| | | | Oncogene, 22, 2192-2205) | |
| 3.9 | nucleolin | NM_005381 | | (Grinstein et al., 2002, J. Exp. |
| | | | | Med., 196, 1067-1078 |
| 3.9 | EEF1A1 | NM_001402 | | (Su et al., 1998, Proc. Natl. |
| | | | | Acad. Sco. USA, 95, 1764-1769) |
| 3.9 | L8 | SEQ ID NO: 8 | | |
| 3.9 | cyclin B1 | NM_031966 | (Soria et al., 2000, Supra) | |
| 3.8 | YWHAG | NM_012479 | (Kikuchi et al., 2003, | |
| | | | Oncogene, 22, 2192-2205) | |
| 3.8 | BNIP3 | NM_004052 | | (Lai et al., 2003, Br. J. Cancer, |
| | | | | 88, 270-276) |
| 3.8 | UNRIP | NM_007178 | | (Matsuda et al., 2000, Supra) |
| 3.8 | HSP90 | X15183 | (Zhong et al., 2003, Supra) | |
| 3.8 | TRIP12 | NM_004238 | | |
| 3.6 | L9 | SEQ ID NO: 9 | | |
| 3.5 | KPNA2 | BC005978 | | (Le Roux & Moroianu, 2003, |
| | | | | Supra) |
| 3.5 | TUBG1 | NM_001070 | | |
| 3.4 | L10 | SEQ ID NO10 | | |
| 3.4 | RPL19 | NM_000981 | | (Henry et al., 1993, Cancer |
| | | | | Res., 53, 1403-1408) |
| 3.4 | CTP synthase | NM_001905 | | (Hansel et al., 2003, Am. J. |
| | | | | Pathol., 163, 217-229) |
| 3.4 | NSEP1 | NM_004559 | | |
| 3.4 | v-Myc | NM_002467 | (Zajac-Kaye, 2001, Supra) | |
| 3.4 | NOL5A | NM_006392 | | |
| 3.4 | PSEN1 | AF416717 | | (Farquhar et al., 2003, |
| | | | | Neurosci, Lett., 346, 53-36) |
| 3.3 | S100 A11 protein | NM_005620 | | (Deloulme et al., 2000, J. Biol. |
| | | | | Chem., 275, 35302-35310) |
| 3.3 | L11 | SEQ ID NO11 | | |
| 3.3 | DNAJC8 | NM_014280 | | |
| 3.3 | MORF4L2 | NM_012286 | | |
| 3.3 | RPL28 | NM_000991 | | |
| 3.2 | AASDHPPT | NM_015423 | | |
| 3.2 | calnexin | NM_001746 | (Garcia-Lora et al., 2003, | |
| | | | Int.J. Cancer, 106, 521-527) | |
| 3.2 | TOP2A | NM_001067 | (Wikman et al., 2002, Supra) | |
| 3.2 | SET translocation | NM_003011 | | |
| 3.1 | NIFIE 14 | NM_032635 | | |
| 3.1 | ubiquitin carrier protein | M91670 | | |
| 3.1 | RRM-2 | NM_001034 | | (Xue et al., 2003, Supra) |
| 3.1 | phosphoglycerate kinase 1 | NM_000291 | (Yazawa et al., 2003, Pathol, | |
| | | | Int., 53, 58-65) | |
| 3 | actin, gamma | NM_001614 | (Redondo et al., 1998, Supra) | |
| 3 | karyopherin beta 1 | NM_002265 | | (Le Roux & Moroianu, 2003, |
| | | | | Supra) |
| 3 | GSTP1 | NM_000852 | | (Li et al., 2003, Supra) |
| 3 | RPL37 | D23661 | | (Loging & Reisman, 1999, |
| | | | | Supra) |
| 2.9 | PSAT1 | NM_021154 | | |
| 2.9 | CLCP1 | NM_080927 | (Koshikawa et al., 2002, | |
| | | | Oncogene, 21, 2822-2828) | |
| 2.9 | CYP1B1 | NM_000782 | (Hukkanen et al., 2000, Am. | |
| | | | J. Respir. Cell Mol. Biol., 22, | |
| | | | 360-366) | |
| 2.9 | FTH1 | NM_002032 | | (Pietsch et al., 2003, Supra) |
| 2.9 | PAI-1 | NM_015640 | (Robert et al., 1999, Supra) | |
| 2.9 | L12 | SEQ ID NO12 | | |
| 2.9 | RPS16 | NM_001020 | | (Karan et al., 2002, |
| | | | | Carcinogenesis, 23, 967-975) |
| 2.9 | F-box protein | AY040878 | | (Cong et al., 2003, BMC Mol. |
| | | | | Biol., 4, 10) |
| 2.9 | HNRPA1 | NM_002136 | | (Sykes et al., 2003, Supra) |
| 2.9 | RRM1 | NM_001033 | (Pitterle et al., 1999, Supra) | |
| 2.9 | LDHB | NM_002300 | | (Maekawa et al., 2003, Supra) |
| 2.9 | osteopontin | J04765 | (Zhang et al., 2001, Supra) | |
| 2.9 | Ras G3BP | BC006997 | (Silini et al., 1994, Supra) | |
| 2.8 | ACTN-1 | BT007207 | (Echchakir et al., 2001) | |
| 2.8 | L13 | SEQ ID NO13 | | |
| 2.8 | CENPF | NM_016343 | (Rattner et al., 1997, Supra) | |
| 2.8 | E2F6 | NM_135465 | | (Cullen et al., 2003, Cancer |
| | | | | Res.., 63, 5513-5520) |
| 2.8 | PGM1 | NM_002633 | (Tsybikova et al., 1996, | |
| | | | Supra) | |
| 2.8 | integrin beta 5 | NM_002213 | (Smythe et al., 1995, Supra) | |
| 2.8 | MSCP | NM_016612 | | |
| 2.8 | RPS3 | NM_001005 | (McDoniels-Silvers et al., | |
| | | | 2002, Supra) | |
| 2.8 | LDB1 | NM_003893 | | (Mizunuma et al., 2003, Supra) |
| 2.8 | RAC2 | NM_002872 | | (Gabig et al., 1995, Blood, 85, |
| | | | | 804-811) |
| 2.6 | PC4 | NM_006713 | (Latif et al., 1997, Supra) | |
| 2.6 | PTP4A2 | NM_003479 | | (Cates et al., 1996, Cancer |
| | | | | Lett., 110, 49-55) |
| 2.5 | CCT5 | NM_012073 | (Kikuchi et al., 2003, | |
| | | | Oncogene, 22, 2192-2205) | |
| 2.5 | L14 | SEQ ID NO14 | | |
| 2.4 | CD59 | X17198 | (Niehans et al., 1996, Supra) | |
| 2.4 | HNRPA2 | NM_002137 | | (Yan-Sanders et al., 2002, |
| | | | | Supra) |
| 2.4 | GRO1 | NM_001511 | (Taxman et al., 2003, Supra) | |
| 2.3 | L15 | SEQ ID NO15 | | |
| 2.3 | CNAP1 | NM_014865 | | |
| 2.3 | L16 | SEQ ID NO16 | | |
| 2.3 | MCM6 | NM_005915 | | |
| 2.2 | ATP5B | NM_001686 | | |
| 2.2 | L17 | SEQ ID NO17 | | |
| 2.2 | HMG-1 | NM_002128 | (Schraml et al., 1997, Supra) | |
| 2.2 | RPS26 | X77770 | | |
| 2.2 | amphiregulin | NM_001657 | (Hurbin et al., 2002, J. Biol. | |
| | | | Chem., 277, 49127-49133) | |
| 2.1 | L18 | SEQ ID NO18 | | |
| 2.1 | L19 | SEQ ID NO19 | | |
| 2.1 | MAGEA6 | NM_175868 | | (Dalerba et al., 2001, Int. J. |
| | | | | Cancer, 93, 85-90) |
| 2 | tropomyosin 3 | NM_152263 | | |
| 2 | MKRN-1 | NM_013446 | | |

TABLE 8
Sequence listings for 99 genes not previously reported in NSCLC and included in GES1a
References

| T:N | Identity | Ref. Seq. | NSCLC | Other Cancers |
|---|---|---|---|---|
| 40.2 | BCMP101 | BC052957 | | (Adam, et al. 2003, J. Biol. |
| | | | | Chem., 278, 6482-6489) |
| 35.4 | keratin hair basic 1 | NM_002281 | | (Cribier et al., 2001, Br.J. |
| | | | | Dermatol., 144, 977-982) |
| 25.1 | signal peptidase complex | NM_014300 | | (Dubuisson et al., 1994, J. |
| | | | | Virol., 68,6147-6160) |
| 22.9 | NAD(P)H dehydrogenase | NM_000903 | | (Cullen et al., 2003, Cancer |
| | | | | Res., 63, 5513-5520) |
| 19.6 | ATIC | NM_004044 | | (Colleoni et al., 2000, Am. J. |
| | | | | Pathol., 156, 781-789) |
| 18.3 | L1 | SEQ ID NO1 | | |
| 13.4 | L2 | SEQ ID NO2 | | |
| 11.1 | CSE1 | NM_001316 | | (Behrens et al., 2003, |
| | | | | Apoptosis, 8, 39-44) |
| 9.9 | aspartate beta-hydroxylase | U03109 | | (Maeda et al., 2003, Supra) |
| 8.2 | GAGE-4 | NM_001474 | | (Dalerba et al., 2001, Int. J. |
| | | | | Cancer, 93, 85-90) |
| 7.3 | METTL3 | NM_019852 | | |
| 7.2 | bZIP | NM_014670 | | (Gombart et al., 2002, Blood, |
| | | | | 99, 1332-1340) |
| 6.9 | ANAPC5 | NM_016237 | | (Yu et al., 1998, Science, 279, |
| | | | | 1219-1222) |
| 6.5 | GAGE8 | NM_001468 | | (Dalerba et al., 2001, Int.J. |
| | | | | Cancer, 93, 85-90) |
| 6.2 | L3 | SEQ ID NO3 | | |
| 6 | L4 | SEQ ID NO4 | | |
| 5.8 | PTTG-1 | NM_004219 | | (Shibata et al., 2002, Jpn. J. |
| | | | | Clin. Oncol., 32, 233-237) |
| 5.7 | tranaketolase | NM_001064 | | (Heinrich et al., 1976, Cancer |
| | | | | Res., 36, 3189-3197) |
| 5.6 | NM23A | NM_000269 | | (Kikuchi et al., 2003, |
| | | | | Oncogene. 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., 273, |
| | | | | 1019-1024) |
| 5.3 | RPS13 | NM_001017 | | (Denis et al., 1993, Int.J. |
| | | | | Cancer, 55, 275-280) |
| 5.3 | RPL8 | Z28407 | | (Luo et al., 2002, Br. J. Cancer, |
| | | | | 87, 339-343) |
| 5.3 | TCF6L1 | NM_003201 | | (Yoshida et al., 2003, Cancer |
| | | | | Res., 63, 3729-3734) |
| 5.3 | L5 | SEQ ID NO5 | | |
| 5.2 | NPM1 | NM_002520 | | (Larramendy et al., 2002. |
| | | | | Supra) |
| 5.2 | H2AFZ | NM_002106 | | |
| 5.1 | RPL5 | NM_000969 | | (Bertram et al., 1998, Eur J. |
| | | | | Cancer, 34, 731-736) |
| 5 | FER1L3 | NM_013451 | | |
| 5 | CYP24A1 | NM_000782 | | |
| 4.9 | TEBP | NM_006601 | | (Ponnazhagan & Kwon, 1992, |
| | | | | Pigment Cell Res., 5, 155-161) |
| 4.8 | RPS10 | NM_001014 | | |
| 4.7 | MAGEA3 | NM_005362 | | (Dalerba et al., 2001, Int. J. |
| | | | | Cancer, 93, 85-90) |
| 4.7 | RPL23A | NM_000984 | | (Jiang et al., 1997, Oncogene, |
| | | | | 14, 473-480) |
| 4.7 | RBM8A | NM_005105 | | (Salicioni et al., 2000, Supra) |
| 4.4 | XRN2 | NM_012255 | | |
| 4.3 | ARPC-1B | NM_005720 | | (Kanner et al., 1990, Proc. Natl. |
| | | | | Acad. Sci. USA, 87, 3328-3332 |
| 4.2 | EIF3S10 | NM_003750 | | (Kress at al., 1998, J. Cancer |
| | | | | Res. Glin. Oncol., 124, 315-320 |
| 4.2 | PKM2 | M26252 | | |
| 4.2 | TRIM16 | NM_006470 | | |
| 4.2 | L6 | SEQ ID NO6 | | (Kanner et al., 1990, Proc. Natl |
| | | | | Acad. Sci. USA, 87, 3328-3332 |
| 4.1 | EIF3S3 | NM_003756 | | |
| 4.1 | L7 | SEQ ID NO7 | | (Kikuchi et al., 2003, |
| | | | | Oncogene, 22, 2192-2205) |
| 4 | tubulin beta 5 | AF141349 | | |
| 4 | PPIA | NM_021130 | | (Grinstein et al., 2002, J. Exp. |
| | | | | Med., 196, 1067-1078) |
| 3.9 | nucleolin | NM_005381 | | (Su et al., 1998. Proc., Natl. |
| | | | | Acad. Sci. USA, 95, 1764-1769) |
| 3.9 | EEF1A1 | NM_001402 | | |
| 3.9 | L8 | SEQ ID NO8 | | (Lai et al., 2003, Br. J. Cancer, |
| | | | | 88, 270-276) |
| 3.8 | BNIP3 | NM_004052 | | (Matsuda et al., 2000, Supra) |
| 3.8 | UNRIP | NM_007178 | | |
| 3.8 | TRIP12 | NM_004238 | | |
| 3.6 | L9 | SEQ ID N09 | | (Le Roux & Moroianu, 2003, |
| | | | | Supra) |
| 3.5 | KPNA2 | BC005978 | | |
| 3.5 | TUBG1 | NM_001070 | | (Farquhar et al., 2003, |
| | | | | Neurosci, Letty., 346, 53-56) |
| 3.4 | PSEN1 | AF416717 | | (Hansel et al., 2003. Am. J. |
| | | | | Pathol., 163, 217-229) |
| 3.4 | CTP synthase | NM_001905 | | (Henry et al., 1993, Cancer |
| | | | | Res., 53, 1403-1408) |
| 3.4 | RPL19 | NM_000981 | | |
| 3.4 | L10 | SEQ ID NO10 | | |
| 3.4 | NSEP1 | NM_004559 | | |
| 3.4 | NOL5A | NM_006392 | | (Deloulme et al., 2000, J. Biol. |
| | | | | Chem., 275, 35302-35310) |
| 3.3 | S100 A11 protein | NM_005620 | | |
| 3.3 | L11 | SEQ ID NO11 | | |
| 3.3 | DNAJC8 | NM_014280 | | |
| 3.3 | MORF4L2 | NM_012286 | | |
| 3.3 | RPL28 | NM_000991 | | |
| 3.2 | AASDHPPT | NM_015423 | | |
| 3.1 | RRM-2 | NM_001034 | | (Xue et al., 2003, Supra) |
| 3.1 | NIFIE 14 | NM_032635 | | |
| 3.1 | ubiquitin carrier protein | M91670 | | (Le Roux & Moroianu, 2003. |
| | | | | Supra) |
| 3 | karyopherin beta 1 | NM_002265 | | (Li et al., 2003, Supra) |
| 3 | GSTP1 | NM_000852 | | (Loging & Reisman, 1999, |
| | | | | Supra) |
| 3 | RPL37 | D23661 | | (Cong et al., 2003, BMC Mol. |
| | | | | Biol., 4, 10) |
| 2.9 | F-box protein | AY040878 | | (Karan et al., 2002, |
| | | | | Carcinogenesis, 23, 967-975) |
| 2.9 | RPS16 | NM_001020 | | (Maekawa et al., 2003, Supra) |
| 2.9 | LDHB | NM_002300 | | (Pietsch et al., 2003, Supra) |
| 2.9 | FTHI | NM_002032 | | (Sykes et al., 2003, Supra) |
| 2.9 | HNRPAI | NM_002136 | | |
| 2.9 | PSAT1 | NM_021154 | | |
| 2.9 | L12 | SEQ ID N012 | | (Cullen et al., 2003, Cancer |
| | | | | Res., 63, 5513-5520) |
| 2.8 | E2F6 | NM_135465 | | |
| 2.8 | RAC2 | NM_002872 | | (Gabig et al., 1995. Blood, 85, |
| 2.8 | LDB1 | NM_003893 | | (Mizunuma et al., 2003, Supra) |
| 2.8 | L13 | SEQ ID NO13 | | |
| 2.8 | MSCP | NM_016612 | | |
| 2.6 | PTP4A2 | NM_003479 | | (Cates et al., 1996, Cancer |
| | | | | Lett., 110, 49-55) |
| 2.5 | L14 | SEQ ID NO14 | | |
| 2.4 | HNRPA2 | NM_002137 | | (Yan-Sanders et al., 2002, |
| | | | | Supra) |
| 2.3 | L15 | SEQ ID NO15 | | |
| 2.3 | CNAP1 | NM_014865 | | |
| 2.3 | L16 | SEQ ID NO16 | | |
| 2.3 | MCM6 | NM_005915 | | |
| 2.2 | ATP5B | NM_001686 | | |
| 2.2 | L17 | SEQ ID NO17 | | |
| 2.2 | RPS26 | X77770 | | |
| 2.1 | MAGEA6 | NM_175868 | | (Dalerba et al., 2001, Int. J. |
| | | | | Cancer, 93, 85-90) |
| 2.1 | L18 | SEQ ID NO18 | | |
| 2.1 | L19 | SEQ ID NO19 | | |
| 2 | tropomyosin 3 | NM_152263 | | |
| 2 | MKRN-1 | NM_013446 | | |

TABLE 9
Sequence listings for 46 genes not previously cancer-associated
and included in GES1b

| T:N | Identity | Ref. Seq. |
|---|---|---|
| 18.3 | L1 | SEQ ID NO1 |
| 13.4 | L2 | SEQ ID NO2 |
| 7.3 | METTL3 | NM_019852 |
| 6.2 | L3 | SEQ ID NO3 |
| 6 | L4 | SEQ ID NO4 |
| 5.3 | L5 | SEQ ID NO5 |
| 5.2 | H2AFZ | NM_002106 |
| 5 | FER1L3 | NM_013451 |
| 5 | CYP24A1 | NM_000782 |
| 4.8 | RPS10 | NM_001014 |
| 4.4 | XRN2 | NM_012255 |
| 4.3 | ARPC-1B | NM_005720 |
| 4.2 | TRIM16 | NM_006470 |
| 4.2 | L6 | SEQ ID NO6 |
| 4.1 | L7 | SEQ ID NO7 |
| 4 | PPIA | NM_021130 |
| 3.9 | L8 | SEQ ID NO8 |
| 3.8 | TRIP12 | NM_004238 |
| 3.6 | L9 | SEQ ID NO9 |
| 3.5 | TUBG1 | NM_001070 |
| 3.4 | L10 | SEQ ID NO10 |
| 3.4 | NSEP1 | NM_004559 |
| 3.4 | NOL5A | NM_006392 |
| 3.3 | L11 | SEQ ID NO11 |
| 3.3 | DNAJC8 | NM_014280 |
| 3.3 | MORF4L2 | NM_012286 |
| 3.3 | RPL28 | NM_000991 |
| 3.2 | AASDHPPT | NM_015423 |
| 3.1 | NILFIE 14 | NM_032635 |
| 3.1 | ubiquitin carrier protein | M91670 |
| 2.9 | PSAT1 | NM_021154 |
| 2.9 | L12 | SEQ ID NO12 |
| 2.8 | L13 | SEQ ID NO13 |
| 2.8 | MSCP | NM_016612 |
| 2.5 | L14 | SEQ ID NO14 |
| 2.3 | L15 | SEQ ID NO15 |
| 2.3 | CNAP1 | NM_014865 |
| 2.3 | L16 | SEQ ID NO16 |
| 2.3 | MCM6 | NM_005915 |
| 2.2 | ATP5B | NM_001686 |
| 2.2 | L17 | SEQ ID NO17 |
| 2.2 | RPS26 | X77770 |
| 2.1 | L18 | SEQ ID NO18 |
| 2.1 | L19 | SEQ ID NO19 |
| 2 | tropomyosin 3 | NM_152263 |
| 2 | MKRN-1 | NM_013446 |

TABLE 10
Sequence listings for 53 genes not previously NSCLC-associated and included in GES1c
References

| T:N | Identity | Ref. Seq. | NSCLC | Other Cancers |
|---|---|---|---|---|
| 40.2 | BCMP101 | BC052957 | | (Adam et al. 2003, J. Biol. |
| | | | | Chem., 278, 6482-6489) |
| 35.4 | keratin hair basic 1 | NM_002281 | | (Cribier et al., 2001, Br.J. |
| | | | | Dermatol, 144, 977-982) |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res., 63, 5513-5520) |
| 19.6 | ATIC | NM_004044 | | (Colleoni et al., 2000, Am. J. |
| | | | | Pathol., 156, 781-789) |
| 11.1 | CSE1 | NM_001316 | | (Behrens et al., 2003, |
| | | | | Apoptosis, 8, 39-44) |
| 9.9 | aspartate beta- | U03109 | | (Maeda et al., 2003, Supra) |
| | hydroxylase | | | |
| 8.2 | GAGE-4 | NM_001474 | | (Dalerba et al., 2001, Int. J. |
| | | | | Cancer, 93, 85-90) |
| 7.2 | bZIP | NM_014670 | | (Gombart et al., 2002, Blood, |
| | | | | 99, 1332-1340) |
| 6.9 | ANAPC5 | NM_016237 | | (Yu et al., 1998, Science, 279, |
| | | | | 1219-1222) |
| 6.5 | GAGE8 | NM_001468 | | (Dalerba et al., 2001, Int. J. |
| | | | | Cancer, 93, 85-90) |
| 5.8 | PTTG-1 | NM_004219 | | (Shibata et al., 2002, Jpn. J. |
| | | | | Clin. Oncol., 32, 233-237) |
| 5.7 | transketolase | NM_001064 | | (Heinrich et al., 1976, Cancer |
| | | | | Res., 36, 3189-3197) |
| 5.6 | NM23A | NM_000269 | | (Kikuchi et al., 2003, |
| | | | | Oncogene, 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., 273, |
| | | | | 1019-1024) |
| 5.3 | TCF6L1 | NM_003201 | | (Yoshida et al., 2003, Cancer |
| | | | | Res., 63, 3729-3734) |
| 5.3 | RPL8 | Z28407 | | (Luo et al., 2002, Br. J Cancer, |
| | | | | 87, 339-343) |
| 5.3 | RPS13 | NM_001017 | | (Denis et al., 1993, Int. J. |
| | | | | Cancer, 55, 275-280) |
| 5.2 | NPM1 | NM_002520 | | (Larramendy et al., 2002, |
| | | | | Supra) |
| 5.1 | RPL5 | NM_000969 | | (Bertram et al., 1998, Eur. J. |
| | | | | Cancer, 34, 731-736) |
| 4.9 | TEBP | NM_006601 | | (Ponnazhagan & Kwon, 1992, |
| | | | | Supra) |
| 4.7 | RBM8A | NM_005105 | | (Salicioni et al., 2000, Supra) |
| 4.7 | RPL23A | NM_000984 | | (Jiang et al., 1997, Oncogene, |
| | | | | 14, 473-480) |
| 4.7 | MAGEA3 | NM_005362 | | (Dalerba et al., 2001. Int.J. |
| | | | | Cancer, 93, 85-90) |
| 4.2 | PKM2 | M26252 | | (Kress et al., 1998, J. Cancer |
| | | | | Res. Clin., Oncol., 124, 315-320) |
| 4.2 | EIF3S10 | NM_003750 | | (Kanner et al., 1990, Proc. Natl. |
| | | | | Acad. Sci. USA, 87, 3328-3332) |
| 4.1 | EIF3S3 | NM_003756 | | (Kanner et al., 1990, Proc. Natl. |
| | | | | Acad. Sci. USA, 87, 3328-3332) |
| 4 | tubulin beta 5 | AF141349 | | (Kikuchi et al., 2003, |
| | | | | Oncogene, 22, 2192-2205) |
| 3.9 | EEF1A1 | NM_001402 | | (Su et al., 1998, {Proc. Natl. |
| | | | | Acad. Sci. USA, 95, 1764-1769) |
| 3.9 | nucleolin | NM_005381 | | (Grinstein et al., 2002, J. Exp. |
| | | | | Med., 196, 1067-1078) |
| 3.8 | UNRIP | NM_007178 | | (Matsuda et al., 2000, Supra) |
| 3.8 | BNIP3 | NM_004052 | | (Lai et al., 2003, Br.J. Cancer, |
| | | | | 88, 270-276) |
| 3.5 | KPNA2 | BC005978 | | (Le Roux & Moroianu, 2003, J. |
| | | | | Virol., 77, 2330-2337) |
| 3.4 | RPL19 | NM_000981 | | (Henry et al., 1993, Cancer |
| | | | | Res., 53, 1403-1408) |
| 3.4 | CTP synthase | NM_001905 | | (Hansel et al., 2003, Am. J. |
| | | | | Pathol., 163, 217-229) |
| 3.4 | PSEN1 | AF416717 | | (Farquhar et al., 2003, |
| | | | | Neurosci, Lett., 346, 53-36) |
| 3.3 | S100 A11 protein | NM_005620 | | (Deloulme et al., 2000, J. Biol. |
| | | | | Chem., 275, 35302-35310) |
| 3.1 | RRM-2 | NM_001034 | | (Xue et al., 2003, Supra) |
| 3 | RPL37 | D23661 | | (Loging & Reisman, 1999, |
| | | | | Supra) |
| 3 | GSTP1 | NM_000852 | | (Li et al., 2003, Supra) |
| 3 | karyopherin beta 1 | NM_002265 | | (Le Roux & Moroianu, 2003, |
| | | | | Supra) |
| 2.9 | HNRPA1 | NM_002136 | | (Sykes et al., 2003, Supra) |
| 2.9 | FTH1 | NM_002032 | | (Pietsch et al., 2003, Supra) |
| 2.9 | LDHB | NM_002300 | | (Maekawa et al., 2003, Supra) |
| 2.9 | RPS16 | NM_001020 | | (Karan et al., 2002, |
| | | | | Carcinogenesis, 23, 967-975) |
| 2.9 | F-box protein | AY040878 | | (Cong et al., 2003, BMC Mol. |
| | | | | Biol., 4, 10) |
| 2.8 | LDB1 | NM_003893 | | (Mizunuma et al., 2003, Supra) |
| 2.8 | RAC2 | NM_002872 | | (Gabig et al., 1995, Blood 85, |
| | | | | 804-811) |
| 2.8 | E2F6 | NM_135465 | | (Cullen et al., 2003, Cancer |
| | | | | Res., 63, 5513-5520) |
| 2.6 | PTP4A2 | NM_003479 | | (Cates et al., 1996, Cancer |
| | | | | Lett., 110, 49-55) |
| 2.4 | HNRPA2 | NM_002137 | | (Yan-Sanders et al., 2002, |
| | | | | Supra) |
| 2.1 | MAGEA6 | NM_175868 | | (Dalerba et al., 2001, Int.J. |
| | | | | Cancer, 93, 85-90) |

TABLE 11
Sequence listings for 19 novel genes included in GES1d

| T:N | Identity | Ref. Seq. |
|---|---|---|
| 18.3 | L1 | SEQ ID NO1 |
| 13.4 | L2 | SEQ ID NO2 |
| 6.2 | L3 | SEQ ID NO3 |
| 6 | L4 | SEQ ID NO4 |
| 5.3 | L5 | SEQ ID NO5 |
| 4.2 | L6 | SEQ ID NO6 |
| 4.1 | L7 | SEQ ID NO7 |
| 3.9 | L8 | SEQ ID NO8 |
| 3.6 | L9 | SEQ ID NO9 |
| 3.4 | L10 | SEQ ID NO10 |
| 3.3 | L11 | SEQ ID NO11 |
| 2.9 | L12 | SEQ ID NO12 |
| 2.8 | L13 | SEQ ID NO13 |
| 2.5 | L14 | SEQ ID NO14 |
| 2.3 | L15 | SEQ ID NO15 |
| 2.3 | L16 | SEQ ID NO16 |
| 2.2 | L17 | SEQ ID NO17 |
| 2.1 | L18 | SEQ ID NO18 |
| 2.1 | L19 | SEQ ID NO19 |

TABLE 12
Sequence listings for 13 genes ≧ 10-fold over-expressed in NSCLC and included in GES1e
References

| T:N | Identity | Ref. Seq. | NSCLC | Other Cancers |
|---|---|---|---|---|
| 40.2 | BCMP101 | BC052957 | | (Adam, et al. 2003, J. Biol. |
| | | | | Chem., 278, 6482-6489) |
| 35.4 | keratin hair basic 1 | NM_002281 | | (Cribier et al., 2001, Br. J. |
| | | | | Dermatol., 144, 977-982) |
| 29.7 | aldehyde dehydrogenase 1 | NM_000689 | (Schnier et al., 1999, FEBS | |
| | | | Lett., 454, 100-104) | |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res.., 63, 5513-5520) |
| 22.6 | aldo-keto reductase 1 | NM_020299 | (Palackal et al., 2002, Supra) | |
| 19.6 | ATIC | NM_004044 | | (Colleoni et al., 2000, Am. J. |
| | | | | Pathol., 156, 781-789) |
| 18.3 | L1 | SEQ ID NO1 | | |
| 14.8 | PGP 9.5 | NM_004181 | (Hibi et al., 1999, Am. J. | |
| | | | Pathol., 155, 711-715) | |
| 14.8 | annexin 1 | NM_000700 | (Traverso et al., 1998, Supra) | |
| 13.4 | CXC member 5 | NM_002994 | | |
| 13.4 | L2 | SEQ ID NO2 | | |
| 11.1 | CSE1 | NM_001316 | | (Behrens et al., 2003, |
| | | | | Apoptosis, 8, 39-44) |

TABLE 13
Sequence listings for 45 genes ≧ 5-fold over-expressed in NSCLC and included in GES1f
References

| T:N | Identity | Ref. Seq. | NSCLC | Other Cancers |
|---|---|---|---|---|
| 40.2 | BCMP101 | BC052957 | | (Adam et al. 2003, J. Biol |
| | | | | Chem., 278, 6482-6489) |
| 35.4 | keratin hair basic 1 | NM_002281 | | (Cribier et al., 2001, Br. J. |
| | | | | Dermatol, 144, 977-982) |
| 29.7 | aldehyde dehydrogenase 1 | NM_000689 | (Schnier et al., 1999, FEBS | |
| | | | Lett., 454, 100-104) | |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res., 63, 5513-5520) |
| 22.6 | aldo-keto reductase 1 | NM_020299 | (Palackal et al., 2002, Supra) | |
| 19.6 | ATIC | NM_004044 | | (Colleoni et al., 2000, Am. J. |
| | | | | Pathol., 156, 781-789) |
| 18.3 | L1 | SEQ ID NO1 | | |
| 14.8 | PGP 9.5 | NM_004181 | (Hibi et al., 1999, Am. J. | |
| | | | Pathol., 155, 711-715) | |
| 14.8 | annexin 1 | NM_000700 | (Traverso et al., 1998, Supra) | |
| 13.4 | CXC member 5 | NM_002994 | (Arenberg, et al. 1998, J. Clin. | |
| | | | Invest., 102, 465-472) | |
| 13.4 | L2 | SEQ ID NO2 | | |
| 11.1 | CSE1 | NM_001316 | | (Behrens et al., 2003, |
| | | | | Apoptosis, 8, 39-44) |
| 9.9 | aspartate beta-hydroxylase | U03109 | | (Maeda et al., 2003, Supra) |
| 9.8 | LDHA | NM_005566 | (Beer et al., 2002, Nat. Med., 8, | |
| | | | 816-824) | |
| 8.2 | GAGE-4 | NM_001474 | | (Dalerba et al., 2001, Int. J. |
| | | | | Cancer, 93, 85-90) |
| 7.4 | cytokeratin 18 | NM_000224 | (Young et al., 2002, Supra) | |
| 7.3 | METTL3 | NM_019852 | | |
| 7.2 | bZIP | NM_014670 | | (Gombart et al., 2002, Blood |
| | | | | 99, 1332-1340) |
| 7 | calpastatin | AF327443 | (Gil-Parrado et al., 2002, J. | |
| | | | Biol. Chem., 277, 27217-27226) | |
| 6.9 | ANAPC5 | NM_016237 | | (Yu et al., 1998, Science, 279, |
| | | | | 1219-1222) |
| 6.8 | cysteine-rich inducer 61 | NM_001554 | (Tong et al., 2001, Supra) | |
| 6.7 | TFP inhibitor 2 | NM_006528 | (Ganapathi et al., 1988, Br.J. | |
| | | | Cancer, 58, 335-340) | |
| 6.6 | thioredoxin reductase 1 | AJ001050 | (Soini et al., 2001, Clin. Cancer | |
| | | | Res., 7, 1750-1757) | |
| 6.5 | met proto-oncogene | NM_000245 | (Lorenzato et al., 2002, Cancer | |
| | | | Res., 62, 7025-7030) | |
| 6.5 | GAGE8 | NM_001468 | | (Dalerba et al., 2001, Int.J. |
| | | | | Cancer, 93, 85-90) |
| 6.2 | L3 | SEQ ID NO3 | | |
| 6 | L4 | SEQ ID NO4 | | |
| 5.9 | splicing factor3 | NM_003017 | (Kikuchi et al., 2003, | |
| | | | Oncogene, 22, 2192-2205) | |
| 5.9 | K-aplha-1 tubulin | NM_032704 | (Rao et al., 2001, Supra) | |
| 5.8 | PTTG-1 | NM_004219 | | (Shibata et al., 2002, Jpn. J. |
| | | | | Clin. Oncol., 32, 233-237) |
| 5.7 | transketolase | NM_001064 | | (Heinrich et al., 1976, Cancer |
| | | | | Res., 36, 3189-3197 |
| 5.6 | thioredoxin | NM_003329 | (Soini et al., 2001, Clin. Cancer | |
| | | | Res., 7,1750-1757) | |
| 5.6 | NM23A | NM_000269 | | (Kikuchi et al., 2003, |
| | | | | Oncogene, 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., 273, |
| | | | | 1019-1024) |
| 5.3 | TCF6L1 | NM_003201 | | (Yoshida et al., 2003, Cancer |
| | | | | Res., 63, 3729-3734) |
| 5.3 | RPL8 | Z28407 | | (Luo et al., 2002, Br. J. Cancer, |
| | | | | 87, 339-343) |
| 5.3 | RPS13 | NM_001017 | | (Denis et al., 1993, Int.J. |
| | | | | Cancer, 55, 275-280) |
| 5.3 | L5 | SEQ ID NO5 | | |
| 5.2 | NPM1 | NM_002520 | | (Larramendy et al., 2002, |
| | | | | Supra) |
| 5.2 | H2AFZ | NM_002106 | | |
| 5.1 | RPL5 | NM_000969 | | (Bertram et al., 1998, Eur. J. |
| | | | | Cancer, 34, 731-736) |
| 5 | FER1L3 | NM_013451 | | |
| 5 | CYP24A1 | NM_000782 | | |

TABLE 14
Sequence listings for 66 genes ≧ 4-fold over-expressed in NSCLC and included in GES1g
References

| T:N | Identity | Ref. Seq. | NSCLC | Other Cancers |
|---|---|---|---|---|
| 40.2 | BCMP101 | BC052957 | | (Adam et al. 2003, J. Biol. |
| | | | | Chem., 278, 6482-6489) |
| 35.4 | keratin hair basic 1 | NM_002281 | | (Cribier et al., 2001, Br. J. |
| | aldehyde dehydrogenase | | | Dermatol., 144, 977-982) |
| 29.7 | 1 | NM_000689 | (Schnier et al., 1999, FEBS Lett., | |
| | | | 454, 100-104) | |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res., 63, 5513-5520) |
| 22.6 | aldo-keto reductase 1 | NM_020299 | (Palackal et al., 2002, Supra) | |
| 19.6 | ATIC | NM_004044 | | (Colleoni et al., 2000, Am. J. |
| | | | | Pathol., 156, 781-789) |
| 18.3 | L1 | SEQ ID NO1 | | |
| 14.8 | PGP 9.5 | NM_004181 | (Hibi et al., 1999. Am. J. Pathol., | |
| | | | 155, 711-715 | |
| 14.8 | annexin 1 | NM_000700 | (Traverso et al., 1998, Supra) | |
| 13.4 | CXC member 5 | NM_002994 | (Arenberg, et al. 1998, J. Clin. | |
| | | | Invest., 102, 465-472) | |
| 13.4 | L2 | SEQ ID NO2 | | |
| 11.1 | CSE1 | NM_001316 | | (Behrens et al., 2003, |
| | | | | Apoptosis, 8, 39-44) |
| 9.9 | aspartate beta- | U03109 | | (Maeda et al., 2003, Supra) |
| | hydroxylase | | | |
| 9.8 | LDHA | NM_005566 | (Beer et al., 2002, Nat. Med., 8, | |
| | | | 816-824) | |
| 8.2 | GAGE-4 | NM_001474 | | (Dalerba et al., 2001, Int. J. |
| | | | | Cancer, 93, 85-90) |
| 7.4 | cytokeratin 18 | NM_000224 | (Young et al., 2002, Supra) | |
| 7.3 | METTL3 | NM_019852 | | |
| 7.2 | bZIP | NM_014670 | | (Gombart et al., 2002, Blood |
| | | | | 99, 1332-1340) |
| 7 | calpastatin | AF327443 | (Gil-Parrado et al., 2002, J. Biol. | |
| | | | Chem., 277, 27217-27226) | |
| 6.9 | ANAPC5 | NM_016237 | | (Yu et al., 1998, Science, 279, |
| | | | | 1219-1222) |
| 6.8 | cysteine-rich inducer 61 | NM_001554 | (Tong et al., 2001, Supra) | |
| 6.7 | TFP inhibitor 2 | NM_006528 | (Ganapathi et al., 1988, Br. J. | |
| | | | Cancer, 58, 335-340) | |
| 6.6 | thioredoxin reductase 1 | AJ001050 | (Soini et al., 2001. Clin. Cancer | |
| | | | Res., 7, 1750-1757) | |
| 6.5 | met proto-oncogene | NM_000245 | (Lorenzato et al., 2002, Cancer | |
| | | | Res., 62, 7025-7030) | |
| 6.5 | GAGE8 | NM_001468 | | (Dalerba et al., 2001, Int.J. |
| | | | | Cancer, 93, 85-90) |
| 6.2 | L3 | SEQ ID NO3 | | |
| 6 | L4 | SEQ ID NO4 | | |
| 5.9 | splicing factor3 | NM_003017 | (Kikuchi et al., 2003, Oncogene, | |
| | | | 22, 2192-2205) | |
| 5.9 | K-aplha-1 tubulin | NM_032704 | (Rao et al., 2001, Supra) | |
| 5.8 | PTTG-1 | NM_004219 | | (Shibata et al., 2002, Jpn. J. |
| | | | | Clin. Oncol., 32, 233-237) |
| 5.7 | transketolase | NM_001064 | | (Heinrich et al., 1976, Cancer |
| | | | | Res., 36, 3189-3197) |
| 5.6 | thioredoxin | NM_003329 | (Soini et al., 2001, Clin. Cancer | |
| | | | Res., 7,1750-1757) | |
| 5.6 | NM23A | NM_000269 | | (Kikuchi et al., 2003, |
| | | | | Oncogene, 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., 273, |
| | | | | 1019-1024) |
| 5.3 | TCF6L1 | NM_003201 | | (Yoshida et al., 2003, Cancer |
| | | | | Res., 63, 3729-3734) |
| 5.3 | RPL8 | Z28407 | | (Luo et al., 2002, Br. J. Cancer, |
| | | | | 87, 339-343) |
| 5.3 | RPS13 | NM_001017 | | (Denis et al., 1993, Int. J. |
| | | | | Cancer, 55, 275-280) |
| 5.3 | L5 | SEQ ID NO5 | | |
| 5.2 | NPM1 | NM_002520 | | (Larramendy et al., 2002, |
| | | | | Supra) |
| 5.2 | H2AFZ | NM_002106 | | |
| 5.1 | RPL5 | NM_000969 | | (Bertram et al., 1998, Eur. J. |
| | | | | Cancer, 34, 731-736) |
| 5 | FER1L3 | NM_013451 | | |
| 5 | CYP24A1 | NM_000782 | | |
| 4.9 | enolase 1 | NM_001428 | (Ferrigno et al., 2003, Lung | |
| | | | Cancer. 41, 311-320) | |
| 4.9 | TEBP | NM_006601 | | (Ponnazhagan & Kwon, 1992, |
| | | | | Supra) |
| 4.8 | CD98 | NM_002394 | (Rintoul et al., 2002, Supra) | |
| 4.8 | RPS10 | NM_001014 | | |
| 4.7 | CD29 | NM_002211 | (Oshita et al., 2002, Anticancer | |
| | | | Res., 22, 1065-1070) | |
| 4.7 | RBM8A | NM_005105 | | (Salicioni et al., 2000, Supra) |
| 4.7 | RPL23A | NM_000984 | | (Jiang et al., 1997, Oncogene, |
| | | | | 14, 473-480) |
| 4.7 | MAGEA3 | NM_005362 | | (Dalerba et al., 2001, Int. J. |
| | | | | Cancer, 93, 85-90) |
| 4.5 | cytokeratin 8 | NM_002273 | (Fukunaga et al., 2002, Lung | |
| | | | Cancer, 38, 31-38) | |
| 4.4 | XRN2 | NM_012255 | | |
| 4.3 | ARPC-1B | NM_005720 | | |
| 4.2 | HSP70 | M11717 | (Malusecka et al., 2001, | |
| | | | Anticancer Res., 21, 1015-1021) | |
| 4.2 | PKM2 | M26252 | | (Kress et al., 1998, J. Cancer |
| | | | | Res. Clin. Oncol., 124, 315-320) |
| 4.2 | EIF3S10 | NM_003750 | | (Kanner et al., 1990. Proc. Natl. |
| | | | | Acad. Sci. USA, 87, 3328-3332) |
| 4.2 | TRIM16 | NM_006470 | | |
| 4.2 | L6 | SEQ ID NO6 | | |
| 4.1 | cytokeratin 7 | NM_005556 | (Kummar et al., 2002. Br. J. | |
| | | | Cancer, 86, 1884-1887) | |
| 4.1 | EIF3S3 | NM_003756 | | (Kanner et al., 1990, Proc. Natl |
| | | | | Acad. Sci. USA, 87, 3328-3332) |
| 4.1 | L7 | SEQ ID NO7 | | |
| 4 | tubulin beta 5 | AF141349 | | (Kikuchi et al., 2003, |
| | | | | Oncogene, 22, 2192-2205) |
| 4 | PPIA | NM_021130 | | |

TABLE 15
Sequence listings for 103 genes ≧ 3-fold over-expressed in NSCLC and included in GES 1 h
References

| T:N | Identity | Ref. Seq. | NSCLC | Other Cancers |
|---|---|---|---|---|
| 40.2 | BCMP101 | BC052957 | | (Adam et al. 2003, J. Biol. |
| | | | | Chem., 278, 6482-6489) |
| 35.4 | keratin hair basic 1 | NM_002281 | | (Cribier et al., 2001, Br. J. |
| | | | | Dermatol., 144 977-982 |
| 29.7 | aldehyde dehydrogenase 1 | NM_000689 | (Schnier et al., 1999, FEBS | |
| | | | Lett., 454, 100-104) | |
| 25.1 | signal peptidase complex | NM_014300 | | (Dubuisson et al., 1994, J. |
| | | | | Virol., 68, 6147-6160) |
| 22.9 | NAD(P)H dehydrogenase | NM_000903 | | (Cullen et al., 2003, Cancer |
| | | | | Res., 63, 5513-5520) |
| 22.6 | aldo-keto reductase 1 | NM_020299 | (Palackal et al., 2002, Supra) | |
| 19.6 | ATIC | NM_004044 | | (Colleoni et al., 2000, Am. J. |
| | | | | Pathol., 156, 781-789) |
| 18.3 | L1 | SEQ ID NO1 | (Hibi et al., 1999, Am. J. | |
| | | | Pathol., 155, 711-715) | |
| 14.8 | PGP 9.5 | NM_004181 | (Traverso et al., 1998, Supra) | |
| 14.8 | annexin 1 | NM_000700 | (Arenberg, et al. 1998. J. Clin. | |
| | | | Invest., 102, 465-472) | |
| 13.4 | CXC member 5 | NM_002994 | | (Behrens et al., 2003, |
| | | | | Apoptosis, 8, 39-44) |
| 13.4 | L2 | SEQ ID NO2 | | |
| 11.1 | CSE1 | NM_001316 | (Maeda et al., 2003, Supra) | |
| 9.9 | aspartate beta-hydroxylase | U03109 | | |
| 9.8 | LDHA | NM_005566 | (Beer et al., 2002, Nat. Med., 8, | |
| | | | 816-824) | |
| 8.2 | GAGE-4 | NM_001474 | | (Dalerba et al., 2001, Int. J. |
| | | | | Cancer, 93, 85-90) |
| 7.4 | cytokeratin 18 | NM_000224 | (Young et al., 2002, Supra) | |
| 7.3 | METTL3 | NM_019852 | | |
| 7.2 | bZIP | NM_14670 | | (Gombart et al., 2002, Blood |
| | | | | 99, 1332-1340) |
| 7 | calpastatin | AF327443 | (Gil-Parrado et al., 2002, J. | |
| | | | Biol Chem., 277, 27217-27226) | |
| 6.9 | ANAPC5 | NM_016237 | | (Yu et al., 1998, Science, 279, |
| | | | | 1219-1222) |
| 6.8 | cysteine-rich inducer 61 | NM_001554 | (Tong et al., 2001, Supra) | |
| 6.7 | TFP inhibitor 2 | NM_006528 | (Ganapathi et al., 1988, Br. J. | |
| | | | Cancer, 58, 335-340) | |
| 6.6 | thioredoxin reductase 1 | AJ001050 | (Soini et al., 2001, Clin. Cancer | |
| | | | Res., 7, 1750-1757) | |
| 6.5 | met proto-oncogene | NM_000245 | (Lorenzato et al., 2002, Supra) | |
| 6.5 | GAGE8 | NM_001468 | | (Dalerba et al., 2001, Int. J. |
| | | | | Cancer, 93, 85-90) |
| 6.2 | L3 | SEQ ID NO3 | | |
| 6 | L4 | SEQ ID NO4 | | |
| 5.9 | splicing factor3 | NM_003017 | (Kikuchi et al., 2003, | |
| | | | Oncogene, 22, 2192-2205) | |
| 5.9 | K-aplha-1 tubulin | NM_032704 | (Rao et al., 2001, Supra) | |
| 5.8 | PTTG-1 | NM_004219 | | (Shibata et al., 2002, Jpn. J. |
| | | | | Clin. Oncol., 32, 233-237) |
| 5.7 | transketolase | NM_001064 | | (Heinrich et al., 1976 Cancer |
| | | | | Res., 36, 3189-3197) |
| 5.6 | thioredoxin | NM_003329 | (Soini et al., 2001, Clin. Cancer | |
| | | | Res., 7, 1750-1757) | |
| 5.6 | NM23A | NM_000269 | | (Kikuchi et al., 2003, |
| | | | | Oncogene, 22, 2192-2205) |
| 5.5 | MAD2L1 | NM_014628 | | (Percy et al., 2000, Supra) |
| 5.4 | epiregulin | NM_001432 | | (Zhu et al., 2000, Biochem. |
| | | | | Biophys. Res. Commun., |
| | | | | 273, 1019-1024) |
| 5.3 | TCF6L1 | NM_003201 | | (Yoshida et al., 2003, Cancer |
| | | | | Res., 63, 3729-3734) |
| 5.3 | RPL8 | Z28407 | | (Luo et al., 2002, Br. J. |
| | | | | Cancer, 87, 339-343) |
| 5.3 | RPS13 | NM_001017 | | (Denis et al., 1993, Int.J. |
| | | | | Cancer, 55, 275-280) |
| 5.3 | L5 | SEQ ID NO5 | | |
| 5.2 | NPM1 | NM_002520 | | (Larramendy et al., 2002, |
| | | | | Supra) |
| 5.2 | H2AFZ | NM_002106 | | (Bertram et al., 1998, Eur. J. |
| | | | | Cancer, 34, 731-736) |
| 5.1 | RPL5 | NM_000969 | | |
| 5 | FER1L3 | NM_013451 | | |
| 5 | CYP24A1 | NM_000782 | (Ferrigno et al., 2003, Lung | |
| | | | Cancer, 41, 311-320) | |
| 4.9 | enolase 1 | NM_001428 | | (Ponnazhagan & Kwon, 1992, |
| | | | | Supra) |
| 4.9 | TEBP | NM_006601 | | |
| 4.8 | CD98 | NM_002394 | (Rintoul et al., 2002, Supra) | |
| 4.8 | RPS10 | NM_001014 | | |
| 4.7 | CD29 | NM_002211 | (Oshita et al., 2002, Supra) | |
| 4.7 | RBM8A | NM_005105 | | (Salicioni et al., 2000, Supra) |
| 4.7 | RPL23A | NM_000984 | | (Jiang et al., 1997, Ocongene, |
| | | | | 14, 473-480) |
| 4.7 | MAGEA3 | NM_005362 | | (Dalerba et al., 2001, Int. J. |
| | | | | Cancer, 93, 85-90) |
| 4.5 | cytokeratin 8 | NM_002273 | (Fukunaga et al., 2002, Lung | |
| | | | Cancer, 38, 31-38) | |
| 4.4 | XRN2 | NM_012255 | | |
| 4.3 | ARPC-1B | NM_005720 | | |
| 4.2 | HSP70 | M11717 | (Malusecka et al., 2001, Supra) | |
| 4.2 | PKM2 | M26252 | | (Kress et al., 1998, J. Cancer |
| | | | | Res. Clin. Oncol., 124, 315-320) |
| 4.2 | EIF3S10 | NM_003750 | | (Kanner et al., 1990, Proc. Natl |
| | | | | Acad. Sci. USA, 87, 3328-3332) |
| 4.2 | TRIM16 | NM_006470 | | |
| 4.2 | L6 | SEQ ID NO6 | | |
| 4.1 | cytokeratin 7 | NM_005556 | (Kummar et al., 2002. Br. J. | |
| | | | Cancer, 86, 1884-1887) | |
| 4.1 | EIF3S3 | NM_003756 | | (Kanner et al., 1990, Proc. Natl. |
| | | | | Acad. Sci. USA, 87, 3328-3332 |
| 4.1 | L7 | SEQ ID NO7 | | |
| 4 | tubulin beta 5 | AF141349 | | (Kikuchi et al., 2003, |
| | | | | Oncogene, 22, 2192-2205) |
| 4 | PPIA | NM_021130 | | |
| 3.9 | CCT7 | NM_006431 | (Kikuchi et al., 2003, | |
| | | | Oncogene, 22, 2192-2205) | |
| 3.9 | cyclin B1 | NM_031966 | (Soria et al., 2000, Supra) | |
| 3.9 | EEFIA1 | NM_001402 | | (Su et al., 1998, Proc. Natl. |
| | | | | Acad. Sci. USA, 95, 1764-1769) |
| 3.9 | nucleolin | NM_005381 | | (Grinstein et al., 2002, J. Exp. |
| | | | | Med., 196, 1067-1078) |
| 3.9 | L8 | SEQ ID NO8 | | |
| 3.8 | YWHAG | NM_012479 | (Kikuchi et al., 2003, | |
| | | | Oncogene, 22, 2192-2205) | |
| 3.8 | HSP90 | X15183 | (Zhong et al., 2003, Supra) | |
| 3.8 | UNRIP | NM_007178 | | (Matsuda et al., 2000, Supra) |
| 3.8 | BNIP3 | NM_004052 | | (Lai et al., 2003, Br. J. Cancer, |
| | | | | 88, 270-276) |
| 3.8 | TRIP12 | NM_004238 | | |
| 3.6 | L9 | SEQ ID NO9 | | |
| 3.5 | KPNA2 | BC005978 | | (Le Roux & Moroianu, 2003, |
| | | | | Supra) |
| 3.5 | TUBG1 | NM_001070 | | |
| 3.4 | v-Myc | NM_002467 | (Zajac-Kaye, 2001, Supra) | |
| 3.4 | RPL19 | NM_000981 | | (Henry et al., 1993, Cancer |
| | | | | Res., 53, 1403-1408) |
| 3.4 | CTP synthase | NM_001905 | | (Hansel et al., 2003, Am. J. |
| | | | | Pathol., 163, 217-229) |
| 3.4 | PSEN1 | AF416717 | | (Farquhar et al., 2003, |
| | | | | Neurosci, Lett., 346, 53-36) |
| 3.4 | NSEP1 | NM_004559 | | |
| 3.4 | NOL5A | NM_006392 | | |
| 3.4 | L10 | SEQ ID NO10 | | |
| 3.3 | S100 A11 protein | NM_005620 | | (Deloulme et al., 2000, J. Biol. |
| | | | | Chem., 275, 35302-35310) |
| 3.3 | RPL28 | NM_000991 | | |
| 3.3 | MORF4L2 | NM_012286 | | |
| 3.3 | DNAJC8 | NM_014280 | | |
| 3.3 | L11 | SEQ ID NO11 | | |
| 3.2 | SET translocation | NM_003011 | | |
| 3.2 | calnexin | NM_001746 | (Garcia-Lora et al., 2003, Int.J. | |
| | | | Cancer, 106, 521-527) | |
| 3.2 | TOP2A | NM_001067 | (Wikman et al., 2002, Supra) | |
| 3.2 | AASDHPPT | NM_015423 | | |
| 3.1 | phosphoglycerate kinase 1 | NM_000291 | (Yazawa et al., 2003, Pathol. | |
| | | | Int. 53, 58-65) | |
| 3.1 | RRM-2 | NM_001034 | | (Xue et al., 2003, Supra) |
| 3.1 | ubiquitin carrier protein | NM_91670 | | |
| 3.1 | NIFIE 14 | NM_032635 | | |
| 3.1 | actin, gamma | NM_001614 | (Redondo et al., 1998, Supra) | |
| 3 | RPL37 | D23661 | | (Loging & Reisman, 1999, |
| | | | | Supra) |
| 3 | GSTP1 | NM_000852 | | (Li et al., 2003, Supra) |
| 3 | karyopherin beta 1 | NM_002265 | | (Le Roux & Moroianu, 2003, |
| | | | | Supra) |

### DISCUSSION

Gene expression profiling provides a systematic approach for studying the mechanisms associated with progression from normal to metastatic disease. As described herein, SSH and cDNA microarray techiniques were utilized to identify novel lung cancer-associated antigens (L1-L19). Combining SSH and cDNA microarray methodology provides a rapid and effective approach for high-throughput screening and identification of novel tumor associated antigens (TAAs). The principle of SSH allows for the preferential amplification of differentially expressed sequences while suppressing those present at equal abundance within the original mRNA populations chosen for comparison (Diatchenko et al., 1996, Proc. Natl. Acad. Sci. USA, 93,6025-6030). The high level of enrichment, low level of background, and efficient normalization of sequences makes this an attractive approach for the rapid identification of novel targets.

The L molecules of the present invention and variants thereof may be used to advantage as diagnostic, prognostic, and/or therapeutic targets for lung cancer and other cancers in which L genes are aberrantly regulated or expressed. Notably, L genes display tumor-selective expression in lung cancer, and other cancers, while displaying minimal expression in normal tissues. The elevated levels of tumor-selective expression of the L genes indicate that L nucleic acid sequences, encoded polypeptides and antibodies thereto, and methods of use thereof may be utilized effectively to detect up-regulated expression of L genes, the detection of which serves as a diagnostic and/or prognostic indicator of cancer. Moreover, the L molecules of the invention and compounds identified using the methods of the invention which are capable of modulating L gene expression levels and/or activity also provide novel reagents with which to treat cancer patients. Such treatment modalities may be administered to a patient to ameliorate the symptoms of the disease, inhibit the disease by, for example, reducing tumor burden, and/or inhibit the progression of the disease by, for example, preventing metastasis.

The identification of L gene (L) nucleic and amino acid sequences, antibodies immunospecific for L molecules, methods wherein these L molecules and reagents are used, and L activity modulating compounds identified using such methods provide valuable tools with which to investigate the mechanism(s) involved in lung cancer development and progression. As demonstrated herein, gene expression profiling studies using SSH and arrays are useful for identifying novel cancer-selective genes, such as L1-L19, whose role(s) in lung cancer onset/progression have been heretofore previously unrecognized. Additional studies, based on the novel findings set forth herein, may elucidate the functional role of tumor associated antigens in biochemical pathways and mechanisms involved in carcinogenesis and disease progression. Such information may then be applied to the design of improved and novel therapeutic regimens for the treatment of lung cancer and other L gene-specific cancers.

SSH is a powerful technique used to identify novel differentially expressed transcripts (Diatchenko et al., 1996, Proc. Natl. Acad. Sci. USA 93, 60225-6030). SSH has been previously described in identifying genes associated with various cancers including: renal cancer (Stassar et al., 2001, Br. J. Cancer, 85,1372-1382), liver cancer (Miyasaka et al., 2001, Br. J. Cancer, 85, 228-234), prostate cancer (Porkka & Visakorpi, 2001, J. Pathol., 193, 73-79), colon cancer (Hufton et al., 1999, FEBS Lett., 463, 77-82), breast cancer (Yang et al., 1999, Nucl. Acids Res., 27, 1517-1523), and lung cancer (Bangur, et al. 2002, Oncogene, 21, 3814-3825). As described herein above, a combination of SSH and expression array profiling was used to identify NSCLC-specific genes. Quantitative PCR, cancer profiling arrays, and SAGE were used to confirm over-expression for some of the most interesting novel genes. These independent validation tools provided additional expression data for multiple tumor types.

The data presented herein identify a unique set of 46 genes with no previously described association with any neoplasm, including a set of 19 novel L genes. As described herein, each of these 46 candidate genes exhibits NSCLC-specificity. Five of these NSCLC-specific genes L4, L5, L7, L11, and L16 were chosen for further investigation based on the degree of tumor over-expression observed and overall lack of sequence homology with previously “known” genes. The experimental and in silico results described herein for the L4, L5, L7, L11, and L16 genes, confirmed tumor-selectivity in multiple carcinoma types including: LA (lung, colon, ovarian, rectal, and stomach); L5 (lung, brain, breast, kidney, ovarian, prostate, stomach, and uterine); L7 (lung, breast, kidney, and uterine); L11 (lung, breast, colon, ovarian, pancreatic, prostate, and uterine); and L16 (lung, breast, colon, ovarian, stomach, and uterine). These data, generated utilizing a combination of NSCLC-specific expression arrays, QPCR, CPA, and SAGE, confirmed and strengthened the identification of a bona fide subset of unique tumor-selective targets.

Each of the 46 genes identified with no previously described cancer-association is a potential diagnostic, prognostic, and/or therapeutic target for NSCLC, and/or other L positive cancer. In a preferred embodiment, each of the 19 novel L genes is a potential target molecule for diagnostic, prognostic, and/or therapeutic applications pertaining to L positive cancers, such as, but not limited to NSCLC. Subsets and combinations of the 46 genes whose up-regulation is correlated with various cancers, may also be used to advantage as combinatorial targets for diagnostic, prognostic, and/or therapeutic applications pertaining to L positive cancers.

A set of 53 genes known to be associated with cancers distinct from NSCLC was also identified herein. This set differs significantly from those recognized in previous NSCLC studies (Beer et al., 2002, Nat. Med., 8, 816-824; Bhattacharjee et al., 2001, Proc. Natl Acad. Sci. USA, 98, 13790-13795; Garber et al., 2001, Proc. Natl Acad. Sci. USA, 98, 13784-13789; Heighway et al., 2002, Oncogene, 21, 7749-7763; Nacht et al., 2001, Proc. Natl. Acad. Sci. USA, 98, 15203-15208). These genes have not been previously linked to NSCLC. Keratin hair basic 1 protein (Cribier et al., 2001, Br. J. Dermatol, 144, 977-982), for example, displayed the highest NSCLC-specific expression among these genes. CSE-1, a known apoptosis regulator (Behrens et al., 2003, Apoptosis, 8, 39-44), was also linked NSCLC. Pituitary tumor-transforming gene (PTTG1), whose elevated expression was discovered herein to be correlated with NSCLC, is known to be elevated in multiple tumors and induces in vitro transformation and in vivo tumor formation (Zhang et al., 1999, Mol. Endocrinol., 13, 156-166). Other genes in this novel set of 52 genes associated with NSCLC, include: epiregulin, which is over-expressed in pancreatic cancer and functions by stimulating cancer cell growth (Zhu et al., 2000, Biochem. Biophys. Res. Commun., 273,1019-1024); nucleolin, a transcriptional activator of HPV-18 in cervical cancer (Grinstein et al., 2002, J. Exp. Med., 196, 1067-1078); and LDB1, which is over-expressed in oral carcinoma (Mizunuma et al., 2003, Br. J. Cancer,88, 1543-1548). Ribosomal proteins RPL8 (Luo et al., 2002, Br. J. Cancer, 87,339-343), RPS13 (Denis et al., 1993, Int. J. Cancer, 55, 275-280), RPL5 (Bertram et al., 1998, Eur. J. Cancer, 34, 731-736), RPL23A (Jiang et al., 1997, Oncogene, 14, 473-480), RPL19 (Henry et al., 1993, Cancer Res., 53, 1403-1408), RPL37 (Loging & Reisman, 1999, Cancer Epidemiol. Biomarkers Prev., 8, 1011-1016), and RPS16 (Karan et al., 2002, Carcinogenesis, 23, 967-975) have been previously reported in association with multiple cancers. The data described herein, therefore, demonstrate the utility of the present approach in discovering previously unidentified NSCLC-specific genes.

The overall effectiveness of the present discovery approach and validity of the NSCLC-associated gene lists identified were further corroborated by the identification of genes previously described as NSCLC-specific. Of the 48 previously described NSCLC-associated genes in lists described herein, LDHA was the most abundant. PGP9.5 has been previously described as highly over-expressed in NSCLC (Hibi et al., 1999, Am. J. Pathol., 155, 711-715). PGP9.5 is a ubiquitin hydrolase expressed in neuronal tissues (Wilkinson et al., 1989, Science,246, 670-673) with a potential role in cancer development involving a contribution to the growth of somatic cells by increasing deubiquitination of cyclins (Spataro et al., 1998, Br. J. Cancer, 77, 448-455). CXC-5 is an angiogenic factor in NSCLC. Cysteine-rich protein 61 functions as a tumor suppressor gene in NSCLC (Tong et al., 2001, J. Biol. Chem., 276, 47709-47714). Cytokeratin 7 (Kummar et al., 2002, Br. J. Cancer, 86, 1884-1887), cytokeratin 8 (Fukunaga et al., 2002, Lung Cancer, 38, 31-38), and cytokeratin 18 (Young et al., 2002, Lung Cancer, 36, 133-141) have been described as potential diagnostic markers for NSCLC. Leucocyte antigens CD98 (Rintoul et al., 2002, Mol. Biol. Cell., 13, 2841-2852), CD29 (Oshita et al., 2002, Anticancer Res., 22, 1065-1070), CD59 (Niehans et al., 1996, Pathol., 149, 129-142), and ITGB5 (Smythe et al., 1995, Cancer Metastasis Rev., 14, 229-239) are proposed lung cancer-specific targets. Cyclin B1 is an early stage marker for NSCLC (Soria et al., 2000, Cancer Res., 60, 4000-4004). Splicing factor 3, CCT7, CCT5, and YWHAG were previously identified using NSCLC-specific arrays (Kikuchi et al., 2003, Oncogene, 22, 2192-2205).

As described in detail herein above, a combination of cDNA subtraction and array profiling was used successfully to identify a set of novel genes (L1-L19), the functional spectrum of which includes a correlation to cancer. The novel genes identified herein comprise a subset of a larger collection of 46 genes, the up-regulated expression of each of which was correlated with cancer. Notably, none of the 46 genes of this larger set were previously linked to a cancer. This set of 46 genes provides a genetic signature of useful diagnostic and therapeutic targets for NSCLC, or other “L positive” cancers. Further characterization of subsets of these target genes may provide valuable insight into the etiology of NSCLC, or other cancers.

### REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of diagnosing cancer in a subject comprising detecting or measuring an L gene product in a sample derived from said subject, wherein the L gene product is:
(a) an RNA corresponding to one of SEQ ID NOs:1-19, or a nucleic acid derived thereof;
(b) a protein comprising one of SEQ ID NOs:20-38;
(c) a nucleic acid comprising a sequence hybridizable to one of SEQ ID NOs:1-19 or a complement thereof under conditions of high stringency, or a protein comprising a sequence encoded by said hybridizable sequence; or
(d) a nucleic acid at least 90% homologous to one of SEQ ID NOs:1-19 or a complement thereof, or a protein encoded thereby;
wherein detecting elevated levels of the L gene product compared to L gene product levels in a non-cancerous sample or a pre-determined standard value for a noncancerous sample indicates the presence of cancer in the subject.

2. The method of claim 1, wherein the cancer is lung cancer or any L positive cancer.

3. The method of claim 1, wherein the L gene product is a protein comprising one of SEQ ID NOs: 20-38.

4. The method of claim 1, wherein the L gene product is an mRNA corresponding to one of SEQ ID NOs: 1-19.

5. The method of claim 1, wherein an antibody immunologically specific for an L gene product is used for detecting or measuring the L gene product.

6. The method of claim 5, wherein the antibody immunospecifically binds to one of SEQ ID NOs: 20-38.

7. The method of claim 1, wherein an oligonucleotide specific for an L gene product is used for detecting or measuring the L gene product.

8. The method of claim 7, wherein the oligonucleotide is a DNA oligonucleotide.

9. A method for treating a cancer in a subject, comprising administering to the subject a therapeutically effective amount of a compound capable of antagonizing an L gene product, wherein said L gene product is:
(a) an RNA corresponding to one of SEQ ID NOs:1-19, or a nucleic acid derived thereof;
(b) a protein comprising one of SEQ ID NOs: 20-38;
(c) a nucleic acid comprising a sequence hybridizable to one of SEQ ID NOs: 1-19 or a complement thereof under conditions of high stringency, or a protein comprising a sequence encoded by said hybridizable sequence; or
(d) a nucleic acid at least 90% homologous to one of SEQ ID NOs:1-19 or a complement thereof as determined using an NBLAST algorithm, or a protein encoded thereby.

10. The method of claim 9, wherein the compound decreases expression of the L gene product and wherein the L gene product is a protein comprising one of SEQ ID NOs: 20-38.

11. The method of claim 9, wherein the compound decreases expression of the L gene product and wherein the L gene product is an RNA corresponding to one of SEQ ID NOs: 1-19.

12. The method of claim 9, wherein the cancer is lung cancer or any L positive cancer.

13. The method of claim 9, wherein the compound capable of antagonizing an L gene product is a protein.

14. The method of claim 9, wherein the compound capable of antagonizing an L gene product is a peptide.

15. The method of claim 9, wherein the compound is an antibody immunologically specific for an L gene product.

16. A pharmaceutical composition comprising:
(a) an antibody immunologically specific for a protein comprising one of SEQ ID NOs: 20-38; or an L gene product, wherein said gene product is:
(i) an RNA corresponding to one of SEQ ID NOs: 1-19, or a nucleic acid derived thereof;
(ii) a protein comprising one of SEQ ID NOs: 20-38;
(iii) a nucleic acid comprising a sequence hybridizable to one of SEQ ID NOs: 1-19 or a complement thereof under conditions of high stringency, or a protein comprising a sequence encoded by said hybridizable sequence;
(iv) a nucleic acid at least 90% homologous to one of SEQ ID NOs: 1-19 or a complement thereof, or a protein encoded thereby; and
(b) a pharmaceutically acceptable carrier.

17. The pharmaceutical composition of claim 16, wherein the composition is formulated for delivery as an aerosol, for parenteral delivery, or for oral delivery.

18. The pharmaceutical composition of claim 16, wherein the L gene product is an mRNA corresponding to one of SEQ ID NOs: 1-19.

19. The pharmaceutical composition of claim 16, wherein the L gene product is a protein comprising one of SEQ ID NOs: 20-38.

20. The pharmaceutical composition of claim 16, wherein the L gene product is purified.


SEQUENCE LISTING (50 sequences)

SEQ ID NO: 1 - DNA, 975 bp, Homo sapiens
atggggaatcttcttaaagttttgacatgcacagaccttgagcaggggccaaattttttccttgattttgaaaatgcccagcctacagagtctgagaaggaaatttataatcaggtgaatgtagtattaaaagatgcagaaggcatcttggaggacttgcagtcatacagaggagctggccacgaaatacgagaggcaatccagcatccagcagatgagaagttgcaagagaaggcatggggtgcagttgttccactagtaggcaaattaaagaaattttacgaattttctcagaggttagaagcagcattaagaggtcttctgggagccttaacaagtaccccatattctcccacccagcatctagagcgagagcaggctcttgctaaacagtttgcagaaattcttcatttcacactccggtttgatgaactcaagatgacaaatcctgccatacagaatgatttcagctattatagaagaacattgagtcgtatgaggattaaaaatgtaccggcagaaggagaaaatgaagtaaataatgaattggcaaatcgaatgtctttgttttatgctgaggcaactccaatgctgaaaaccttgagtgatgccacaacaaaatttgtatcagagaataaaaatttaccaatagaaaataccacagattgtttaagcacaatggctagtgtatgcagagtcatgctggaaacaccggaatacagaagcagatttacaaatgaagagacagtgtcattctgcttgagggtaatggtgggtgtcataatactctatgaccacgtacatccagtgggagcatttgctaaaacttccaaaattgatatgaaaggttgtatcaaagttcttaaggaccaacctcctaatagtgtggaaggtcttctaaatgctctcaggtacacaacaaaacatttgaatgatgagactacctccaagcaaattaaatccatgctgcaataa

SEQ ID NO: 2 - DNA, 1935 bp, Homo sapiens
atggcaaagcccagccacagcagctatgtccttcagcagctaaacaaccaaagagaatggggttttctctgtgactgctgtattgcaattgatgacatttactttcaagcacacaaggcagttctagctgcctgtagctcctattttagaatgtttttcatgaaccatcagcatagtactgcacaactgaatctcagcaacatgaaaattagtgcagaatgttttgatctcattttgcagtttatgtatttaggaaaaattatgacagctccctccagttttgagcagtttaaagtggcaatgaactacctacagctatacaatgttcctgactgtttagaagacatccaggatgcagattgttctagttcaaaatgttcctcttctgcttccagcaaacagaacagcaaaatgatatttggggtaagaatgtatgaagatactgtggctcgaaatggcaatgaagccaacaggtggtgtgcagagccaagttcaacggtaaatacaccacataatagagaggctgatgaagagtctttacaattaggtaattttcctgagccactatttgatgtatgtaaaaaaagttccgtgtccaaattatctaatccaaaagaacgtgtgtcaagacgctttgggcggagttttacctgtgatagctgtggatttggctttagctgtgaaaaattattagatgagcatgtgctaacctgtactaacagacatttataccaaaacacaagatcttaccatagaatagtagatattagagatggaaaagacagtaacatcaaagctgaatttggtgaaaaagattcttccaaaacattttctgcacagacggacaaatacagaggagacacaagccaggctgctgatgattcagcttcaaccactggaagcagaaaaagtagcacagtggagtctgaaatagcaagcgaagagaaaagcagagctgctgagaggaaaaggattattattaagatggagccagaagatattcctacagatgaactgaaagactttaacattattaaagttactgataaagactgtaatgaatccactgacaatgatgaattagaagatgaacctgaagagccattttatagatactatgttgaagaagatgtcagcataaaaaaaagtggtaggaaaactctaaaacctcgaatgtcagtaagtgctgatgaaagaggtggtttagagaatatgaggccccctaacaacagcagtccagtacaagaggatgctgaaaatgcatcttgtgagctgtgtggacttacaataaccgaggaggacctgtcatctcattacttagccaaacacattgaaaatatctgtgcatgtggtaaatgtggacaaatacttgtaaagggtaggcagcttcaggaacatgctcaacgatgtggcgagccccaagatctgaccatgaatgggttaggaaatactgaggagaaaatggacttggaagagaatcctgatgagcagtccgaaataagagatatgtttgttgaaatgctggatgattttagggacaatcattaccagataaacagtatccaaaaaaagcagttatttaaacattctgcctgcccttttcgatgtcctaattgtggccagcgttttgaaactgaaaatctagtggttgaacatatgtctagctgcttagatcaagatatgtttaagagtgccatcatggaagaaaatgaaagagatcacagacgaaagcatttttgtaatctgtgtggaaaaggattttatcagcggtgtcacttaagagaacactatactgttcatactaaggaaaaacagtttgtttgtcaaacatgtggaaagcagtttttaagagagcgtcagttgcgactgcacaatgatatgcacaaaggcatggccagtggtgaaatagggccttctaaacctgtggagaagtga

SEQ ID NO: 3 - DNA, 861 bp, Homo sapiens
atgatggccactccgaaccagaccgcctgtaatgcagagtcaccagtggccctggaggaggccaagacctctggtgccccggggagcccccaaacaccccctgagcgtcatgactctggtggttccctgcccctgacaccgcggatggagagccactcagaggatgaagatcttgctggggctgtcggtggcctgggctggaacagtaggagtccccggacccagagcccagggggctgctcagcggaggctgtgctggcccggaagaaacaccgtcggcggccatcgaagcgcaaaaggcactggcgaccctacctggagctgagctgggctgagaaacaacagcgggatgagaggcagagccagagggcctcccgggtccgcgaagagatgttcgccaaaggccagcccgtggccccctacaacaccacccagttcctgatgaatgacagggacccggaggagcccaacttggatgtgccccatgggatctcccacccaggttccagtggggagagtgaggccggggacagtgatgggcggggccgagcgcacggtgagttccagcggaaggacttctctgagacttacgaacgcttccacaccgagagcctgcagggccgcagcaagcaggagctggtgcgagactacctggagctggagaagcggctgtcgcaggcggaggaggagactaggaggctgcagcagctgcaggcgtgcaccggccagcagtcctgccgccaggtggaggagctggctgccgaggtccagaggctccggaccgaaaaccagcggcttcgtcaggagaaccagatgtggaaccgagagggctgccgctgtgatgaggagccgggtacctag

SEQ ID NO: 4 - DNA, 666 bp, Homo sapiens
atgtttggttttcacaagccaaagatgtaccgaagtatagagggctgctgtatttgcagagctaagtcctccagttctcgattcactgacagtaaacgctatgaaaaggacttccagagctgttttggattgcatgagactcgttcaggagacatctgcaatgcctgtgtcctgcttgtgaaaagatggaagaagttgccagcaggatcaaaaaaaaactggaatcatgtggtagatgcaagggctggacccagtctaaagactacattgaaaccaaagaaagtgaaaactctatctgggaacaggataaaaagcaaccagatcagtaaactgcagaaggaatttaaacgtcataattctgatgctcacagtaccacctcaagtgcctccccagctcaatctccttgttacagtaaccagtcagatgacggctcagatacagagatggcttctggttctaacagaacaccagttttttcctttttagatctcacttactggaaaagacagaagatatgttgtgggatcatctataaaggccgttttggggaagtcctcattgacacacatctcttcaagccttgctgcagcaataagaaagcagctgctgagaagccagaggagcaggggccagagcctctgcccatctccactcaggagtggtga

SEQ ID NO: 5 - DNA, 336 bp, Homo sapiens
atggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatag

SEQ ID NO: 6 - DNA, 408 bp, Homo sapiens
atggcggagaagtttgaccacctagaggagcacctggagaagttcgtggagaacattcggcagctcggcatcatcgtcagtgacttccagcccagcagccaggccgggctcaaccaaaagctgaattttattgttactggcttacaggatattgacaagtgcagacagcagcttcatgatattactgtaccgttagaagtttttgaatatatagatcaaggtcgaaatccccagctctacaccaaagagtgcctggagagggctctagctaaaaatgagcaagttaaaggcaagatcgacaccatgaagaaatttaaaagcctgttgattcaagaactttctaaagtatttccggaagacatggctaagtatcgaagcatccggggggaggatcacccgccttcttaa

SEQ ID NO: 7 - DNA, 1902 bp, Homo sapiens
atgccactgactcccactgtccagggcttccagtggactctccgaggccctgatgtagaaacttccccattcggtgcaccaagagcagcctcacatggtgtgggccgacatcaagagctgcgagatccaacagtccctggccccacctcttctgccacaaacgtcagcatggtggtatctgccggcccttggtccggtgagaaggcagagatgaacattctagaaatcaacaagaaatcgcgcccccagctggcagagaacaaacagcagttcagaaacctcaaacagaaatgtcttgtaactcaagtggcctacttcctggccaaccggcaaaataattacgactatgaagactgcaaagacctcataaaatctatgctgagggatgagcggctgctcacagaagagaagcttgcagaggagctcgggcaagctgaggagctcaggcaatataaagtcctggttcactctcaggaacgagagctgacccagttaagggagaagttacaggaagggagagatgcctcccgctcattgaatcagcatctccaggccctcctcactccggatgagccggacaactcccagggacgggacctccgagaacagctggctgagggatgtaggctggcacagcacctcgtccaaaagctcagcccagaaaatgatgacgatgaggatgaagatgttaaagttgaggaggctgagaaagtacaggaattatatgcccccagggaggtgcagaaggctgaagaaaaggaagtccctgaggactcactggaggagtgtgccatcacttgttcaaatagccaccacccttgtgagtccaaccagccttacgggaacaccagaatcacatttgaggaagaccaagtcgactcaactctcattgactcatcctctcatgatgaatggttggatgctgtatgcattatcccagaaaatgaaagtgatcatgagcaagaggaagaaaaagggccagtgtctcccaggaatctgcaggagtctgaagaggaggaagccccccaggagtcctgggatgaaggtgattggactctctcaattcctcctgacatgtctgcctcataccagtctgacaggagcacctttcactcagtagaggaacagcaagtcggcttggctcttgacataggcagacattggtgtgatcaagtgaaaaaggaggaccaagaggccacaagtcccaggctcagcagggagctgctggatgagaaagagcctgaagtcttgcaggactcactggatagattttattcaactccttttgagtacctggaactgcctgacttatgccagccctacagaagtgacttttactcattgcaggaacaacaccttggcttggctcttgacttggacagaatgaaaaaggaccaagaagaggaagaagaccaaggcccaccatgccccaggctcagcagagagctgccggaggtagtagagcctgaggacttgcaggactcactggatagatggtattcgactcctttcagttatccagaactgcctgattcatgccagccctacggaagttgcttttactcattggaggaagaacacgttggcttttctcttgacgtggatgaaattgaaaagtaccaagaaggggaagaagatcaaaagccaccatgccccaggctcaacgaggtgctgatggaagcagaagagcctgaagtcttgcaggactcactggatagatgttattcgactacttcaacttactttcaactacatgcctcattccagcagtacagaagtgccttttactcatttgaggaacaggacgtcagcttggcccttgacgtggacaataggttttttactttgacagtgataaggcaccacctggccttccagatgggagtcatattcccacactaa

SEQ ID NO: 8 - DNA, 828 bp, Homo sapiens
atgcaaaacaacgaaattataaagcctgccaaatacttctcagaattggaaaagagcatcctgctggctttagtagaaaagtataaatatgtgctggaatgtaagaaaagtgatgcgcgaactattgcccttaagcagcgtacctggcaggcgctggcccacgaatacaactctcagcccagcgtgtccctgcgggatttcaaacagctgaagaagtgctgggagaacatcaaggctcggaccaaaaaaattatggcccatgaaaggagagagaaagtgaaacggagcgtcagccctctcctgagtacccacgtcctagggaaggagaagatcgccagcatgctgccggagcagctctacttcctgcagagccccccggaggaggagcccgaataccaccccgacgcctcagcccaagaatcatttgctgtttcaaatagagaactgtgcgatgatgagaaagagttcatacattttccagtatgtgaggggacctctcaacctgaaccctcgtgttcagctgtcagaataacagccaataaaaactacaggagcaaaacctctcaggaaggtgctttaaaaaagatgcatgaggaagaacaccatcaacaaatgtccatcttacaactgcaactgatacaaatgaatgaggtgcatgtggccaaaatccagcagatagagcgagagtgtgagatggcagaggaggaacacaggataaaaatggaagttctcaataaaaagaagatgtattgggaaagaaaactacaaacttttaccaaggaatggcctgtttcctcatttaaccggccctttcccaattcgccctaa

SEQ ID NO: 9 - DNA, 1791 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagggagaacctaaagaagtagttaaacctgtgcccattacatctcctgctgtgtccaaagtcacttccacaaacaacatggcctacaataaggcaccacggccttttggttctgtgtcttcaccaaaagtcacatccatcccatcaccatcgtctgccttcaccccagcccatgcgaccacctcatcacatgcttccccttcacccgtggctgccgtcactcctcccctgttcgctgcatctggactgcatgctaatgccaatcttagtgctgaccagtctccatctgcactgagcgctggtaaaactgcagttaatgtcccacggcagcccacagtcaccagcgtgtgttccgagacttctcaggagctagcagagggacagagaagaggatcccagggtgacagtaaacagcaaaatggcccaccaagaaaacacattgtggagcgctatacagagttttatcatgtacccactcacagtgatgccagcaagaagagactgattgaggatactgaagactggcgtccaagaactggaacaactcagtctcgctctttccgaatccttgcccagatcactgggactgaacatttgaaagaatctgaagccgataatacaaagaaggcaaataactctcaggagccttctccgcagttggcttccttggtagcttccacacggagcatgcccgagagcctggacagcccaacctctggcagaccaggggttaccagcctcacaactgcagctgccttcaagcctgtaggatccactggcgtcatcaagtcaccaagctggcaacggccaaaccaaggagtaccttccactggaagaatctcaaacagcgctacttactcaggatcagtggcaccagccaactcagctttgggacaaacccagccaagtgaccaggacactttagtgcaaagagctgagcacattccagcagggaaacgaactccgatgtgcgcccattgtaaccaggtcatcagaggaccattcttagtggcactggggaaatcttggcacccagaagaattcaactgcgctcactgcaaaaatacaatggcctacattggatttgtagaggagaaaggagccctgtattgtgagctgtgctatgagaaattctttgcccctgaatgtggtcgatgccaaaggaagatccttggagaagtcatcaatgcgttgaaacaaacttggcatgtttcctgttttgtgtgtgtagcctgtggaaagcccattcggaacaatgtttttcacttggaggatggtgaaccctactgtgagactgattattatgccctctttggtactatatgccatggatgtgaatttcccatagaagctggtgacatgttcctggaagctctgggctacacctggcatgacacttgctttgtatgctcagtgtgttgtgaaagtttggaaggtcagacctttttctccaagaaggacaagcccctgtgtaagaaacatgctcattctgtgaatttttga

SEQ ID NO: 10 - DNA, 978 bp, Homo sapiens
atgtctgacctgcaggcggctgaggggccgggctcctggagccccacagcccgcccagggtcggctggcggcgtcggggactgccagggagtggaggggagccaggcggccgcctcagagaatgaagatctagaaaacaaggatacctctttattggcttctgccaccgatccagaaccctgctcctcaccccacaggccacagatggtatctccagtgagtaaggatgccacggaagatctgcggaaagcaactggtcctttggaggctcaggccttggtgaaacaggatttgctgcctgcagaccaggcccaggtcctcaatgagatggctaagtatcaagttccacagaggtctggggacatcgttatgatccagtctgaacatacaggagctatagatgttctttcagctgatttggaatctgcagatcttctgggggaccacaggaaagtctccccacctctgatggctcctccatgcatctggacctttgccaaggtgaaggaattcaaaagcaagctgggcaaagagaagaacagccgtctggtggtgaagcgtggtgaggtggtgaccatccgggtacctactcatccagaggggaagcgtgtctgctgggagtttgcgaccgatgactatgacattggctttggagtttattttgactggacccctgtaactagcactgacataactgtgcaggtcagtgattccagtgacgatgaggatgaagaagaggaagaggaggaagagattgaagaacccgttccagctggagatgtggagagaggctccaggagctccttgcggggtcgctatggggaggtcatgcctgtgtaccggcgggacagccaccgagacgtgcaggctggcagccatgactaccctggtgagggcatctacctgctcaagttcgacaactcctactccctgctgcgcaacaagactctctacttccacatctactacaccagctga

SEQ ID NO: 11 - DNA, 573 bp, Homo sapiens
atgttccaggtcccggatagcgagggcggccgcgccggctccagggccatgaagcccccaggaggagaatcgagcaatctttttggaagtccagaagaagccactccttccagcaggcctaataggatggcatctaatatttttggaccaacagaagaacctcagaacatacccaagaggacaaatcccccagggggtaaaggaagtggtatctttgacgaatcaacccccgtgcagactcgacagcacctgaacccacctggagggaagaccagcgacatttttgggtctccggtcactgccacttcacgcttggcacacccaaacaaacccaaggatcatgttttcttatgtgaaggagaagaaccaaaatcggatcttaaagctgcaaggagcatcccggctggagcagagccaggtgagaaaggcagcgccagaaaagcaggccccgccaaggagcaggagcccatgcccacagtcgacagccatgagccccggctggggccgcggcctcgctctcacaacaaggtcctgaacccaccgggaggcaaatccagcatctccttctactaa

SEQ ID NO: 12 - DNA, 1473 bp, Homo sapiens
atggaggattcggcctcggcctcgctgtcttctgcagccgctactggaacctccacctcgactccagcggccccgacagcacggaagcagctggataaagaacaggttagaaaggcagtggacgctctcttgacgcattgcaagtccaggaaaaacaattatgggttgcttttgaatgagaatgaaagtttatttttaatggtggtattatggaaaattccaagtaaagaactgagggtcagattgaccttgcctcatagtattcgatcagattcagaagatatctgtttatttacgaaggatgaacccaattcaactcctgaaaagacagaacagttttatagaaagcttttaaacaagcatggaattaaaaccgtttctcagattatctccctccaaactctaaagaaggaatataaatcctatgaagccaagctccgccttctgagcagttttgatttcttccttactgatgccagaattaggcggctcttaccctcactcattgggagacatttctatcaaagaaagaaagttccagtatctgtaaaccttctgtccaagaatttatcaagagagatcaatgactgtataggtggaacagtcttaaacatttctaaaagtggttcttgcagtgctatacgtattggtcacgttggaatgcaaattgagcacatcattgaaaacattgttgctgtcaccaaaggactttcagaaaaattgccagagaagtgggagagcgtgaaactcctgtttgtgaaaactgagaaatcggctgcacttcccatcttttcctcgtttgtcagcaattgggatgaagccaccaaaagatctttgcttaataagaagaaaaaagaggcaaggagaaaacgaagagaaagaaattttgaaaaacaaaaggagaggaagaagaagaggcagcaggctaggaagactgcatcagttcttagtaaagatgatgtggcacctgaaagtggtgatactacagtgaagaaacctgaatcaaagaaggaacagaccccagagcatgggaagaaaaaacgtggcagaggaaaagcccaagttaaagcaacaaatgaatccgaagacgaaatcccacagctggtaccaataggaaagaagactccagctaatgaaaaagtagagattcaaaaacatgccacaggaaagaagtctccagcaaagagtcctaatcccagcacacctcgtgggaagaaaagaaaggctttgccagcatctgagaccccaaaagctgcagagtctgagaccccagggaaaagcccagagaagaagccaaaaatcaaagaagaggcagtgaaggaaaaaagtccttcgctggggaaaaaagatgcgagacagactccaaaaaagccagaggccaagtttttcaccactcctagtaaatctgtgagaaaagcttcccacacccccaaaaaatggcccaaaaaacccaaagtaccccagtcgacctaa

SEQ ID NO: 13 - DNA, 1299 bp, Homo sapiens
atggcgggcctgggcctgggctccgccgttcccgtgtggctggccgaggacgacctcggctgcatcatctgccaggggctgctggactggcccgccacgctgccctgcggccacagcttctgccgccactgcctggaggccctgtggggcgcccgcgacgcccgccgctgggcctgccccacttgccgccagggcgccgcgcagcagccgcacctgcggaagaacacgctactgcaggacctggccgacaagtaccgccgcgccgcacgcgagatacaggcgggctccgaccctgcccactgcccctgcccgggctccagttccctctccagcgcggccgcgaggccccggcgccgcccggaactgcagcgggtggcagtagagaagagcatcacagaagttgctcaggagctgacagagctggtggaacatcttgtagacattgtcagaagcctgcagaatcagaggcccctatcagaatctggaccagacaacgaactgagcatcctgggcaaggctttttcttctggggtggatctttccatggcttctccaaagctggtgacttccgacacagctgcagggaaaatcagagatattctccatgacctagaagaaattcaggaaaaattacaagaaagcgtcacctggaaagaggctcctgaagcacaaatgcagggagaactcctggaagccccgtcttcctcctcatgcccattgcctgaccagagccaccctgcactcaggagagcttctcggtttgctcagtgggccatccatccaacctttaacttgaagagcctttcctgcagcctggaggggtccaaggattcccgtacagtgactgtgtctcaccgcccacaaccctatcgctggaactgtgaaaggttttctaccagccaggtcttatgttcccaggccctgtcttctggaaagcattactgggaagtggacactaggaattgcagccactgggcagttggggtggcttcctgggagatgagccgcgaccaggtcctgggaaggactatggactcttgttgtgtggaatggaaggggactagccagctctctgcatggcacatggtcaaggaaactgtccttggctcagacagacctggggtggtgggcatctggctgaaccttgaggagggaaagcttgccttctattcagtggacaatcaggagaagcttctgtatgagtgtaccatctctgcctcctctcctttgtaccctgccttctggctgtatggcttacatcctggaaattacctgataataaagcaagtaaaggtgtaa

SEQ ID NO: 14 - DNA, 2160 bp, Homo sapiens
atggcagcgctggaggaagaattcacgttgtcttcggtagtcctgagcgccgggcctgaaggactcctaggcgtggagcagagcgacaaaacagaccagtttctagtgacagacagcggcaggacagtcatcctctataaggtttctgatcagaaacccttggggagctggtcagtgaaacaaggtcaaattataacatgtccagctgtgtgcaactttcaaactggagagtatgttgttgtacacgataataaggttttaagaatatggaataatgaagatgtaaacctggataaagtatttaaagctacattgtcagcagaagtatataggatactttcagtgcaagggacagaacccttggtgctcttcaaggaaggtgctgttcgtggtttagaggccttgcttgcagacccccagcagaaaattgaaactgttatctctgatgaagaagtgattaaatggacaaagtttttcgtagtattcagacatcctgttttaatttttattactgaaaaacatggaaattactttgcttacgtgcaaatgtttaactcacgtatcttaaccaaatatacactcttacttggacaagacgaaaactctgttataaagagttttactgcatctgtagatcggaaattcatctctttgatgtcattaagctctgatggttgtatatatgaaaccttgataccaatacgtccagctgacccagaaaaaaatcagagcttagttaaatcactgctgctcaaggctgttgtatctggtaacgctcgaaatggagttgcactcactgccctggatcaggatcacgtcgcagtcctaggaagtccactagcagcttctaaggaatgcctctctgtatggaacataaaatttcaaacactacagacttcaaaagagttaccacaagggaccagtggtcaactctggtattatggagaacatttgtttatgctacatggaaaatctctaactgtgattccatacaagtgtgaagtgtcatcattagcaggtgctcttggaaaactcaagcatagtcaagatccaggaactcatgtcgtgtcccattttgtaaactgggagacacctcaaggatgtggacttgggttccagaactcagagcagtcaagaagaattttaaggagacgaaaaattgaagtgagtttacagccagaggttccaccatccaaacaacttttgtcaaccataatgaaagattcagaaaaacacattgaagtagaagtacggaaatttttggctctgaagcagacacctgactttcatactgtcattggggacacagtaacaggacttctggaaaggtgtaaagcagaaccatcattttatccccggaactgtctgatgcagcttatccaaacgcatgtgctttcttacagtttgtgccccgacttaatggagattgccttaaaaaagaaagatgtacagttgttacaactctgtctacagcagttccctgacattcctgaatcagtcacctgtgcttgcttaaaaattttcttgagcattggtgatgacagtcttcaagaaacagatgttaatatggagtcagtttttgactatagtataaattctgtacatgatgagaaaatggaagagcaaactgaaattcttcaaaatggcttcaatcctgaagaagataaatgcaataactgtgatcaagagttaaataaaaagccccaggacgaaacaaaggagagcacttcatgccctgtggtacaaaaaagagcagctctacttaatgcaattcttcattcagcatatagcgagacatttcttctgcctcatttgaaagacatcccagcacagcatatcacgctgtttcttaagtatttgtatttcctgtacctgaagtgtagcgaaaatgctactatgactcttcctggaatacacccacctaccttgaaccagattatggattggatatgtctacttctggatgcgaattttactgttgttgtaatgatgccagaagcaaagaggctactgataaatctttacaagcttgtaaaatctcagatatctgtttattctgagctcaacaagattgaagtaagttttcgggagctacagaaattaaatcaagaaaagaataatagaggattatattcaattgaagtgctggagctcttctga

SEQ ID NO: 15 - DNA, 690 bp, Homo sapiens
atgtgggcggcggggcgctgggggcctacttttccctcttcctacgccggtttctctgctgactgcagacccaggtctcggccctcctcggactcctgctcagtccctatgacgggcgcacgtgggcaggggctggaggtggtgcgctcgccgtcgccgccgctgccgctgagctgcagcaattccaccaggtcgctgttgtctccccttggccaccagagcttccagtttgacgaggacgacggtgacggggaggatgaggaagacgtggatgatgaggaagacgtggatgaagatgcccatgattcagaggccaaagtggcgagcctgagaggaatggagttacaggggtgcgccagcactcaggttgaatcagaaaataaccaagaagaacagaaacaggtgcgcttaccagaaagccgcctgacaccatgggaggtgtggtttattggcaaagaaaaagaagaacgtgaccggctgcaactgaaagctctagaggaattaaatcaacaactagaaaaaagaaaagaaatggaagaacgtgaaaaaagaaagataattgctgaagaaaagcacaaggaatgggttcagaaaaagaatgagcaagtaaggaggggaaaatggatacacacattgacatctcttttgcaaaatatttcttcctattatacttcattacctaggttttaa

SEQ ID NO: 16 - DNA, 4173 bp, Homo sapiens
atggtggttctccgcagcagcttggagctgcacaaccactccgcggcctcggccacgggctccttggacctgtccagtgacttcctcagtctggagcacatcggccggaggcggctccgctcggccggcgcggcgcagaagaaacccgcggcgaccacagccaaagcgggcgatgggtcatcagttaaggaagttgaaacctaccaccggacacgtgctttaagatctttgagaaaagatgcacagaattcttcagattctagttttgagaagaatgtggaaataacggagcaacttgctaatggcaggcattttacaaggcagttggccagacagcaggctgataaaaaaaaagaagagcacagagaagacaaagtgattccagttactcggtcattgagggctagaaacatcgttcaaagtacagaacacttacatgaagataatggtgatgttgaagtgcgtcgaagttgtaggattagaagtcgttatagtggtgtaaaccagtccatgctgtttgacaaacttataactaacactgctgaagctgtacttcaaaaaatggatgacatgaagaagatgcgtagacagcgaatgagagaacttgaagacttgggagtgtttaatgaaacagaagaaagcaatcttaatatgtacacaagaggaaaacagaaagatattcaaagaactgatgaagaaacaactgataatcaagaaggcagtgtggagtcatctgaagagggtgaagaccaagaacatgaagatgatggtgaagatgaagatgatgaagatgatgatgatgatgacgatgatgatgatgatgatgatgatgaagatgatgaagatgaagaagatggagaagaagagaatcagaagcgatattatcttagacagagaaaagctactgtttactatcaggctccattggaaaaacctcgtcaccagagaaagcccaacatattttatagtggcccagcttctcctgcaagaccaagataccgattatcttccgcaggaccaagaagtccttactgtaaacgaatgaacaggcgaaggcatgcaatccacagtagtgactcgacttcatcttcctcctctgaagatgaacagcactttgagaggcggaggaaaaggagtcgtaatagggctatcaataggtgcctcccactaaattttcggaaagatgaattaaaaggcatttataaagatcgaatgaaaattggagcaagccttgccgatgttgatccaatgcaactagattcttcagtacgatttgatagtgttggtggcctgtctaatcatatagcagctctaaaagagatggtggtgtttccattactttatccagaagtctttgaaaaatttaaaattcaacccccaagaggttgtttgttttatgggccacctggaactggaaagactctggttgccagagcacttgccaatgagtgcagtcaaggggataaaagagtagcatttttcatgaggaaaggtgctgattgtctaagtaaatgggtaggagaatctgaaagacagctacgattgctgtttgatcaggcctatcagatgcgcccatcaattattttttttgacgaaattgatggtctggctccagtacggtcaagcaggcaagatcagattcacagttctattgtttccaccctgctagctcttatggatggattggacagcagaggggaaattgtggtcattggtgctacgaacaggctagattctatagatcctgctttacgaaggcctggtcgctttgatagagaattcctctttagcctgcctgataaagaggctcgaaaagagattctaaagattcacaccagggattggaatcccaaaccactggacacatttttagaagagctagcagaaaactgtgttggatactgtggagcagatattaaatcaatatgtgctgaagctgctttatgtgctttacgacgacgctacccacagatctataccactagtgagaaactgcagttggatctctcttcaattaatatctcagctaaggatttcgaggtagctatgcaaaagatgataccagcctcccaaagagctgtgacatcacctgggcaggcactgtccaccgttgtgaaaccactcctgcaaaacactgttgacaagattttagaagccctgcagagagtatttccacatgcagaattcagaacaaataaaacattagactcagatatttcttgtcctctgctagaaagtgacttggcttacagtgatgatgatgttccatcagtttatgaaaatggactttctcagaaatcttctcataaggcaaaagacaattttaattttcttcatttgaatagaaatgcttgttaccaacctatgtcttttcgaccaagaatattgatagtaggagaaccaggatttgggcaaggttctcacttggcaccagctgtcattcatgctttggaaaagtttactgtatatacattagacattcctgttctttttggagttagtactacatcccctgaagaaacatgtgcccaggtgattcgtgaagctaagagaacagcaccaagtatagtgtatgttcctcatatccacgtgtggtgggaaatagttggaccgacacttaaagccacatttaccacattattacagaatattccttcatttgctccagttttactacttgcaacttctgacaaaccccattccgctttgccagaagaggtgcaagaattgtttatccgtgattatggagagatttttaatgtccagttaccggataaagaagaacggacaaaattttttgaagatttaattctaaaacaagctgctaagcctcctatatcaaaaaagaaagcagttttgcaggctttggaggtactcccagtagcaccaccacctgagccaagatcactgacagcagaagaagtgaaacgactagaagaacaagaagaagatacatttagagaactgaggattttcttaagaaatgttacacataggcttgctattgacaagcgattccgagtgtttactaagcctgttgaccctgatgaggttcctgattatgtcactgtaataaagcaaccaatggacctttcatctgtaatcagtaaaattgatctacacaagtatctgactgtgaaagactatttgagagatattgatctaatctgtagtaatgccttagaatacaatccagatagagatcctggagatcgtcttattaggcatagagcctgtgctttaagagatactgcctatgccataattaaagaagaacttgatgaagactttgagcagctctgtgaagaaattcaggaatctagaaagaaaagaggttgtagctcctccaaatatgccccgtcttactaccatgtgatgccaaagcaaaattccactcttgttggtgataaaagatcagacccagagcagaatgaaaagctaaagacaccgagtactcctgtggcttgcagcactcctgctcagttgaagaggaaaattcgcaaaaagtcaaactggtacttaggcaccataaaaaagcgaaggaagatttcacaggcaaaggatgatagccagaatgccatagatcacaaaattgagagtgatacagaggaaactcaagacacaagtgtagatcataatgagaccggaaacacaggagagtcttcggtggaagaaaatgaaaaacagcaaaatgcctctgaaagcaaactggaattgagaaataattcaaatacttgtaatatagagaatgagcttgaagactctaggaagactacagcatgtacagaattgagagacaagattgcttgtaatggagatgcttctagctctcagataatacatatttctgatgaaaatgaaggaaaagaaatgtgtgttctgcgaatgactcgagctagacgttcccaggtagaacagcagcagctcatcactgttgaaaaggctttggcaattctttctcagcctacaccctcacttgttgtggatcatgagcgattaaaaaatcttttgaagactgttgttaaaaaaagtcaaaactacaacatatttcagttggaaaatttgtatgcagtaatcagccaatgtatttatcggcatcgcaaggaccatgataaaacatcacttattcagaaaatggagcaagaggtagaaaacttcagttgttccagatga

SEQ ID NO: 17 - DNA, 723 bp, Homo sapiens
atgcctgaagatgtgaagaacttttacctgatgaccaatggcttccacatgacatggagtgtgaagctggatgagcacatcattccactgggaagcatggcaattaacagcatctcaaaactgactcagctcacccagtcttccatgtattcacttcctaatgcacccactctggcagacctggaggacgatacacatgaagccagtgatgatcagccagagaagcctcactttgactctcgcagtgtgatatttgagctggattcatgcaatggcagtgggaaagtttgccttgtctacaaaagtgggaaaccagcattagcagaagacactgagatctggttcctggacagagcgttatactggcattttctcacagacacctttactgcctattaccgcctgctcatcacccacctgggcctgccccagtggcaatatgccttcaccagctatggcattagcccacaggccaagcaatggttcagcatgtataaacctatcacctacaacacaaacctgctcacagaagagaccgactcctttgtgaataagctagatcccagcaaagtgtttaagagcaagaacaagatcgtaatcccaaaaaagaaagggcctgtgcagcctgcaggtggccagaaagggccctcaggaccctccggtccctccacttcctccacttctaaatcctcctctggctctggaaaccccacccggaagtga

SEQ ID NO: 18 - DNA, 2790 bp, Homo sapiens
atgactaaaaaaagaaaacgccaacatgattttcaaaaagtgaaattgaaagttggtaaaaagaagcccaagttacaaaatgctactcctacaaactttaaaacaaagactatacatctgcctgagcaactcaaagaggatggaacacttccaacaaacaatagaaaacttaacataaaggatttgctgtcacagatgcatcactacaatgctggggttaaacaaagtgctcttcttggacttaaagaccttttgtctcaatacccatttataattgatgcacacctttcaaacatattaagtgaagtgactgctgtgtttacagataaagatgctaatgtacgattagcagcagttcaacttcttcaattcctggcccccaaaatacgagctgaacaaatttctccattttttcctttggtaagtgcccatctctctagtgccatgactcacattactgaaggaattcaggaggactctttaaaagttttggacattctgctggaacagtacccagctctaattactggccgtagcagcatattgcttaagaattttgtagaacttatttctcatcagcagctgtccaaaggactgataaatagagacagatcccagtcctggatactttctgtaaatcctaatcggagactcacttctcagcaatggaggctgaaagtcttagtgagactcagtaaattccttcaggccttggcagatggatccagtaggttgagagaaagtgaaggacttcaggaacagaaagaaaatccccatgccactagcaactccatttttatcaactggaaggaacatgccaacgaccagcaacacatccaggtttatgaaaatgggggttcacagccaaatgtcagttcacagttcaggctacggtatctggttggaggactgagtggtgtggatgaaggcctgtcatctactgaaaacctgaaaggatttattgagataataattccattgctaattgaatgctgggttgaagctgtacctccacaactagctactcctgttgggaatggtatagaacgagaacctctacaggttatgcagcaagttcttaatattatttcccttctgtggaaactctctaaacaacaggatgaaacccataaattggagtcatggcttcgaaagaactaccttattgattttaaacaccattttatgagtcgttttccatatgtcttaaaagaaataaccaagcacaaaaggaaagagccaaataaaagcatcaagcattgcacagttctctccaataacatagatcgtctcttactgaatttaacactgtctgatatcatggtctccctggcaaatgcgtcaaccttgcagaaggattgcagttggatagaaatgataaggaaatttgtaacagagacccttgaagatggctctaggctaaatagtaagcaactgaacagattgctgggagtatcctggaggttaatgcaaatacagccaaacagagaggacacagagactcttattaaggcagtttatacattatatcagcagaggggccttatccttccagttcggactttgttattgaagtttttcagtaaaatctatcagacagaagaactgagatcttgtagattcagatatcgtagtaaagtgttatcccgttggctggctggcttaccattgcaacttgctcatcttggctcccgaaatcctgagctctctacacagcttatcgatatcattcataccgctgcagcacgagcaaataaagaattactaaaaagtttacaagctactgccctccgaatttatgatccacaagaaggtgctgtggtggttctccctgcagactctcagcagcgtttggttcagcttgtatatttcctacccagtctgccggctgatttgctttctcggttaagtcgttgctgtattatgggaagactcagttcaagtttggctgccatgcttatcgggatactgcacatgagatcatcattttctgggtggaagtattcagctaaagactggttgatgagtgatgtagactatttcagcttcttattttccacacttacagggttttcgaaagaggagttgacttggcttcagagccttcgaggagttcctcatgtcatccagacacagctttcccctgtgcttctctaccttacagatttggatcaatttttacaccactgggatgtaacagaggcagtttttcacagtttattggttattcctgcccgaagtcagaactttgacatcttgcaaagtgccatcagtaagcatttggttgggttgactgtaattcctgacagcacggctggctgtgtttttggtgttatctgtaagctcctggatcatacttgtgtagttagtgagactctactgccatttctggcttcttgttgctacagtcttctttattttctgctcactatagagaaaggggaagcagaacatctaagaaagagggacaagctgtggggggtctgtgtctccatcctggctctcttgcctcgagtcctcaggttgatgctgcagagcctgcgggtgaacagagttgggcctgaggagctgcctgttgtgggccagctgcttcgactgctgcttcagcatgcacccctcaggactcatatgttgaccaatgcgatcttggtgcagcagatcatcaagaatatcacgacattgaagagtggaagtgttcaggaacagtggctcacagacttacattactgctttaacgtgtatatcactgggcatccccaagggcccagtgcactggctacagtgtattga

SEQ ID NO: 19 - DNA, 1518 bp, Homo sapiens
atgctcagcaacatgccaggcacagctgcaggctccagtgggcgcggcatctccatcagccccagtgctggtcagatgcagatgcagcaccgtaccaacctgatggccaccctcagctatgggcaccgtcccttgtccaagcagctgagtgctgacagtgcagaggctcacagcttgaacgtgaatcggttctcccctgctaactacgaccaggcgcatttacacccccatctgttttcggaccagtcccggggttcccccagcagctacagcccttcaacaggagtggggttctctccaacccaagccctgaaagtccctccacttgaccaattccccaccttccctcccagtgcacatcagcagccgccacactataccacgtcggcactacagcaggccctgctgtctcccacgccgccagactatacaagacaccagcaggtaccccacatccttcaaggactgctttctccccggcattcgctcaccggccactcggacatccggctgcccccaacagagtttgcacagctcattaaaaggcagcagcaacaacggcagcagcagcagcaacagcagcaacagcaagaataccaggaactgttcaggcacatgaaccaaggggatgcggggagtctggctcccagccttgggggacagagcatgacagagcgccaggctttatcttatcaaaatgctgactcttatcaccatcacaccagcccccagcatctgctacaaatcagggcacaagaatgtgtctcacaggcttcctcacccaccccgccccacgggtatgctcaccagccggcactgatgcattcagagagcatggaggaggactgctcgtgtgagggggccaaggatggcttccaagacagtaagagttcaagtacattgaccaaaggttgccatgacagccctctgctcttgagtaccggtggacctggggaccctgaatctttgctaggaactgtgagtcatgcccaagaattggggatacatccctatggtcatcagccaactgctgcattcagtaaaaataaggtgcccagcagagagcctgtcatagggaactgcatggatagaagttctccaggacaagcagtggagctgccggatcacaatgggctcgggtacccagcacgcccctccgtccatgagcaccacaggccccgggccctccagagacaccacacgatccagaacagcgacgatgcttatgtacagctggataacttgccaggaatgagtctcgtggctgggaaagcacttagctctgcccggatgtcggatgcagttctcagtcagtcttcgctcatgggcagccagcagtttcaggatggggaaaatgaggaatgtggggcaagcctgggaggtcatgagcacccagacctgagtgatggcagccagcatttaaactcctcttgctatccatctacgtgtattacagacattctgctcagctacaagcaccccgaagtctccttcagcatggagcaggcaggcgtgtaa

SEQ ID NO: 20 - protein, 324 aa, Homo sapiens
MGNLLKVLTCTDLEQGPNFFLDFENAQPTESEKEIYNQVNVVLKDAEGILEDLQSYRGAGHEIREAIQHPADEKLQEKAWGAVVPLVGKLKKFYEFSQRLEAALRGLLGALTSTPYSPTQHLEREQALAKQFAEILHFTLRFDELKMTNPAIQNDFSYYRRTLSRMRIKNVPAEGENEVNNELANRMSLFYAEATPMLKTLSDATTKFVSENKNLPIENTTDCLSTMASVCRVMLETPEYRSRFTNEETVSFCLRVMVGVIILYDHVHPVGAFAKTSKIDMKGCIKVLKDQPPNSVEGLLNALRYTTKHLNDETTSKQIKSMLQ

SEQ ID NO: 21 - protein, 644 aa, Homo sapiens
MAKPSHSSYVLQQLNNQREWGFLCDCCIAIDDIYFQAHKAVLAACSSYFRMFFMNHQHSTAQLNLSNMKISAECFDLILQFMYLGKIMTAPSSFEQFKVAMNYLQLYNVPDCLEDIQDADCSSSKCSSSASSKQNSKMIFGVRMYEDTVARNGNEANRWCAEPSSTVNTPHNREADEESLQLGNFPEPLFDVCKKSSVSKLSNPKERVSRRFGRSFTCDSCGFGFSCEKLLDEHVLTCTNRHLYQNTRSYHRIVDIRDGKDSNIKAEFGEKDSSKTFSAQTDKYRGDTSQAADDSASTTGSRKSSTVESEIASEEKSRAAERKRIIIKMEPEDIPTDELKDFNIIKVTDKDCNESTDNDELEDEPEEPFYRYYVEEDVSIKKSGRKTLKPRMSVSADERGGLENMRPPNNSSPVQEDAENASCELCGLTITEEDLSSHYLAKHIENICACGKCGQILVKGRQLQEHAQRCGEPQDLTMNGLGNTEEKMDLEENPDEQSEIRDMFVEMLDDFRDNHYQINSIQKKQLFKHSACPFRCPNCGQRFETENLVVEHMSSCLDQDMFKSAIMEENERDHRRKHFCNLCGKGFYQRCHLREHYTVHTKEKQFVCQTCGKQFLRERQLRLHNDMHKGMASGEIGPSKPVEK

SEQ ID NO: 22 - protein, 286 aa, Homo sapiens
MMATPNQTACNAESPVALEEAKTSGAPGSPQTPPERHDSGGSLPLTPRMESHSEDEDLAGAVGGLGWNSRSPRTQSPGGCSAEAVLARKKHRRRPSKRKRHWRPYLELSWAEKQQRDERQSQRASRVREEMFAKGQPVAPYNTTQFLMNDRDPEEPNLDVPHGISHPGSSGESEAGDSDGRGRAHGEFQRKDFSETYERFHTESLQGRSKQELVRDYLELEKRLSQAEEETRRLQQLQACTGQQSCRQVEELAAEVQRLRTENQRLRQENQMWNREGCRCDEEPGT

SEQ ID NO: 23 - protein, 221 aa, Homo sapiens
MFGFHKPKMYRSIEGCCICRAKSSSSRFTDSKRYEKDFQSCFGLHETRSGDICNACVLLVKRWKKLPAGSKKNWNHVVDARAGPSLKTTLKPKKVKTLSGNRIKSNQISKLQKEFKRHNSDAHSTTSSASPAQSPCYSNQSDDGSDTEMASGSNRTPVFSFLDLTYWKRQKICCGIIYKGRFGEVLIDTHLFKPCCSNKKAAAEKPEEQGPEPLPISTQEW

SEQ ID NO: 24 - protein, 111 aa, Homo sapiens
MVRTKADSVPGTYRKVVAARAPRKVLGSSTSATNSTSVSSRKAENKYAGGNPVCVRPTPKWQKGIGEFFRLSPKDSEKENQIPEEAGSSGLGKAKRKACPLQPDHTNDEKE

SEQ ID NO: 25 - protein, 135 aa, Homo sapiens
MAEKFDHLEEHLEKFVENIRQLGIIVSDFQPSSQAGLNQKLNFIVTGLQDIDKCRQQLHDITVPLEVFEYIDQGRNPQLYTKECLERALAKNEQVKGKIDTMKKFKSLLIQELSKVFPEDMAKYRSIRGEDHPPS

SEQ ID NO: 26 - protein, 633 aa, Homo sapiens
MPLTPTVQGFQWTLRGPDVETSPFGAPRAASHGVGRHQELRDPTVPGPTSSATNVSMVVSAGPWSGEKAEMNILEINKKSRPQLAENKQQFRNLKQKCLVTQVAYFLANRQNNYDYEDCKDLIKSMLRDERLLTEEKLAEELGQAEELRQYKVLVHSQERELTQLREKLQEGRDASRSLNQHLQALLTPDEPDNSQGRDLREQLAEGCRLAQHLVQKLSPENDDDEDEDVKVEEAEKVQELYAPREVQKAEEKEVPEDSLEECAITCSNSHHPCESNQPYGNTRITFEEDQVDSTLIDSSSHDEWLDAVCIIPENESDHEQEEEKGPVSPRNLQESEEEEAPQESWDEGDWTLSIPPDMSASYQSDRSTFHSVEEQQVGLALDIGRHWCDQVKKEDQEATSPRLSRELLDEKEPEVLQDSLDRFYSTPFEYLELPDLCQPYRSDFYSLQEQHLGLALDLDRMKKDQEEEEDQGPPCPRLSRELPEVVEPEDLQDSLDRWYSTPFSYPELPDSCQPYGSCFYSLEEEHVGFSLDVDEIEKYQEGEEDQKPPCPRLNEVLMEAEEPEVLQDSLDRCYSTTSTYFQLHASFQQYRSAFYSFEEQDVSLALDVDNRFFTLTVIRHHLAFQMGVIFPH

SEQ ID NO: 27 - protein, 275 aa, Homo sapiens
MQNNEIIKPAKYFSELEKSILLALVEKYKYVLECKKSDARTIALKQRTWQALAHEYNSQPSVSLRDFKQLKKCWENIKARTKKIMAHERREKVKRSVSPLLSTHVLGKEKIASMLPEQLYFLQSPPEEEPEYHPDASAQESFAVSNRELCDDEKEFIHFPVCEGTSQPEPSCSAVRITANKNYRSKTSQEGALKKMHEEEHHQQMSILQLQLIQMNEVHVAKIQQIERECEMAEEEHRIKMEVLNKKKMYWERKLQTFTKEWPVSSFNRPFPNSP

SEQ ID NO: 28 - protein, 596 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKGEPKEVVKPVPITSPAVSKVTSTNNMAYNKAPRPFGSVSSPKVTSIPSPSSAFTPAHATTSSHASPSPVAAVTPPLFAASGLHANANLSADQSPSALSAGKTAVNVPRQPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASLVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 29 - protein, 325 aa, Homo sapiens
MSDLQAAEGPGSWSPTARPGSAGGVGDCQGVEGSQAAASENEDLENKDTSLLASATDPEPCSSPHRPQMVSPVSKDATEDLRKATGPLEAQALVKQDLLPADQAQVLNEMAKYQVPQRSGDIVMIQSEHTGAIDVLSADLESADLLGDHRKVSPPLMAPPCIWTFAKVKEFKSKLGKEKNSRLVVKRGEVVTIRVPTHPEGKRVCWEFATDDYDIGFGVYFDWTPVTSTDITVQVSDSSDDEDEEEEEEEEIEEPVPAGDVERGSRSSLRGRYGEVMPVYRRDSHRDVQAGSHDYPGEGIYLLKFDNSYSLLRNKTLYFHIYYTS

SEQ ID NO: 30 - protein, 190 aa, Homo sapiens
MFQVPDSEGGRAGSRAMKPPGGESSNLFGSPEEATPSSRPNRMASNIFGPTEEPQNIPKRTNPPGGKGSGIFDESTPVQTRQHLNPPGGKTSDIFGSPVTATSRLAHPNKPKDHVFLCEGEEPKSDLKAARSIPAGAEPGEKGSARKAGPAKEQEPMPTVDSHEPRLGPRPRSHNKVLNPPGGKSSISFY

SEQ ID NO: 31 - protein, 490 aa, Homo sapiens
MEDSASASLSSAAATGTSTSTPAAPTARKQLDKEQVRKAVDALLTHCKSRKNNYGLLLNENESLFLMVVLWKIPSKELRVRLTLPHSIRSDSEDICLFTKDEPNSTPEKTEQFYRKLLNKHGIKTVSQIISLQTLKKEYKSYEAKLRLLSSFDFFLTDARIRRLLPSLIGRHFYQRKKVPVSVNLLSKNLSREINDCIGGTVLNISKSGSCSAIRIGHVGMQIEHIIENIVAVTKGLSEKLPEKWESVKLLFVKTEKSAALPIFSSFVSNWDEATKRSLLNKKKKEARRKRRERNFEKQKERKKKRQQARKTASVLSKDDVAPESGDTTVKKPESKKEQTPEHGKKKRGRGKAQVKATNESEDEIPQLVPIGKKTPANEKVEIQKHATGKKSPAKSPNPSTPRGKKRKALPASETPKAAESETPGKSPEKKPKIKEEAVKEKSPSLGKKDARQTPKKPEAKFFTTPSKSVRKASHTPKKWPKKPKVPQST

SEQ ID NO: 32 - protein, 432 aa, Homo sapiens
MAGLGLGSAVPVWLAEDDLGCIICQGLLDWPATLPCGHSFCRHCLEALWGARDARRWACPTCRQGAAQQPHLRKNTLLQDLADKYRRAAREIQAGSDPAHCPCPGSSSLSSAAARPRRRPELQRVAVEKSITEVAQELTELVEHLVDIVRSLQNQRPLSESGPDNELSILGKAFSSGVDLSMASPKLVTSDTAAGKIRDILHDLEEIQEKLQESVTWKEAPEAQMQGELLEAPSSSSCPLPDQSHPALRRASRFAQWAIHPTFNLKSLSCSLEGSKDSRTVTVSHRPQPYRWNCERFSTSQVLCSQALSSGKHYWEVDTRNCSHWAVGVASWEMSRDQVLGRTMDSCCVEWKGTSQLSAWHMVKETVLGSDRPGVVGIWLNLEEGKLAFYSVDNQEKLLYECTISASSPLYPAFWLYGLHPGNYLIIKQVKV

SEQ ID NO: 33 - protein, 719 aa, Homo sapiens
MAALEEEFTLSSVVLSAGPEGLLGVEQSDKTDQFLVTDSGRTVILYKVSDQKPLGSWSVKQGQIITCPAVCNFQTGEYVVVHDNKVLRIWNNEDVNLDKVFKATLSAEVYRILSVQGTEPLVLFKEGAVRGLEALLADPQQKIETVISDEEVIKWTKFFVVFRHPVLIFITEKHGNYFAYVQMFNSRILTKYTLLLGQDENSVIKSFTASVDRKFISLMSLSSDGCIYETLIPIRPADPEKNQSLVKSLLLKAVVSGNARNGVALTALDQDHVAVLGSPLAASKECLSVWNIKFQTLQTSKELPQGTSGQLWYYGEHLFMLHGKSLTVIPYKCEVSSLAGALGKLKHSQDPGTHVVSHFVNWETPQGCGLGFQNSEQSRRILRRRKIEVSLQPEVPPSKQLLSTIMKDSEKHIEVEVRKFLALKQTPDFHTVIGDTVTGLLERCKAEPSFYPRNCLMQLIQTHVLSYSLCPDLMEIALKKKDVQLLQLCLQQFPDIPESVTCACLKIFLSIGDDSLQETDVNMESVFDYSINSVHDEKMEEQTEILQNGFNPEEDKCNNCDQELNKKPQDETKESTSCPVVQKRAALLNAILHSAYSETFLLPHLKDIPAQHITLFLKYLYFLYLKCSENATMTLPGIHPPTLNQIMDWICLLLDANFTVVVMMPEAKRLLINLYKLVKSQISVYSELNKIEVSFRELQKLNQEKNNRGLYSIEVLELF

SEQ ID NO: 34 - protein, 229 aa, Homo sapiens
MWAAGRWGPTFPSSYAGFSADCRPRSRPSSDSCSVPMTGARGQGLEVVRSPSPPLPLSCSNSTRSLLSPLGHQSFQFDEDDGDGEDEEDVDDEEDVDEDAHDSEAKVASLRGMELQGCASTQVESENNQEEQKQVRLPESRLTPWEVWFIGKEKEERDRLQLKALEELNQQLEKRKEMEEREKRKIIAEEKHKEWVQKKNEQVRRGKWIHTLTSLLQNISSYYTSLPRF

SEQ ID NO: 35 - protein, 1390 aa, Homo sapiens
MVVLRSSLELHNHSAASATGSLDLSSDFLSLEHIGRRRLRSAGAAQKKPAATTAKAGDGSSVKEVETYHRTRALRSLRKDAQNSSDSSFEKNVEITEQLANGRHFTRQLARQQADKKKEEHREDKVIPVTRSLRARNIVQSTEHLHEDNGDVEVRRSCRIRSRYSGVNQSMLFDKLITNTAEAVLQKMDDMKKMRRQRMRELEDLGVFNETEESNLNMYTRGKQKDIQRTDEETTDNQEGSVESSEEGEDQEHEDDGEDEDDEDDDDDDDDDDDDDDEDDEDEEDGEEENQKRYYLRQRKATVYYQAPLEKPRHQRKPNIFYSGPASPARPRYRLSSAGPRSPYCKRMNRRRHAIHSSDSTSSSSSEDEQHFERRRKRSRNRAINRCLPLNFRKDELKGIYKDRMKIGASLADVDPMQLDSSVRFDSVGGLSNHIAALKEMVVFPLLYPEVFEKFKIQPPRGCLFYGPPGTGKTLVARALANECSQGDKRVAFFMRKGADCLSKWVGESERQLRLLFDQAYQMRPSIIFFDEIDGLAPVRSSRQDQIHSSIVSTLLALMDGLDSRGEIVVIGATNRLDSIDPALRRPGRFDREFLFSLPDKEARKEILKIHTRDWNPKPLDTFLEELAENCVGYCGADIKSICAEAALCALRRRYPQIYTTSEKLQLDLSSINISAKDFEVAMQKMIPASQRAVTSPGQALSTVVKPLLQNTVDKILEALQRVFPHAEFRTNKTLDSDISCPLLESDLAYSDDDVPSVYENGLSQKSSHKAKDNFNFLHLNRNACYQPMSFRPRILIVGEPGFGQGSHLAPAVIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

SEQ ID NO: 36 - protein, 240 aa, Homo sapiens
MPEDVKNFYLMTNGFHMTWSVKLDEHIIPLGSMAINSISKLTQLTQSSMYSLPNAPTLADLEDDTHEASDDQPEKPHFDSRSVIFELDSCNGSGKVCLVYKSGKPALAEDTEIWFLDRALYWHFLTDTFTAYYRLLITHLGLPQWQYAFTSYGISPQAKQWFSMYKPITYNTNLLTEETDSFVNKLDPSKVFKSKNKIVIPKKKGPVQPAGGQKGPSGPSGPSTSSTSKSSSGSGNPTRK

SEQ ID NO: 37 - protein, 929 aa, Homo sapiens
MTKKRKRQHDFQKVKLKVGKKKPKLQNATPTNFKTKTIHLPEQLKEDGTLPTNNRKLNIKDLLSQMHHYNAGVKQSALLGLKDLLSQYPFIIDAHLSNILSEVTAVFTDKDANVRLAAVQLLQFLAPKIRAEQISPFFPLVSAHLSSAMTHITEGIQEDSLKVLDILLEQYPALITGRSSILLKNFVELISHQQLSKGLINRDRSQSWILSVNPNRRLTSQQWRLKVLVRLSKFLQALADGSSRLRESEGLQEQKENPHATSNSIFINWKEHANDQQHIQVYENGGSQPNVSSQFRLRYLVGGLSGVDEGLSSTENLKGFIEIIIPLLIECWVEAVPPQLATPVGNGIEREPLQVMQQVLNIISLLWKLSKQQDETHKLESWLRKNYLIDFKHHFMSRFPYVLKEITKHKRKEPNKSIKHCTVLSNNIDRLLLNLTLSDIMVSLANASTLQKDCSWIEMIRKFVTETLEDGSRLNSKQLNRLLGVSWRLMQIQPNREDTETLIKAVYTLYQQRGLILPVRTLLLKFFSKIYQTEELRSCRFRYRSKVLSRWLAGLPLQLAHLGSRNPELSTQLIDIIHTAAARANKELLKSLQATALRIYDPQEGAVVVLPADSQQRLVQLVYFLPSLPADLLSRLSRCCIMGRLSSSLAAMLIGILHMRSSFSGWKYSAKDWLMSDVDYFSFLFSTLTGFSKEELTWLQSLRGVPHVIQTQLSPVLLYLTDLDQFLHHWDVTEAVFHSLLVIPARSQNFDILQSAISKHLVGLTVIPDSTAGCVFGVICKLLDHTCVVSETLLPFLASCCYSLLYFLLTIEKGEAEHLRKRDKLWGVCVSILALLPRVLRLMLQSLRVNRVGPEELPVVGQLLRLLLQHAPLRTHMLTNAILVQQIIKNITTLKSGSVQEQWLTDLHYCFNVYITGHPQGPSALATVY

SEQ ID NO: 38 - protein, 505 aa, Homo sapiens
MLSNMPGTAAGSSGRGISISPSAGQMQMQHRTNLMATLSYGHRPLSKQLSADSAEAHSLNVNRFSPANYDQAHLHPHLFSDQSRGSPSSYSPSTGVGFSPTQALKVPPLDQFPTFPPSAHQQPPHYTTSALQQALLSPTPPDYTRHQQVPHILQGLLSPRHSLTGHSDIRLPPTEFAQLIKRQQQQRQQQQQQQQQQEYQELFRHMNQGDAGSLAPSLGGQSMTERQALSYQNADSYHHHTSPQHLLQIRAQECVSQASSPTPPHGYAHQPALMHSESMEEDCSCEGAKDGFQDSKSSSTLTKGCHDSPLLLSTGGPGDPESLLGTVSHAQELGIHPYGHQPTAAFSKNKVPSREPVIGNCMDRSSPGQAVELPDHNGLGYPARPSVHEHHRPRALQRHHTIQNSDDAYVQLDNLPGMSLVAGKALSSARMSDAVLSQSSLMGSQQFQDGENEECGASLGGHEHPDLSDGSQHLNSSCYPSTCITDILLSYKHPEVSFSMEQAGV

SEQ ID NO: 39 - DNA, 45 bp, Artificial Sequence
  Synthetic oligonucleotide
ttttgtacaagctttttttttttttttttttttttttttttttnn

SEQ ID NO: 40 - DNA, 44 bp, Artificial Sequence
  Synthetic oligonucleotide
ctaatacgactcactatagggctcgagcggccgcccgggcaggt

SEQ ID NO: 41 - DNA, 42 bp, Artificial Sequence
  Synthetic oligonucleotide
ctaatacgactcactatagggcagcgtggtcgcggccgaggt

SEQ ID NO: 42 - DNA, 22 bp, Artificial Sequence
  Synthetic oligonucleotide
ctaatacgactcactatagggc

SEQ ID NO: 43 - DNA, 22 bp, Artificial Sequence
  Synthetic oligonucleotide
tcgagcggccgcccgggcaggt

SEQ ID NO: 44 - DNA, 20 bp, Artificial Sequence
  Synthetic oligonucleotide
agcgtggtcgcggccgaggt

SEQ ID NO: 45 - DNA, 20 bp, Artificial Sequence
  Synthetic oligonucleotide
ctgttcctgttggccgagtc

SEQ ID NO: 46 - DNA, 21 bp, Artificial Sequence
  Synthetic oligonucleotide
cgatgcattgttatcattaac

SEQ ID NO: 47 - DNA, 20 bp, Artificial Sequence
  Synthetic oligonucleotide
caccctgagcagctcatcac

SEQ ID NO: 48 - DNA, 20 bp, Artificial Sequence
  Synthetic oligonucleotide
ggccagggtcacatttcacc

SEQ ID NO: 49 - DNA, 17 bp, Artificial Sequence
  Synthetic oligonucleotide
gtaaaacgacggccagt

SEQ ID NO: 50 - DNA, 18 bp, Artificial Sequence
  Synthetic oligonucleotide
caggaaacagctatgacc